(19) United States
(12) Patent Application Publication
(10) Pub. No.: US 2026/0123273 A1
(43) Pub. Date: Apr. 30, 2026

(54) TRIPHENYLENE-TRIAZINE-DIBENZOFURAN/DIBENZOTHIOPHENE DERIVATIVES FOR ORGANIC ELECTROLUMINESCENT DEVICES

(71) Applicant: MERCK PATENT GMBH, DARMSTADT (DE)

(72) Inventors: AMIR HOSSAIN PARHAM, FRANKFURT AM MAIN (DE); CHRISTIAN EHRENREICH, DARMSTADT (DE)

(21) Appl. No.: 18/715,084

(22) PCT Filed: Nov. 29, 2022
(86) PCT No.: PCT/EP2022/083570

§ 371 Date: Feb. 4, 2025

(30) Foreign Application Priority Data
Dec. 2, 2021 (EP) 21211974.7

(51) Int. Cl. H10K 85/60; C07B 59/00; C07D 405/14; C07D 409/14; C09K 11/02; H10K 50/12; H10K 71/16; H10K 101/00; H10K 101/20

(52) U.S. Cl. CPC H10K 85/622; C07B 59/002; C07D 405/14; C07D 409/14; C09K 11/02; H10K 71/166; H10K 85/633; H10K 85/654; H10K 85/657; H10K 85/6572; H10K 85/6574; H10K 85/6576; H10K 50/12; H10K 85/626; H10K 2101/20; H10K 2101/27

(57) ABSTRACT

A compound is set forth as formula (1) as defined herein. A mixture and composition may include the same compound. An organoelectroluminescent device may include an organic layer that includes the compound. A process for depositing the electroluminescent device includes depositing the organic layer by gas phase deposition or from solution.

### TECHNICAL FIELD

The present invention relates to triazine derivatives and electronic devices containing said compounds, especially organic electroluminescent devices containing said compounds as triplet matrix materials, optionally in combination with a further triplet matrix material and suitable phosphorescent emitters, and to suitable mixtures and formulations.

### STATE OF THE ART

Phosphorescent organometallic complexes are frequently used in organic electroluminescent devices (OLEDs). In general terms, there is still a need for improvement in OLEDs, for example with regard to efficiency, operating voltage and lifetime. The properties of phosphorescent OLEDs are not just determined by the triplet emitters used. More particularly, the other materials used, for example matrix materials, are also of particular significance here. Improvements to these materials can thus also lead to distinct improvements in the OLED properties.

According to the prior art, carbazole derivatives, dibenzofuran derivatives, indenocarbazole derivatives, indolocarbazole derivatives, benzofurocarbazole derivatives and benzothienocarbazole derivatives are among the matrix materials used for phosphorescent emitters.

Dibenzofuran-triazine derivatives and/or dibenzothiophene-triazine derivatives containing a triphenylene substituent are described, for example, in US20150349268, KR101959821, KR20190103765, KR20200011378, WO2018093015, WO2019054833, WO2019017731, WO21037401, WO21071247 and WO21180625.

US2017186969 describes an organic light-emitting device, wherein specific monoarylamines that are present in the organic layer may be unsubstituted or partly deuterated, and are especially present in an emitting auxiliary layer.

Specific monoarylamines that may be unsubstituted or partly deuterated are described in published specifications WO2015022051, WO2017148564, WO2018083053, CN112375053, WO2019192954, WO2021156323 and WO21107728.

There is generally still a need for improvement in these materials for use as matrix materials. The problem addressed by the present invention is that of providing improved compounds which are especially suitable for use as matrix material in a phosphorescent OLED. More particularly, it is an object of the present invention to provide matrix materials that lead to an improved lifetime. This is especially true of the use of a low to moderate emitter concentration, i.e. emitter concentrations in the order of magnitude of 3% to 20%, especially of 3% to 15%, since, in particular, device lifetime is limited here.

It has now been found that electroluminescent devices containing compounds of the formula (1) below have improvements over the prior art, especially when the compounds are used as matrix material for phosphorescent dopants.

It has also been found that this problem is solved, and the disadvantages from the prior art are eliminated, by the combination of at least one compound of the formula (1) as first host material and at least one hole-transporting compound of the formula (2) as second host material in a light-emitting layer of an organic electroluminescent device.

### SUMMARY OF THE INVENTION

The present invention firstly provides a compound of formula (1)

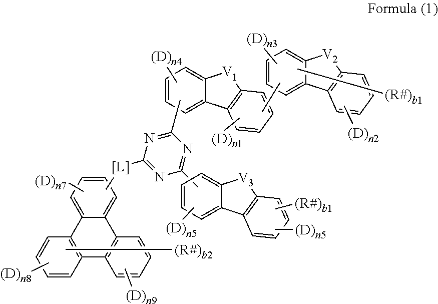

where the symbols and indices used are as follows:
V₁, V₂, V₃ are each independently O or S;
[L] is a single bond or an aromatic or heteroaromatic ring system which has 5 to 30 ring atoms and may be unsubstituted or partly or fully substituted by D;
R # is in each case independently phenyl, 1,2-biphenyl, 1,3-biphenyl or 1,4-biphenyl, which may be unsubstituted or partly or fully substituted by D;
b, b1 are each independently 0 or 1;
n1, n3, n4, n5, n7 are each independently 0, 1, 2 or 3 and
n2, n8, n9 are each independently 0, 1, 2, 3 or 4.

The invention further provides a mixture comprising at least one compound of formula (1) as described above or described as preferred later on, and at least one further compound selected from the group of the matrix materials, phosphorescent emitters, fluorescent emitters and/or emitters that exhibit TADF (thermally activated delayed fluorescence).

The invention further provides a formulation comprising at least one compound of formula (1) as described above or described as preferred later on, or a mixture as described above, and at least one solvent.

The invention further provides an organic electroluminescent device comprising an anode, a cathode and at least one organic layer comprising at least one compound of formula (1) as described above or described as preferred later on.

The invention further provides a process for producing an organic electroluminescent device as described above or as described as preferred hereinafter, characterized in that the organic layer is applied by gas phase deposition or from solution.

### DESCRIPTION OF THE INVENTION

In the present patent application, “D” or “D atom” means deuterium.

An aryl group in the context of this invention contains 6 to 40 ring atoms, preferably carbon atoms. A heteroaryl group in the context of this invention contains 5 to 40 ring atoms, where the ring atoms include carbon atoms and at least one heteroatom, with the proviso that the sum total of carbon atoms and heteroatoms adds up to at least 5. The heteroatoms are preferably selected from N, O and/or S. What is meant here by an aryl group or heteroaryl group is either a simple aromatic cycle, i.e. phenyl, derived from benzene, or a simple heteroaromatic cycle, for example derived from pyridine, pyrimidine or thiophene, or a fused aryl or heteroaryl group, for example derived from naphthalene, anthracene, phenanthrene, quinoline or isoquinoline. An aryl group having 6 to 18 carbon atoms is therefore preferably phenyl, naphthyl, phenanthryl or triphenylenyl, with no restriction in the attachment of the aryl group as substituent. The aryl or heteroaryl group in the context of this invention may bear one or more radicals, where the suitable radical is described below. If no such radical is described, the aryl group or heteroaryl group is unsubstituted.

An aromatic ring system in the context of this invention contains 6 to 40 carbon atoms in the ring system. The aromatic ring system also includes aryl groups as described above. An aromatic ring system having 6 to 18 carbon atoms is preferably selected from phenyl, fully deuterated phenyl, biphenyl, naphthyl, phenanthryl and triphenylenyl.

A heteroaromatic ring system in the context of this invention contains 5 to 40 ring atoms and at least one heteroatom. A preferred heteroaromatic ring system has 9 to 40 ring atoms and at least one heteroatom. The heteroaromatic ring system also includes heteroaryl groups as described above. The heteroatoms in the heteroaromatic ring system are preferably selected from N, O and/or S.

What is meant by an aromatic or heteroaromatic ring system in the context of this invention is a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon or oxygen atom or a carbonyl group. For example, systems such as 9,9′-spirobifluorene, 9,9-diarylfluorene, 9,9-dialkylfluorene, diaryl ethers, stilbene, etc. shall thus also be regarded as aromatic or heteroaromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, are likewise encompassed by the definition of the aromatic or heteroaromatic ring system.

What is meant by an aromatic or heteroaromatic ring system which has 5-40 ring atoms and may be joined to the aromatic or heteroaromatic system via any desired positions is, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

There follows a description of the compounds of the formula (1) and preferred embodiments thereof. The preferred embodiments are also applicable to the mixture of the invention, formulation of the invention and organic electroluminescent device of the invention.

In compounds of the formula (1), R # where it occurs is preferably phenyl, 1,3-biphenyl or 1,4-biphenyl, which may be unsubstituted or partly or fully substituted by D. In compounds of the formula (1), R # where it occurs is more preferably phenyl which may be unsubstituted or partly or fully substituted by D. Phenyl is preferably unsubstituted.

In compounds of the formula (1), b1 is 0 or 1, preferably 0.

In compounds of the formula (1), b2 is 0 or 1, preferably 0.

In compounds of the formula (1), it is preferable when n1, n3, n4, n5 and n7 are greater than 0. In compounds of the formula (1), it is preferable when at least one index n1, n3, n4, n5 or n7 is or at least two or three indices n1, n3, n4, n5 or n7 are greater than 0. In compounds of the formula (1), it is very particularly preferable when all indices n1, n3, n4, n5 and n7 are 0.

In compounds of the formula (1), it is preferable when n2, n8 and n9 are greater than 0. In compounds of the formula (1), it is preferable when at least one index n2, n8 or n9 is or at least two or three indices n2, n8 or n9 are greater than 0. In compounds of the formula (1), it is very particularly preferable when n2, n8 and n9 are 0.

In compounds of the formula (1), [L] is a single bond or an aromatic or heteroaromatic ring system which has 5 to 30 ring atoms and may be unsubstituted or partly or fully substituted by D.

In compounds of the formula (1) or preferred compounds of the formula (1), the linker [L] is preferably a single bond or a linker selected from the group of L-1 to L-20, where the linkers L-1 to L-20 may be unsubstituted or partly or fully substituted by D,

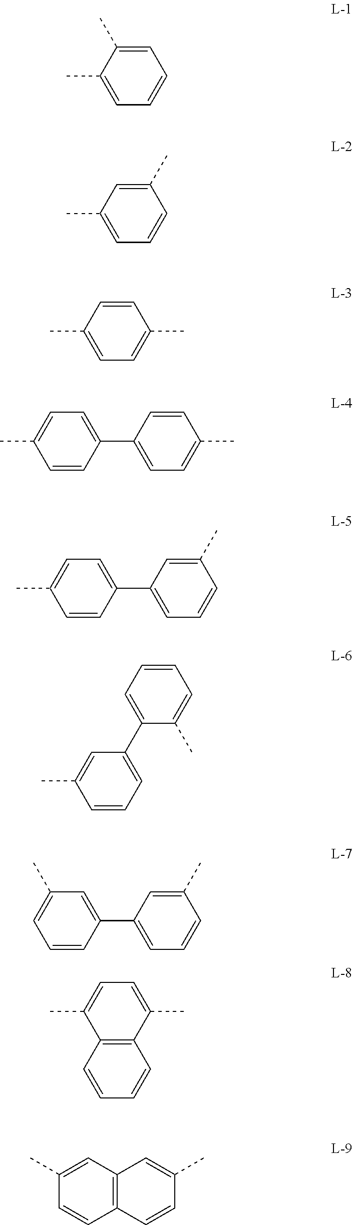

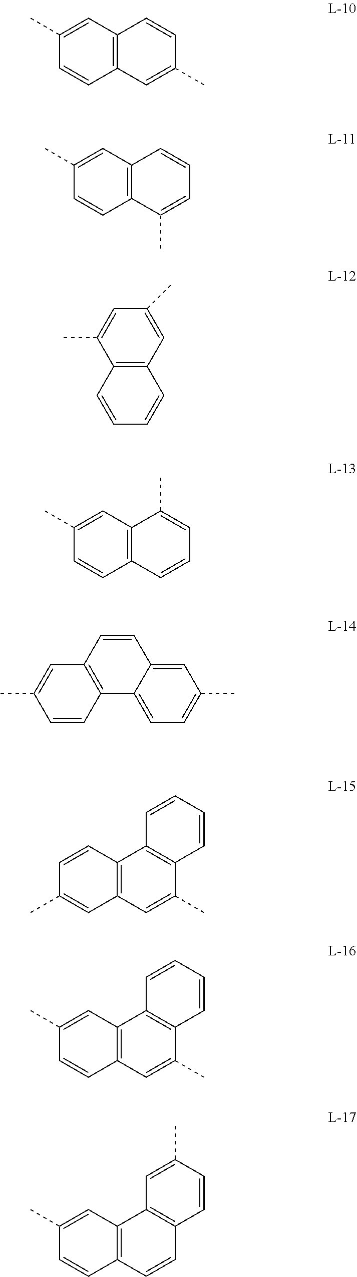

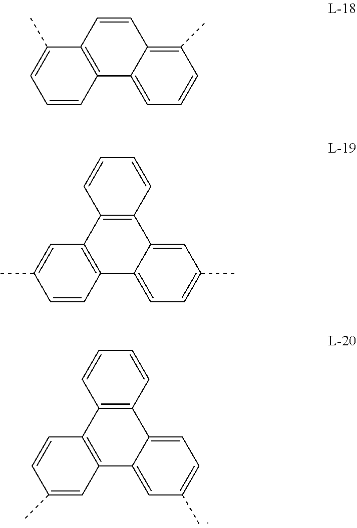

In compounds of the formula (1) or preferred compounds of the formula (1), the linker [L] is more preferably a single bond or an unsubstituted linker L-2, L-3, L-4 or L-7. From the group of linkers L-2, L-3, L4 and L-7, particular preference is given to linkers L-2 and L-3.

In compounds of the formula (1) or preferred compounds of the formula (1), the linker [L] is most preferably a single bond.

In compounds of the formula (1) or preferred compounds of the formula (1), V₃ is preferably O.

The invention therefore further provides compounds of the formula (1) where V₃ is O.

In compounds of the formula (1) or preferred compounds of the formula (1), V₂ is preferably O.

The invention therefore further provides compounds of the formula (1) where V₂ is O.

In one embodiment of the invention, it is preferable when, in compounds of the formula (1) or preferred compounds of the formula (1), V₁, V₂ and V₃ are O.

In compounds of the formula (1) or preferred compounds of the formula (1), it is preferable when at least one dibenzofuran or dibenzothiophene unit bonded to the triazine is bonded in the 1 position of the respective dibenzofuran or dibenzothiophene.

Examples of suitable host materials of the formula (1) are the structures shown below in table 1.

TABLE 1

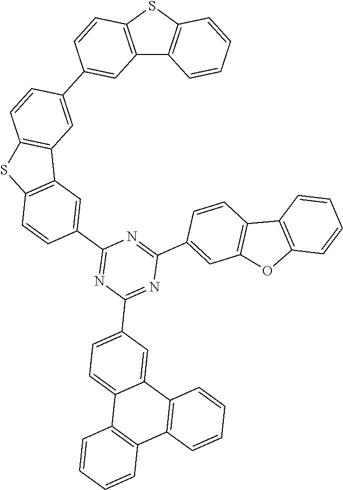 [SMILES: c1ccc2c(c1)oc1cc(-c3nc(-c4ccc5sc6ccc(-c7ccc8sc9ccccc9c8c7)cc6c5c4)nc(-c4ccc5c6ccccc6c6ccccc6c5c4)n3)ccc12]

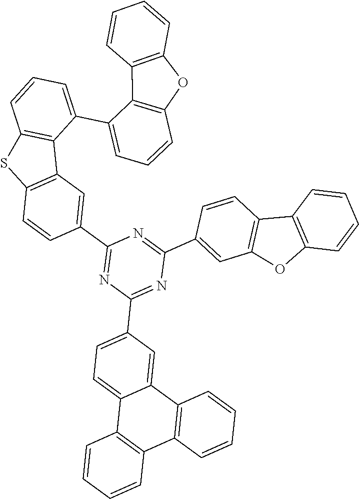 [SMILES: c1ccc2c(c1)oc1cc(-c3nc(-c4ccc5c6ccccc6c6ccccc6c5c4)nc(-c4ccc5sc6cccc(-c7cccc8oc9ccccc9c78)c6c5c4)n3)ccc12]

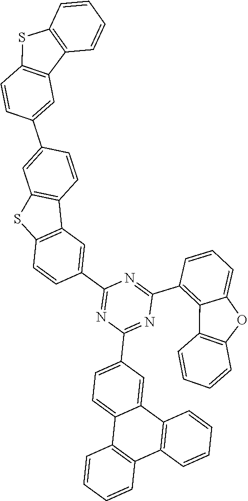 [SMILES: c1ccc2c(c1)oc1cccc(-c3nc(-c4ccc5sc6cc(-c7ccc8sc9ccccc9c8c7)ccc6c5c4)nc(-c4ccc5c6ccccc6c6ccccc6c5c4)n3)c12]

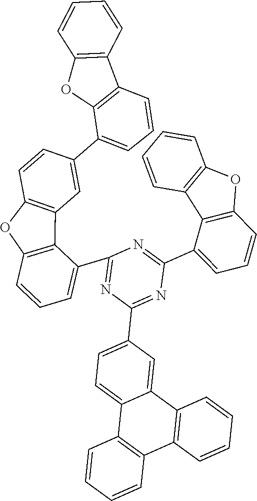 [SMILES: c1ccc2c(c1)oc1c(-c3ccc4oc5cccc(-c6nc(-c7ccc8c9ccccc9c9ccccc9c8c7)nc(-c7cccc8oc9ccccc9c78)n6)c5c4c3)cccc12]

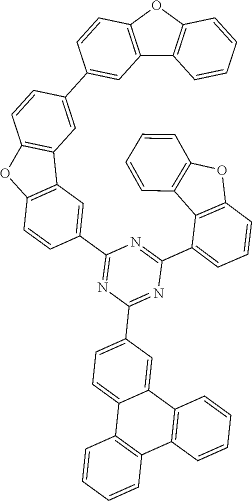 [SMILES: c1ccc2c(c1)oc1ccc(-c3ccc4oc5ccc(-c6nc(-c7ccc8c9ccccc9c9ccccc9c8c7)nc(-c7cccc8oc9ccccc9c78)n6)cc5c4c3)cc12]

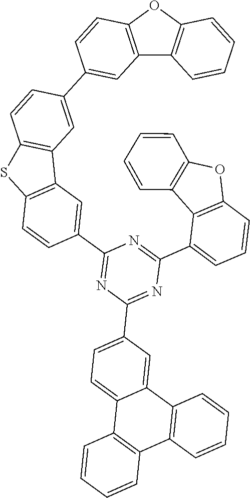 [SMILES: c1ccc2c(c1)oc1ccc(-c3ccc4sc5ccc(-c6nc(-c7ccc8c9ccccc9c9ccccc9c8c7)nc(-c7cccc8oc9ccccc9c78)n6)cc5c4c3)cc12]

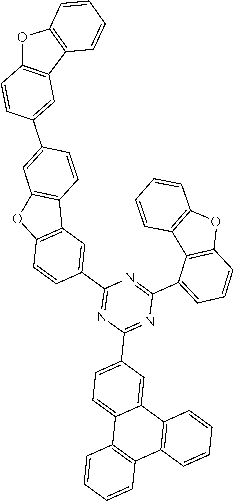 [SMILES: c1ccc2c(c1)oc1ccc(-c3ccc4c(c3)oc3ccc(-c5nc(-c6ccc7c8ccccc8c8ccccc8c7c6)nc(-c6cccc7oc8ccccc8c67)n5)cc34)cc12]

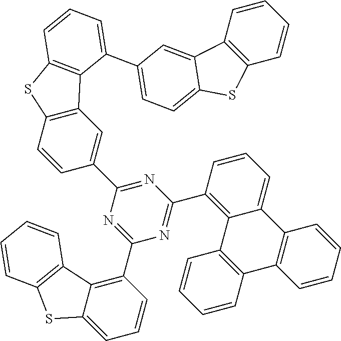 [SMILES: c1ccc2c(c1)sc1ccc(-c3cccc4sc5ccc(-c6nc(-c7cccc8sc9ccccc9c78)nc(-c7cccc8c9ccccc9c9ccccc9c78)n6)cc5c34)cc12]

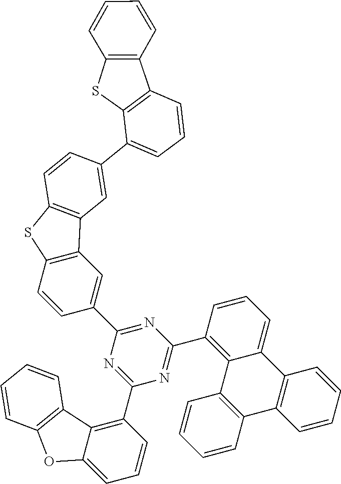 [SMILES: c1ccc2c(c1)oc1cccc(-c3nc(-c4ccc5sc6ccc(-c7cccc8c7sc7ccccc78)cc6c5c4)nc(-c4cccc5c6ccccc6c6ccccc6c45)n3)c12]

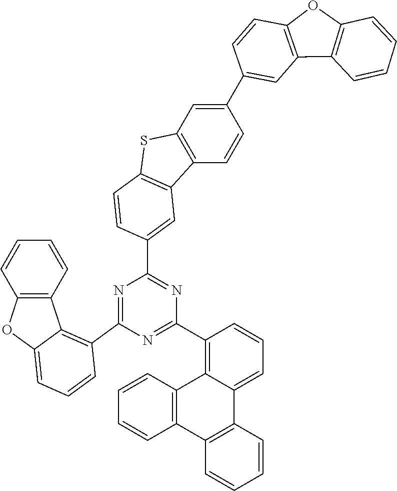 [SMILES: c1ccc2c(c1)oc1ccc(-c3ccc4c(c3)sc3ccc(-c5nc(-c6cccc7oc8ccccc8c67)nc(-c6cccc7c8ccccc8c8ccccc8c67)n5)cc34)cc12]

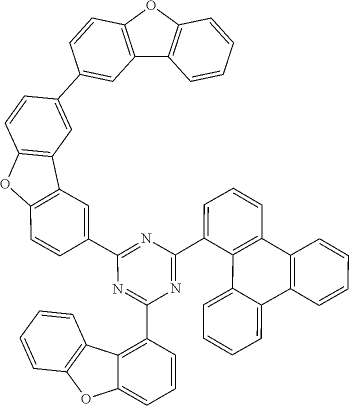 [SMILES: c1ccc2c(c1)oc1ccc(-c3ccc4oc5ccc(-c6nc(-c7cccc8oc9ccccc9c78)nc(-c7cccc8c9ccccc9c9ccccc9c78)n6)cc5c4c3)cc12]

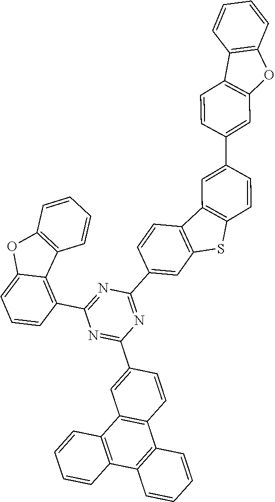 [SMILES: c1ccc2c(c1)oc1cc(-c3ccc4sc5cc(-c6nc(-c7ccc8c9ccccc9c9ccccc9c8c7)nc(-c7cccc8oc9ccccc9c78)n6)ccc5c4c3)ccc12]

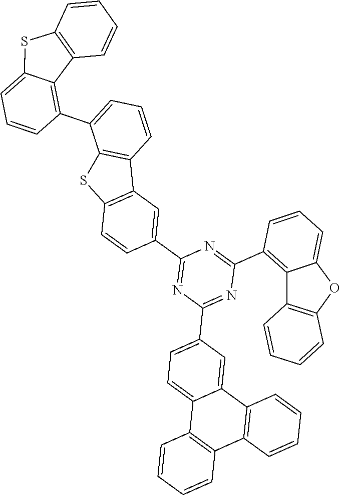 [SMILES: c1ccc2c(c1)oc1cccc(-c3nc(-c4ccc5sc6c(-c7cccc8sc9ccccc9c78)cccc6c5c4)nc(-c4ccc5c6ccccc6c6ccccc6c5c4)n3)c12]

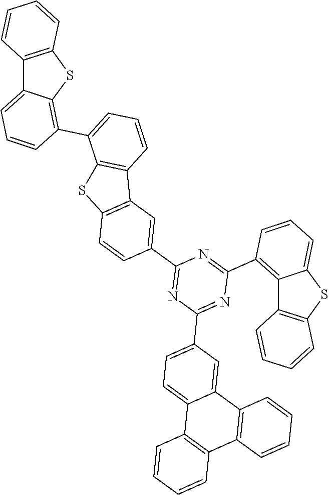 [SMILES: c1ccc2c(c1)sc1c(-c3cccc4c3sc3ccc(-c5nc(-c6ccc7c8ccccc8c8ccccc8c7c6)nc(-c6cccc7sc8ccccc8c67)n5)cc34)cccc12]

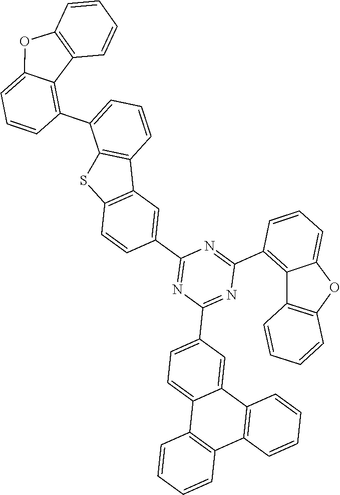 [SMILES: c1ccc2c(c1)oc1cccc(-c3nc(-c4ccc5sc6c(-c7cccc8oc9ccccc9c78)cccc6c5c4)nc(-c4ccc5c6ccccc6c6ccccc6c5c4)n3)c12]

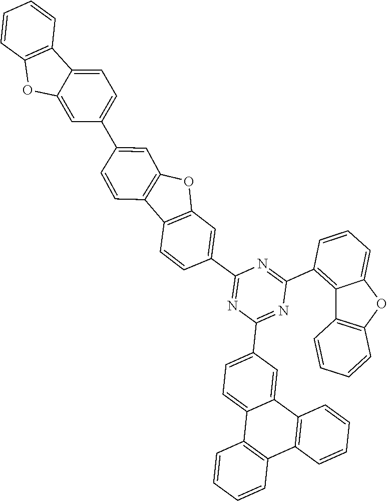 [SMILES: c1ccc2c(c1)oc1cc(-c3ccc4c(c3)oc3cc(-c5nc(-c6ccc7c8ccccc8c8ccccc8c7c6)nc(-c6cccc7oc8ccccc8c67)n5)ccc34)ccc12]

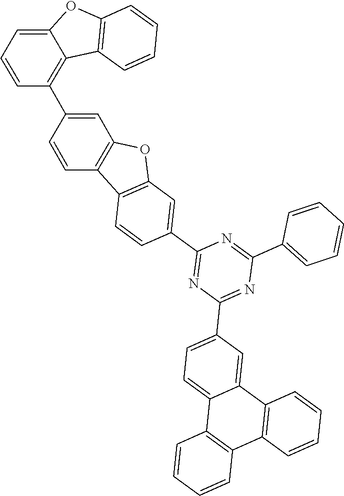 [SMILES: c1ccc(-c2nc(-c3ccc4c(c3)oc3cc(-c5cccc6oc7ccccc7c56)ccc34)nc(-c3ccc4c5ccccc5c5ccccc5c4c3)n2)cc1]

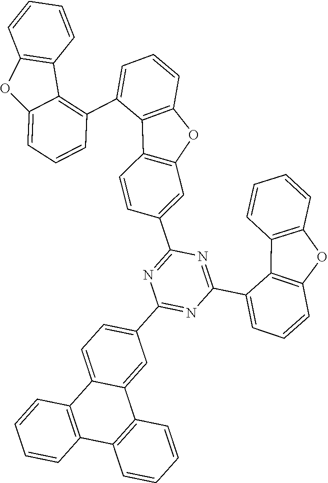 [SMILES: c1ccc2c(c1)oc1cccc(-c3nc(-c4ccc5c(c4)oc4cccc(-c6cccc7oc8ccccc8c67)c45)nc(-c4ccc5c6ccccc6c6ccccc6c5c4)n3)c12]

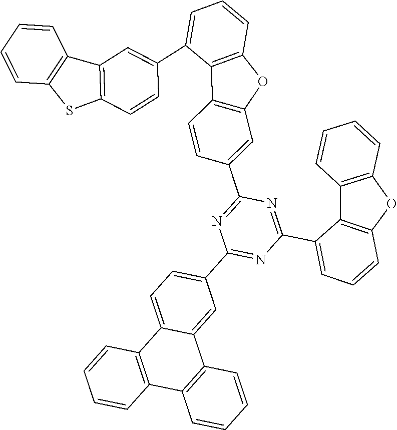 [SMILES: c1ccc2c(c1)oc1cccc(-c3nc(-c4ccc5c(c4)oc4cccc(-c6ccc7sc8ccccc8c7c6)c45)nc(-c4ccc5c6ccccc6c6ccccc6c5c4)n3)c12]

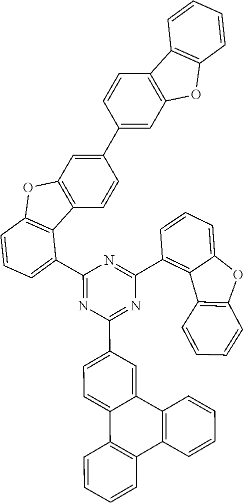 [SMILES: c1ccc2c(c1)oc1cc(-c3ccc4c(c3)oc3cccc(-c5nc(-c6ccc7c8ccccc8c8ccccc8c7c6)nc(-c6cccc7oc8ccccc8c67)n5)c34)ccc12]

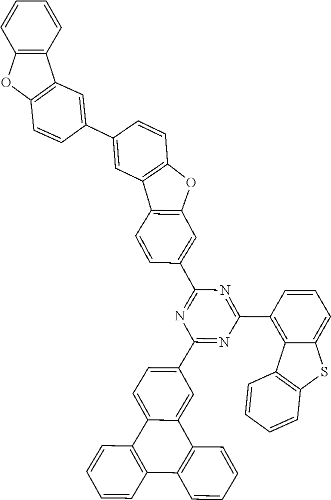 [SMILES: c1ccc2c(c1)oc1ccc(-c3ccc4oc5cc(-c6nc(-c7ccc8c9ccccc9c9ccccc9c8c7)nc(-c7cccc8sc9ccccc9c78)n6)ccc5c4c3)cc12]

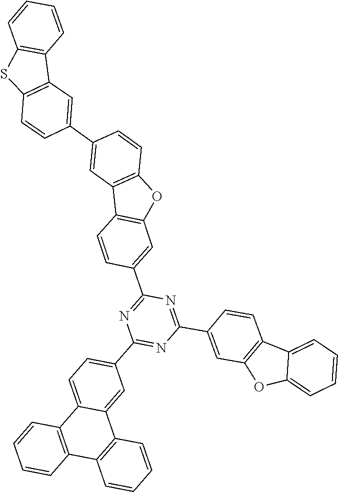 [SMILES: c1ccc2c(c1)oc1cc(-c3nc(-c4ccc5c(c4)oc4ccc(-c6ccc7sc8ccccc8c7c6)cc45)nc(-c4ccc5c6ccccc6c6ccccc6c5c4)n3)ccc12]

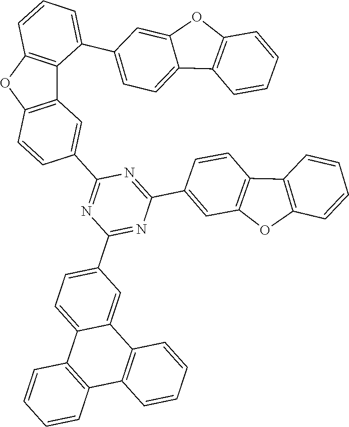 [SMILES: c1ccc2c(c1)oc1cc(-c3nc(-c4ccc5c6ccccc6c6ccccc6c5c4)nc(-c4ccc5oc6cccc(-c7ccc8c(c7)oc7ccccc78)c6c5c4)n3)ccc12]

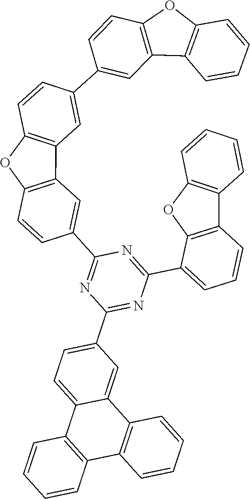 [SMILES: c1ccc2c(c1)oc1ccc(-c3ccc4oc5ccc(-c6nc(-c7ccc8c9ccccc9c9ccccc9c8c7)nc(-c7cccc8c7oc7ccccc78)n6)cc5c4c3)cc12]

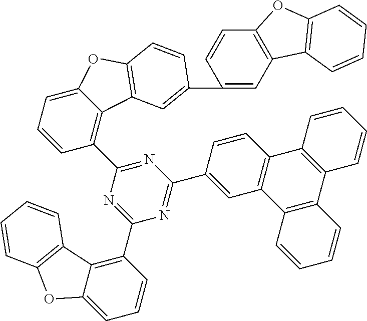 [SMILES: c1ccc2c(c1)oc1ccc(-c3ccc4oc5cccc(-c6nc(-c7ccc8c9ccccc9c9ccccc9c8c7)nc(-c7cccc8oc9ccccc9c78)n6)c5c4c3)cc12]

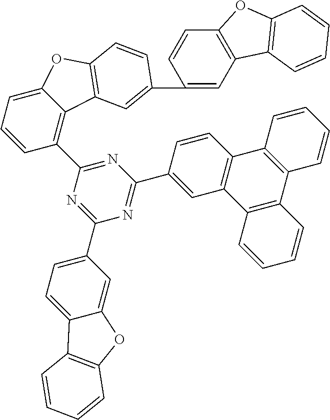 [SMILES: c1ccc2c(c1)oc1cc(-c3nc(-c4ccc5c6ccccc6c6ccccc6c5c4)nc(-c4cccc5oc6ccc(-c7ccc8oc9ccccc9c8c7)cc6c45)n3)ccc12]

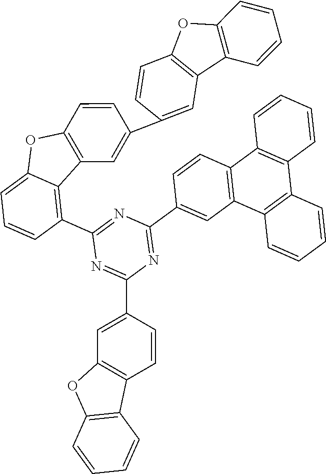 [SMILES: c1ccc2c(c1)oc1cc(-c3nc(-c4ccc5c6ccccc6c6ccccc6c5c4)nc(-c4cccc5oc6ccc(-c7ccc8oc9ccccc9c8c7)cc6c45)n3)ccc12]

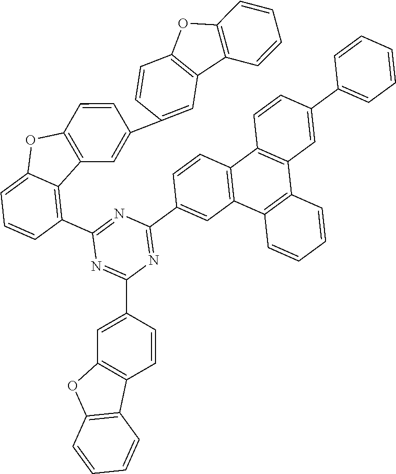 [SMILES: c1ccc(-c2ccc3c4ccc(-c5nc(-c6ccc7c(c6)oc6ccccc67)nc(-c6cccc7oc8ccc(-c9ccc%10oc%11ccccc%11c%10c9)cc8c67)n5)cc4c4ccccc4c3c2)cc1]

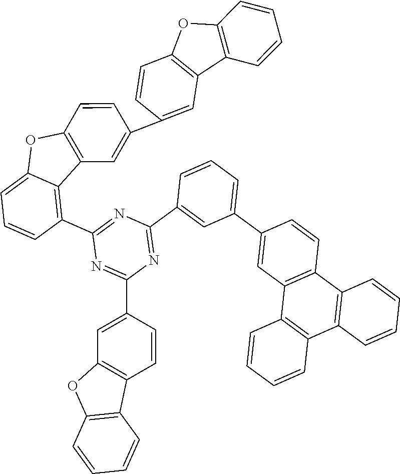 [SMILES: c1cc(-c2ccc3c4ccccc4c4ccccc4c3c2)cc(-c2nc(-c3ccc4c(c3)oc3ccccc34)nc(-c3cccc4oc5ccc(-c6ccc7oc8ccccc8c7c6)cc5c34)n2)c1]

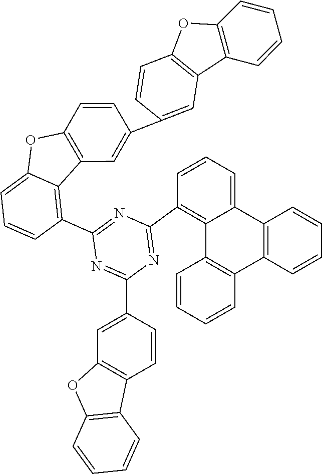 [SMILES: c1ccc2c(c1)oc1cc(-c3nc(-c4cccc5oc6ccc(-c7ccc8oc9ccccc9c8c7)cc6c45)nc(-c4cccc5c6ccccc6c6ccccc6c45)n3)ccc12]

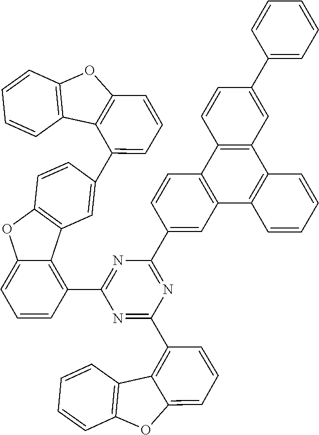 [SMILES: c1ccc(-c2ccc3c4ccc(-c5nc(-c6cccc7oc8ccccc8c67)nc(-c6cccc7oc8ccc(-c9cccc%10oc%11ccccc%11c9%10)cc8c67)n5)cc4c4ccccc4c3c2)cc1]

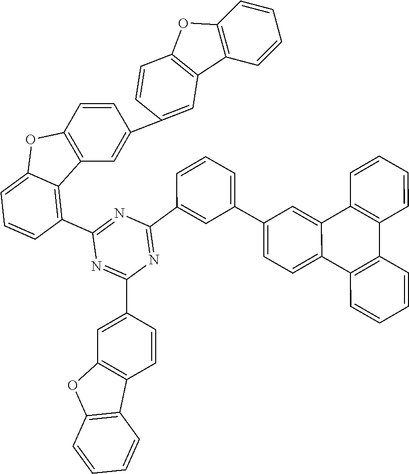 [SMILES: c1cc(-c2ccc3c4ccccc4c4ccccc4c3c2)cc(-c2nc(-c3ccc4c(c3)oc3ccccc34)nc(-c3cccc4oc5ccc(-c6ccc7oc8ccccc8c7c6)cc5c34)n2)c1]

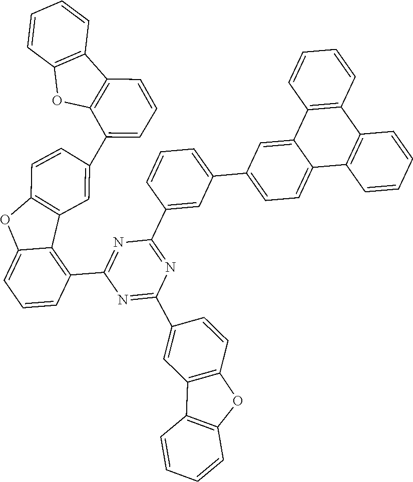 [SMILES: c1cc(-c2ccc3c4ccccc4c4ccccc4c3c2)cc(-c2nc(-c3ccc4oc5ccccc5c4c3)nc(-c3cccc4oc5ccc(-c6cccc7c6oc6ccccc67)cc5c34)n2)c1]

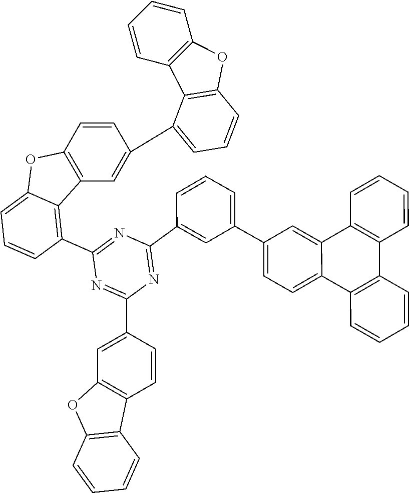 [SMILES: c1cc(-c2ccc3c4ccccc4c4ccccc4c3c2)cc(-c2nc(-c3ccc4c(c3)oc3ccccc34)nc(-c3cccc4oc5ccc(-c6cccc7oc8ccccc8c67)cc5c34)n2)c1]

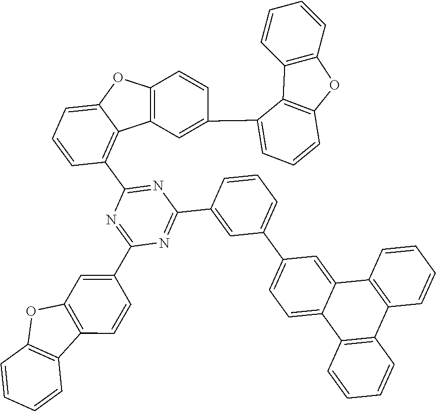 [SMILES: c1cc(-c2ccc3c4ccccc4c4ccccc4c3c2)cc(-c2nc(-c3ccc4c(c3)oc3ccccc34)nc(-c3cccc4oc5ccc(-c6cccc7oc8ccccc8c67)cc5c34)n2)c1]

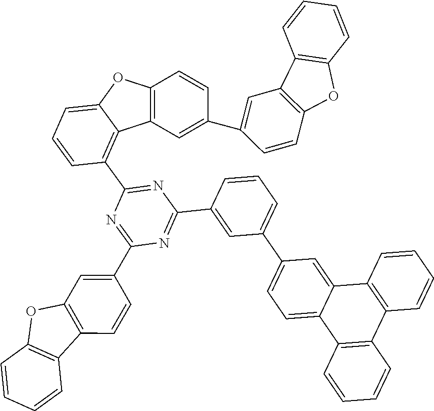 [SMILES: c1cc(-c2ccc3c4ccccc4c4ccccc4c3c2)cc(-c2nc(-c3ccc4c(c3)oc3ccccc34)nc(-c3cccc4oc5ccc(-c6ccc7oc8ccccc8c7c6)cc5c34)n2)c1]

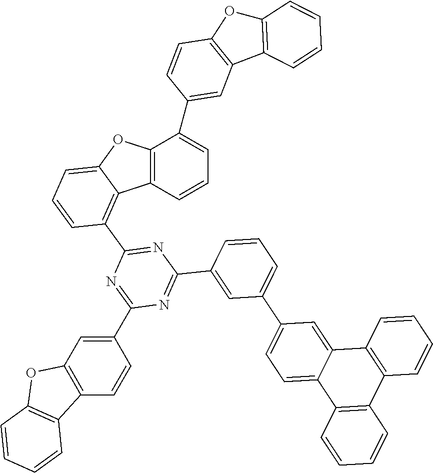 [SMILES: c1cc(-c2ccc3c4ccccc4c4ccccc4c3c2)cc(-c2nc(-c3ccc4c(c3)oc3ccccc34)nc(-c3cccc4oc5c(-c6ccc7oc8ccccc8c7c6)cccc5c34)n2)c1]

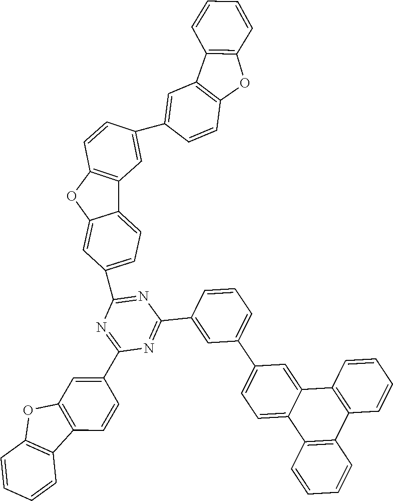 [SMILES: c1cc(-c2ccc3c4ccccc4c4ccccc4c3c2)cc(-c2nc(-c3ccc4c(c3)oc3ccccc34)nc(-c3ccc4c(c3)oc3ccc(-c5ccc6oc7ccccc7c6c5)cc34)n2)c1]

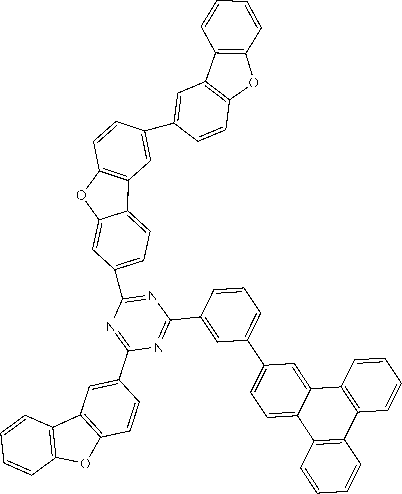 [SMILES: c1cc(-c2ccc3c4ccccc4c4ccccc4c3c2)cc(-c2nc(-c3ccc4c(c3)oc3ccc(-c5ccc6oc7ccccc7c6c5)cc34)nc(-c3ccc4oc5ccccc5c4c3)n2)c1]

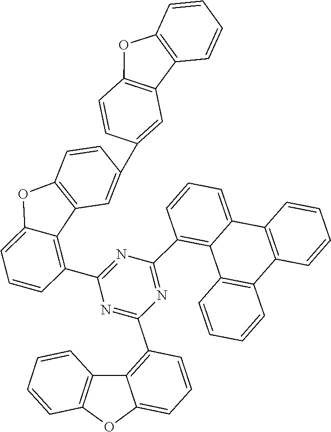 [SMILES: c1ccc2c(c1)oc1ccc(-c3ccc4oc5cccc(-c6nc(-c7cccc8oc9ccccc9c78)nc(-c7cccc8c9ccccc9c9ccccc9c78)n6)c5c4c3)cc12]

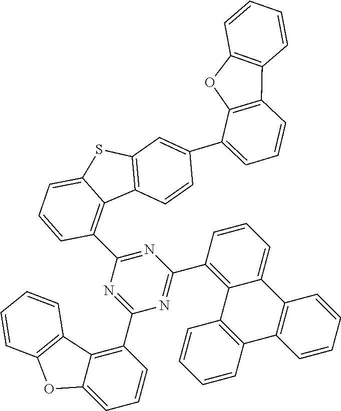 [SMILES: c1ccc2c(c1)oc1c(-c3ccc4c(c3)sc3cccc(-c5nc(-c6cccc7oc8ccccc8c67)nc(-c6cccc7c8ccccc8c8ccccc8c67)n5)c34)cccc12]

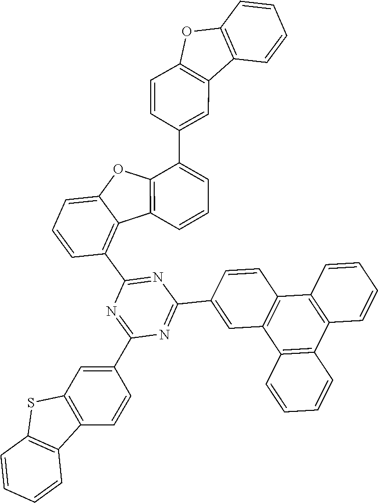 [SMILES: c1ccc2c(c1)oc1ccc(-c3cccc4c3oc3cccc(-c5nc(-c6ccc7c(c6)sc6ccccc67)nc(-c6ccc7c8ccccc8c8ccccc8c7c6)n5)c34)cc12]

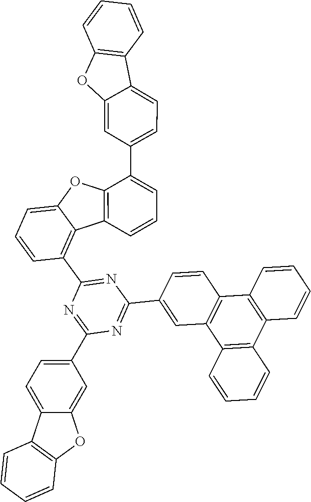 [SMILES: c1ccc2c(c1)oc1cc(-c3nc(-c4ccc5c6ccccc6c6ccccc6c5c4)nc(-c4cccc5oc6c(-c7ccc8c(c7)oc7ccccc78)cccc6c45)n3)ccc12]

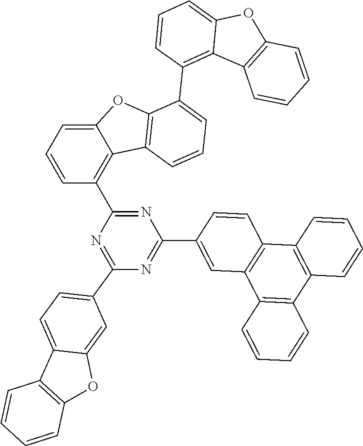 [SMILES: c1ccc2c(c1)oc1cc(-c3nc(-c4ccc5c6ccccc6c6ccccc6c5c4)nc(-c4cccc5oc6c(-c7cccc8oc9ccccc9c78)cccc6c45)n3)ccc12]

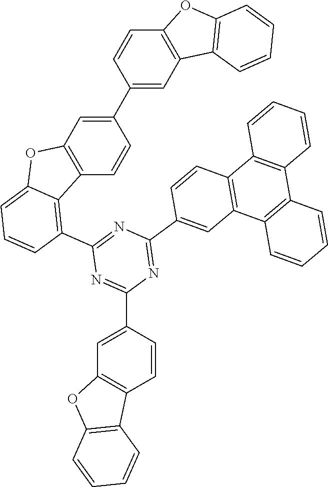 [SMILES: c1ccc2c(c1)oc1cc(-c3nc(-c4ccc5c6ccccc6c6ccccc6c5c4)nc(-c4cccc5oc6cc(-c7ccc8oc9ccccc9c8c7)ccc6c45)n3)ccc12]

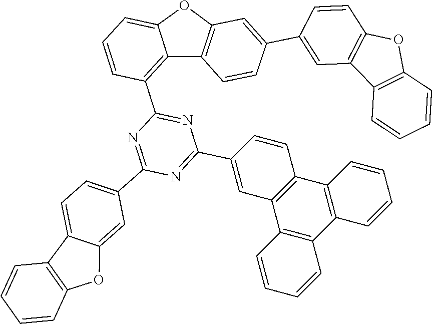 [SMILES: c1ccc2c(c1)oc1cc(-c3nc(-c4ccc5c6ccccc6c6ccccc6c5c4)nc(-c4cccc5oc6cc(-c7ccc8oc9ccccc9c8c7)ccc6c45)n3)ccc12]

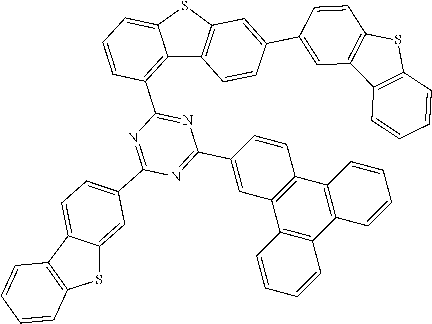 [SMILES: c1ccc2c(c1)sc1cc(-c3nc(-c4ccc5c6ccccc6c6ccccc6c5c4)nc(-c4cccc5sc6cc(-c7ccc8sc9ccccc9c8c7)ccc6c45)n3)ccc12]

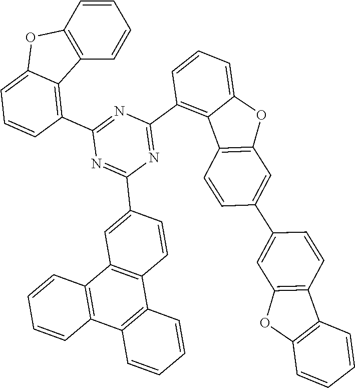 [SMILES: c1ccc2c(c1)oc1cc(-c3ccc4c(c3)oc3cccc(-c5nc(-c6ccc7c8ccccc8c8ccccc8c7c6)nc(-c6cccc7oc8ccccc8c67)n5)c34)ccc12]

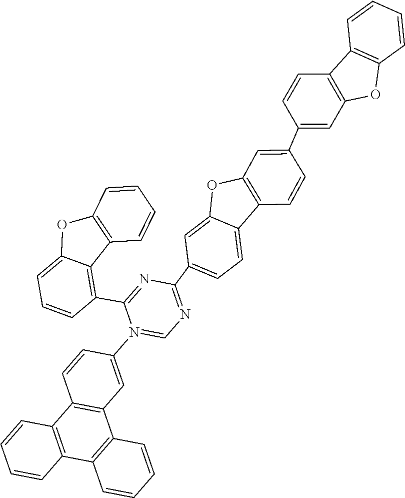

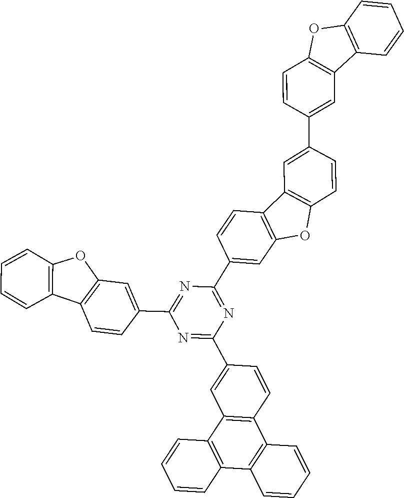 [SMILES: c1ccc2c(c1)oc1cc(-c3nc(-c4ccc5c(c4)oc4ccc(-c6ccc7oc8ccccc8c7c6)cc45)nc(-c4ccc5c6ccccc6c6ccccc6c5c4)n3)ccc12]

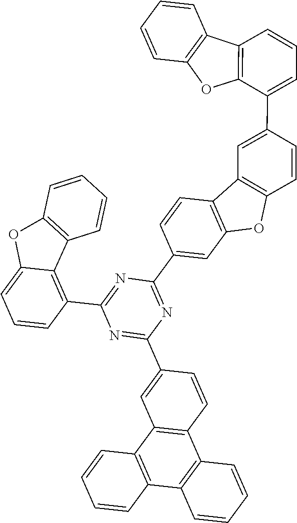 [SMILES: c1ccc2c(c1)oc1c(-c3ccc4oc5cc(-c6nc(-c7ccc8c9ccccc9c9ccccc9c8c7)nc(-c7cccc8oc9ccccc9c78)n6)ccc5c4c3)cccc12]

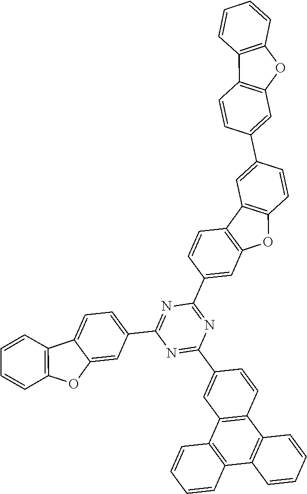 [SMILES: c1ccc2c(c1)oc1cc(-c3ccc4oc5cc(-c6nc(-c7ccc8c(c7)oc7ccccc78)nc(-c7ccc8c9ccccc9c9ccccc9c8c7)n6)ccc5c4c3)ccc12]

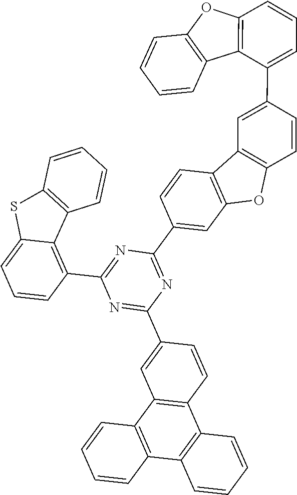 [SMILES: c1ccc2c(c1)oc1cccc(-c3ccc4oc5cc(-c6nc(-c7ccc8c9ccccc9c9ccccc9c8c7)nc(-c7cccc8sc9ccccc9c78)n6)ccc5c4c3)c12]

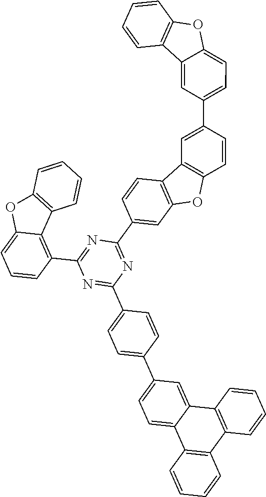 [SMILES: c1ccc2c(c1)oc1ccc(-c3ccc4oc5cc(-c6nc(-c7ccc(-c8ccc9c%10ccccc%10c%10ccccc%10c9c8)cc7)nc(-c7cccc8oc9ccccc9c78)n6)ccc5c4c3)cc12]

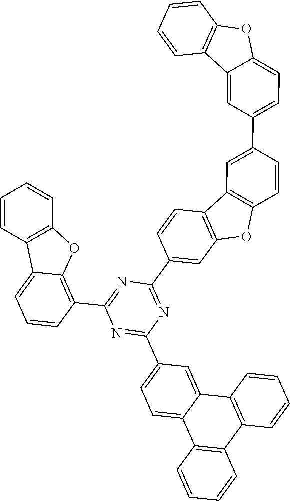 [SMILES: c1ccc2c(c1)oc1ccc(-c3ccc4oc5cc(-c6nc(-c7ccc8c9ccccc9c9ccccc9c8c7)nc(-c7cccc8c7oc7ccccc78)n6)ccc5c4c3)cc12]

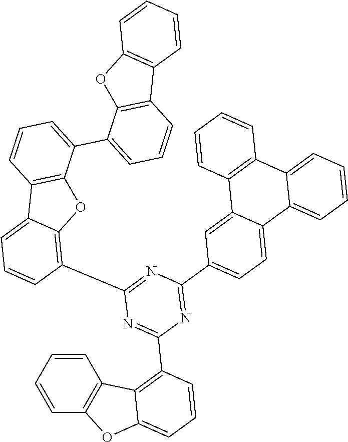 [SMILES: c1ccc2c(c1)oc1c(-c3cccc4c3oc3c(-c5nc(-c6ccc7c8ccccc8c8ccccc8c7c6)nc(-c6cccc7oc8ccccc8c67)n5)cccc34)cccc12]

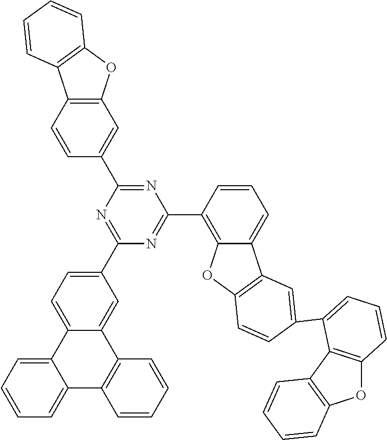 [SMILES: c1ccc2c(c1)oc1cc(-c3nc(-c4ccc5c6ccccc6c6ccccc6c5c4)nc(-c4cccc5c4oc4ccc(-c6cccc7oc8ccccc8c67)cc45)n3)ccc12]

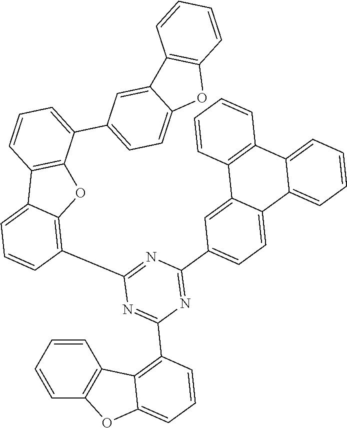 [SMILES: c1ccc2c(c1)oc1ccc(-c3cccc4c3oc3c(-c5nc(-c6ccc7c8ccccc8c8ccccc8c7c6)nc(-c6cccc7oc8ccccc8c67)n5)cccc34)cc12]

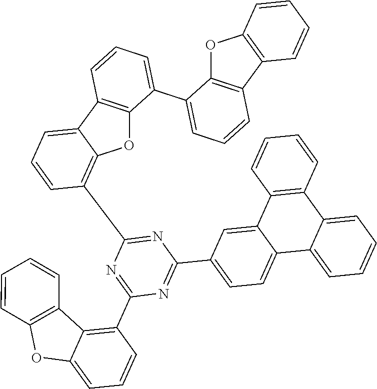 [SMILES: c1ccc2c(c1)oc1c(-c3cccc4c3oc3c(-c5nc(-c6ccc7c8ccccc8c8ccccc8c7c6)nc(-c6cccc7oc8ccccc8c67)n5)cccc34)cccc12]

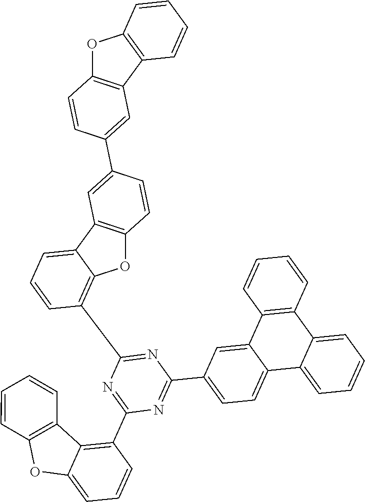 [SMILES: c1ccc2c(c1)oc1ccc(-c3ccc4oc5c(-c6nc(-c7ccc8c9ccccc9c9ccccc9c8c7)nc(-c7cccc8oc9ccccc9c78)n6)cccc5c4c3)cc12]

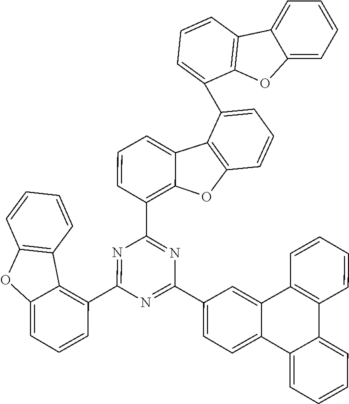 [SMILES: c1ccc2c(c1)oc1c(-c3cccc4oc5c(-c6nc(-c7ccc8c9ccccc9c9ccccc9c8c7)nc(-c7cccc8oc9ccccc9c78)n6)cccc5c34)cccc12]

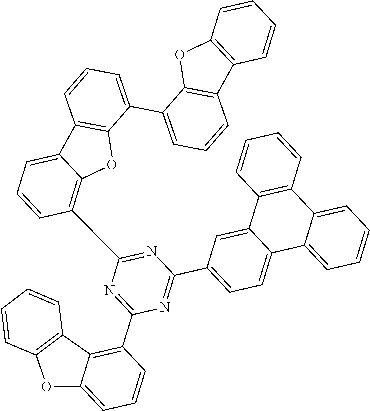 [SMILES: c1ccc2c(c1)oc1c(-c3cccc4c3oc3c(-c5nc(-c6ccc7c8ccccc8c8ccccc8c7c6)nc(-c6cccc7oc8ccccc8c67)n5)cccc34)cccc12]

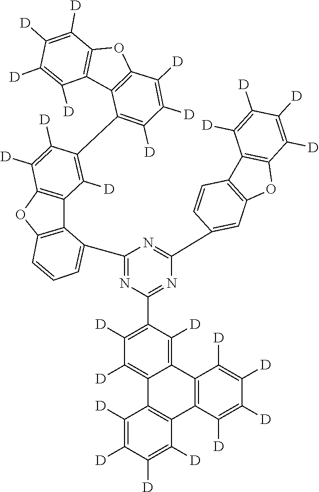 [SMILES: [2H]c1c([2H])c([2H])c2c(oc3cc(-c4nc(-c5c([2H])c([2H])c6c7c([2H])c([2H])c([2H])c([2H])c7c7c([2H])c([2H])c([2H])c([2H])c7c6c5[2H])nc(-c5cccc6oc7c([2H])c([2H])c(-c8c([2H])c([2H])c([2H])c9oc%10c([2H])c([2H])c([2H])c([2H])c%10c89)c([2H])c7c56)n4)ccc32)c1[2H]]

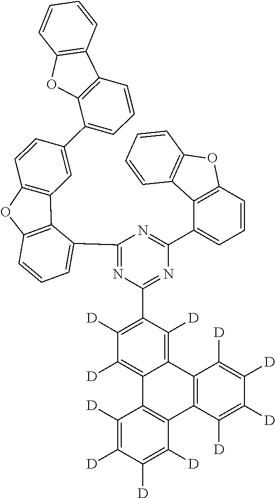 [SMILES: [2H]c1c([2H])c([2H])c2c(c1[2H])c1c([2H])c([2H])c([2H])c([2H])c1c1c([2H])c(-c3nc(-c4cccc5oc6ccccc6c45)nc(-c4cccc5oc6ccc(-c7cccc8c7oc7ccccc78)cc6c45)n3)c([2H])c([2H])c21]

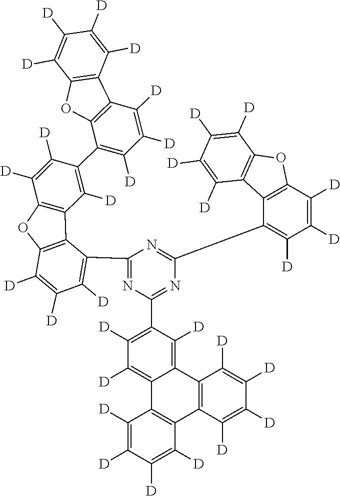 [SMILES: [2H]c1c([2H])c([2H])c2c(oc3c(-c4c([2H])c([2H])c5oc6c([2H])c([2H])c([2H])c(-c7nc(-c8c([2H])c([2H])c9c%10c([2H])c([2H])c([2H])c([2H])c%10c%10c([2H])c([2H])c([2H])c([2H])c%10c9c8[2H])nc(-c8c([2H])c([2H])c([2H])c9oc%10c([2H])c([2H])c([2H])c([2H])c%10c89)n7)c6c5c4[2H])c([2H])c([2H])c([2H])c32)c1[2H]]

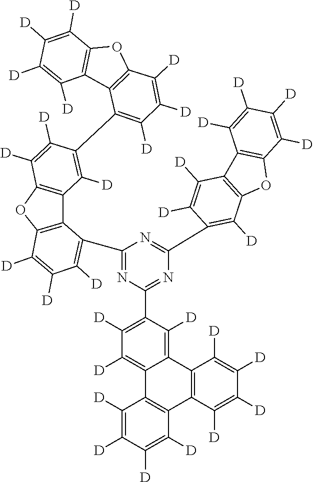 [SMILES: [2H]c1c([2H])c([2H])c2c(oc3c([2H])c(-c4nc(-c5c([2H])c([2H])c6c7c([2H])c([2H])c([2H])c([2H])c7c7c([2H])c([2H])c([2H])c([2H])c7c6c5[2H])nc(-c5c([2H])c([2H])c([2H])c6oc7c([2H])c([2H])c(-c8c([2H])c([2H])c([2H])c9oc%10c([2H])c([2H])c([2H])c([2H])c%10c89)c([2H])c7c56)n4)c([2H])c([2H])c32)c1[2H]]

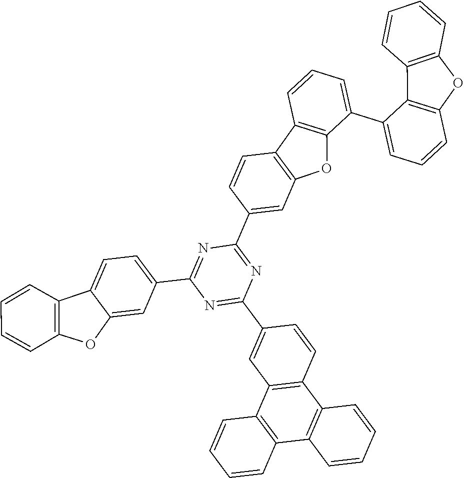 [SMILES: c1ccc2c(c1)oc1cc(-c3nc(-c4ccc5c(c4)oc4c(-c6cccc7oc8ccccc8c67)cccc45)nc(-c4ccc5c6ccccc6c6ccccc6c5c4)n3)ccc12]

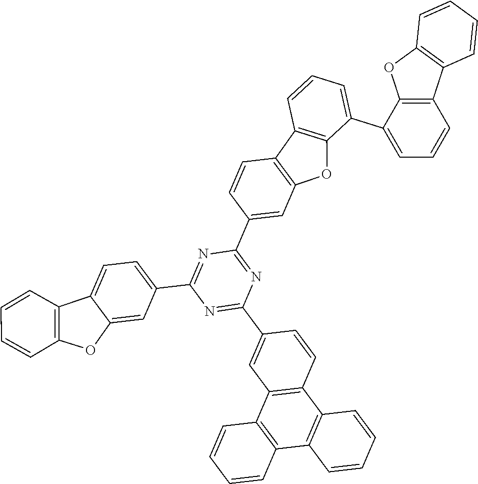 [SMILES: c1ccc2c(c1)oc1cc(-c3nc(-c4ccc5c(c4)oc4c(-c6cccc7c6oc6ccccc67)cccc45)nc(-c4ccc5c6ccccc6c6ccccc6c5c4)n3)ccc12]

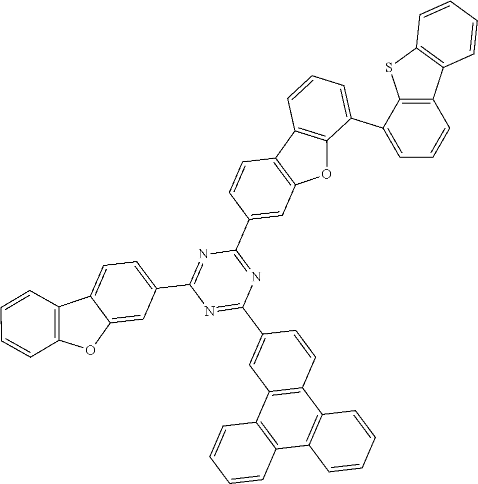 [SMILES: c1ccc2c(c1)oc1cc(-c3nc(-c4ccc5c(c4)oc4c(-c6cccc7c6sc6ccccc67)cccc45)nc(-c4ccc5c6ccccc6c6ccccc6c5c4)n3)ccc12]

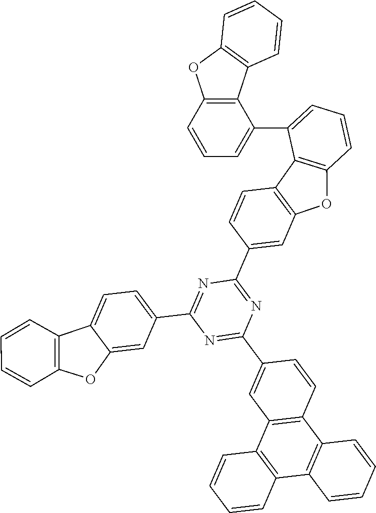 [SMILES: c1ccc2c(c1)oc1cc(-c3nc(-c4ccc5c(c4)oc4cccc(-c6cccc7oc8ccccc8c67)c45)nc(-c4ccc5c6ccccc6c6ccccc6c5c4)n3)ccc12]

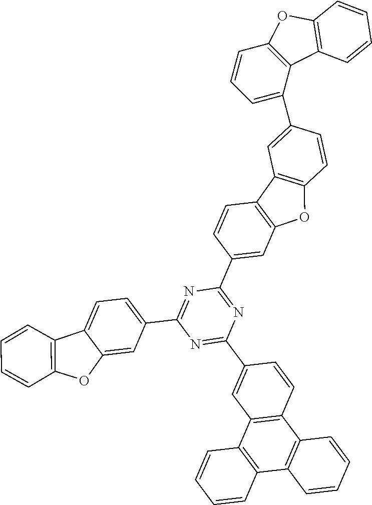 [SMILES: c1ccc2c(c1)oc1cc(-c3nc(-c4ccc5c(c4)oc4ccc(-c6cccc7oc8ccccc8c67)cc45)nc(-c4ccc5c6ccccc6c6ccccc6c5c4)n3)ccc12]

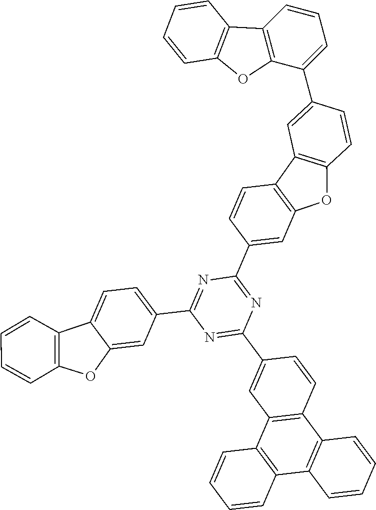 [SMILES: c1ccc2c(c1)oc1cc(-c3nc(-c4ccc5c(c4)oc4ccc(-c6cccc7c6oc6ccccc67)cc45)nc(-c4ccc5c6ccccc6c6ccccc6c5c4)n3)ccc12]

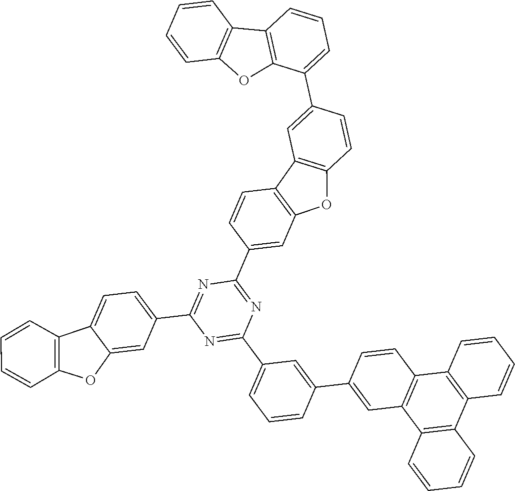 [SMILES: c1cc(-c2ccc3c4ccccc4c4ccccc4c3c2)cc(-c2nc(-c3ccc4c(c3)oc3ccccc34)nc(-c3ccc4c(c3)oc3ccc(-c5cccc6c5oc5ccccc56)cc34)n2)c1]

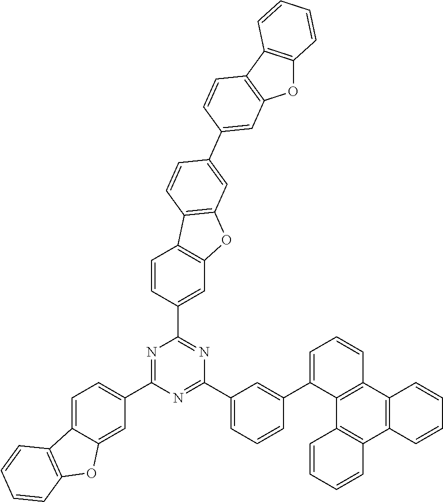 [SMILES: c1cc(-c2nc(-c3ccc4c(c3)oc3ccccc34)nc(-c3ccc4c(c3)oc3cc(-c5ccc6c(c5)oc5ccccc56)ccc34)n2)cc(-c2cccc3c4ccccc4c4ccccc4c23)c1]

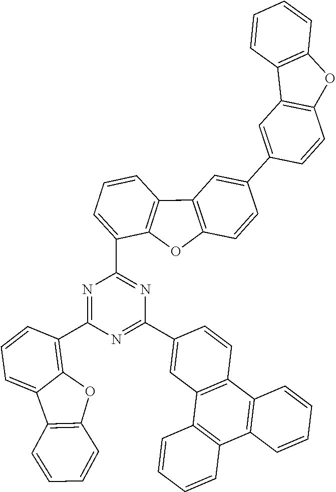 [SMILES: c1ccc2c(c1)oc1ccc(-c3ccc4oc5c(-c6nc(-c7ccc8c9ccccc9c9ccccc9c8c7)nc(-c7cccc8c7oc7ccccc78)n6)cccc5c4c3)cc12]

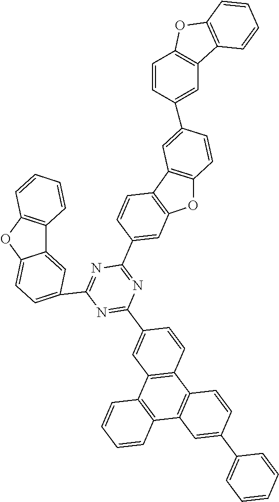 [SMILES: c1ccc(-c2ccc3c4ccc(-c5nc(-c6ccc7c(c6)oc6ccc(-c8ccc9oc%10ccccc%10c9c8)cc67)nc(-c6ccc7oc8ccccc8c7c6)n5)cc4c4ccccc4c3c2)cc1]

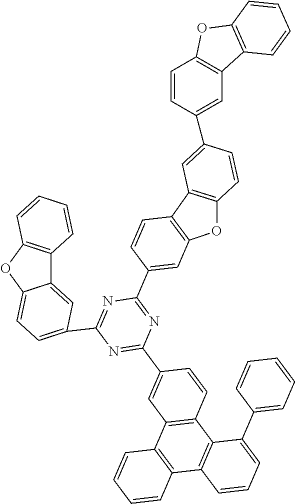 [SMILES: c1ccc(-c2cccc3c4ccccc4c4cc(-c5nc(-c6ccc7c(c6)oc6ccc(-c8ccc9oc%10ccccc%10c9c8)cc67)nc(-c6ccc7oc8ccccc8c7c6)n5)ccc4c23)cc1]

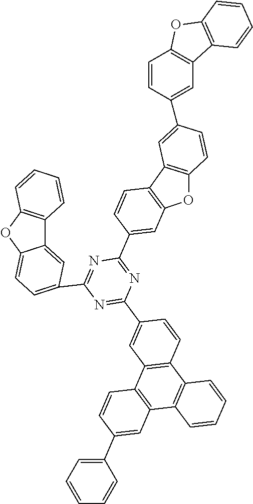 [SMILES: c1ccc(-c2ccc3c(c2)c2ccccc2c2ccc(-c4nc(-c5ccc6c(c5)oc5ccc(-c7ccc8oc9ccccc9c8c7)cc56)nc(-c5ccc6oc7ccccc7c6c5)n4)cc23)cc1]

Particularly suitable compounds of the formulae (1), (1a), (1b), (1c), (1d), (1e), (1f), (1g), (1h) and (1i) are the compounds EG1 to EG18 in table 2.

TABLE 2

| | |
|---|---|
| 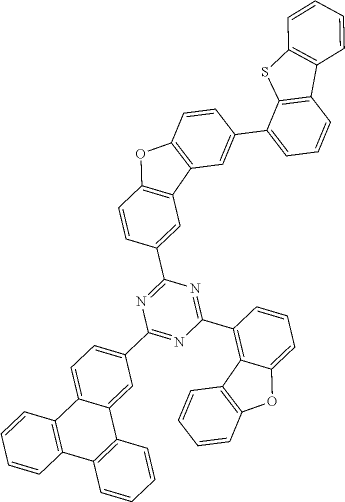 [SMILES: c1ccc2c(c1)oc1cccc(-c3nc(-c4ccc5oc6ccc(-c7cccc8c7sc7ccccc78)cc6c5c4)nc(-c4ccc5c6ccccc6c6ccccc6c5c4)n3)c12] | EG1 |
| 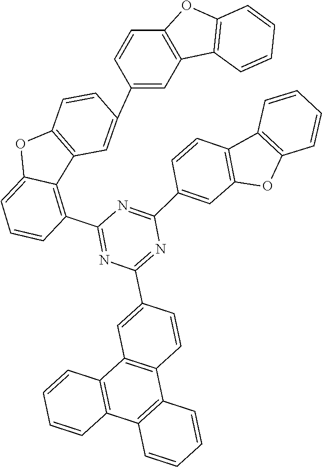 [SMILES: c1ccc2c(c1)oc1cc(-c3nc(-c4ccc5c6ccccc6c6ccccc6c5c4)nc(-c4cccc5oc6ccc(-c7ccc8oc9ccccc9c8c7)cc6c45)n3)ccc12] | EG2 |
| 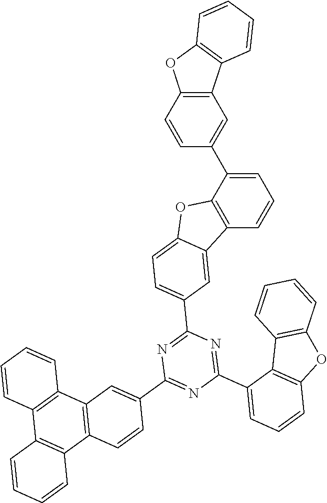 [SMILES: c1ccc2c(c1)oc1ccc(-c3cccc4c3oc3ccc(-c5nc(-c6ccc7c8ccccc8c8ccccc8c7c6)nc(-c6cccc7oc8ccccc8c67)n5)cc34)cc12] | EG3 |
| 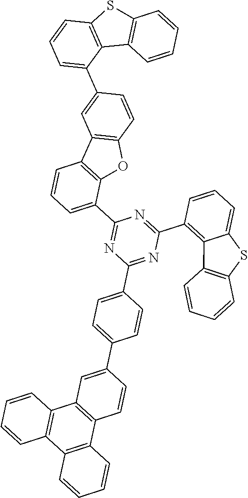 [SMILES: c1ccc2c(c1)sc1cccc(-c3ccc4oc5c(-c6nc(-c7ccc(-c8ccc9c%10ccccc%10c%10ccccc%10c9c8)cc7)nc(-c7cccc8sc9ccccc9c78)n6)cccc5c4c3)c12] | EG4 |
| 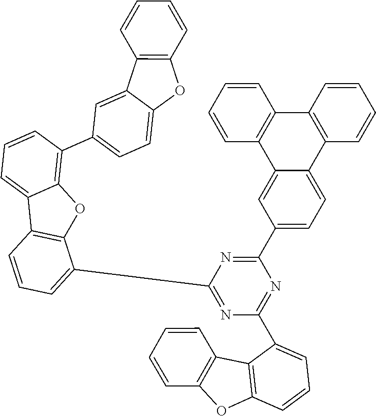 [SMILES: c1ccc2c(c1)oc1ccc(-c3cccc4c3oc3c(-c5nc(-c6ccc7c8ccccc8c8ccccc8c7c6)nc(-c6cccc7oc8ccccc8c67)n5)cccc34)cc12] | EG5 |
| 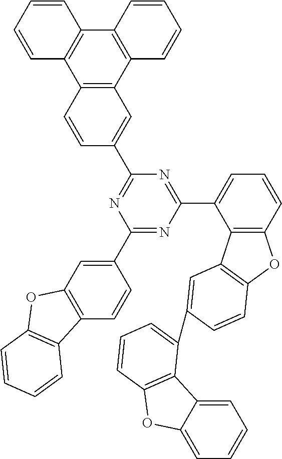 [SMILES: c1ccc2c(c1)oc1cc(-c3nc(-c4ccc5c6ccccc6c6ccccc6c5c4)nc(-c4cccc5oc6ccc(-c7cccc8oc9ccccc9c78)cc6c45)n3)ccc12] | EG6 |
| 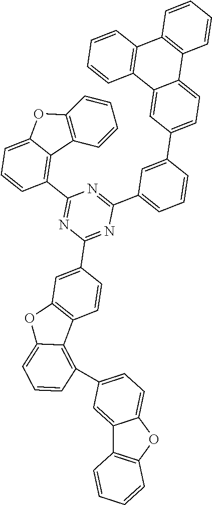 [SMILES: c1cc(-c2ccc3c4ccccc4c4ccccc4c3c2)cc(-c2nc(-c3ccc4c(c3)oc3cccc(-c5ccc6oc7ccccc7c6c5)c34)nc(-c3cccc4oc5ccccc5c34)n2)c1] | EG7 |
| 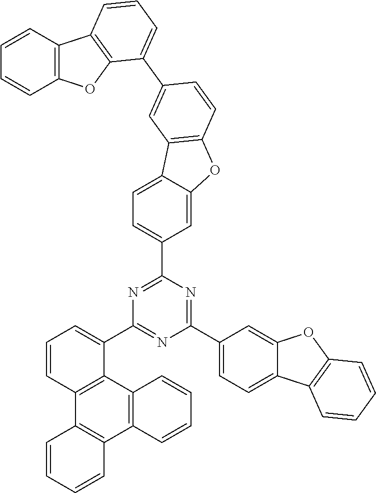 [SMILES: c1ccc2c(c1)oc1cc(-c3nc(-c4ccc5c(c4)oc4ccc(-c6cccc7c6oc6ccccc67)cc45)nc(-c4cccc5c6ccccc6c6ccccc6c45)n3)ccc12] | EG8 |
| 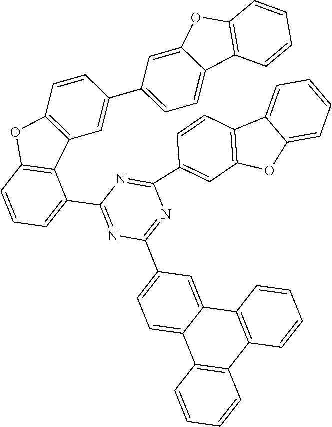 [SMILES: c1ccc2c(c1)oc1cc(-c3ccc4oc5cccc(-c6nc(-c7ccc8c(c7)oc7ccccc78)nc(-c7ccc8c9ccccc9c9ccccc9c8c7)n6)c5c4c3)ccc12] | EG9 |
| 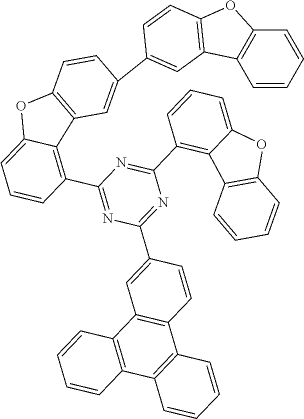 [SMILES: c1ccc2c(c1)oc1ccc(-c3ccc4oc5cccc(-c6nc(-c7ccc8c9ccccc9c9ccccc9c8c7)nc(-c7cccc8oc9ccccc9c78)n6)c5c4c3)cc12] | EG10 |
| 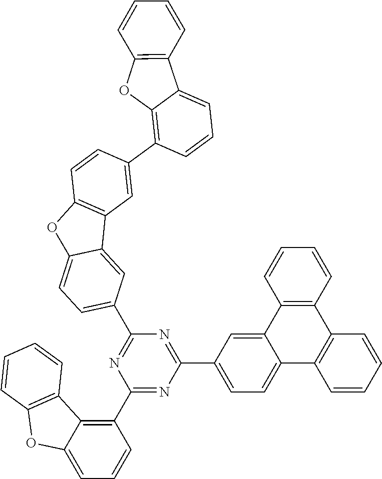 [SMILES: c1ccc2c(c1)oc1c(-c3ccc4oc5ccc(-c6nc(-c7ccc8c9ccccc9c9ccccc9c8c7)nc(-c7cccc8oc9ccccc9c78)n6)cc5c4c3)cccc12] | EG11 |
| 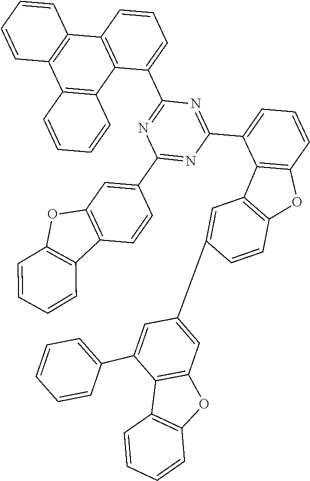 [SMILES: c1ccc(-c2cc(-c3ccc4oc5cccc(-c6nc(-c7ccc8c(c7)oc7ccccc78)nc(-c7cccc8c9ccccc9c9ccccc9c78)n6)c5c4c3)cc3oc4ccccc4c23)cc1] | EG12 |
| 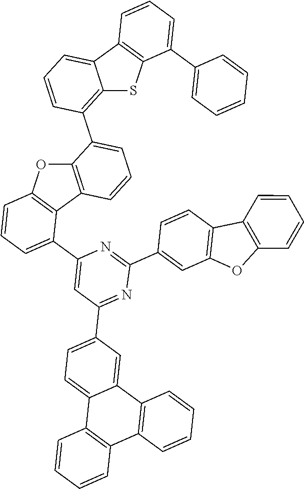 [SMILES: c1ccc(-c2cccc3c2sc2c(-c4cccc5c4oc4cccc(-c6cc(-c7ccc8c9ccccc9c9ccccc9c8c7)nc(-c7ccc8c(c7)oc7ccccc78)n6)c45)cccc23)cc1] | EG13 |
| 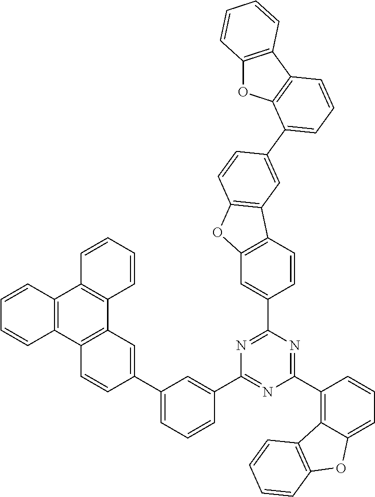 [SMILES: c1cc(-c2ccc3c4ccccc4c4ccccc4c3c2)cc(-c2nc(-c3ccc4c(c3)oc3ccc(-c5cccc6c5oc5ccccc56)cc34)nc(-c3cccc4oc5ccccc5c34)n2)c1] | EG14 |
| 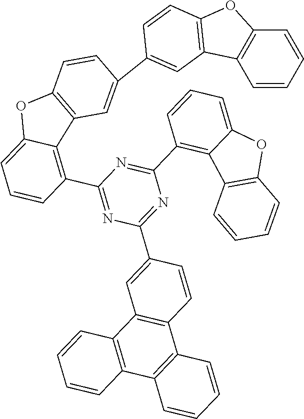 [SMILES: c1ccc2c(c1)oc1ccc(-c3ccc4oc5cccc(-c6nc(-c7ccc8c9ccccc9c9ccccc9c8c7)nc(-c7cccc8oc9ccccc9c78)n6)c5c4c3)cc12] | EG15 |
| 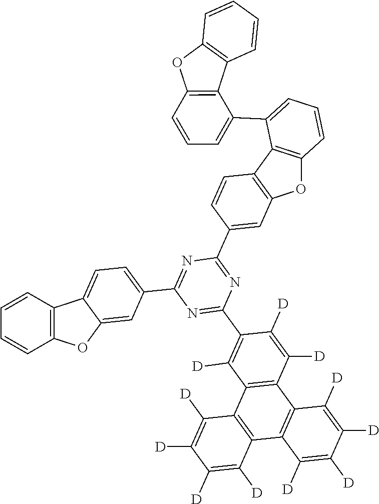 [SMILES: [2H]c1c([2H])c([2H])c2c(c1[2H])c1c([2H])c([2H])c([2H])c([2H])c1c1c([2H])c(-c3nc(-c4ccc5c(c4)oc4ccccc45)nc(-c4ccc5c(c4)oc4cccc(-c6cccc7oc8ccccc8c67)c45)n3)c([2H])c([2H])c21] | EG16 |
| 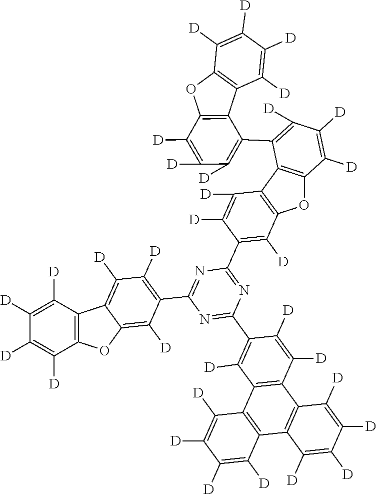 [SMILES: [2H]c1c([2H])c([2H])c2c(oc3c([2H])c(-c4nc(-c5c([2H])c([2H])c6c(oc7c([2H])c([2H])c([2H])c(-c8c([2H])c([2H])c([2H])c9oc%10c([2H])c([2H])c([2H])c([2H])c%10c89)c76)c5[2H])nc(-c5c([2H])c([2H])c6c7c([2H])c([2H])c([2H])c([2H])c7c7c([2H])c([2H])c([2H])c([2H])c7c6c5[2H])n4)c([2H])c([2H])c32)c1[2H]] | EG17 |
| 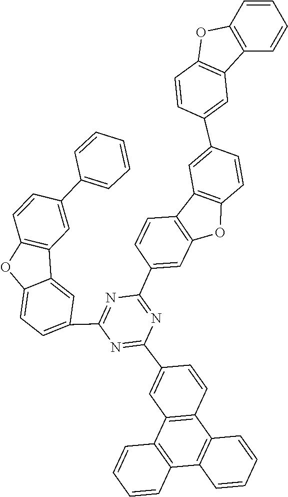 [SMILES: c1ccc(-c2ccc3oc4ccc(-c5nc(-c6ccc7c(c6)oc6ccc(-c8ccc9oc%10ccccc%10c9c8)cc67)nc(-c6ccc7c8ccccc8c8ccccc8c7c6)n5)cc4c3c2)cc1] | EG18 |

The compounds of the invention can be prepared by synthesis steps known to those skilled in the art, for example bromination, Suzuki coupling, Ullmann coupling, Hartwig-Buchwald coupling, etc.

In the synthesis schemes which follow, the compounds are shown with a small number of substituents to simplify the structures. This does not rule out the presence of any desired further substituents in the processes. The methods shown for synthesis of the compounds of the invention should be regarded as illustrative. The person skilled in the art will be able to develop alternative synthesis routes within the scope of his common knowledge in the art.

An illustrative implementation is given by the schemes which follow, without any intention that these should impose a restriction. The component steps of the individual schemes may be combined with one another as desired.

Compounds of the formula (1) can be prepared, for example, according to scheme 1 below, where W in each case, according to formula (1), is V₁ or V₂ and R is (R #)_{b1} or (R #)_{b2}, H or D.

Ar in scheme 1 is

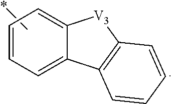

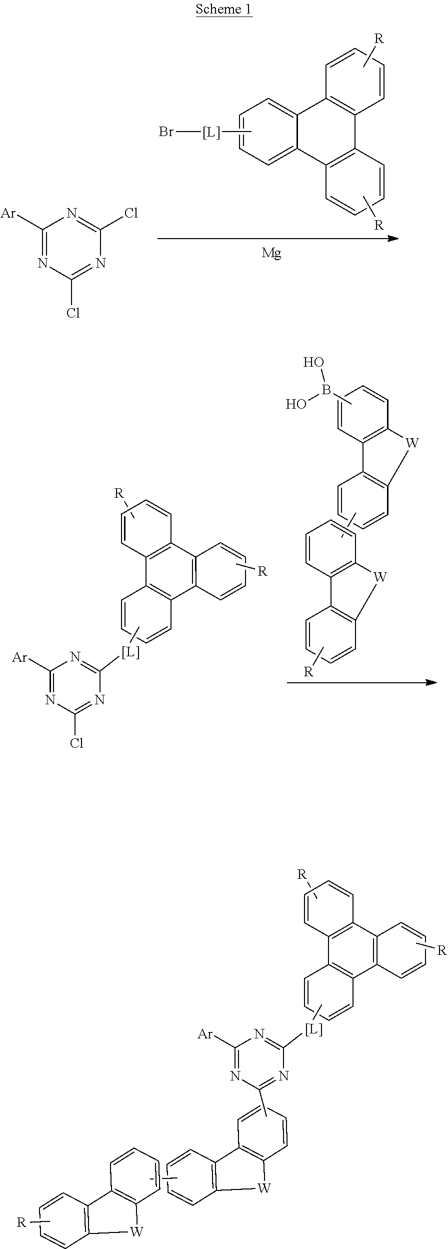

Compounds of the formula (1) in which [L] denotes a single bond can be prepared, for example, according to scheme 2 below, where W in each case, according to formula (1), is V₁ or V₂ and R is (R #)_{b1} or (R #)_{b2}. The person skilled in the art will be capable of correspondingly adjusting the preparation of compounds of the formula (1) with a linker [L] according to scheme 2.

Ar in scheme 2 is

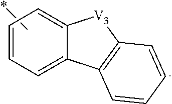

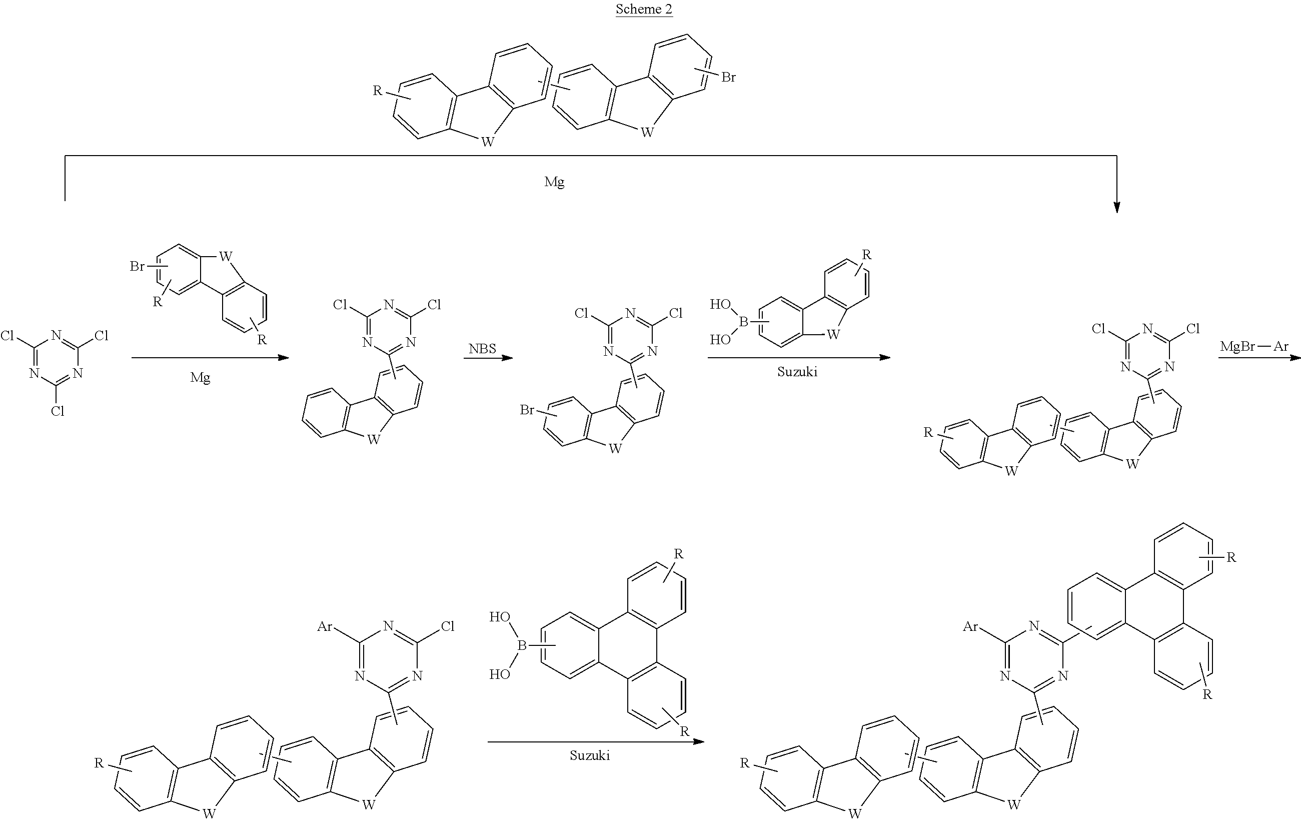

It is possible by these processes, if necessary followed by purification, for example recrystallization or sublimation, to obtain the compounds of the formula (1) in high purity, preferably more than 99% (determined by means of 1H NMR and/or HPLC).

Suitable methods of deuterating the host material 1 are known to those skilled in the art. Suitable methods are described hereinafter and are also correspondingly applicable to the host material 1.

For the processing of the compounds of the invention from liquid phase, for example by spin-coating or by printing methods, formulations of the compounds of the invention or of mixtures of compounds of the invention with further functional materials, such as matrix materials, fluorescent emitters, phosphorescent emitters and/or emitters that exhibit TADF, are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl) ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The inventive compounds of the formula (1), as described above or described as preferred, are suitable for use in an organic electroluminescent device, especially as matrix material.

When the compound of the invention is used as matrix material or, synonymously, host material in an emitting layer, it is preferably used in combination with a further compound.

The invention therefore further provides a mixture comprising at least one compound of the formula (1) or at least one preferred compound of the formula (1) or a compound from table 1 or one of compounds EG1 to EG18 and at least one further compound selected from the group of the matrix materials, phosphorescent emitters, fluorescent emitters and/or emitters that exhibit TADF (thermally activated delayed fluorescence). Suitable matrix materials and emitters that can be used in this mixture of the invention are described hereinafter.

The present invention likewise further provides a formulation comprising at least one compound of the invention, as described above, or a mixture of the invention, as described above, and at least one solvent. The solvent may be an abovementioned solvent or a mixture of these solvents.

The present invention further provides an organic electroluminescent device comprising an anode, a cathode and at least one organic layer, comprising at least one compound of the formula (1), or at least one preferred compound of the formula (1), or a compound from table 1 or one of compounds EG1 to EG18.

The organic electroluminescent device (synonymous with organic electroluminescence device) of the invention is, for example, an organic light-emitting transistor (OLET), an organic field quench device (OFQD), an organic light-emitting electrochemical cell (OLEC, LEC, LEEC), an organic laser diode (O-laser) or an organic light-emitting diode (OLED). The organic electroluminescent device of the invention is especially an organic light-emitting diode or an organic light-emitting electrochemical cell. The device of the invention is more preferably an OLED.

The organic layer of the device of the invention preferably comprises, as well as a light-emitting layer (EML), a hole injection layer (HIL), a hole transport layer (HTL), a hole blocker layer (HBL), an electron transport layer (ETL), an electron injection layer (EIL), an exciton blocker layer, an electron blocker layer and/or charge generation layers. It is also possible for the device of the invention to include two or more layers from this group, preferably selected from EML, HIL, HTL, ETL, EIL and HBL. It is likewise possible for interlayers having an exciton-blocking function, for example, to be introduced between two emitting layers.

If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The device may also comprise inorganic materials or else layers formed entirely from inorganic materials.

It presents no difficulties at all to the person skilled in the art to consider a multitude of materials known in the prior art in order to select suitable materials for use in the above-described layers of the organic electroluminescent device. The person skilled in the art here will reflect in a customary manner on the chemical and physical properties of materials, since he knows that the materials interact with one another in an organic electroluminescent device. This relates, for example, to the energy levels of the orbitals (HOMO, LUMO) or else the triplet and singlet energy levels, but also other material properties.

The inventive compound of the formula (1) as described above or as described as preferred can be used in different layers, according to the exact structure. Preference is given to an organic electroluminescent device comprising a compound of formula (1) or the above-recited preferred embodiments in an emitting layer as matrix material for fluorescent emitters, phosphorescent emitters or for emitters that exhibit TADF (thermally activated delayed fluorescence), especially for phosphorescent emitters. In addition, the compound of the invention can also be used in an electron transport layer and/or in a hole transport layer and/or in an exciton blocker layer and/or in a hole blocker layer. Particular preference is given to using the compound of the invention as matrix material in an emitting layer or as electron transport material or hole blocker material in an electron transport layer or hole blocker layer.

The present invention further provides an organic electroluminescent device as described above, wherein the organic layer comprises at least one light-emitting layer comprising the at least one compound of the formula (1), or the at least one preferred compound of the formula (1), or a compound from table 1 or one of compounds EG1 to EG18.

In one embodiment of the invention, for the device of the invention, a further matrix material is selected in the light-emitting layer, and this is used together with compounds of the formula (1) as described above or described as preferred or with the compounds from table 1 or the compounds EG1 to EG18.

The present invention accordingly further provides an organic electroluminescent device as described above, wherein the organic layer comprises at least one light-emitting layer comprising the at least one compound of the formula (1), or the at least one preferred compound of the formula (1), or a compound from table 1 or one of compounds EG1 to EG18, and a further matrix material.

Suitable matrix materials that can be used in combination with the compounds of the invention are aromatic ketones, aromatic phosphine oxides or aromatic sulfoxides or sulfones, triarylamines, carbazole derivatives, biscarbazoles, indolocarbazole derivatives, indenocarbazole derivatives, azacarbazole derivatives, bipolar matrix materials, azaboroles or boronic esters, triazine derivatives, zinc complexes, diazasilole or tetraazasilole derivatives, diazaphosphole derivatives, bridged carbazole derivatives, triphenylene derivatives or dibenzofuran derivatives. It is likewise possible for a further phosphorescent emitter having shorter-wavelength emission than the actual emitter to be present as co-host in the mixture, or a compound not involved in charge transport to a significant extent, if at all, for example a wide band-gap compound.

What is meant herein by a wide-bandgap material is a material within the scope of the disclosure of U.S. Pat. No. 7,294,849 which is characterized by a band gap of at least 3.5 eV, the band gap meaning the gap between the HOMO and LUMO energy of a material.

Particularly suitable matrix materials that are advantageously combined in a mixed matrix system with compounds of the formula (1) as described above or described as preferred may be selected from the compounds of the formulae (6), (7), (8), (9), (10) or (11), as described hereinafter.

The invention accordingly further provides an organic electroluminescent device comprising an anode, a cathode and at least one organic layer comprising at least one light-emitting layer, wherein the at least one light-emitting layer comprises at least one compound of the formula (1) as matrix material 1, as described above or as described as preferred, and at least one compound of the formulae (6), (7), (8), (9) or (10) as matrix material 2.

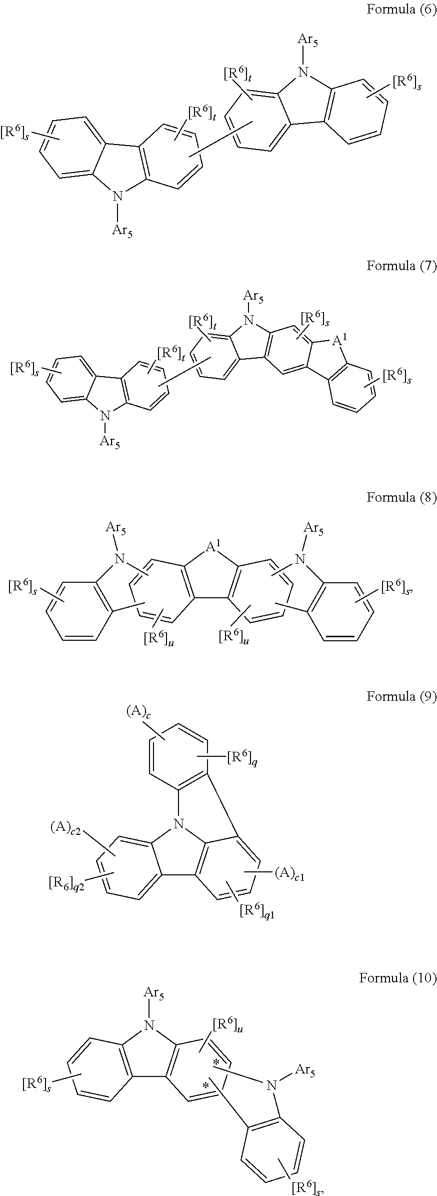

where the symbols and indices used are as follows:
A¹ is C (R⁷)₂, NR⁷, O or S;
A at each instance is independently a group of the formula (3) or (4),

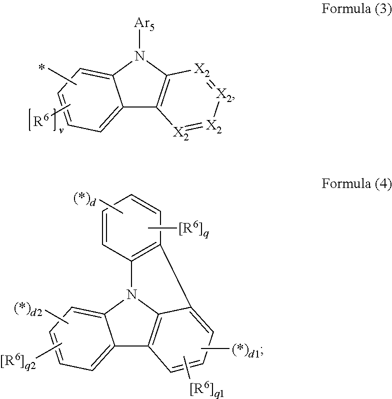

X₂ is the same or different at each instance and is CH, CR⁶ or N, where not more than 2 symbols X₂ can be N;
* indicates the binding site to the formula (9);
R⁶ at each instance is the same or different and is D, CN, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 ring atoms and may be substituted in each case by one or more R⁷ radicals; it is also possible here for two Re radicals together to form an aromatic, heteroaromatic, aliphatic or heteroaliphatic ring system;
Ar is the same or different at each instance and is an aromatic or heteroaromatic ring system which has 5 to 40 ring atoms and may be substituted by one or more R⁷ radicals;
Ar⁵ is the same or different at each instance and is an aromatic or heteroaromatic ring system which has 5 to 40 ring atoms and may be substituted by one or more R⁷ radicals;
R⁷ is the same or different at each instance and is D, F, Cl, Br, I, N (R⁸)₂, CN, NO₂, OR⁸, SR⁸, Si(R⁸)₃, B (OR⁸)₂, C(═O)R⁸, P(═O)(R⁸)₂, S(═O)R⁸, S(═O)₂R⁸, OSOR⁸, a straight-chain alkyl group having 1 to 20 carbon atoms or an alkenyl or alkynyl group having 2 to 20 carbon atoms or a branched or cyclic alkyl group having 3 to carbon atoms, where the alkyl, alkenyl or alkynyl group may in each case be substituted by one or more R⁸ radicals, where one or more nonadjacent CH₂ groups may be replaced by Si(R⁸)₂, C═O, NR⁸, O, S or CONR⁸, or an aromatic or heteroaromatic ring system which has 5 to 40 ring atoms and may be substituted in each case by one or more R⁸ radicals; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
c, c1, c2 at each instance are each independently 0 or 1, where the sum total of the indices at each instance c+c1+c2 is 1;
d, d1, d2 at each instance are each independently 0 or 1, where the sum total of the indices at each instance d+d1+d2 is 1;
q, q1, q2 at each instance are each independently 0 or 1;
S is the same or different at each instance and is 0, 1, 2, 3 or 4;
t is the same or different at each instance and is 0, 1, 2 or 3;
u is the same or different at each instance and is 0, 1 or 2; and
V is 0 or 1.

In compounds of the formulae (6), (7), (8) and (10), s is preferably 0 or 1, more preferably 0.

In compounds of the formulae (6), (7) and (8), t is preferably 0 or 1, more preferably 0.

In compounds of the formulae (6), (7), (8) and (10), u is preferably 0 or 1, more preferably 0.

The sum total of the indices s, t and u in compounds of the formulae (6), (7), (8) and (10) is preferably not more than 6, especially preferably not more than 4 and more preferably not more than 2.

In compounds of the formula (9), c, c1, c2 at each instance are each independently 0 or 1, where the sum total of the indices at each instance c+c1+c2 is 1. c2 is preferably defined as 1.

In a preferred embodiment of the compounds of the formulae (6), (7), (8) and (10) that can be combined in accordance with the invention with compounds of formula (1), R⁶ is the same or different at each instance and is selected from the group consisting of D, F, CN, NO₂, Si(R⁷)₃, B (OR⁷)₂, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a preferred embodiment of the compounds of the formulae (6), (7), (8) and (10) that can be combined in accordance with the invention with compounds of formula (1), as described above, R⁶ is the same or different at each instance and is selected from the group consisting of D and an aromatic heteroaromatic ring system which has 6 to 30 aromatic ring atoms and may be substituted by one or more R⁷ radicals. A preferred R⁷ radical is the N (Ar)₂ group.

Preferably, Ars in compounds of the formulae (6), (7), (8) and (10) is selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorenyl which may be joined via the 1, 2, 3 or 4 position, spirobifluorenyl which may be joined via the 1, 2, 3 or 4 position, naphthyl, especially 1- or 2-bonded naphthyl, or radicals derived from indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. Ars is preferably unsubstituted.

When A¹ in formula (7) or (8) is NR⁷, the substituent R⁷ bonded to the nitrogen atom is preferably an aromatic or heteroaromatic ring system which has 5 to 24 aromatic ring atoms and may also be substituted by one or more R⁸ radicals. In a particularly preferred embodiment, this substituent R⁷ is the same or different at each instance and is an aromatic or heteroaromatic ring system having 6 to 24 aromatic ring atoms, especially having 6 to 18 aromatic ring atoms. Preferred embodiments of R′ are phenyl, biphenyl, terphenyl and quaterphenyl, which are preferably unsubstituted, and radicals derived from triazine, pyrimidine and quinazoline, which may be substituted by one or more R⁸ radicals.

When A¹ in formula (7) or (8) is C (R⁷)₂, the substituents R⁷ bonded to this carbon atom are preferably the same or different at each instance and are a linear alkyl group having 1 to carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁷ is a methyl group or a phenyl group. In this case, the R⁷ radicals together may also form a ring system, which leads to a spiro system.

In a preferred embodiment of the compounds of the formulae (6), (7), (8), (9) and (10), these compounds are partly or fully deuterated, more preferably fully deuterated.

The preparation of the compounds of the formulae (6), (7), (8), (9) and (10) is generally known, and some of the compounds are commercially available.

Compounds of the formula (9) are, for example, in WO2021180614, pages 110 to 119, especially as examples on pages 120 to 127. The preparation thereof is disclosed in WO2021180614 on page 128, and in the synthesis examples on pages 214 to 218.

The invention also further provides an organic electroluminescent device comprising an anode, a cathode and at least one organic layer comprising at least one light-emitting layer, wherein the at least one light-emitting layer comprises at least one compound of the formula (1) as matrix material 1, as described above or as described as preferred, and at least one compound of the formula (11):

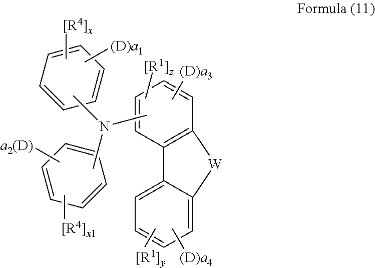

where the symbols and indices used are as follows:
W is O, S, C (R)₂, N—Ar¹;
R is in each case independently a straight-chain or branched alkyl group which has 1 to 4 carbon atoms and may be partly or fully deuterated, or an unsubstituted or partly or fully deuterated aromatic ring system having 6 to 18 carbon atoms, where two substituents R together with the carbon atom to which they are bonded may form a mono- or polycyclic, aliphatic or aromatic or heteroaromatic, unsubstituted, partly deuterated or fully deuterated ring system which may be substituted by one or more substituents R⁵;
Ar¹ is the same or different at each instance and is an aromatic or heteroaromatic ring system which has 5 to 30 ring atoms and may be substituted by one or more R⁵ radicals; at the same time, two Ar¹ radicals bonded to the same nitrogen atom, phosphorus atom or boron atom may also be bridged to one another by a single bond or a bridge selected from C (R⁵)₂, O or S;
R¹ is the same or different at each instance and is selected from the group consisting of F, Cl, Br, I, CN, NO₂, C(═O)R′, P(═O)(Ar¹)₂, P(Ar¹)₂, B(Ar¹)₂, Si(Ar¹)₃, Si(R′)₃, a straight-chain alkyl, alkoxy or thioalkyl group having 1 to 20 carbon atoms and a branched or cyclic alkyl, alkoxy or thioalkyl group having 3 to carbon atoms and an alkenyl group having 2 to 20 carbon atoms, each of which may be substituted by one or more R′ radicals, where one or more nonadjacent CH₂ groups may be replaced by R′C═CR′, Si(R′)₂, C═O, C═S, C═NR′, P(═O)(R′), SO, SO₂, NR′, O, S or CONR′ and where one or more hydrogen atoms may be replaced by D, F, Cl, Br, I, CN or NO₂;
R′ is the same or different at each instance and is an aliphatic, aromatic heteroaromatic organic radical, especially a hydrocarbyl radicals, having 1 to 20 carbon atoms;
R⁴ is the same or different at each instance and is selected from the group consisting of F, Cl, Br, I, CN, NO₂, N(Ar₁)₂, NH₂, N(R⁵)₂, C(═O)Ar₁, C(═O)H, C(═O)R⁵, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R⁵ radicals, where one or more nonadjacent CH₂ groups may be replaced by HC═CH, R⁵C═CR⁵, C═C, Si(R⁵)₂, Ge(R⁵)₂, Sn(R⁵)₂, C═O, C═S, C═Se, C═NR⁵, P(═O)(R⁵), SO, SO₂, NH, NR⁵, O, S, CONH or CONR⁵ and where one or more hydrogen atoms may be replaced by D, F, C, Br, I, CN or NO₂, an aromatic or heteroaromatic ring system which has 5 to 60 ring atoms and may be substituted in each case by one or more R⁵ radicals, an aryloxy or heteroaryloxy group which has 5 to 60 ring atoms and may be substituted by one or more R⁵ radicals, or a combination of these systems, where it is optionally possible for two or more adjacent substituents R⁴ to form a monocyclic or polycyclic, aliphatic, aromatic or heteroaromatic ring system which may be substituted by one or more R⁵ radicals;
R⁵ is the same or different at each instance and is selected from the group consisting of D, F, CN, a straight-chain alkyl group having 1 to 20 carbon atoms or a branched or cyclic alkyl group having 3 to 20 carbon atoms, where one or more nonadjacent CH₂ groups may be replaced by 0 or S and where one or more hydrogen atoms may be replaced by D, F or CN, or an aromatic or heteroaromatic ring system which has 5 to 30 ring atoms and in which one or more hydrogen atoms may be replaced by D, F, C, Br, I or CN and which may be substituted by one or more alkyl groups each having 1 to 4 carbon atoms; it is possible here for two or more adjacent substituents R⁵ together to form a mono-or polycyclic, aliphatic ring system;
x, x1 at each instance are independently 0, 1, 2, 3 or 4;
y, z are each independently 0, 1 or 2;
a1, a2 are each independently 0, 1, 2, 3, 4 or 5;
a3 is 0, 1, 2 or 3;
a4 is 0, 1, 2, 3 or 4.

The preparation of the triarylamines of the formula (11) is known to the person skilled in the art, and some of the compounds are commercially available.

The compounds of the formulae (6), (7), (8), (9), (10) and (11) are preferably partly deuterated or fully deuterated.

In compounds of the formula (11) as described above, the sum total of the indices a₁+a₂+a₃+a₄ is preferably selected from 3, 4, 5, 6, 7, 8, 9, 10, 11, 12, 13, 14, 15, 16 and 17. This further matrix material is accordingly at least partly deuterated on each N-bonded substituent. In a preferred embodiment, two of the N-bonded substituents are partly deuterated and the third N-bonded substituent is fully deuterated. In a further preferred embodiment, two of the N-bonded substituents are fully deuterated and the third N-bonded substituent is partly deuterated. In a further preferred embodiment, each N-bonded substituent is fully deuterated.

In a preferred embodiment of the further matrix material, the latter is a mixture of deuterated compounds of the formula (11) as described above or described as preferred hereinafter, where the degree of deuteration of the compounds of the formula (11) is at least 50% to 90%, preferably 70% to 100%. Corresponding deuteration methods are known to the person skilled in the art and are described, for example, in KR2016041014, WO2017122988, KR202005282, KR101978651 and WO2018110887 or in Bulletin of the Chemical Society of Japan, 2021, 94(2), 600-605 or Asian Journal of Organic Chemistry, 2017, 6(8), 1063-1071.

A suitable method of deuterating an arylamine or a heteroarylamine by exchange of one or more hydrogen atoms for deuterium atoms is a treatment of the arylamine or a heteroarylamine to be deuterated in the presence of a platinum catalyst or palladium catalyst and a deuterium source. The term “deuterium source” means any compound that contains one or more deuterium atoms and is able to release them under suitable conditions.

The platinum catalyst is preferably dry platinum on charcoal, preferably 5% dry platinum on charcoal. The palladium catalyst is preferably dry palladium on charcoal, preferably 5% dry palladium on charcoal. A suitable deuterium source is D₂O, benzene-d6, chloroform-d, acetonitrile-d3, acetone-d6, acetic acid-d4, methanol-d4, toluene-d8. A preferred deuterium source is D₂O or a combination of D₂O and a fully deuterated organic solvent.

A particularly preferred deuterium source is the combination of D₂O with a fully deuterated organic solvent, where the fully deuterated solvent here is not restricted. Particularly suitable fully deuterated solvents are benzene-d6 and toluene-d8. A particularly preferred deuterium source is a combination of 020 and toluene-d8. The reaction is preferably conducted with heating, more preferably with heating to temperatures between 1000° and 200° C. In addition, the reaction is preferably conducted under pressure.

Preferred compounds of the formula ((1) are represented by the formulae (11a), (11b), (11c), (11d), (11e), (11f), (11g), (11h), (11i), (11j), (11k), (11l), (11m), (11n), (11o) and (11p):

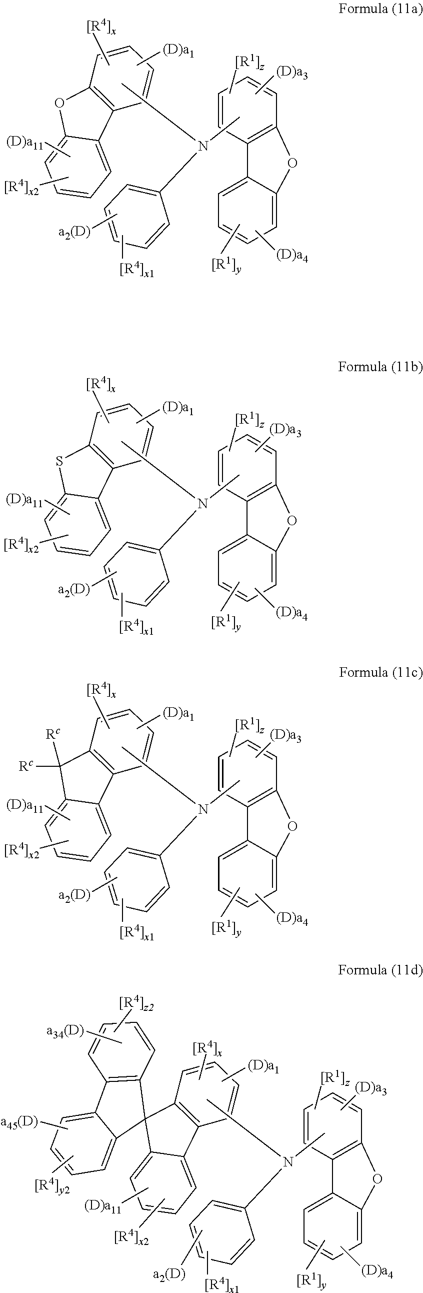

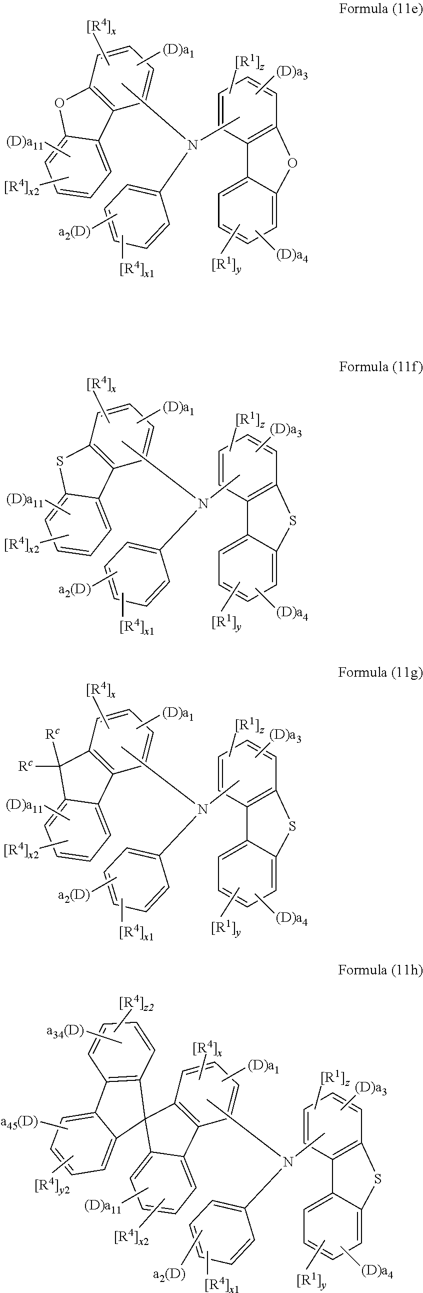

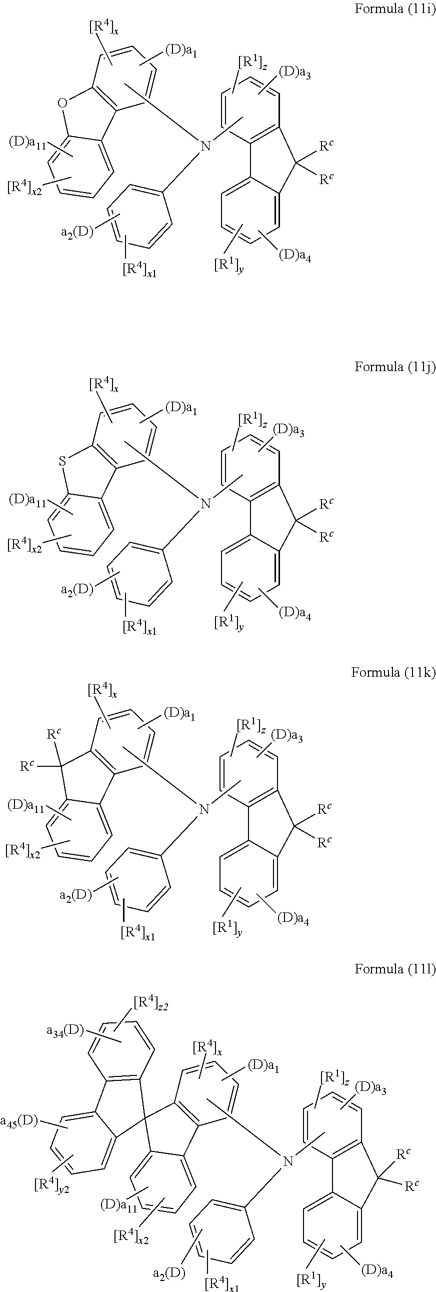

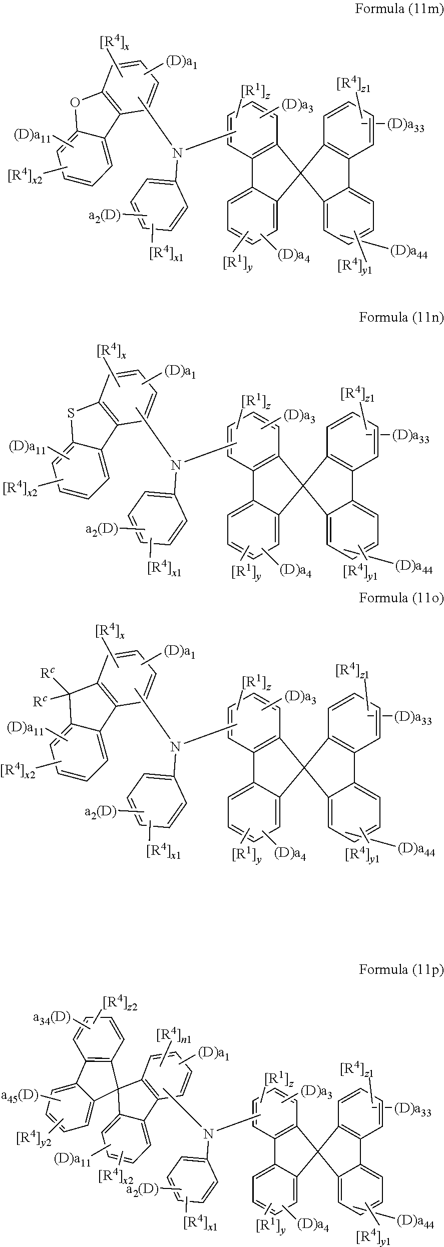

where a₁, a₂, a₃, a₄, x, x1, y, z, R¹ and R⁴ have a definition given above or given as preferred hereinafter and
R^{c} is in each case independently a straight-chain or branched alkyl group which has 1 to 4 carbon atoms and may be partly or fully deuterated, or an unsubstituted or partly or fully deuterated aromatic ring system having 6 to 18 carbon atoms;
x2 is 0, 1, 3 or 3;
y1, z1 are each independently 0, 1 or 2;
y1, z1, y2, z2 are each independently 0, 1 or 2, preferably 0;
a₁₁ is 0, 1, 2, 3 or 4;
a₃₃, a₄₄ are each independently 0, 1, 2, 3 or 4 and
a₃₄, a₄₅ are each independently 0, 1, 2, 3 or 4.

R^{c} is preferably the same and is a straight-chain or branched alkyl group which has 1 to 4 carbon atoms and may be partly or fully deuterated, or an unsubstituted or partly or fully deuterated phenyl.

In the compounds of the formulae (11), (11a), (11b), (11c), (11d), (11e), (11f), (11g), (11h), (11i), (11j), (11k), (11l), (11m), (11n), (110) and (11p), y+z is preferably 0.

The nitrogen atom in compounds of the formulae (11), (11a), (11b), (11c), (11d), (11e), (11f), (11g), (11h), (11i), (11j), (11k), (11l), (11m), (11n), (110) and (11p) is bonded in the 1 position to dibenzofuran or dibenzothiophene groups or bonded in the 4 position to fluorene or spirobifluorene groups.

Preferably, R⁴ in compounds of the formulae (11), (11a), (11b), (11c), (11d), (11e), (11f), (11g), (11h), (11i), (11j), (11k), (11l), (11m), (11n), (110) and (11p) is selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorenyl which may be joined via the 1, 2, 3 or 4 position, spirobifluorenyl which may be joined via the 1, 2, 3 or 4 position, naphthyl, especially 1- or 2-bonded naphthyl, or radicals derived from indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, phenanthrene or triphenylene, each of which may be substituted by one or more R⁵ radicals. Preferably, R⁴ is unsubstituted.

More preferably, the compounds of the formulae (6), (9), (10) and (11) are used as further matrix material.

Particularly suitable compounds of the formulae (6), (7), (8), (9), (10) and (11) that are selected in accordance with the invention and are preferably used in combination with at least one compound of the formula (1) in the electroluminescent device of the invention are the compounds H1 to H54 in table 3.

TABLE 3

| | |
|---|---|
| 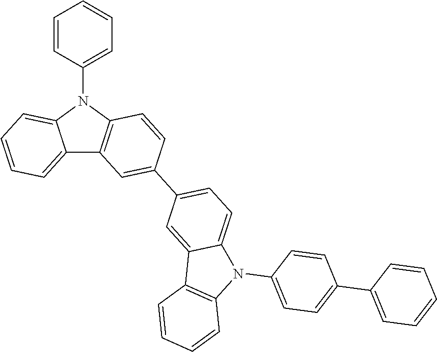 [SMILES: c1ccc(-c2ccc(-n3c4ccccc4c4cc(-c5ccc6c(c5)c5ccccc5n6-c5ccccc5)ccc43)cc2)cc1] | H1 |
| 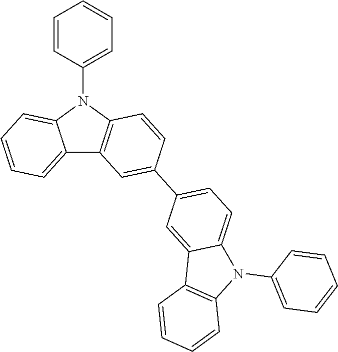 [SMILES: c1ccc(-n2c3ccccc3c3cc(-c4ccc5c(c4)c4ccccc4n5-c4ccccc4)ccc32)cc1] | H2 |
| 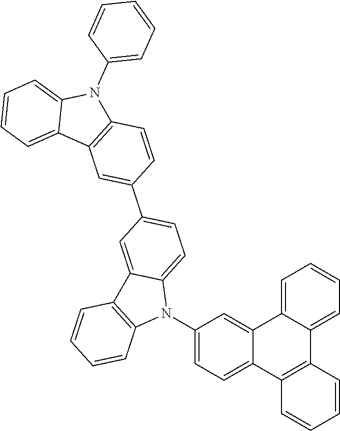 [SMILES: c1ccc(-n2c3ccccc3c3cc(-c4ccc5c(c4)c4ccccc4n5-c4ccc5c6ccccc6c6ccccc6c5c4)ccc32)cc1] | H3 |
| 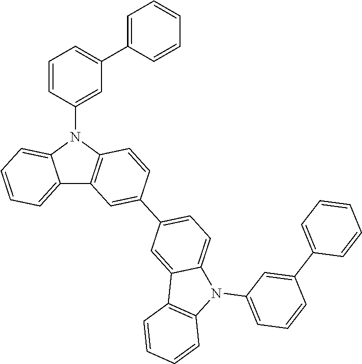 [SMILES: c1ccc(-c2cccc(-n3c4ccccc4c4cc(-c5ccc6c(c5)c5ccccc5n6-c5cccc(-c6ccccc6)c5)ccc43)c2)cc1] | H4 |
| 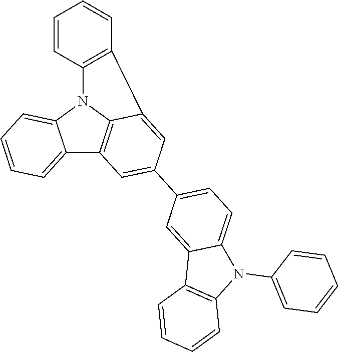 [SMILES: c1ccc(-n2c3ccccc3c3cc(-c4cc5c6ccccc6n6c7ccccc7c(c4)c56)ccc32)cc1] | H5 |
| 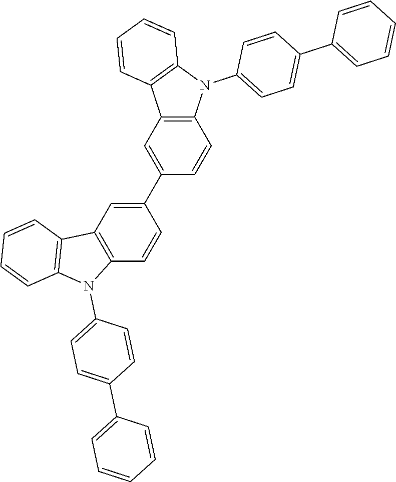 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | H6 |
| 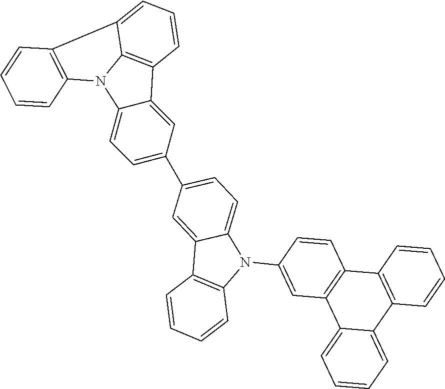 [SMILES: c1ccc2c(c1)c1ccccc1c1cc(-n3c4ccccc4c4cc(-c5ccc6c(c5)c5cccc7c8ccccc8n6c75)ccc43)ccc21] | H7 |
| 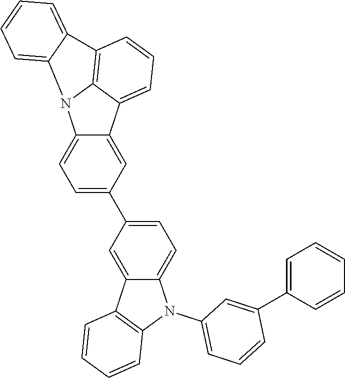 [SMILES: c1ccc(-c2cccc(-n3c4ccccc4c4cc(-c5ccc6c(c5)c5cccc7c8ccccc8n6c75)ccc43)c2)cc1] | H8 |
| 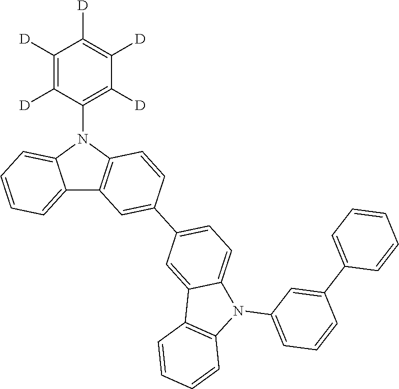 [SMILES: [2H]c1c([2H])c([2H])c(-n2c3ccccc3c3cc(-c4ccc5c(c4)c4ccccc4n5-c4cccc(-c5ccccc5)c4)ccc32)c([2H])c1[2H]] | H9 |
| 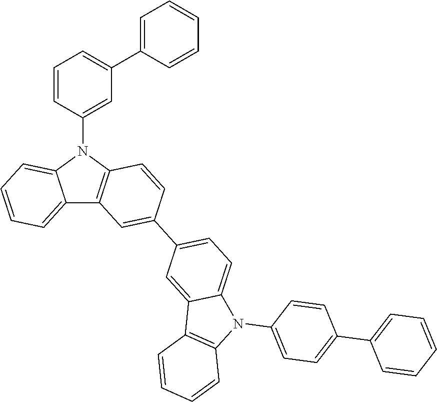 [SMILES: c1ccc(-c2ccc(-n3c4ccccc4c4cc(-c5ccc6c(c5)c5ccccc5n6-c5cccc(-c6ccccc6)c5)ccc43)cc2)cc1] | H10 |
| 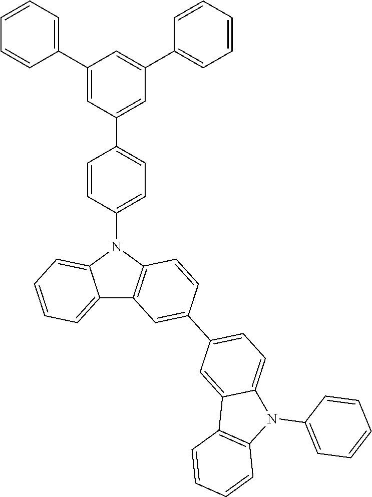 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1] | H11 |
| 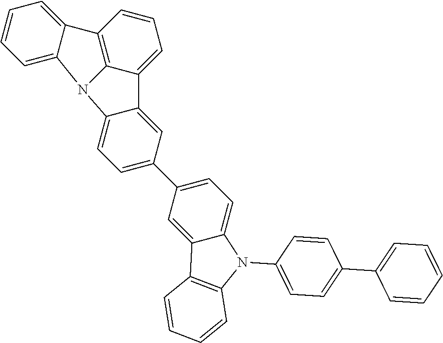 [SMILES: c1ccc(-c2ccc(-n3c4ccccc4c4cc(-c5ccc6c(c5)c5cccc7c8ccccc8n6c75)ccc43)cc2)cc1] | H12 |
| 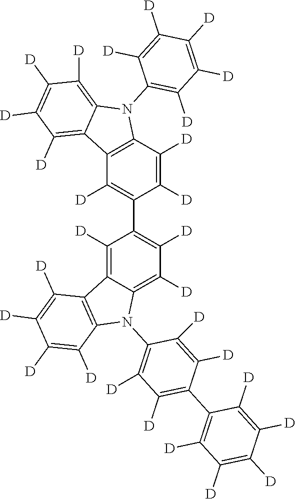 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]] | H13 |
| 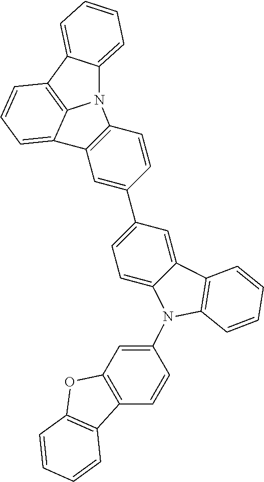 [SMILES: c1ccc2c(c1)oc1cc(-n3c4ccccc4c4cc(-c5ccc6c(c5)c5cccc7c8ccccc8n6c75)ccc43)ccc12] | H14 |
| 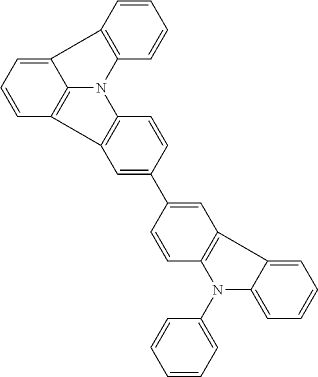 [SMILES: c1ccc(-n2c3ccccc3c3cc(-c4ccc5c(c4)c4cccc6c7ccccc7n5c64)ccc32)cc1] | H15 |
| 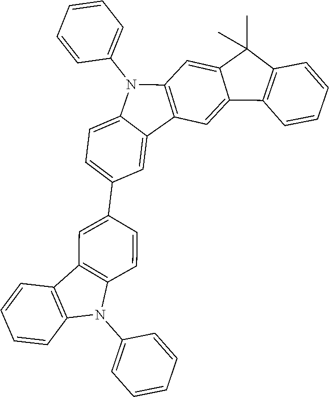 [SMILES: CC1(C)c2ccccc2-c2cc3c4cc(-c5ccc6c(c5)c5ccccc5n6-c5ccccc5)ccc4n(-c4ccccc4)c3cc21] | H16 |
| 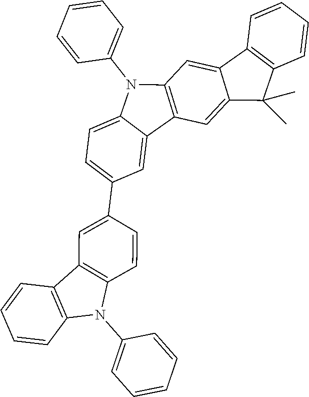 [SMILES: CC1(C)c2ccccc2-c2cc3c(cc21)c1cc(-c2ccc4c(c2)c2ccccc2n4-c2ccccc2)ccc1n3-c1ccccc1] | H17 |
| 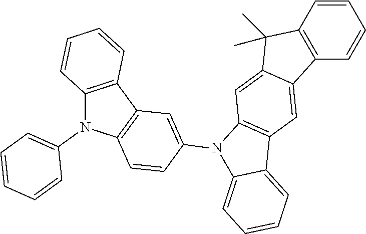 [SMILES: CC1(C)c2ccccc2-c2cc3c4ccccc4n(-c4ccc5c(c4)c4ccccc4n5-c4ccccc4)c3cc21] | H18 |
| 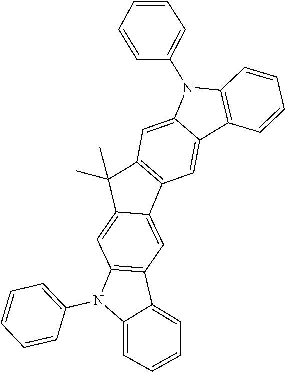 [SMILES: CC1(C)c2cc3c(cc2-c2cc4c5ccccc5n(-c5ccccc5)c4cc21)c1ccccc1n3-c1ccccc1] | H19 |
| 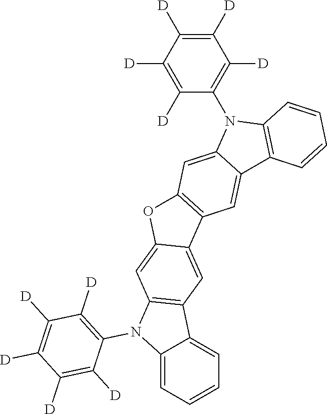 [SMILES: [2H]c1c([2H])c([2H])c(-n2c3ccccc3c3cc4c(cc32)oc2cc3c(cc24)c2ccccc2n3-c2c([2H])c([2H])c([2H])c([2H])c2[2H])c([2H])c1[2H]] | H20 |
| 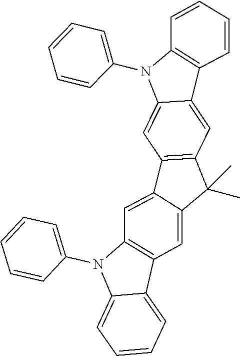 [SMILES: CC1(C)c2cc3c4ccccc4n(-c4ccccc4)c3cc2-c2cc3c(cc21)c1ccccc1n3-c1ccccc1] | H21 |
| 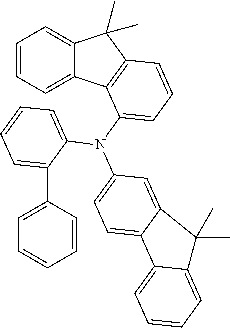 [SMILES: CC1(C)c2ccccc2-c2ccc(N(c3ccccc3-c3ccccc3)c3cccc4c3-c3ccccc3C4(C)C)cc21] | H22 |
| 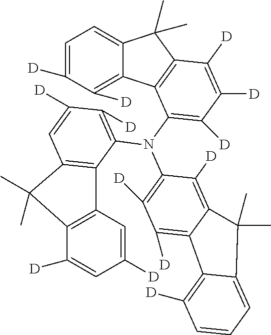 | H23 |
| 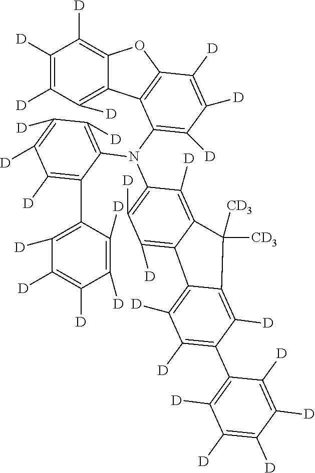 [SMILES: [2H]c1c([2H])c([2H])c(-c2c([2H])c([2H])c3c(c2[2H])C(C([2H])([2H])[2H])(C([2H])([2H])[2H])c2c([2H])c(N(c4c([2H])c([2H])c([2H])c([2H])c4-c4c([2H])c([2H])c([2H])c([2H])c4[2H])c4c([2H])c([2H])c([2H])c5oc6c([2H])c([2H])c([2H])c([2H])c6c45)c([2H])c([2H])c2-3)c([2H])c1[2H]] | H24 |
| 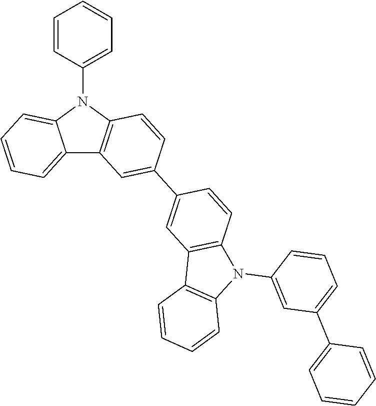 [SMILES: c1ccc(-c2cccc(-n3c4ccccc4c4cc(-c5ccc6c(c5)c5ccccc5n6-c5ccccc5)ccc43)c2)cc1] | H25 |
| 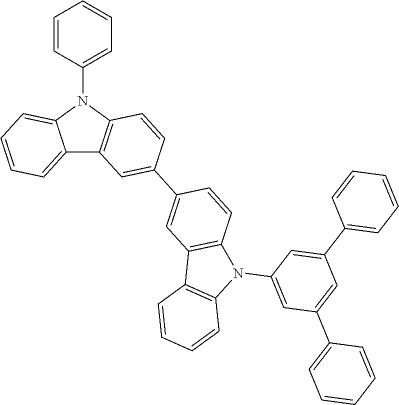 [SMILES: c1ccc(-c2cc(-c3ccccc3)cc(-n3c4ccccc4c4cc(-c5ccc6c(c5)c5ccccc5n6-c5ccccc5)ccc43)c2)cc1] | H26 |
| 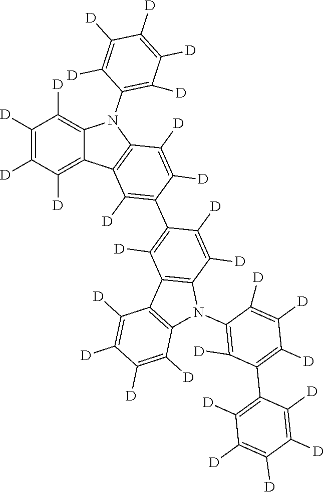 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]] | H27 |
| 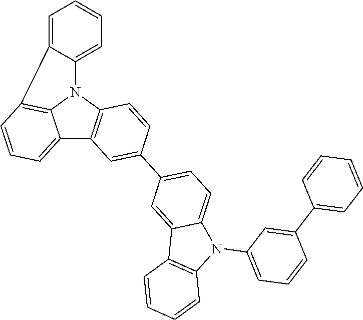 [SMILES: c1ccc(-c2cccc(-n3c4ccccc4c4cc(-c5ccc6c(c5)c5cccc7c8ccccc8n6c75)ccc43)c2)cc1] | H28 |
| 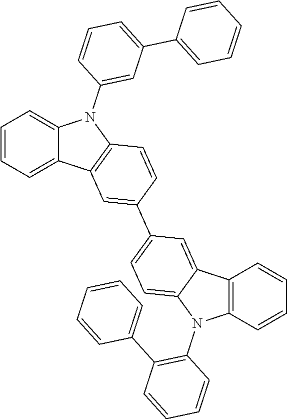 [SMILES: c1ccc(-c2cccc(-n3c4ccccc4c4cc(-c5ccc6c(c5)c5ccccc5n6-c5ccccc5-c5ccccc5)ccc43)c2)cc1] | H29 |
| 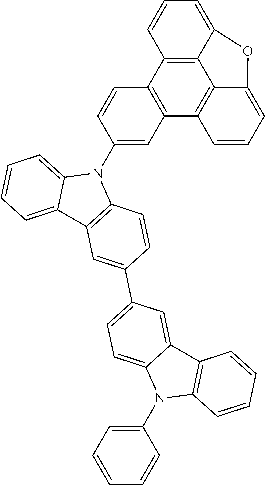 [SMILES: c1ccc(-n2c3ccccc3c3cc(-c4ccc5c(c4)c4ccccc4n5-c4ccc5c(c4)c4cccc6oc7cccc5c7c64)ccc32)cc1] | H30 |
| 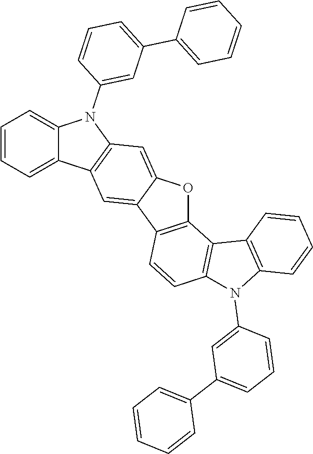 [SMILES: c1ccc(-c2cccc(-n3c4ccccc4c4cc5c(cc43)oc3c5ccc4c3c3ccccc3n4-c3cccc(-c4ccccc4)c3)c2)cc1] | H31 |
| 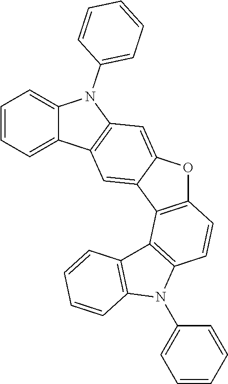 [SMILES: c1ccc(-n2c3ccccc3c3cc4c(cc32)oc2ccc3c(c5ccccc5n3-c3ccccc3)c24)cc1] | H32 |
| 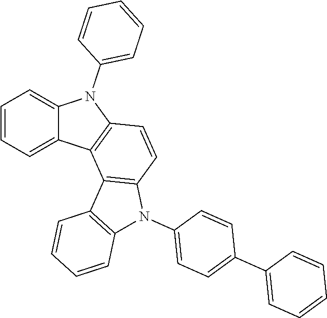 [SMILES: c1ccc(-c2ccc(-n3c4ccccc4c4c5c6ccccc6n(-c6ccccc6)c5ccc43)cc2)cc1] | H33 |
| 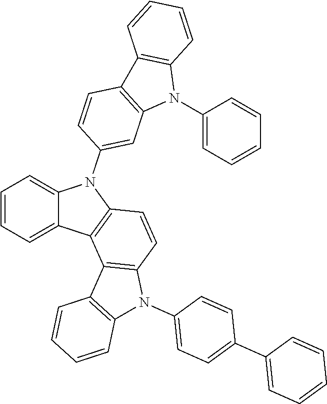 [SMILES: c1ccc(-c2ccc(-n3c4ccccc4c4c5c6ccccc6n(-c6ccc7c8ccccc8n(-c8ccccc8)c7c6)c5ccc43)cc2)cc1] | H34 |
| 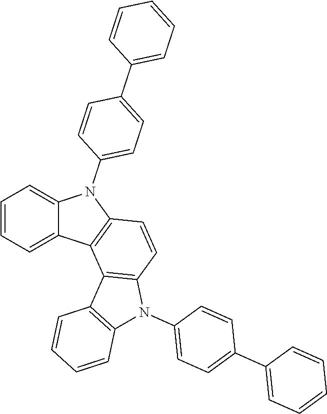 [SMILES: c1ccc(-c2ccc(-n3c4ccccc4c4c5c6ccccc6n(-c6ccc(-c7ccccc7)cc6)c5ccc43)cc2)cc1] | H35 |
| 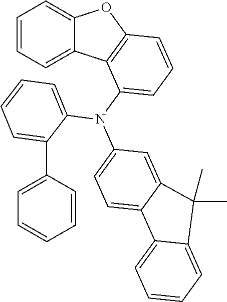 [SMILES: CC1(C)c2ccccc2-c2ccc(N(c3ccccc3-c3ccccc3)c3cccc4oc5ccccc5c34)cc21] | H36 |
| 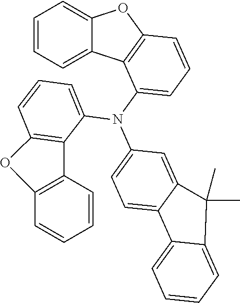 [SMILES: CC1(C)c2ccccc2-c2ccc(N(c3cccc4oc5ccccc5c34)c3cccc4oc5ccccc5c34)cc21] | H37 |
| 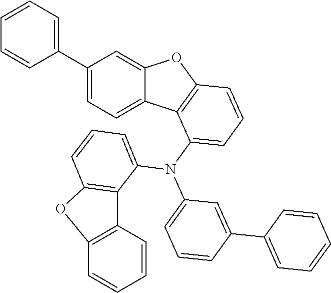 [SMILES: c1ccc(-c2cccc(N(c3cccc4oc5ccccc5c34)c3cccc4oc5cc(-c6ccccc6)ccc5c34)c2)cc1] | H38 |
| 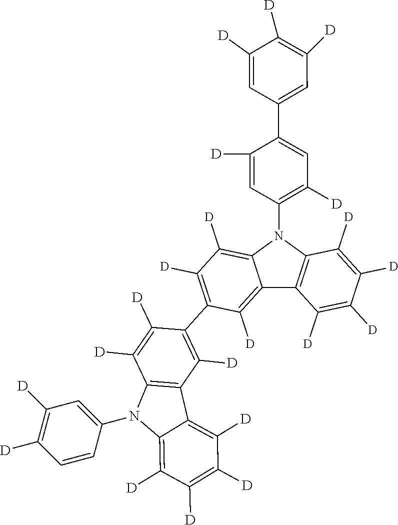 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc([2H])c(-c5cc([2H])c([2H])c([2H])c5)cc4[2H])c([2H])c([2H])c32)cc1[2H]] | H39 |
| 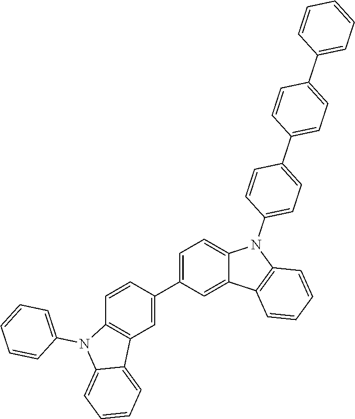 [SMILES: c1ccc(-c2ccc(-c3ccc(-n4c5ccccc5c5cc(-c6ccc7c(c6)c6ccccc6n7-c6ccccc6)ccc54)cc3)cc2)cc1] | H40 |
| 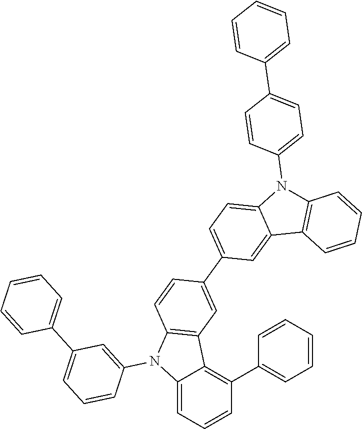 [SMILES: c1ccc(-c2ccc(-n3c4ccccc4c4cc(-c5ccc6c(c5)c5c(-c7ccccc7)cccc5n6-c5cccc(-c6ccccc6)c5)ccc43)cc2)cc1] | H41 |
| 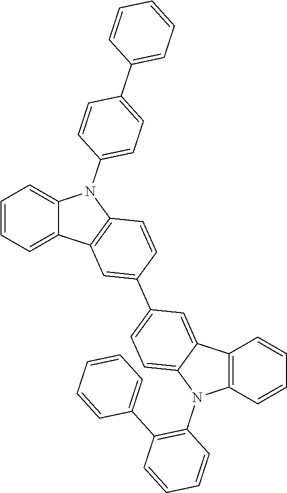 [SMILES: c1ccc(-c2ccc(-n3c4ccccc4c4cc(-c5ccc6c(c5)c5ccccc5n6-c5ccccc5-c5ccccc5)ccc43)cc2)cc1] | H42 |
| 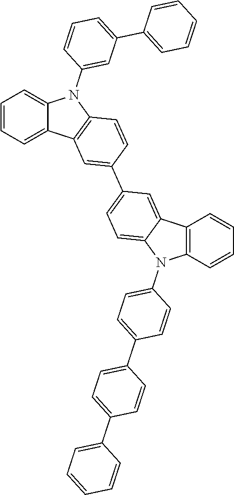 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] | H43 |
| 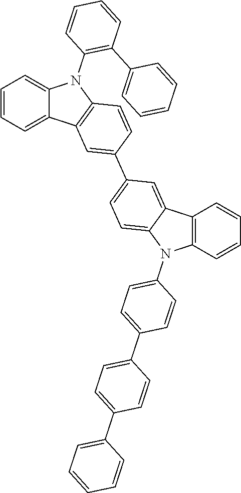 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] | H44 |
| 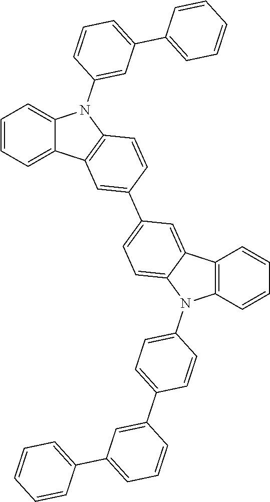 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1] | H45 |
| 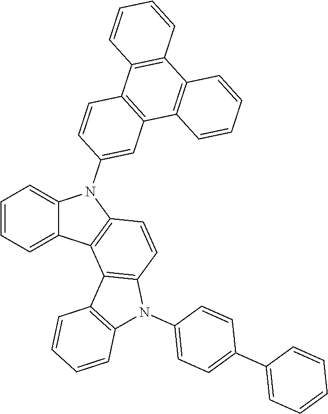 [SMILES: c1ccc(-c2ccc(-n3c4ccccc4c4c5c6ccccc6n(-c6ccc7c8ccccc8c8ccccc8c7c6)c5ccc43)cc2)cc1] | H46 |
| 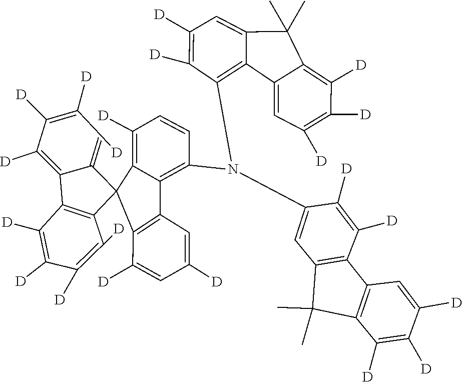 [SMILES: [2H]C([2H])=C([2H])/C([2H])=C1\C(=C)C2(c3c([2H])cc([2H])cc3-c3c(N(c4cc5c(c([2H])c4[2H])-c4cc([2H])c([2H])c([2H])c4C5(C)C)c4c([2H])c([2H])cc5c4-c4cc([2H])c([2H])c([2H])c4C5(C)C)ccc([2H])c32)c2c([2H])c([2H])c([2H])c([2H])c21] | H47 |
| 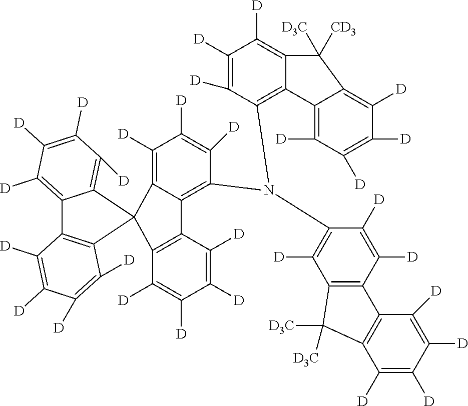 [SMILES: [2H]c1c([2H])c([2H])c2c(c1[2H])-c1c([2H])c([2H])c([2H])c([2H])c1C21c2c([2H])c([2H])c([2H])c([2H])c2-c2c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c([2H])c4c3-c3c([2H])c([2H])c([2H])c([2H])c3C4(C([2H])([2H])[2H])C([2H])([2H])[2H])c([2H])c([2H])c([2H])c21] | H48 |
| 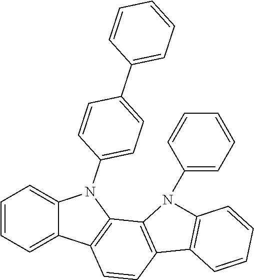 [SMILES: c1ccc(-c2ccc(-n3c4ccccc4c4ccc5c6ccccc6n(-c6ccccc6)c5c43)cc2)cc1] | H49 |
| 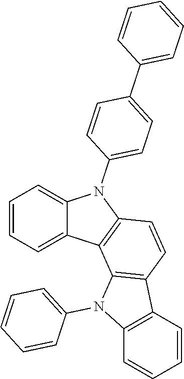 [SMILES: c1ccc(-c2ccc(-n3c4ccccc4c4c3ccc3c5ccccc5n(-c5ccccc5)c34)cc2)cc1] | H50 |
| 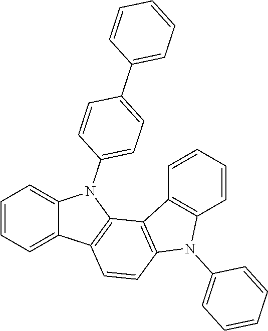 [SMILES: c1ccc(-c2ccc(-n3c4ccccc4c4ccc5c(c6ccccc6n5-c5ccccc5)c43)cc2)cc1] | H51 |
| 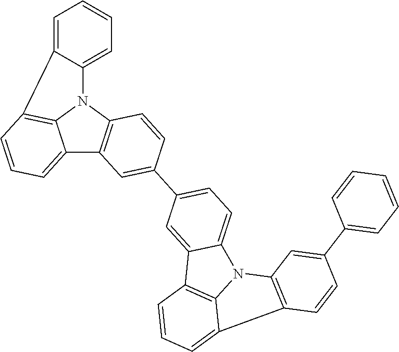 [SMILES: c1ccc(-c2ccc3c4cccc5c6cc(-c7ccc8c(c7)c7cccc9c%10ccccc%10n8c97)ccc6n(c3c2)c54)cc1] | H52 |
| 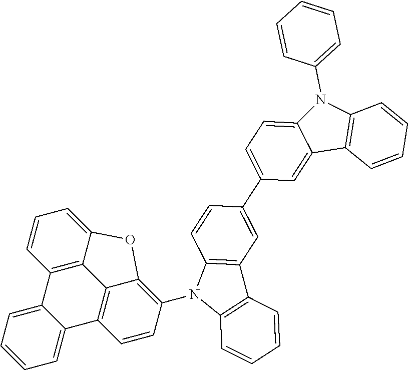 [SMILES: c1ccc(-n2c3ccccc3c3cc(-c4ccc5c(c4)c4ccccc4n5-c4ccc5c6ccccc6c6cccc7oc4c5c76)ccc32)cc1] | H53 |
| 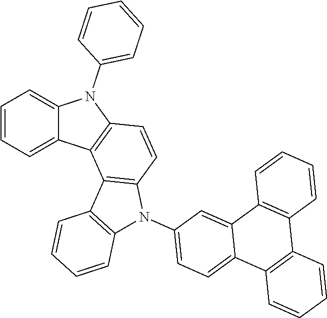 [SMILES: c1ccc(-n2c3ccccc3c3c4c5ccccc5n(-c5ccc6c7ccccc7c7ccccc7c6c5)c4ccc32)cc1] | H54 |

The aforementioned host materials of the formula (1) and the embodiments thereof that are described as preferred or the compounds from table 1 and compounds EG1 to EG18 can be combined as desired in the device of the invention with the cited matrix materials/host materials of the formulae (6), (7), (8), (9), (10) and (11) and the preferred embodiments thereof or compounds Hi to H54.

Very particularly preferred mixtures of the compounds of the formula (1) with the host materials of the formulae (6), (7), (8), (9), (10) and (11) for the device of the invention are obtained by combination of compounds EG1 to EG1B with compounds H1 to H54 as shown hereinafter in table 4.

TABLE 4

| | | | | | | | | |
|---|---|---|---|---|---|---|---|---|
| M1 | EG1 | H1 | M2 | EG2 | H1 | M3 | EG3 | H1 |
| M4 | EG4 | H1 | M5 | EG5 | H1 | M6 | EG6 | H1 |
| M7 | EG7 | H1 | M8 | EG8 | H1 | M9 | EG9 | H1 |
| M10 | EG10 | H1 | M11 | EG11 | H1 | M12 | EG12 | H1 |
| M13 | EG13 | H1 | M14 | EG14 | H1 | M15 | EG15 | H1 |
| M16 | EG16 | H1 | M17 | EG17 | H1 | M18 | EG18 | H1 |
| M19 | EG1 | H2 | M20 | EG2 | H2 | M21 | EG3 | H2 |
| M22 | EG4 | H2 | M23 | EG5 | H2 | M24 | EG6 | H2 |
| M25 | EG7 | H2 | M26 | EG8 | H2 | M27 | EG9 | H2 |
| M28 | EG10 | H2 | M29 | EG11 | H2 | M30 | EG12 | H2 |
| M31 | EG13 | H2 | M32 | EG14 | H2 | M33 | EG15 | H2 |
| M34 | EG16 | H2 | M35 | EG17 | H2 | M36 | EG18 | H2 |
| M37 | EG1 | H3 | M38 | EG2 | H3 | M39 | EG3 | H3 |
| M40 | EG4 | H3 | M41 | EG5 | H3 | M42 | EG6 | H3 |
| M43 | EG7 | H3 | M44 | EG8 | H3 | M45 | EG9 | H3 |
| M46 | EG10 | H3 | M47 | EG11 | H3 | M48 | EG12 | H3 |
| M49 | EG13 | H3 | M50 | EG14 | H3 | M51 | EG15 | H3 |
| M52 | EG16 | H3 | M53 | EG17 | H3 | M54 | EG18 | H3 |
| M55 | EG1 | H4 | M56 | EG2 | H4 | M57 | EG3 | H4 |
| M58 | EG4 | H4 | M59 | EG5 | H4 | M60 | EG6 | H4 |
| M61 | EG7 | H4 | M62 | EG8 | H4 | M63 | EG9 | H4 |
| M64 | EG10 | H4 | M65 | EG11 | H4 | M66 | EG12 | H4 |
| M67 | EG13 | H4 | M68 | EG14 | H4 | M69 | EG15 | H4 |
| M70 | EG16 | H4 | M71 | EG17 | H4 | M72 | EG18 | H4 |
| M73 | EG1 | H5 | M74 | EG2 | H5 | M75 | EG3 | H5 |
| M76 | EG4 | H5 | M77 | EG5 | H5 | M78 | EG6 | H5 |
| M79 | EG7 | H5 | M80 | EG8 | H5 | M81 | EG9 | H5 |
| M82 | EG10 | H5 | M83 | EG11 | H5 | M84 | EG12 | H5 |
| M85 | EG13 | H5 | M86 | EG14 | H5 | M87 | EG15 | H5 |
| M88 | EG16 | H5 | M89 | EG17 | H5 | M90 | EG18 | H5 |
| M91 | EG1 | H6 | M92 | EG2 | H6 | M93 | EG3 | H6 |
| M94 | EG4 | H6 | M95 | EG5 | H6 | M96 | EG6 | H6 |
| M97 | EG7 | H6 | M98 | EG8 | H6 | M99 | EG9 | H6 |
| M100 | EG10 | H6 | M101 | EG11 | H6 | M102 | EG12 | H6 |
| M103 | EG13 | H6 | M104 | EG14 | H6 | M105 | EG15 | H6 |
| M106 | EG16 | H6 | M107 | EG17 | H6 | M108 | EG18 | H6 |
| M109 | EG1 | H7 | M110 | EG2 | H7 | M111 | EG3 | H7 |
| M112 | EG4 | H7 | M113 | EG5 | H7 | M114 | EG6 | H7 |
| M115 | EG7 | H7 | M116 | EG8 | H7 | M117 | EG9 | H7 |
| M118 | EG10 | H7 | M119 | EG11 | H7 | M120 | EG12 | H7 |
| M121 | EG13 | H7 | M122 | EG14 | H7 | M123 | EG15 | H7 |
| M124 | EG16 | H7 | M125 | EG17 | H7 | M126 | EG18 | H7 |
| M127 | EG1 | H8 | M128 | EG2 | H4 | M129 | EG3 | H4 |
| M130 | EG4 | H8 | M131 | EG5 | H8 | M132 | EG6 | H8 |
| M133 | EG7 | H8 | M134 | EG8 | H8 | M135 | EG9 | H8 |
| M136 | EG10 | H8 | M137 | EG11 | H8 | M138 | EG12 | H8 |
| M139 | EG13 | H8 | M140 | EG14 | H8 | M141 | EG15 | H8 |
| M142 | EG16 | H8 | M143 | EG17 | H8 | M144 | EG18 | H8 |
| M145 | EG1 | H9 | M146 | EG2 | H9 | M147 | EG3 | H9 |
| M148 | EG4 | H9 | M149 | EG5 | H9 | M150 | EG6 | H9 |
| M151 | EG7 | H9 | M152 | EG8 | H9 | M153 | EG9 | H9 |
| M154 | EG10 | H9 | M155 | EG11 | H9 | M156 | EG12 | H9 |
| M157 | EG13 | H9 | M158 | EG14 | H9 | M159 | EG15 | H9 |
| M160 | EG16 | H9 | M161 | EG17 | H9 | M162 | EG18 | H9 |
| M163 | EG1 | H10 | M164 | EG2 | H10 | M165 | EG3 | H10 |
| M166 | EG4 | H10 | M167 | EG5 | H10 | M168 | EG6 | H10 |
| M169 | EG7 | H10 | M170 | EG8 | H10 | M171 | EG9 | H10 |
| M172 | EG10 | H10 | M173 | EG11 | H10 | M174 | EG12 | H10 |
| M175 | EG13 | H10 | M176 | EG14 | H10 | M177 | EG15 | H10 |
| M178 | EG16 | H10 | M179 | EG17 | H10 | M180 | EG18 | H10 |
| M181 | EG1 | H11 | M182 | EG2 | H11 | M183 | EG3 | H11 |
| M184 | EG4 | H11 | M185 | EG5 | H11 | M186 | EG6 | H11 |
| M187 | EG7 | H11 | M188 | EG8 | H11 | M189 | EG9 | H11 |
| M190 | EG10 | H11 | M191 | EG11 | H11 | M192 | EG12 | H11 |
| M193 | EG13 | H11 | M194 | EG14 | H11 | M195 | EG15 | H11 |
| M196 | EG16 | H11 | M197 | EG17 | H11 | M198 | EG18 | H11 |
| M199 | EG1 | H12 | M200 | EG2 | H12 | M201 | EG3 | H12 |
| M202 | EG4 | H12 | M203 | EG5 | H12 | M204 | EG6 | H12 |
| M205 | EG7 | H12 | M206 | EG8 | H12 | M207 | EG9 | H12 |
| M208 | EG10 | H12 | M209 | EG11 | H12 | M210 | EG12 | H12 |
| M211 | EG13 | H12 | M212 | EG14 | H12 | M213 | EG15 | H12 |
| M214 | EG16 | H12 | M215 | EG17 | H12 | M216 | EG18 | H12 |
| M217 | EG1 | H13 | M218 | EG2 | H13 | M219 | EG3 | H13 |
| M220 | EG4 | H13 | M221 | EG5 | H13 | M222 | EG6 | H13 |
| M223 | EG7 | H13 | M224 | EG8 | H13 | M225 | EG9 | H13 |
| M226 | EG10 | H13 | M227 | EG11 | H13 | M228 | EG12 | H13 |
| M229 | EG13 | H13 | M230 | EG14 | H13 | M231 | EG15 | H13 |
| M232 | EG16 | H13 | M233 | EG17 | H13 | M234 | EG18 | H13 |
| M235 | EG1 | H14 | M236 | EG2 | H14 | M237 | EG3 | H14 |
| M238 | EG4 | H14 | M239 | EG5 | H14 | M240 | EG6 | H14 |
| M241 | EG7 | H14 | M242 | EG8 | H14 | M243 | EG9 | H14 |
| M244 | EG10 | H14 | M245 | EG11 | H14 | M246 | EG12 | H14 |
| M247 | EG13 | H14 | M248 | EG14 | H14 | M249 | EG15 | H14 |
| M250 | EG16 | H14 | M251 | EG17 | H14 | M252 | EG18 | H14 |
| M253 | EG1 | H15 | M254 | EG2 | H15 | M255 | EG3 | H15 |
| M256 | EG4 | H15 | M257 | EG5 | H15 | M258 | EG6 | H15 |
| M259 | EG7 | H15 | M260 | EG8 | H15 | M261 | EG9 | H15 |
| M262 | EG10 | H15 | M263 | EG11 | H15 | M264 | EG12 | H15 |
| M265 | EG13 | H15 | M266 | EG14 | H15 | M267 | EG15 | H15 |
| M268 | EG16 | H15 | M269 | EG17 | H15 | M270 | EG18 | H15 |
| M271 | EG1 | H16 | M272 | EG2 | H16 | M273 | EG3 | H16 |
| M274 | EG4 | H16 | M275 | EG5 | H16 | M276 | EG6 | H16 |
| M277 | EG7 | H16 | M278 | EG8 | H16 | M279 | EG9 | H16 |
| M280 | EG10 | H16 | M281 | EG11 | H16 | M282 | EG12 | H16 |
| M283 | EG13 | H16 | M284 | EG14 | H16 | M285 | EG15 | H16 |
| M286 | EG16 | H16 | M287 | EG17 | H16 | M288 | EG18 | H16 |
| M289 | EG1 | H17 | M290 | EG2 | H17 | M291 | EG3 | H17 |
| M292 | EG4 | H17 | M293 | EG5 | H17 | M294 | EG6 | H17 |
| M295 | EG7 | H17 | M296 | EG8 | H17 | M297 | EG9 | H17 |
| M298 | EG10 | H17 | M299 | EG11 | H17 | M300 | EG12 | H17 |
| M301 | EG13 | H17 | M302 | EG14 | H17 | M303 | EG15 | H17 |
| M304 | EG16 | H17 | M305 | EG17 | H17 | M306 | EG18 | H17 |
| M307 | EG1 | H18 | M308 | EG2 | H18 | M309 | EG3 | H18 |
| M310 | EG4 | H18 | M311 | EG5 | H18 | M312 | EG6 | H18 |
| M313 | EG7 | H18 | M314 | EG8 | H18 | M315 | EG9 | H18 |
| M316 | EG10 | H18 | M317 | EG11 | H18 | M318 | EG12 | H18 |
| M319 | EG13 | H18 | M320 | EG14 | H18 | M321 | EG15 | H18 |
| M322 | EG16 | H18 | M323 | EG17 | H18 | M324 | EG18 | H18 |
| M325 | EG1 | H19 | M326 | EG2 | H19 | M327 | EG3 | H19 |
| M328 | EG4 | H19 | M329 | EG5 | H19 | M330 | EG6 | H19 |
| M331 | EG7 | H19 | M332 | EG8 | H19 | M333 | EG9 | H19 |
| M334 | EG10 | H19 | M335 | EG11 | H19 | M336 | EG12 | H19 |
| M337 | EG13 | H19 | M338 | EG14 | H19 | M339 | EG15 | H19 |
| M340 | EG16 | H19 | M341 | EG17 | H19 | M342 | EG18 | H19 |
| M343 | EG1 | H20 | M344 | EG2 | H20 | M345 | EG3 | H20 |
| M346 | EG4 | H20 | M347 | EG5 | H20 | M348 | EG6 | H20 |
| M349 | EG7 | H20 | M350 | EG8 | H20 | M351 | EG9 | H20 |
| M352 | EG10 | H20 | M353 | EG11 | H20 | M354 | EG12 | H20 |
| M355 | EG13 | H20 | M356 | EG14 | H20 | M357 | EG15 | H20 |
| M358 | EG16 | H20 | M359 | EG17 | H20 | M360 | EG18 | H20 |
| M361 | EG1 | H21 | M362 | EG2 | H21 | M363 | EG3 | H21 |
| M364 | EG4 | H21 | M365 | EG5 | H21 | M366 | EG6 | H21 |
| M367 | EG7 | H21 | M368 | EG8 | H21 | M369 | EG9 | H21 |
| M370 | EG10 | H21 | M371 | EG11 | H21 | M372 | EG12 | H21 |
| M373 | EG13 | H21 | M374 | EG14 | H21 | M375 | EG15 | H21 |
| M376 | EG16 | H21 | M377 | EG17 | H21 | M378 | EG18 | H21 |
| M379 | EG1 | H22 | M380 | EG2 | H22 | M381 | EG3 | H22 |
| M382 | EG4 | H22 | M383 | EG5 | H22 | M384 | EG6 | H22 |
| M385 | EG7 | H22 | M386 | EG8 | H22 | M387 | EG9 | H22 |
| M388 | EG10 | H22 | M389 | EG11 | H22 | M390 | EG12 | H22 |
| M391 | EG13 | H22 | M392 | EG14 | H22 | M393 | EG15 | H22 |
| M394 | EG16 | H22 | M395 | EG17 | H22 | M396 | EG18 | H22 |
| M397 | EG1 | H23 | M398 | EG2 | H23 | M399 | EG3 | H23 |
| M400 | EG4 | H23 | M401 | EG5 | H23 | M402 | EG6 | H23 |
| M403 | EG7 | H23 | M404 | EG8 | H23 | M405 | EG9 | H23 |
| M406 | EG10 | H23 | M407 | EG11 | H23 | M408 | EG12 | H23 |
| M409 | EG13 | H23 | M410 | EG14 | H23 | M411 | EG15 | H23 |
| M412 | EG16 | H23 | M413 | EG17 | H23 | M414 | EG18 | H23 |
| M415 | EG1 | H24 | M416 | EG2 | H24 | M417 | EG3 | H24 |
| M418 | EG4 | H24 | M419 | EG5 | H24 | M420 | EG6 | H24 |
| M421 | EG7 | H24 | M422 | EG8 | H24 | M423 | EG9 | H24 |
| M424 | EG10 | H24 | M425 | EG11 | H24 | M426 | EG12 | H24 |
| M427 | EG13 | H24 | M428 | EG14 | H24 | M429 | EG15 | H24 |
| M430 | EG16 | H24 | M431 | EG17 | H24 | M432 | EG18 | H24 |
| M433 | EG1 | H25 | M434 | EG2 | H25 | M435 | EG3 | H25 |
| M436 | EG4 | H25 | M437 | EG5 | H25 | M438 | EG6 | H25 |
| M439 | EG7 | H25 | M440 | EG8 | H25 | M441 | EG9 | H25 |
| M442 | EG10 | H25 | M443 | EG11 | H25 | M444 | EG12 | H25 |
| M445 | EG13 | H25 | M446 | EG14 | H25 | M447 | EG15 | H25 |
| M448 | EG16 | H25 | M449 | EG17 | H25 | M450 | EG18 | H25 |
| M451 | EG1 | H26 | M452 | EG2 | H26 | M453 | EG3 | H26 |
| M454 | EG4 | H26 | M455 | EG5 | H26 | M456 | EG6 | H26 |
| M457 | EG7 | H26 | M458 | EG8 | H26 | M459 | EG9 | H26 |
| M460 | EG10 | H26 | M461 | EG11 | H26 | M462 | EG12 | H26 |
| M463 | EG13 | H26 | M464 | EG14 | H26 | M465 | EG15 | H26 |
| M466 | EG16 | H26 | M467 | EG17 | H26 | M468 | EG18 | H26 |
| M469 | EG1 | H27 | M470 | EG2 | H27 | M471 | EG3 | H27 |
| M472 | EG4 | H27 | M473 | EG5 | H27 | M474 | EG6 | H27 |
| M475 | EG7 | H27 | M476 | EG8 | H27 | M477 | EG9 | H27 |
| M478 | EG10 | H27 | M479 | EG11 | H27 | M480 | EG12 | H27 |
| M481 | EG13 | H27 | M482 | EG14 | H27 | M483 | EG15 | H27 |
| M484 | EG16 | H27 | M485 | EG17 | H27 | M486 | EG18 | H27 |
| M487 | EG1 | H28 | M488 | EG2 | H28 | M489 | EG3 | H28 |
| M490 | EG4 | H28 | M491 | EG5 | H28 | M492 | EG6 | H28 |
| M493 | EG7 | H28 | M494 | EG8 | H28 | M495 | EG9 | H28 |
| M496 | EG10 | H28 | M497 | EG11 | H28 | M498 | EG12 | H28 |
| M499 | EG13 | H28 | M500 | EG14 | H28 | M501 | EG15 | H28 |
| M502 | EG16 | H28 | M503 | EG17 | H28 | M504 | EG18 | H28 |
| M505 | EG1 | H29 | M506 | EG2 | H29 | M507 | EG3 | H29 |
| M508 | EG4 | H29 | M509 | EG5 | H29 | M510 | EG6 | H29 |
| M511 | EG7 | H29 | M512 | EG8 | H29 | M513 | EG9 | H29 |
| M514 | EG10 | H29 | M515 | EG11 | H29 | M516 | EG12 | H29 |
| M517 | EG13 | H29 | M518 | EG14 | H29 | M519 | EG15 | H29 |
| M520 | EG16 | H29 | M521 | EG17 | H29 | M522 | EG18 | H29 |
| M523 | EG1 | H30 | M524 | EG2 | H30 | M525 | EG3 | H30 |
| M526 | EG4 | H30 | M527 | EG5 | H30 | M528 | EG6 | H30 |
| M529 | EG7 | H30 | M530 | EG8 | H30 | M531 | EG9 | H30 |
| M532 | EG10 | H30 | M533 | EG11 | H30 | M534 | EG12 | H30 |
| M535 | EG13 | H30 | M536 | EG14 | H30 | M537 | EG15 | H30 |
| M538 | EG16 | H30 | M539 | EG17 | H30 | M540 | EG18 | H30 |
| M541 | EG1 | H31 | M542 | EG2 | H31 | M543 | EG3 | H31 |
| M544 | EG4 | H31 | M545 | EG5 | H31 | M546 | EG6 | H31 |
| M547 | EG7 | H31 | M548 | EG8 | H31 | M549 | EG9 | H31 |
| M550 | EG10 | H31 | M551 | EG11 | H31 | M552 | EG12 | H31 |
| M553 | EG13 | H31 | M554 | EG14 | H31 | M555 | EG15 | H31 |
| M556 | EG16 | H31 | M557 | EG17 | H31 | M558 | EG18 | H31 |
| M559 | EG1 | H32 | M560 | EG2 | H32 | M561 | EG3 | H32 |
| M562 | EG4 | H32 | M563 | EG5 | H32 | M564 | EG6 | H32 |
| M565 | EG7 | H32 | M566 | EG8 | H32 | M567 | EG9 | H32 |
| M568 | EG10 | H32 | M569 | EG11 | H32 | M570 | EG12 | H32 |
| M571 | EG13 | H32 | M572 | EG14 | H32 | M573 | EG15 | H32 |
| M574 | EG16 | H32 | M575 | EG17 | H32 | M576 | EG18 | H32 |
| M577 | EG1 | H33 | M578 | EG2 | H33 | M579 | EG3 | H33 |
| M580 | EG4 | H33 | M581 | EG5 | H33 | M582 | EG6 | H33 |
| M583 | EG7 | H33 | M584 | EG8 | H33 | M585 | EG9 | H33 |
| M586 | EG10 | H33 | M587 | EG11 | H33 | M588 | EG12 | H33 |
| M589 | EG13 | H33 | M590 | EG14 | H33 | M591 | EG15 | H33 |
| M592 | EG16 | H33 | M593 | EG17 | H33 | M594 | EG18 | H33 |
| M595 | EG1 | H34 | M596 | EG2 | H34 | M597 | EG3 | H34 |
| M598 | EG4 | H34 | M599 | EG5 | H34 | M600 | EG6 | H34 |
| M601 | EG7 | H34 | M602 | EG8 | H34 | M603 | EG9 | H34 |
| M604 | EG10 | H34 | M605 | EG11 | H34 | M606 | EG12 | H34 |
| M607 | EG13 | H34 | M608 | EG14 | H34 | M609 | EG15 | H34 |
| M610 | EG16 | H34 | M611 | EG17 | H34 | M612 | EG18 | H34 |
| M613 | EG1 | H35 | M614 | EG2 | H35 | M615 | EG3 | H35 |
| M616 | EG4 | H35 | M617 | EG5 | H35 | M618 | EG6 | H35 |
| M619 | EG7 | H35 | M620 | EG8 | H35 | M621 | EG9 | H35 |
| M622 | EG10 | H35 | M623 | EG11 | H35 | M624 | EG12 | H35 |
| M625 | EG13 | H35 | M626 | EG14 | H35 | M627 | EG15 | H35 |
| M628 | EG16 | H35 | M629 | EG17 | H35 | M630 | EG18 | H35 |
| M631 | EG1 | H36 | M632 | EG2 | H36 | M633 | EG3 | H36 |
| M634 | EG4 | H36 | M635 | EG5 | H36 | M636 | EG6 | H36 |
| M637 | EG7 | H36 | M638 | EG8 | H36 | M639 | EG9 | H36 |
| M640 | EG10 | H36 | M641 | EG11 | H36 | M642 | EG12 | H36 |
| M643 | EG13 | H36 | M644 | EG14 | H36 | M645 | EG15 | H36 |
| M646 | EG16 | H36 | M647 | EG17 | H36 | M648 | EG18 | H36 |
| M649 | EG1 | H37 | M650 | EG2 | H37 | M651 | EG3 | H37 |
| M652 | EG4 | H37 | M653 | EG5 | H37 | M654 | EG6 | H37 |
| M655 | EG7 | H37 | M656 | EG8 | H37 | M657 | EG9 | H37 |
| M658 | EG10 | H37 | M659 | EG11 | H37 | M660 | EG12 | H37 |
| M661 | EG13 | H37 | M662 | EG14 | H37 | M663 | EG15 | H37 |
| M664 | EG16 | H37 | M665 | EG17 | H37 | M666 | EG18 | H37 |
| M667 | EG1 | H38 | M668 | EG2 | H38 | M669 | EG3 | H38 |
| M670 | EG4 | H38 | M671 | EG5 | H38 | M672 | EG6 | H38 |
| M673 | EG7 | H38 | M674 | EG8 | H38 | M675 | EG9 | H38 |
| M676 | EG10 | H38 | M677 | EG11 | H38 | M678 | EG12 | H38 |
| M679 | EG13 | H38 | M680 | EG14 | H38 | M681 | EG15 | H38 |
| M682 | EG16 | H38 | M683 | EG17 | H38 | M684 | EG18 | H38 |
| M685 | EG1 | H39 | M686 | EG2 | H39 | M687 | EG3 | H39 |
| M688 | EG4 | H39 | M689 | EG5 | H39 | M690 | EG6 | H39 |
| M691 | EG7 | H39 | M692 | EG8 | H39 | M693 | EG9 | H39 |
| M694 | EG10 | H39 | M695 | EG11 | H39 | M696 | EG12 | H39 |
| M697 | EG13 | H39 | M698 | EG14 | H39 | M699 | EG15 | H39 |
| M700 | EG16 | H39 | M701 | EG17 | H39 | M702 | EG18 | H39 |
| M703 | EG1 | H40 | M704 | EG2 | H40 | M705 | EG3 | H40 |
| M706 | EG4 | H40 | M707 | EG5 | H40 | M708 | EG6 | H40 |
| M709 | EG7 | H40 | M710 | EG8 | H40 | M711 | EG9 | H40 |
| M712 | EG10 | H40 | M713 | EG11 | H40 | M714 | EG12 | H40 |
| M715 | EG13 | H40 | M716 | EG14 | H40 | M717 | EG15 | H40 |
| M718 | EG16 | H40 | M719 | EG17 | H40 | M720 | EG18 | H40 |
| M721 | EG1 | H41 | M722 | EG2 | H41 | M723 | EG3 | H41 |
| M724 | EG4 | H41 | M725 | EG5 | H41 | M726 | EG6 | H41 |
| M727 | EG7 | H41 | M728 | EG8 | H41 | M729 | EG9 | H41 |
| M730 | EG10 | H41 | M731 | EG11 | H41 | M732 | EG12 | H41 |
| M733 | EG13 | H41 | M734 | EG14 | H41 | M735 | EG15 | H41 |
| M736 | EG16 | H41 | M737 | EG17 | H41 | M738 | EG18 | H41 |
| M739 | EG1 | H42 | M740 | EG2 | H42 | M741 | EG3 | H42 |
| M742 | EG4 | H42 | M743 | EG5 | H42 | M744 | EG6 | H42 |
| M745 | EG7 | H42 | M746 | EG8 | H42 | M747 | EG9 | H42 |
| M748 | EG10 | H42 | M749 | EG11 | H42 | M750 | EG12 | H42 |
| M751 | EG13 | H42 | M752 | EG14 | H42 | M753 | EG15 | H42 |
| M754 | EG16 | H42 | M755 | EG17 | H42 | M756 | EG18 | H42 |
| M757 | EG1 | H43 | M758 | EG2 | H43 | M759 | EG3 | H43 |
| M760 | EG4 | H43 | M761 | EG5 | H43 | M762 | EG6 | H43 |
| M763 | EG7 | H43 | M764 | EG8 | H43 | M765 | EG9 | H43 |
| M766 | EG10 | H43 | M767 | EG11 | H43 | M768 | EG12 | H43 |
| M769 | EG13 | H43 | M770 | EG14 | H43 | M771 | EG15 | H43 |
| M772 | EG16 | H43 | M773 | EG17 | H43 | M774 | EG18 | H43 |
| M775 | EG1 | H44 | M776 | EG2 | H44 | M777 | EG3 | H44 |
| M778 | EG4 | H44 | M779 | EG5 | H44 | M780 | EG6 | H44 |
| M781 | EG7 | H44 | M782 | EG8 | H44 | M783 | EG9 | H44 |
| M784 | EG10 | H44 | M785 | EG11 | H44 | M786 | EG12 | H44 |
| M787 | EG13 | H44 | M788 | EG14 | H44 | M789 | EG15 | H44 |
| M790 | EG16 | H44 | M791 | EG17 | H44 | M792 | EG18 | H44 |
| M793 | EG1 | H45 | M794 | EG2 | H45 | M795 | EG3 | H45 |
| M796 | EG4 | H45 | M797 | EG5 | H45 | M798 | EG6 | H45 |
| M799 | EG7 | H45 | M800 | EG8 | H45 | M801 | EG9 | H45 |
| M802 | EG10 | H45 | M803 | EG11 | H45 | M804 | EG12 | H45 |
| M805 | EG13 | H45 | M806 | EG14 | H45 | M807 | EG15 | H45 |
| M808 | EG16 | H45 | M809 | EG17 | H45 | M810 | EG18 | H45 |
| M811 | EG1 | H46 | M812 | EG2 | H46 | M813 | EG3 | H46 |
| M814 | EG4 | H46 | M815 | EG5 | H46 | M816 | EG6 | H46 |
| M817 | EG7 | H46 | M818 | EG8 | H46 | M819 | EG9 | H46 |
| M820 | EG10 | H46 | M821 | EG11 | H46 | M822 | EG12 | H46 |
| M823 | EG13 | H46 | M824 | EG14 | H46 | M825 | EG15 | H46 |
| M826 | EG16 | H46 | M827 | EG17 | H46 | M828 | EG18 | H46 |
| M829 | EG1 | H47 | M830 | EG2 | H47 | M831 | EG3 | H47 |
| M832 | EG4 | H47 | M833 | EG5 | H47 | M834 | EG6 | H47 |
| M835 | EG7 | H47 | M836 | EG8 | H47 | M837 | EG9 | H47 |
| M838 | EG10 | H47 | M839 | EG11 | H47 | M840 | EG12 | H47 |
| M841 | EG13 | H47 | M842 | EG14 | H47 | M843 | EG15 | H47 |
| M844 | EG16 | H47 | M845 | EG17 | H47 | M846 | EG18 | H47 |
| M847 | EG1 | H48 | M848 | EG2 | H48 | M849 | EG3 | H48 |
| M850 | EG4 | H48 | M851 | EG5 | H48 | M852 | EG6 | H48 |
| M853 | EG7 | H48 | M854 | EG8 | H48 | M855 | EG9 | H48 |
| M856 | EG10 | H48 | M857 | EG11 | H48 | M858 | EG12 | H48 |
| M859 | EG13 | H48 | M860 | EG14 | H48 | M861 | EG15 | H48 |
| M862 | EG16 | H48 | M863 | EG17 | H48 | M864 | EG18 | H48 |
| M865 | EG1 | H49 | M866 | EG2 | H49 | M867 | EG3 | H49 |
| M868 | EG4 | H49 | M869 | EG5 | H49 | M870 | EG6 | H49 |
| M871 | EG7 | H49 | M872 | EG8 | H49 | M873 | EG9 | H49 |
| M874 | EG10 | H49 | M875 | EG11 | H49 | M876 | EG12 | H49 |
| M877 | EG13 | H49 | M878 | EG14 | H49 | M879 | EG15 | H49 |
| M880 | EG16 | H49 | M881 | EG17 | H49 | M882 | EG18 | H49 |
| M883 | EG1 | H50 | M884 | EG2 | H50 | M885 | EG3 | H50 |
| M886 | EG4 | H50 | M887 | EG5 | H50 | M888 | EG6 | H50 |
| M889 | EG7 | H50 | M890 | EG8 | H50 | M891 | EG9 | H50 |
| M892 | EG10 | H50 | M893 | EG11 | H50 | M894 | EG12 | H50 |
| M895 | EG13 | H50 | M896 | EG14 | H50 | M897 | EG15 | H50 |
| M898 | EG16 | H50 | M899 | EG17 | H50 | M900 | EG18 | H50 |
| M901 | EG1 | H51 | M902 | EG2 | H51 | M903 | EG3 | H51 |
| M904 | EG4 | H51 | M905 | EG5 | H51 | M906 | EG6 | H51 |
| M907 | EG7 | H51 | M908 | EG8 | H51 | M909 | EG9 | H51 |
| M910 | EG10 | H51 | M911 | EG11 | H51 | M912 | EG12 | H51 |
| M913 | EG13 | H51 | M914 | EG14 | H51 | M915 | EG15 | H51 |
| M916 | EG16 | H51 | M917 | EG17 | H51 | M918 | EG18 | H51 |
| M919 | EG1 | H52 | M920 | EG2 | H52 | M921 | EG3 | H52 |
| M922 | EG4 | H52 | M923 | EG5 | H52 | M924 | EG6 | H52 |
| M925 | EG7 | H52 | M926 | EG8 | H52 | M927 | EG9 | H52 |
| M928 | EG10 | H52 | M929 | EG11 | H52 | M930 | EG12 | H52 |
| M931 | EG13 | H52 | M932 | EG14 | H52 | M933 | EG15 | H52 |
| M934 | EG16 | H52 | M935 | EG17 | H52 | M936 | EG18 | H52 |
| M937 | EG1 | H53 | M938 | EG2 | H53 | M939 | EG3 | H53 |
| M940 | EG4 | H53 | M941 | EG5 | H53 | M942 | EG6 | H53 |
| M943 | EG7 | H53 | M944 | EG8 | H53 | M945 | EG9 | H53 |
| M946 | EG10 | H53 | M947 | EG11 | H53 | M948 | EG12 | H53 |
| M949 | EG13 | H53 | M950 | EG14 | H53 | M951 | EG15 | H53 |
| M952 | EG16 | H53 | M953 | EG17 | H53 | M954 | EG18 | H53 |
| M955 | EG1 | H54 | M956 | EG2 | H54 | M957 | EG3 | H54 |
| M958 | EG4 | H54 | M959 | EG5 | H54 | M960 | EG6 | H54 |
| M961 | EG7 | H54 | M962 | EG8 | H54 | M963 | EG9 | H54 |
| M964 | EG10 | H54 | M965 | EG11 | H54 | M966 | EG12 | H54 |
| M967 | EG13 | H54 | M968 | EG14 | H54 | M969 | EG15 | H54 |
| M970 | EG16 | H54 | M971 | EG17 | H54 | M972 | EG18 | H54 |

The concentration of the host material of the formula (1) as described above or described as preferred in the mixture of the invention or in the light-emitting layer of the device of the invention is in the range from 5% by weight to 90% by weight, preferably in the range from 10% by weight to 85% by weight, more preferably in the range from 20% by weight to 85% by weight, even more preferably in the range from 30% by weight to 80% by weight, very especially preferably in the range from 20% by weight to 60% by weight and most preferably in the range from 30% by weight to 50% by weight, based on the overall mixture or based on the overall composition of the light-emitting layer.

The concentration of the post material of one of the formulae (6), (7), (8), (9), (10) and (11) as described above or described as preferred in the mixture of the invention or in the light-emitting layer of the device of the invention is in the range from 10% by weight to 95% by weight, preferably in the range from 15% by weight to 90% by weight, more preferably in the range from 15% by weight to 80% by weight, even more preferably in the range from 20% by weight to 70% by weight, very especially preferably in the range from 40% by weight to 80% by weight and most preferably in the range from 50% by weight to 70% by weight, based on the overall mixture or based on the overall composition of the light-emitting layer.

The present invention also relates to a mixture which, as well as the aforementioned host materials of the formula (1), called host material 1 hereinafter, and the host material of one of the formulae (6), (7), (8), (9), (10) and (11), called host material 2 hereinafter, as described above or described as preferred, especially mixtures M1 to M972, also comprises at least one phosphorescent emitter.

The present invention also relates to an organic electroluminescent device as described above or described as preferred, wherein the light-emitting layer, as well as the aforementioned host materials of the formulae (1) and one of the formulae (6), (7), (8), (9), (10) and (11), as described above or described as preferred, especially the material combinations M1 to M972, also comprises at least one phosphorescent emitter.

The term “phosphorescent emitters” typically encompasses compounds where the light is emitted through a spin-forbidden transition from an excited state having higher spin multiplicity, i.e. a spin state >1, for example through a transition from a triplet state or a state having an even higher spin quantum number, for example a quintet state. This preferably means a transition from a triplet state.

Suitable phosphorescent emitters (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent emitters.

In general, all phosphorescent complexes as used for phosphorescent OLEDs according to the prior art and as known to those skilled in the art in the field of organic electroluminescent devices are suitable.

Preferred phosphorescent emitters according to the present invention conform to the formula (IIIa)

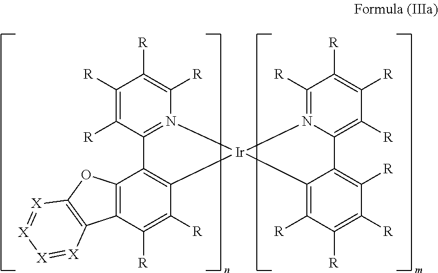

where the symbols and indices for this formula (IIIa) are defined as follows:
n+m is 3, n is 1 or 2, m is 2 or 1,
X is N or CR,
R is H, D, or a branched or linear alkyl group having 1 to 10 carbon atoms or a partly or fully deuterated branched or linear alkyl group having 1 to 10 carbon atoms or a cycloalkyl group which has 4 to 7 carbon atoms and may be partly or fully substituted by deuterium.

The invention accordingly further provides an organic electroluminescent device as described above or described as preferred, characterized in that the light-emitting layer, as well as the host materials 1 and 2, comprises at least one phosphorescent emitter conforming to the formula (IIIa) as described above.

In emitters of the formula (IIIa), n is preferably 1 and m is preferably 2.

In emitters of the formula (IIIa), preferably one X is selected from N and the other X are CR.

In emitters of the formula (IIIa), at least one R is preferably different than H. In emitters of the formula (IIIa), preferably two R are different than H and have one of the other definitions given above for the emitters of the formula (IIIa).

Preferred phosphorescent emitters according to the present invention conform to the formulae (I), (II), (III), (IV) or (V)

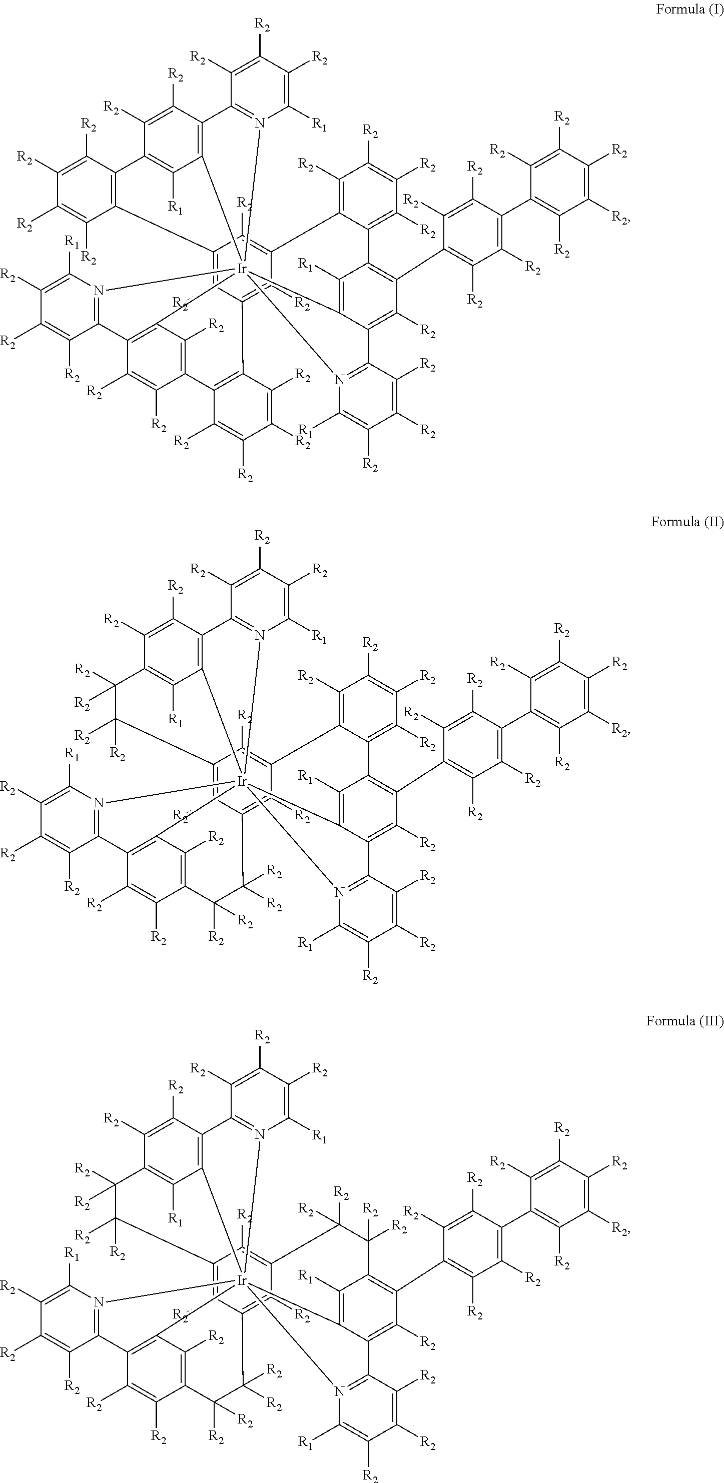

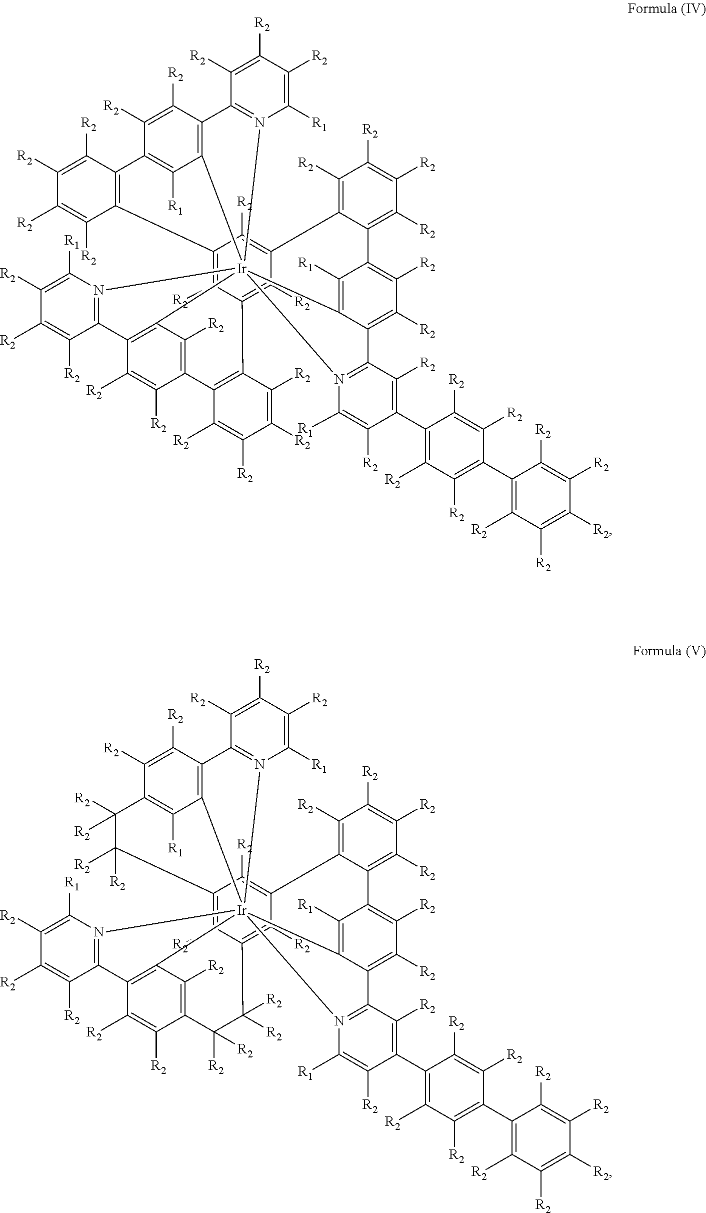

where the symbols and indices for these formulae (I), (II), (III), (IV) and (V) are defined as follows:
R₁ is H or D, R₂ is H, D, or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred phosphorescent emitters according to the present invention conform to the formulae (VI), (VII) or (VIII)

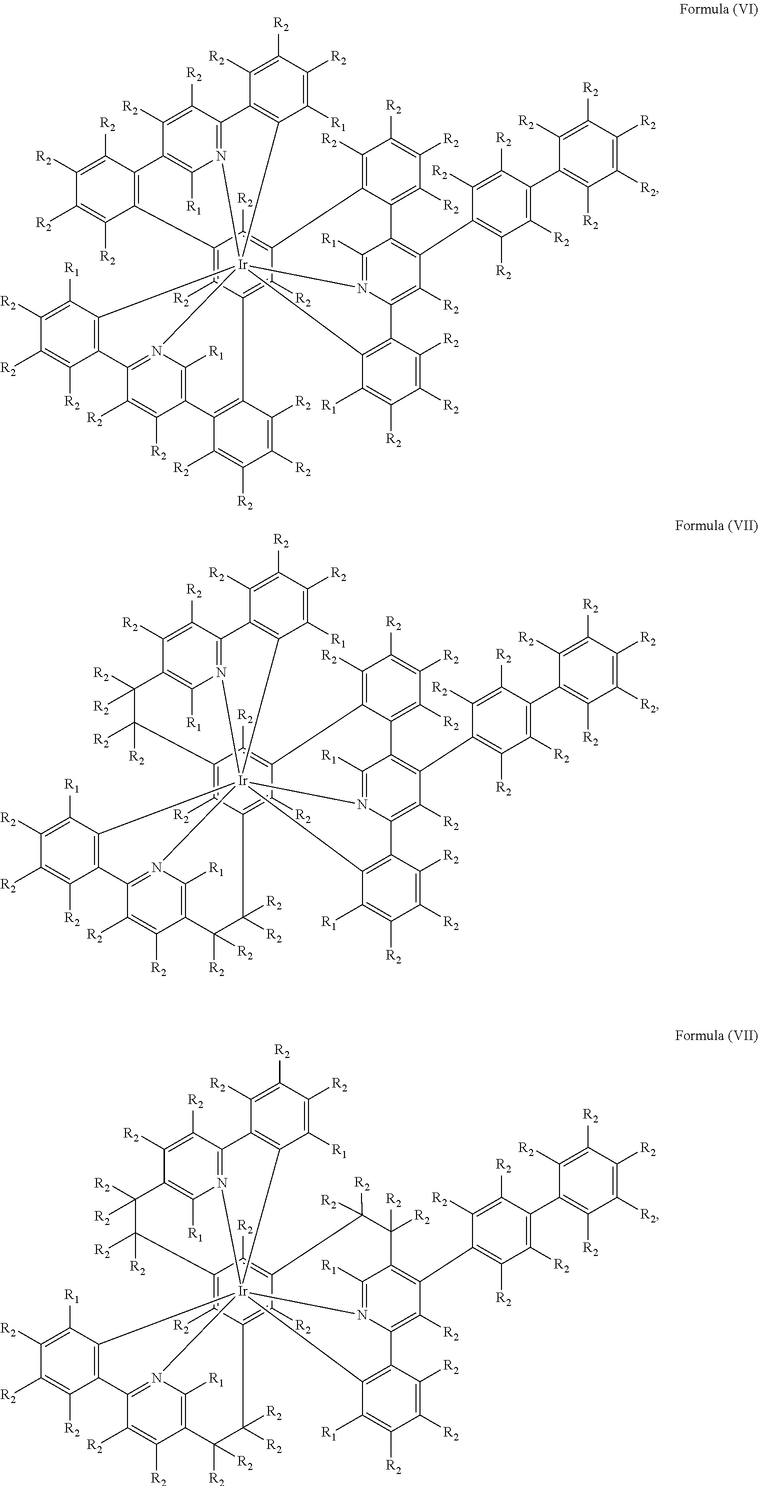

where the symbols and indices for these formulae (VI), (VII) and (VIII) are defined as follows:
R₁ is H or D, R₂ is H, D, F or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred examples of phosphorescent emitters are described in WO2019007867 on pages 120 to 126 in table 5, and on pages 127 to 129 in table 6. The emitters are incorporated into description by this reference.

Particularly preferred examples of phosphorescent emitters are listed in table 5 below.

TABLE 5

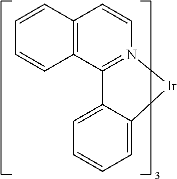

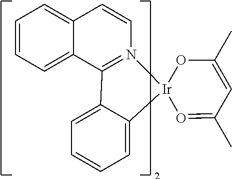

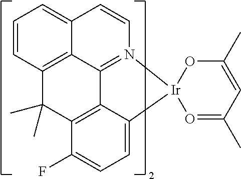

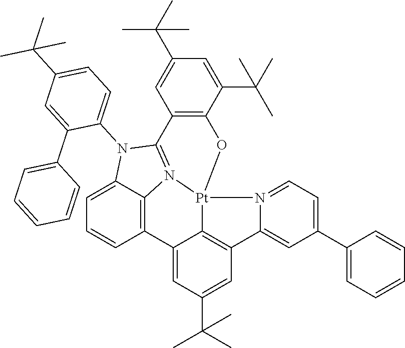

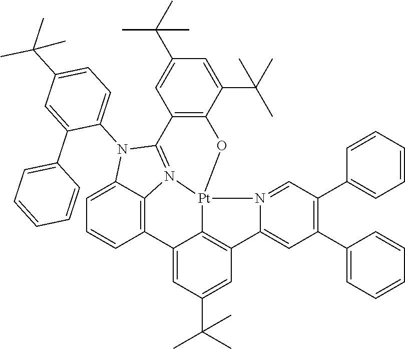

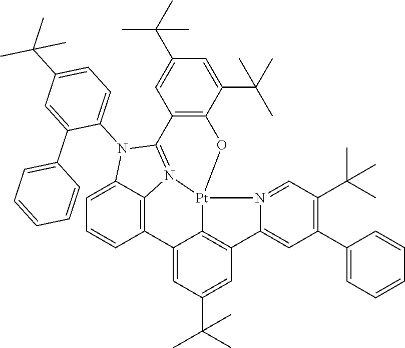

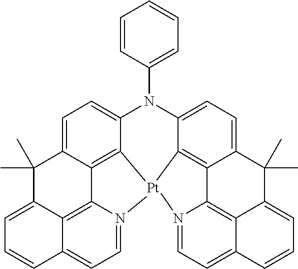

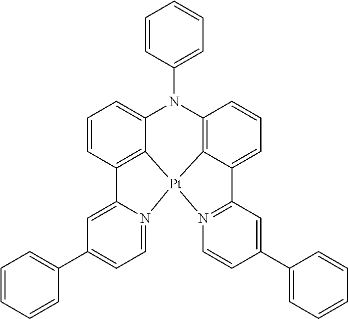

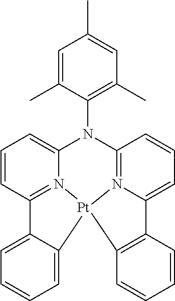

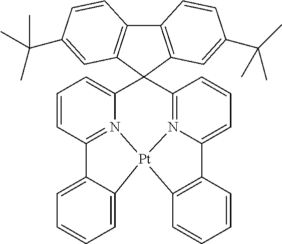

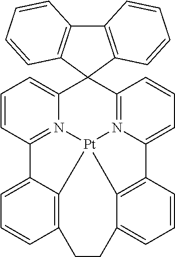

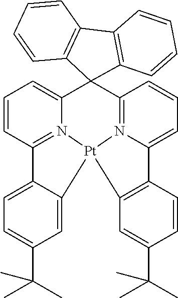

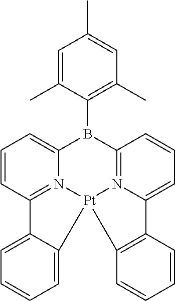

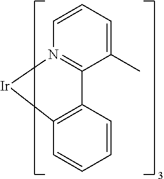

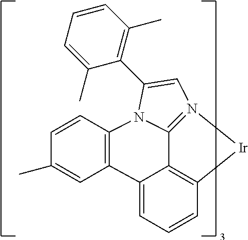

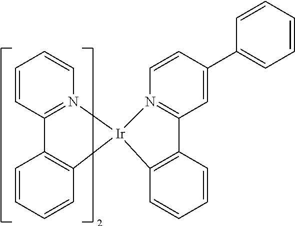

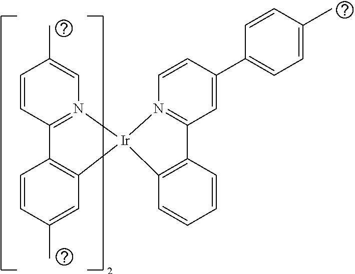

[symbol]

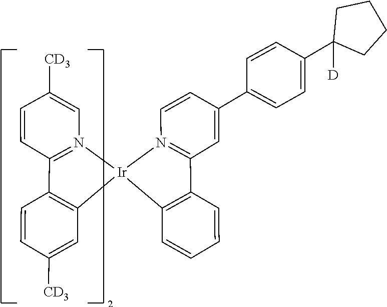

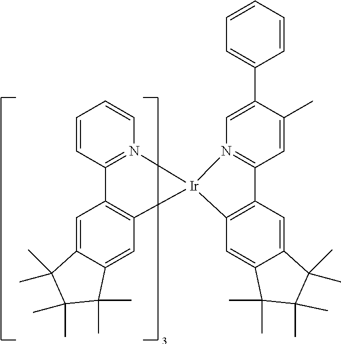

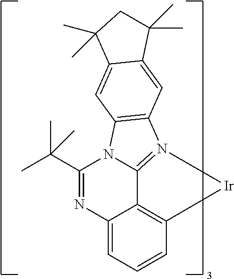

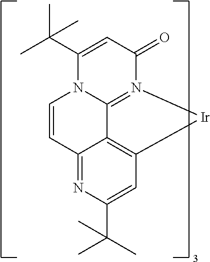

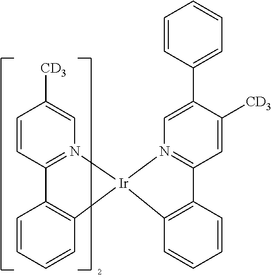

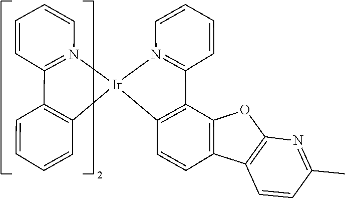

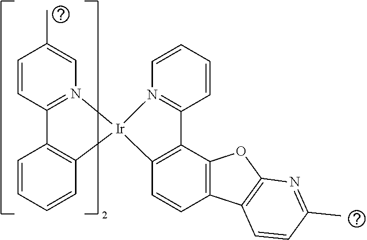

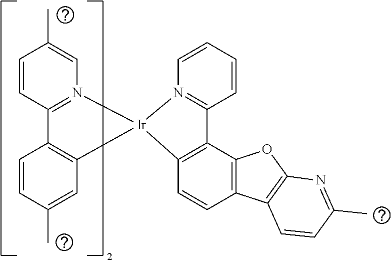

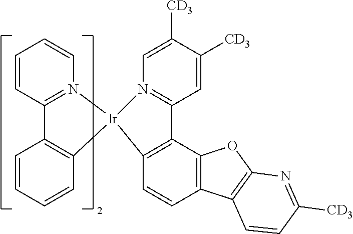

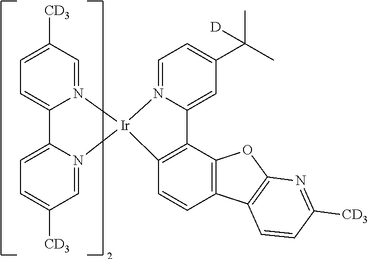

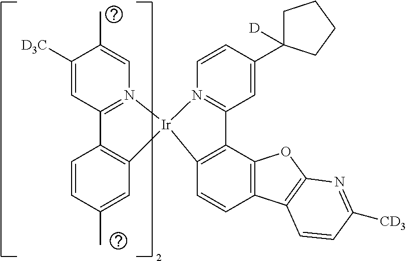

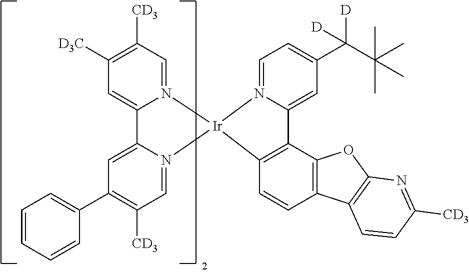

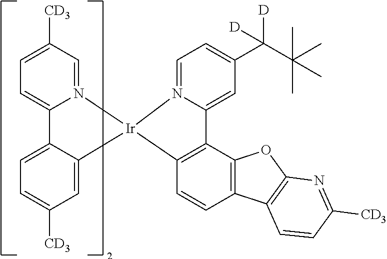

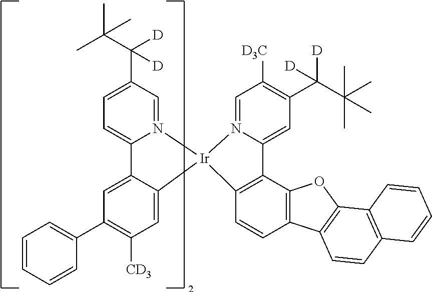

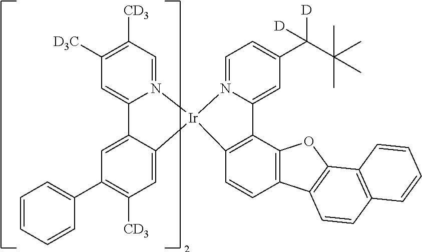

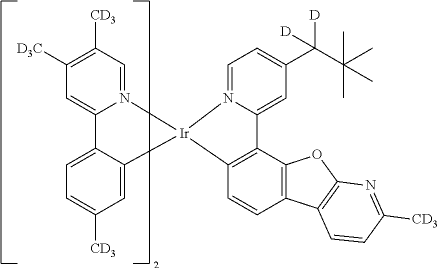

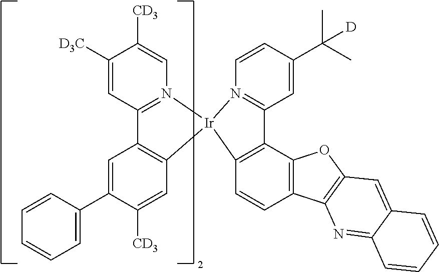

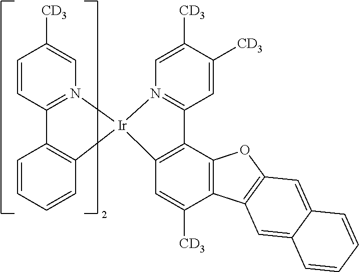

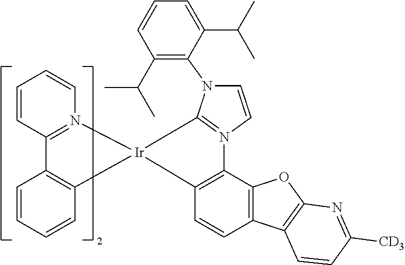

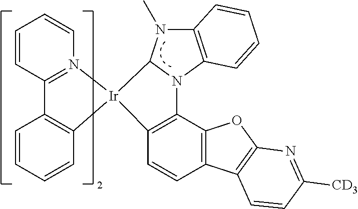

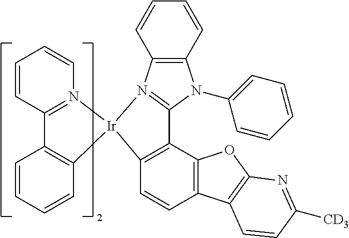

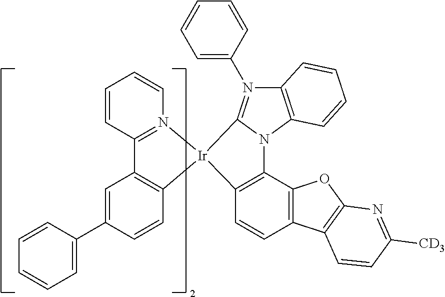

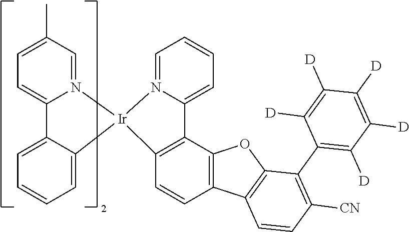

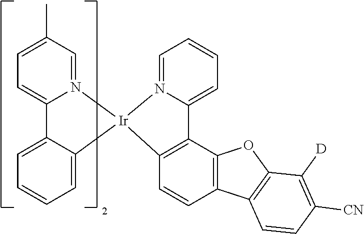

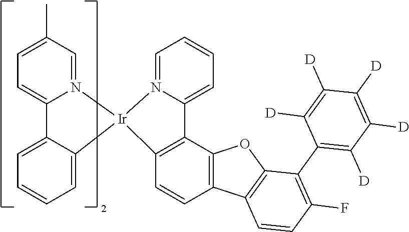

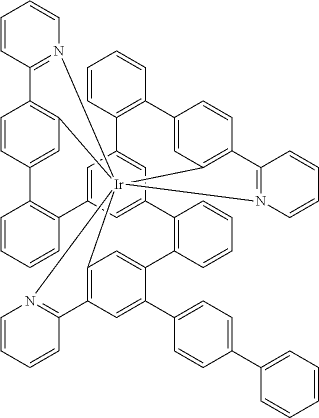

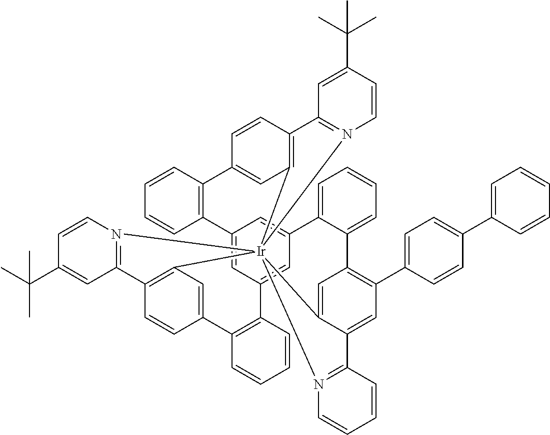

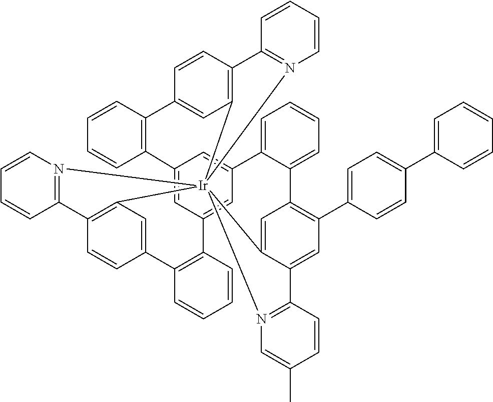

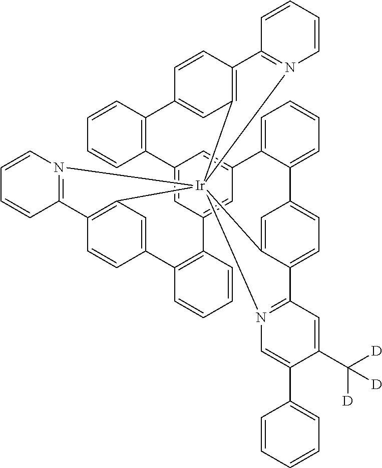

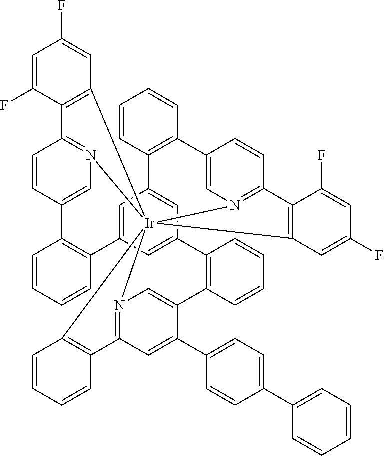

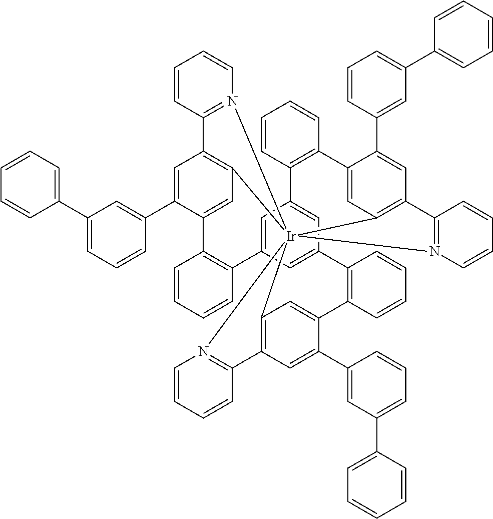

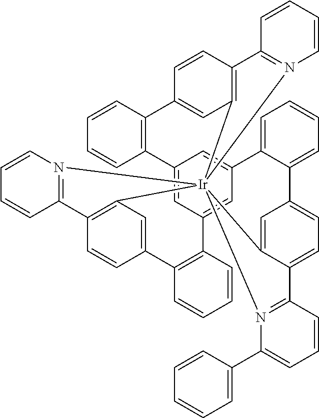

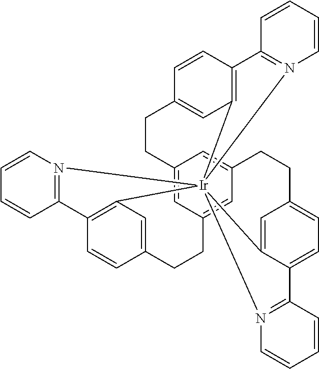

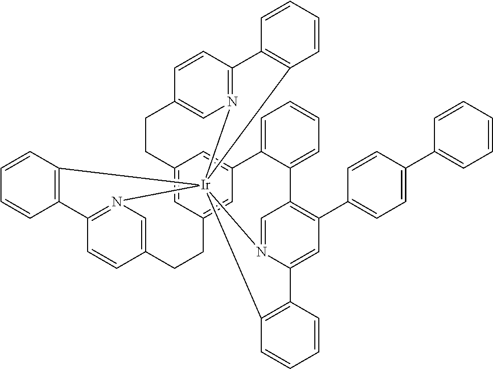

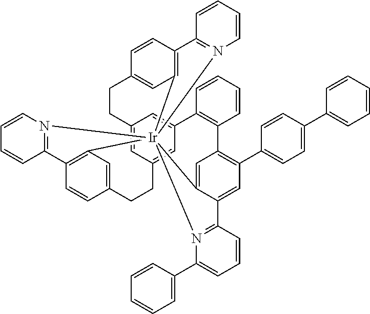

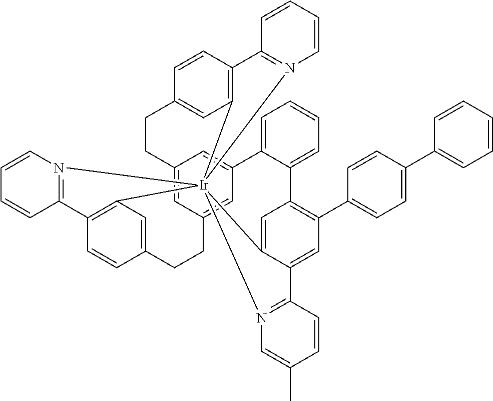

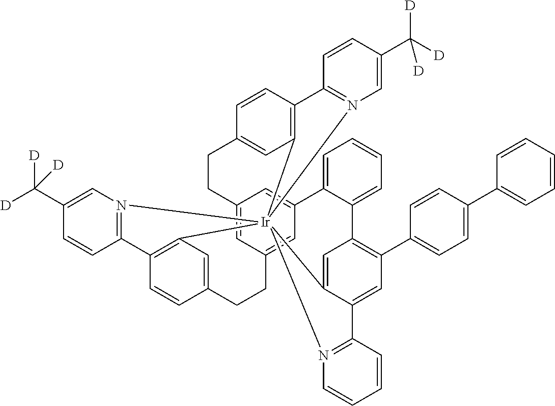

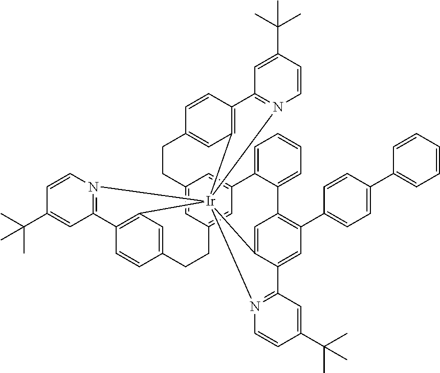

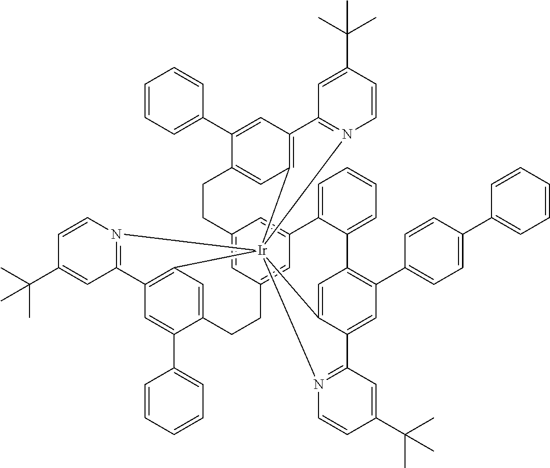

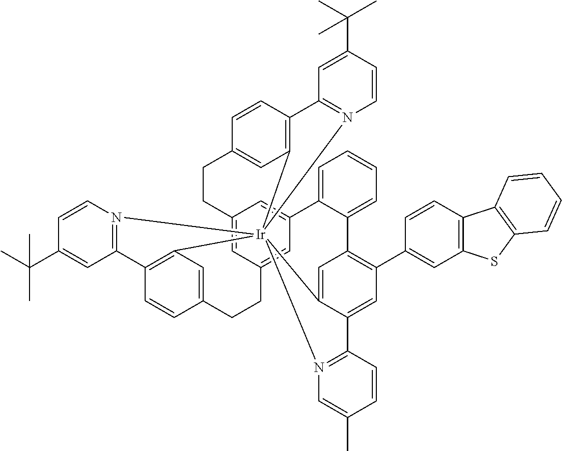

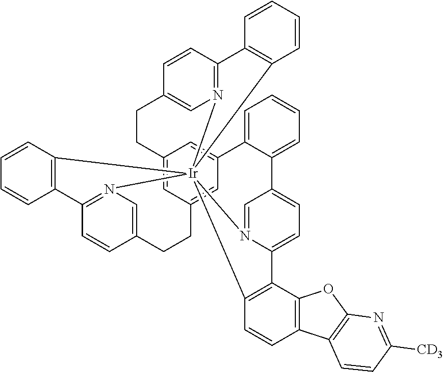

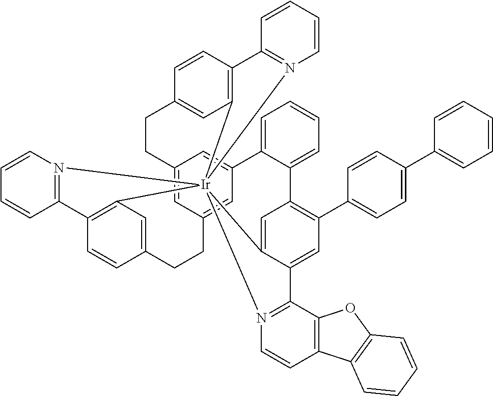

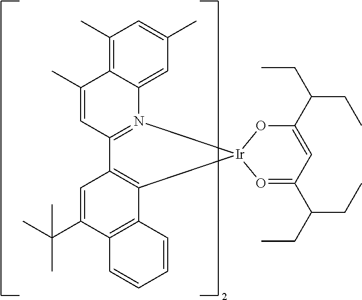

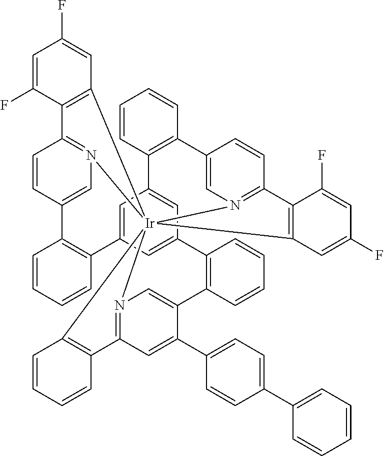

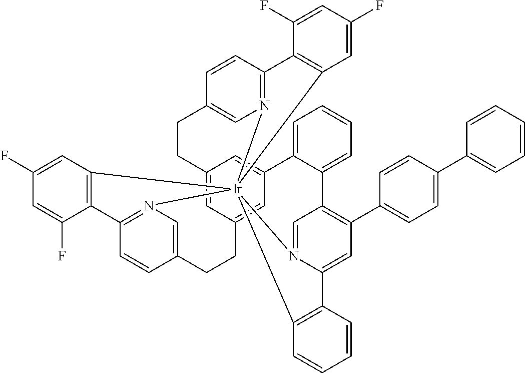

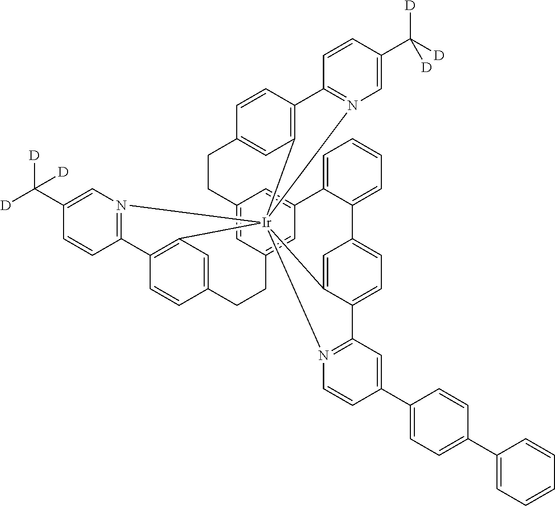

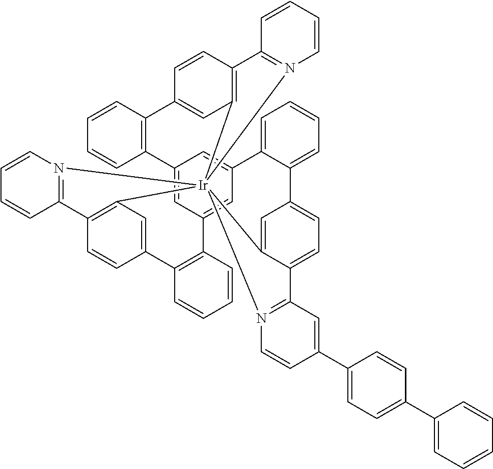

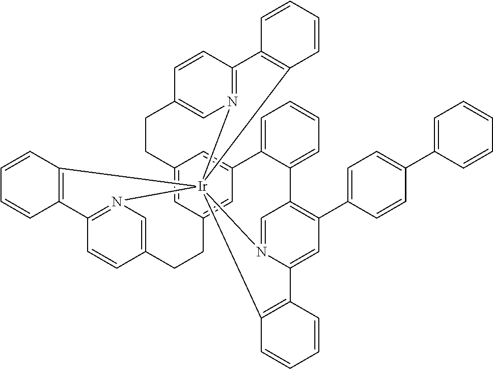

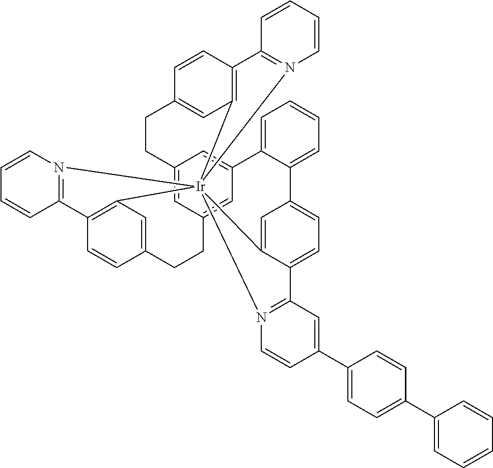

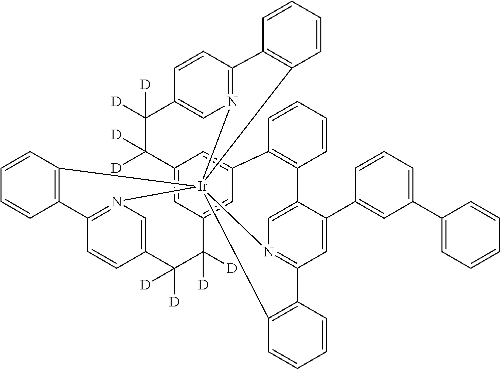

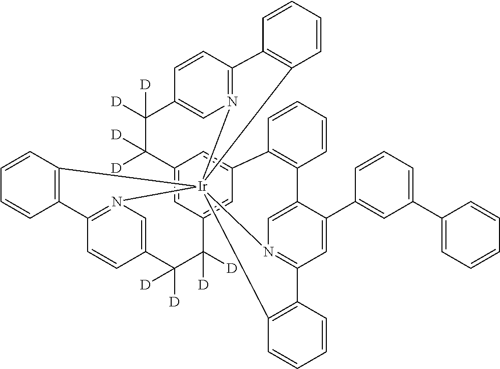

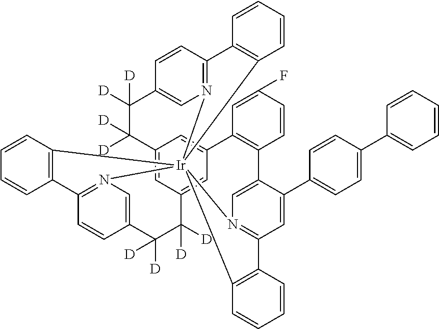

[symbol]

indicates data missing or illegible when filed

In the mixtures of the invention or in the light-emitting layer of the device of the invention, any mixture selected from the sum of the mixtures M1 to M972 is preferably combined with a compound of the formula (IIIa) or a compound of the formulae (I) to (VIII) or a compound from table 5.

The light-emitting layer in the organic electroluminescent device of the invention, comprising at least one phosphorescent emitter, is preferably an infrared-emitting or yellow-, orange-, red-, green-, blue- or ultraviolet-emitting layer, more preferably a yellow- or green-emitting layer and most preferably a green-emitting layer.

What is meant here by a yellow-emitting layer is a layer having a photoluminescence maximum within the range from 540 to 570 nm. What is meant by an orange-emitting layer is a layer having a photoluminescence maximum within the range from 570 to 600 nm. What is meant by a red-emitting layer is a layer having a photoluminescence maximum within the range from 600 to 750 nm. What is meant by a green-emitting layer is a layer having a photoluminescence maximum within the range from 490 to 540 nm. What is meant by a blue-emitting layer is a layer having a photoluminescence maximum within the range from 440 to 490 nm. The photoluminescence maximum of the layer is determined here by measuring the photoluminescence spectrum of the layer having a layer thickness of 50 nm at room temperature, said layer having the inventive combination of the host materials of the formula (1) and one of the formulae (6), (7), (8), (9), (10) and (11) and the appropriate emitter.

The photoluminescence spectrum of the layer is recorded, for example, with a commercial photoluminescence spectrometer.

The photoluminescence spectrum of the emitter chosen is generally measured in oxygen-free solution, 10⁻⁵ molar, at room temperature, a suitable solvent being any in which the chosen emitter dissolves in the concentration mentioned. Particularly suitable solvents are typically toluene or 2-methyl-THF, but also dichloromethane. Measurement is effected with a commercial photoluminescence spectrometer. The triplet energy T1 in eV is determined from the photoluminescence spectra of the emitters. First the peak maximum PImax. (in nm) of the photoluminescence spectrum is determined. The peak maximum PImax. (in nm) is then converted to eV by: E(T1 in eV)=1240/E(T1 in nm)=1240/PLmax. (in nm).

Preferred phosphorescent emitters are accordingly yellow emitters, preferably of the formula (IIIa), of the formulae (I) to (VIII) or from table 5, the triplet energy T₁ of which is preferably ˜2.3 eV to ˜2.1 eV.

Preferred phosphorescent emitters are accordingly green emitters, preferably of the formula (IIIa), of the formulae (I) to (VIII) or from table 5, the triplet energy T₁ of which is preferably ˜2.5 eV to ˜2.3 eV.

Particularly preferred phosphorescent emitters are accordingly green emitters, preferably of the formula (IIIa), of the formulae (I) to (VIII) or from table 5 as described above, the triplet energy T₁ of which is preferably ˜2.5 eV to ˜2.3 eV.

Most preferably, green emitters, preferably of the formula (IIIa), of the formulae (I) to (VIII) or from table 5, as described above, are selected for the mixture of the invention or emitting layer of the invention.

It is also possible for fluorescent emitters to be present in the light-emitting layer of the device of the invention or in the mixture of the invention.

Preferred fluorescent emitting compounds are selected from the class of the arylamines, where preferably at least one of the aromatic or heteroaromatic ring systems of the arylamine is a fused ring system, more preferably having at least 14 ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

In a further preferred embodiment of the invention, the at least one light-emitting layer of the organic electroluminescent device, as well as the host materials 1 and 2 as described above or described as preferred, may comprise further host materials or matrix materials, called mixed matrix systems. The mixed matrix systems preferably comprise three or four different matrix materials, more preferably three different matrix materials (in other words, one further matrix component in addition to the host materials 1 and 2 as described above). Particularly suitable matrix materials which can be used in combination as matrix component in a mixed matrix system are selected from wide-band gap materials, bipolar host materials, electron transport materials (ETM) and hole transport materials (HTM). Preferably, the mixed matrix system is optimized for an emitter of the formula (IIIa), the formulae (I) to (VIII), or from table 5.

In one embodiment of the present invention, the mixture, aside from the constituents of the host material of the formula (1) and the host material 2 as described above, does not comprise any further constituents, i.e. functional materials. These are material mixtures that are used as such for production of the light-emitting layer. These mixtures are also referred to as premix systems that are used as the sole material source in the vapor deposition of the host materials for the light-emitting layer and have a constant mixing ratio in the vapor deposition. In this way, it is possible in a simple and rapid manner to achieve the vapor deposition of a layer with homogeneous distribution of the components without the need for precise actuation of a multitude of material sources.

In an alternative embodiment of the present invention, the mixture, aside from the constituents of the host material of the formula (1) and the host material 2 as described above, also comprises a phosphorescent emitter, as described above. In the case of a suitable mixing ratio in the vapor deposition, this mixture may also be used as the sole material source as described above.

The components or constituents of the light-emitting layer of the device of the invention may thus be processed by vapor deposition or from solution. The material combination of host materials 1 and 2 as described above or described as preferred, optionally with the phosphorescent emitter as described above or described as preferred, are provided for that purpose in a formulation containing at least one solvent. Suitable formulations have been described above.

The light-emitting layer in the device of the invention, according to the preferred embodiments and the emitting compound, contains preferably between 99.9% and 1% by volume, further preferably between 99% and 10% by volume, especially preferably between 98% and 60% by volume, very especially preferably between 97% and 80% by volume, of matrix material composed of at least one compound of the formula (1) and at least one compound of the one of the formulae (6), (7), (8), (9), (10) and (11) according to the preferred embodiments, based on the overall composition of emitter and matrix material. Correspondingly, the light-emitting layer in the device of the invention preferably contains between 0.1% and 99% by volume, further preferably between 1% and 90% by volume, more preferably between 2% and 40% by volume, most preferably between 3% and 20% by volume, of the emitter based on the overall composition of the light-emitting layer composed of emitter and matrix material. If the compounds are processed from solution, preference is given to using the corresponding amounts in % by weight rather than the above-specified amounts in % by volume.

The light-emitting layer in the device of the invention, according to the preferred embodiments and the emitting compound, preferably contains the host material 1 and the host material 2 in a percentage by volume ratio between 3:1 and 1:3, preferably between 1:2.5 and 1:1, more preferably between 1:2 and 1:1. If the compounds are processed from solution, preference is given to using the corresponding ratio in % by weight rather than the above-specified ratio in % by volume.

The present invention also relates to an organic electroluminescent device as described above or described as preferred, wherein the organic layer comprises a hole injection layer (HIL) and/or a hole transport layer (HTL), the hole-injecting material and hole-transporting material of which belongs to the class of the arylamines. Preferred compounds with hole transport function that do not conform to one of the formulae for the host material 2, preferably for use in a hole injection layer, a hole transport layer, an electron blocker layer and/or as additional matrix material in the emitting layer of the invention, are shown in table 6 below. The compounds in table 6, as the structures show, are non-deuterated compounds.

TABLE 6 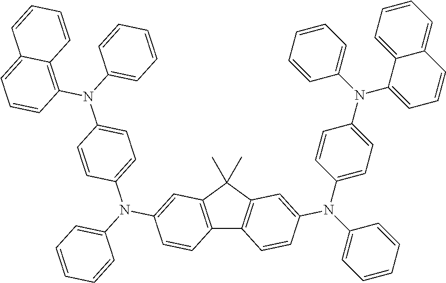 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21]
HT-1 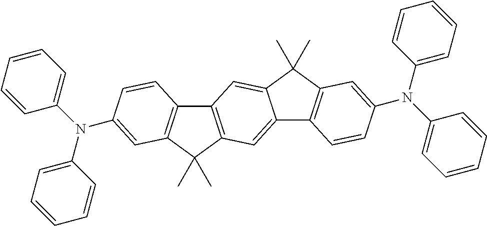 [SMILES: CC1(C)c2cc(N(c3ccccc3)c3ccccc3)ccc2-c2cc3c(cc21)-c1ccc(N(c2ccccc2)c2ccccc2)cc1C3(C)C]
HT-2 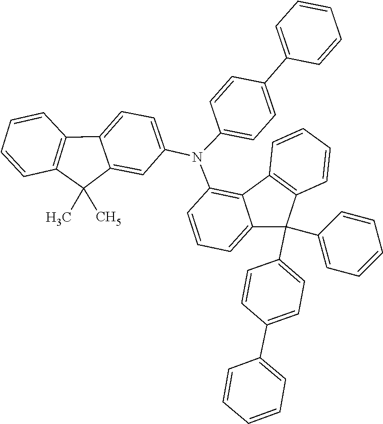
HT-3 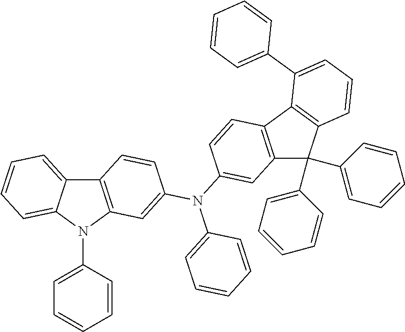 [SMILES: c1ccc(-c2cccc3c2-c2ccc(N(c4ccccc4)c4ccc5c6ccccc6n(-c6ccccc6)c5c4)cc2C3(c2ccccc2)c2ccccc2)cc1]
HT-4 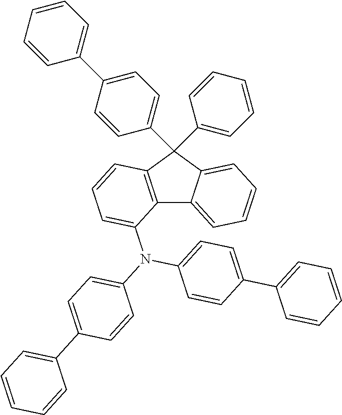 [SMILES: c1ccc(-c2ccc(N(c3ccc(-c4ccccc4)cc3)c3cccc4c3-c3ccccc3C4(c3ccccc3)c3ccc(-c4ccccc4)cc3)cc2)cc1]
HT-5 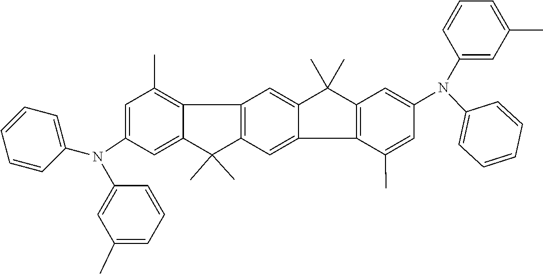 [SMILES: Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1]
HT-6 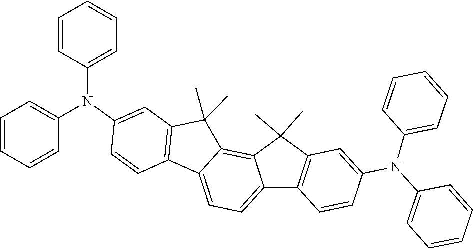 [SMILES: CC1(C)c2cc(N(c3ccccc3)c3ccccc3)ccc2-c2ccc3c(c21)C(C)(C)c1cc(N(c2ccccc2)c2ccccc2)ccc1-3]
HT-7 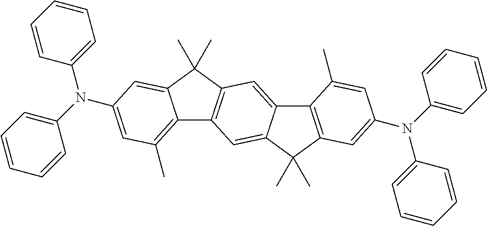 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C]
HT-8 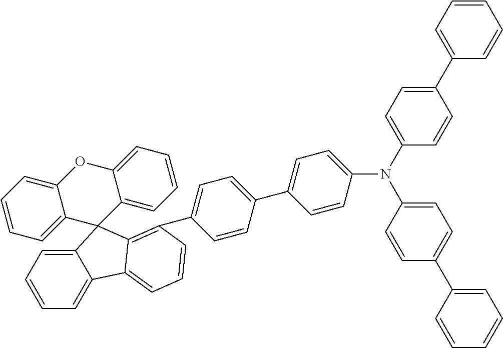 [SMILES: c1ccc(-c2ccc(N(c3ccc(-c4ccccc4)cc3)c3ccc(-c4ccc(-c5cccc6c5C5(c7ccccc7Oc7ccccc75)c5ccccc5-6)cc4)cc3)cc2)cc1]
HT-9 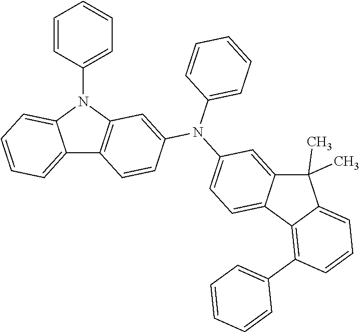 [SMILES: CC1(C)c2cc(N(c3ccccc3)c3ccc4c5ccccc5n(-c5ccccc5)c4c3)ccc2-c2c(-c3ccccc3)cccc21]
HT-10 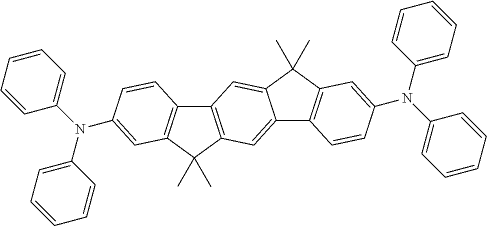 [SMILES: CC1(C)c2cc(N(c3ccccc3)c3ccccc3)ccc2-c2cc3c(cc21)-c1ccc(N(c2ccccc2)c2ccccc2)cc1C3(C)C]
HT-11 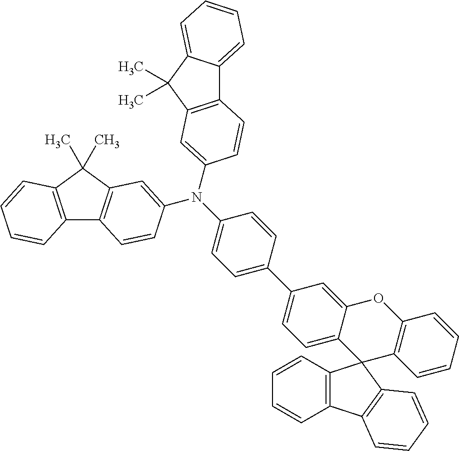 [SMILES: CC1(C)c2ccccc2-c2ccc(N(c3ccc(-c4ccc5c(c4)Oc4ccccc4C54c5ccccc5-c5ccccc54)cc3)c3ccc4c(c3)C(C)(C)c3ccccc3-4)cc21]
HT-12 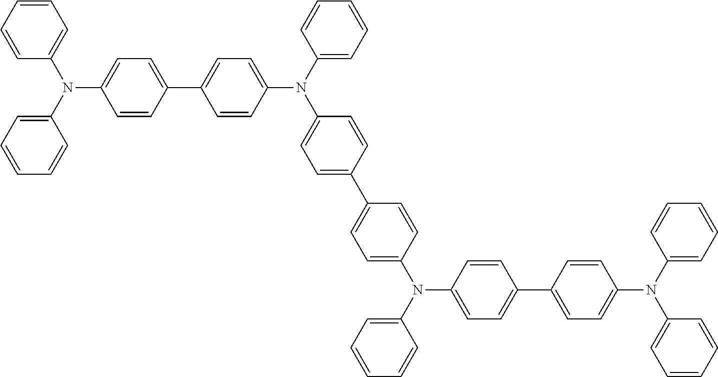 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]
HT-13 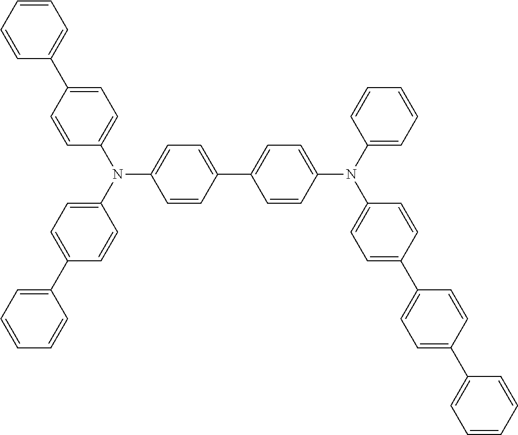 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]
HT-14 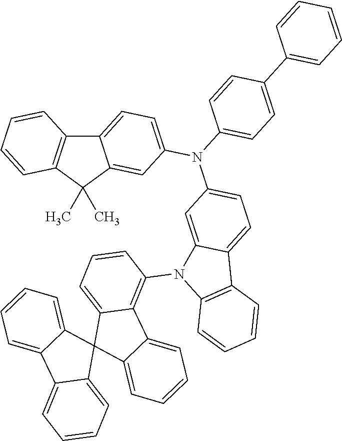 [SMILES: CC1(C)c2ccccc2-c2ccc(N(c3ccc(-c4ccccc4)cc3)c3ccc4c5ccccc5n(-c5cccc6c5-c5ccccc5C65c6ccccc6-c6ccccc65)c4c3)cc21]
HT-15 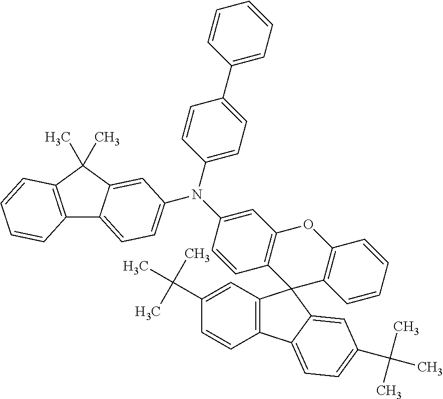 [SMILES: CC(C)(C)c1ccc2c(c1)C1(c3ccccc3Oc3cc(N(c4ccc(-c5ccccc5)cc4)c4ccc5c(c4)C(C)(C)c4ccccc4-5)ccc31)c1cc(C(C)(C)C)ccc1-2]
HT-16 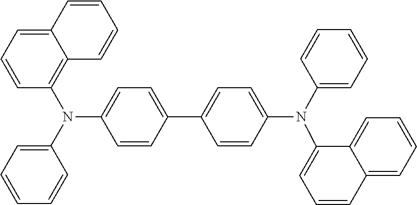 [SMILES: c1ccc(N(c2ccc(-c3ccc(N(c4ccccc4)c4cccc5ccccc45)cc3)cc2)c2cccc3ccccc23)cc1]
HT-17 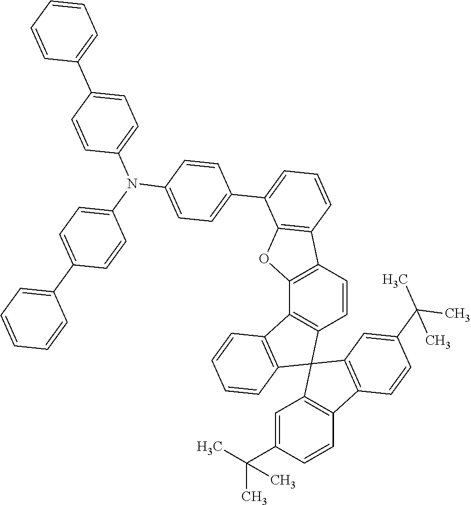 [SMILES: CC(C)(C)c1ccc2c(c1)C1(c3cc(C(C)(C)C)ccc3-2)c2ccccc2-c2c1ccc1c2oc2c(-c3ccc(N(c4ccc(-c5ccccc5)cc4)c4ccc(-c5ccccc5)cc4)cc3)cccc21]
HT-18 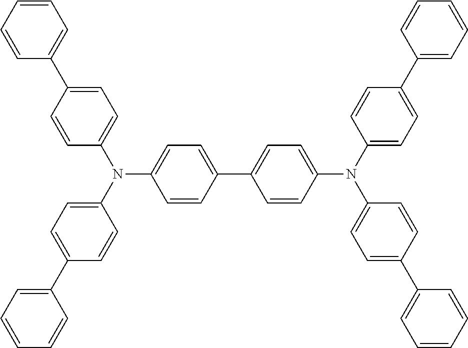 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]
HT-19 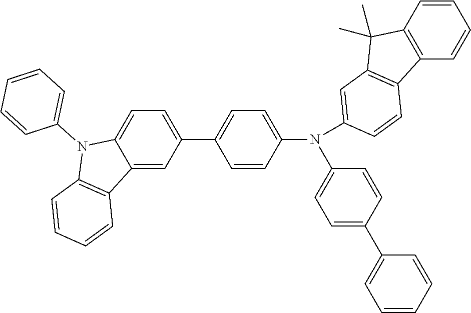 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
HT-20 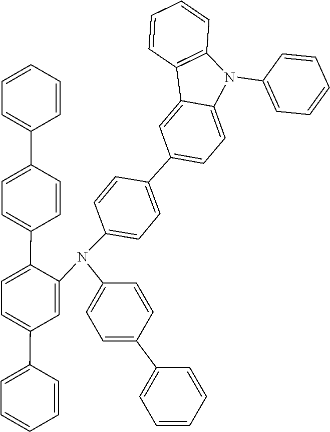 [SMILES: c1ccc(-c2ccc(-c3ccc(-c4ccccc4)cc3N(c3ccc(-c4ccccc4)cc3)c3ccc(-c4ccc5c(c4)c4ccccc4n5-c4ccccc4)cc3)cc2)cc1]
HT-21 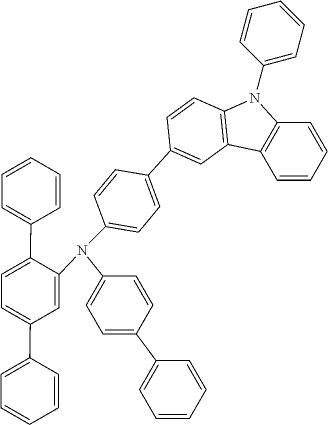 [SMILES: c1ccc(-c2ccc(N(c3ccc(-c4ccc5c(c4)c4ccccc4n5-c4ccccc4)cc3)c3cc(-c4ccccc4)ccc3-c3ccccc3)cc2)cc1]
HT-22 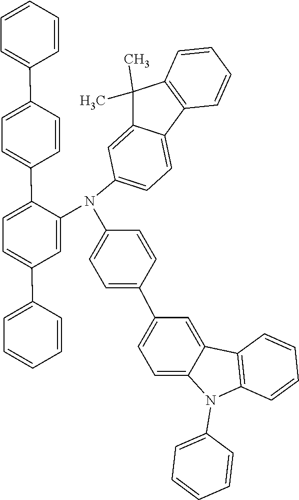 [SMILES: CC1(C)c2ccccc2-c2ccc(N(c3ccc(-c4ccc5c(c4)c4ccccc4n5-c4ccccc4)cc3)c3cc(-c4ccccc4)ccc3-c3ccc(-c4ccccc4)cc3)cc21]
HT-23 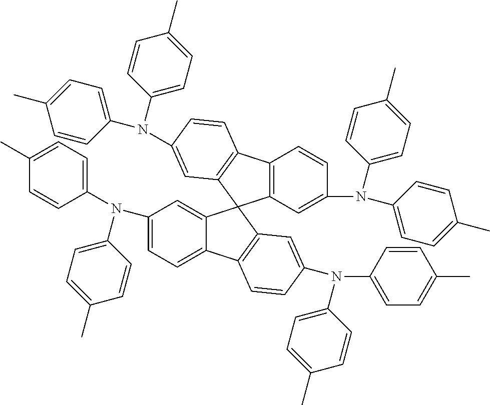
HT-24 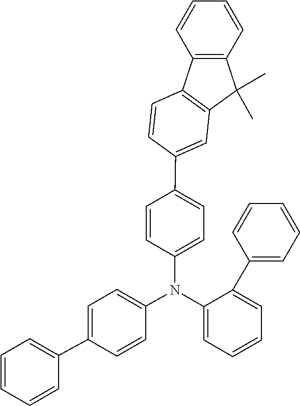 [SMILES: CC1(C)c2ccccc2-c2ccc(-c3ccc(N(c4ccc(-c5ccccc5)cc4)c4ccccc4-c4ccccc4)cc3)cc21]
HT-25 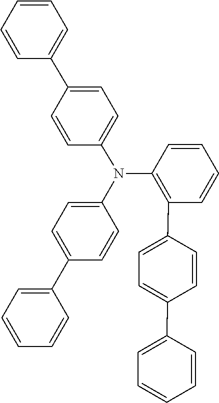 [SMILES: c1ccc(-c2ccc(-c3ccccc3N(c3ccc(-c4ccccc4)cc3)c3ccc(-c4ccccc4)cc3)cc2)cc1]
HT-26 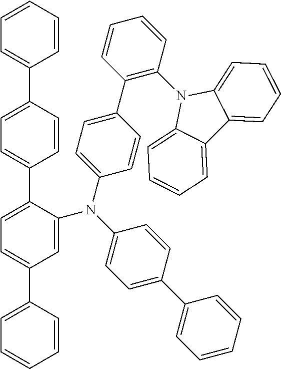 [SMILES: c1ccc(-c2ccc(-c3ccc(-c4ccccc4)cc3N(c3ccc(-c4ccccc4)cc3)c3ccc(-c4ccccc4-n4c5ccccc5c5ccccc54)cc3)cc2)cc1]
HT-27 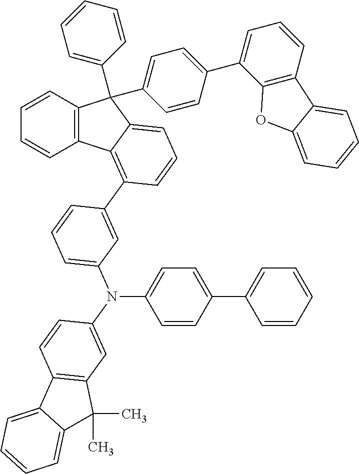 [SMILES: CC1(C)c2ccccc2-c2ccc(N(c3ccc(-c4ccccc4)cc3)c3cccc(-c4cccc5c4-c4ccccc4C5(c4ccccc4)c4ccc(-c5cccc6c5oc5ccccc56)cc4)c3)cc21]
HT-28 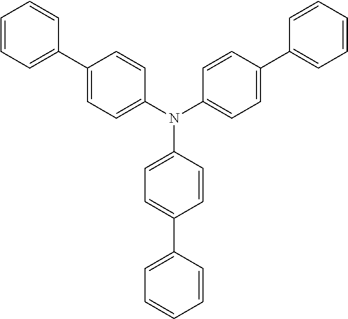 [SMILES: c1ccc(-c2ccc(N(c3ccc(-c4ccccc4)cc3)c3ccc(-c4ccccc4)cc3)cc2)cc1]
HT-29 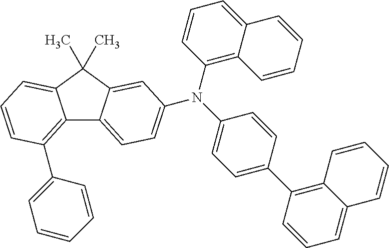 [SMILES: CC1(C)c2cc(N(c3ccc(-c4cccc5ccccc45)cc3)c3cccc4ccccc34)ccc2-c2c(-c3ccccc3)cccc21]
HT-30 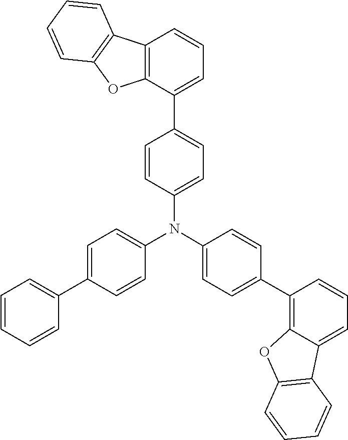 [SMILES: c1ccc(-c2ccc(N(c3ccc(-c4cccc5c4oc4ccccc45)cc3)c3ccc(-c4cccc5c4oc4ccccc45)cc3)cc2)cc1]
HT-31 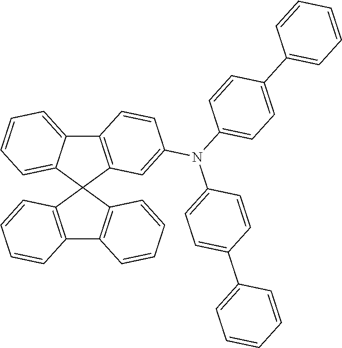 [SMILES: c1ccc(-c2ccc(N(c3ccc(-c4ccccc4)cc3)c3ccc4c(c3)C3(c5ccccc5-c5ccccc53)c3ccccc3-4)cc2)cc1]
HT-32 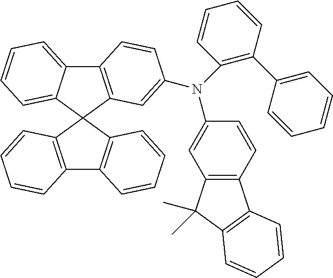 [SMILES: CC1(C)c2ccccc2-c2ccc(N(c3ccc4c(c3)C3(c5ccccc5-c5ccccc53)c3ccccc3-4)c3ccccc3-c3ccccc3)cc21]
HT-33 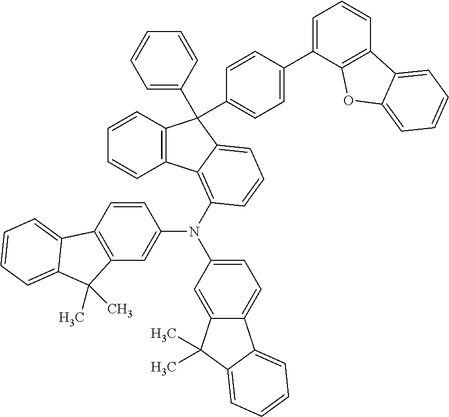 [SMILES: CC1(C)c2ccccc2-c2ccc(N(c3ccc4c(c3)C(C)(C)c3ccccc3-4)c3cccc4c3-c3ccccc3C4(c3ccccc3)c3ccc(-c4cccc5c4oc4ccccc45)cc3)cc21]
HT-34 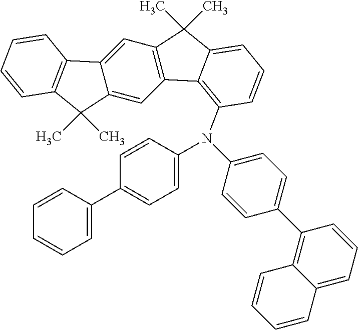 [SMILES: CC1(C)c2ccccc2-c2cc3c(cc21)-c1c(N(c2ccc(-c4ccccc4)cc2)c2ccc(-c4cccc5ccccc45)cc2)cccc1C3(C)C]
HT-35 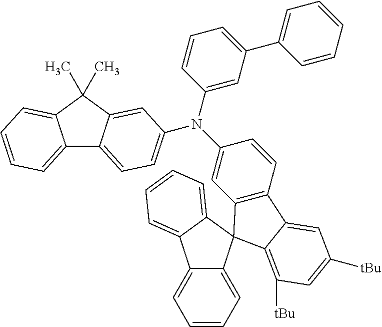 [SMILES: CC(C)(C)c1cc2c(c(C(C)(C)C)c1)C1(c3ccccc3-c3ccccc31)c1cc(N(c3cccc(-c4ccccc4)c3)c3ccc4c(c3)C(C)(C)c3ccccc3-4)ccc1-2]
HT-36 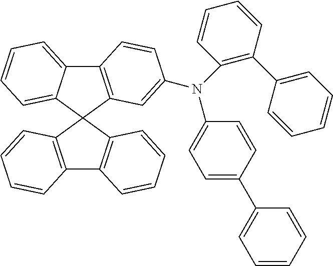 [SMILES: c1ccc(-c2ccc(N(c3ccc4c(c3)C3(c5ccccc5-c5ccccc53)c3ccccc3-4)c3ccccc3-c3ccccc3)cc2)cc1]
HT-37 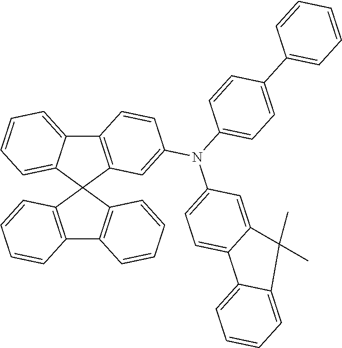 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]
HT-38 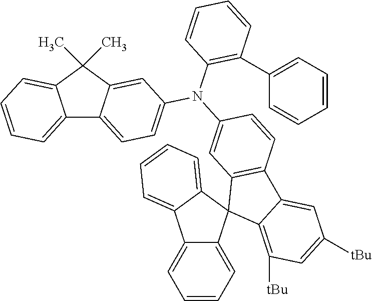 [SMILES: CC(C)(C)c1cc2c(c(C(C)(C)C)c1)C1(c3ccccc3-c3ccccc31)c1cc(N(c3ccc4c(c3)C(C)(C)c3ccccc3-4)c3ccccc3-c3ccccc3)ccc1-2]
HT-39 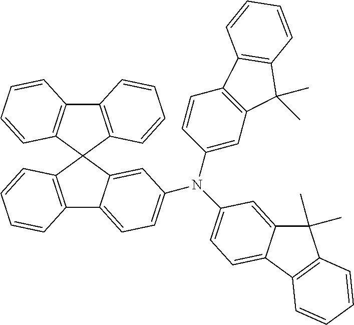 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21]
HT-40 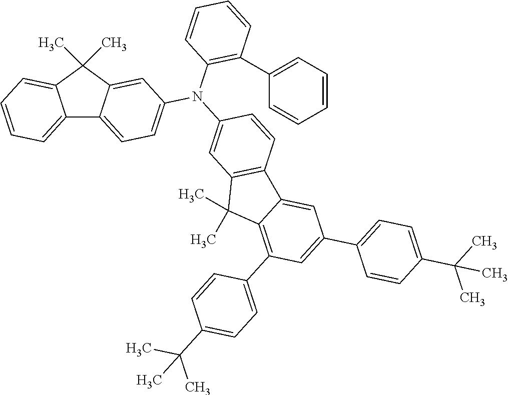 [SMILES: CC(C)(C)c1ccc(-c2cc(-c3ccc(C(C)(C)C)cc3)c3c(c2)-c2ccc(N(c4ccc5c(c4)C(C)(C)c4ccccc4-5)c4ccccc4-c4ccccc4)cc2C3(C)C)cc1]
HT-41 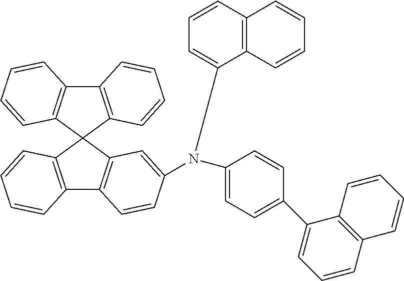 [SMILES: c1ccc2c(c1)-c1ccccc1C21c2ccccc2-c2ccc(N(c3ccc(-c4cccc5ccccc45)cc3)c3cccc4ccccc34)cc21]
HT-42 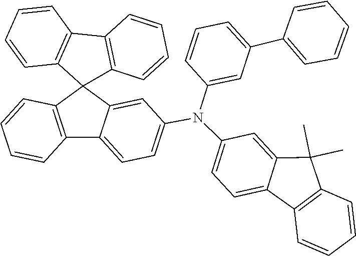 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21]
HT-43 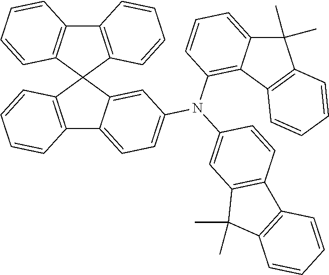 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21]
HT-44 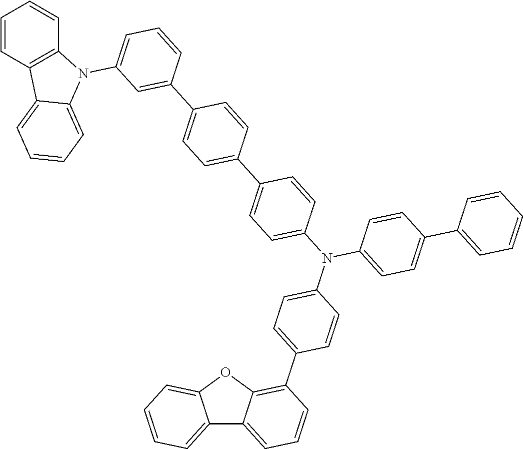 [SMILES: c1ccc(-c2ccc(N(c3ccc(-c4ccc(-c5cccc(-n6c7ccccc7c7ccccc76)c5)cc4)cc3)c3ccc(-c4cccc5c4oc4ccccc45)cc3)cc2)cc1]
HT-45 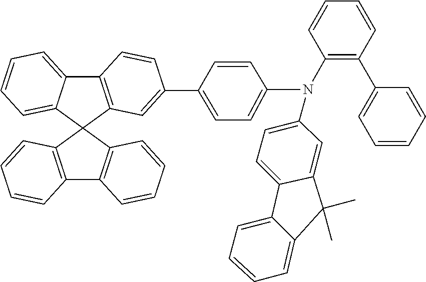 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]
HT-46 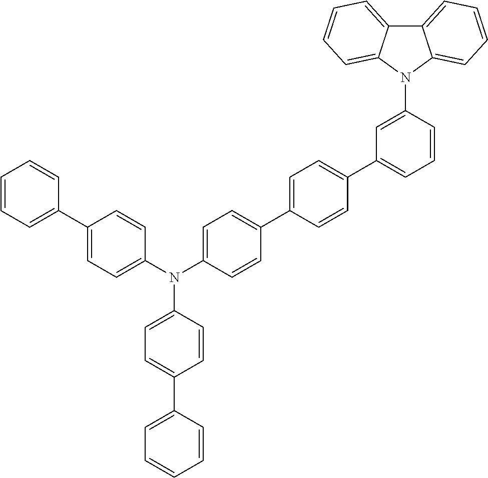 [SMILES: c1ccc(-c2ccc(N(c3ccc(-c4ccccc4)cc3)c3ccc(-c4ccc(-c5cccc(-n6c7ccccc7c7ccccc76)c5)cc4)cc3)cc2)cc1]
HT-47 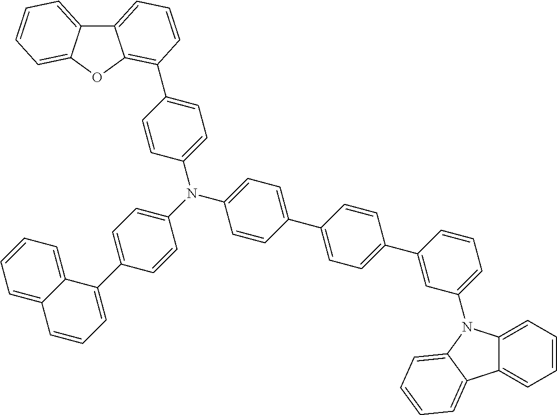 [SMILES: c1cc(-c2ccc(-c3ccc(N(c4ccc(-c5cccc6ccccc56)cc4)c4ccc(-c5cccc6c5oc5ccccc56)cc4)cc3)cc2)cc(-n2c3ccccc3c3ccccc32)c1]
HT-48 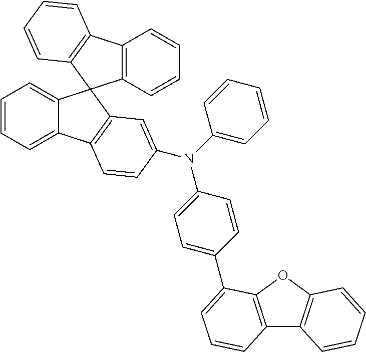 [SMILES: c1ccc(N(c2ccc(-c3cccc4c3oc3ccccc34)cc2)c2ccc3c(c2)C2(c4ccccc4-c4ccccc42)c2ccccc2-3)cc1]
HT-49 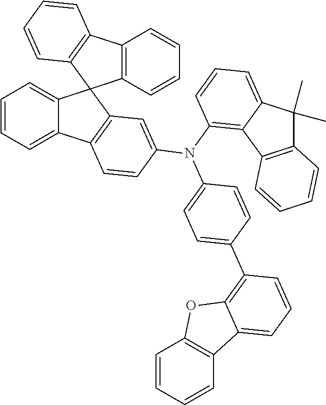 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]
HT-50 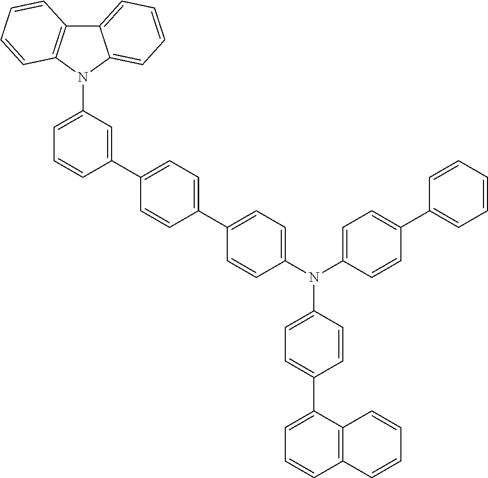 [SMILES: c1ccc(-c2ccc(N(c3ccc(-c4ccc(-c5cccc(-n6c7ccccc7c7ccccc76)c5)cc4)cc3)c3ccc(-c4cccc5ccccc45)cc3)cc2)cc1]
HT-51 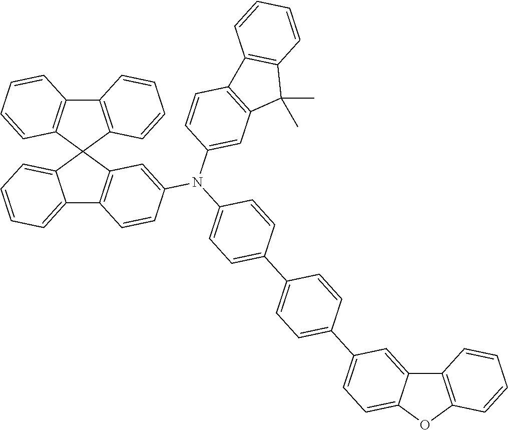 [SMILES: CC1(C)c2ccccc2-c2ccc(N(c3ccc(-c4ccc(-c5ccc6oc7ccccc7c6c5)cc4)cc3)c3ccc4c(c3)C3(c5ccccc5-c5ccccc53)c3ccccc3-4)cc21]
HT-52 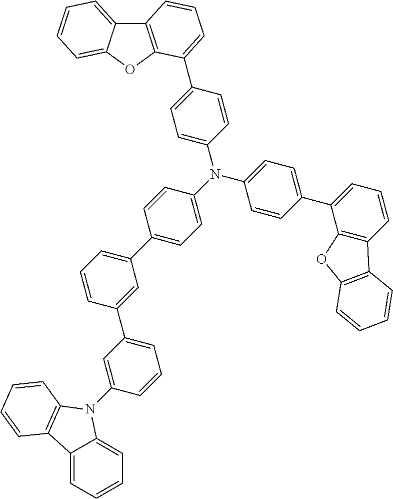 [SMILES: c1cc(-c2ccc(N(c3ccc(-c4cccc5c4oc4ccccc45)cc3)c3ccc(-c4cccc5c4oc4ccccc45)cc3)cc2)cc(-c2cccc(-n3c4ccccc4c4ccccc43)c2)c1]
HT-53 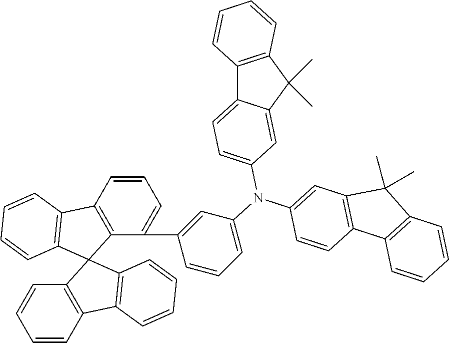 [SMILES: CC1(C)c2ccccc2-c2ccc(N(c3cccc(-c4cccc5c4C4(c6ccccc6-c6ccccc64)c4ccccc4-5)c3)c3ccc4c(c3)C(C)(C)c3ccccc3-4)cc21]
HT-54 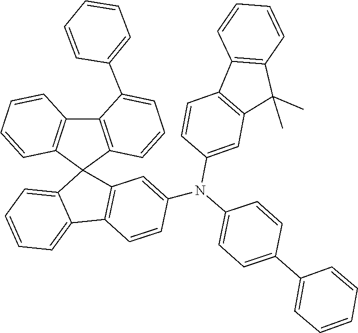 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21]
HT-55 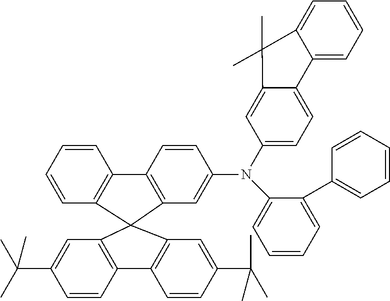 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]
HT-56 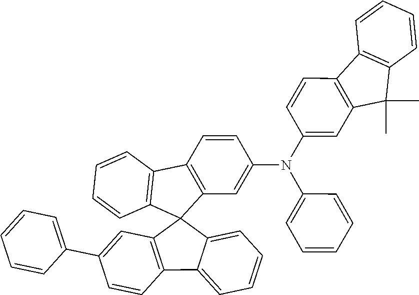 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21]
HT-57 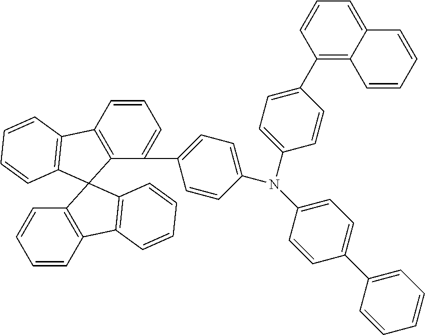 [SMILES: c1ccc(-c2ccc(N(c3ccc(-c4cccc5c4C4(c6ccccc6-c6ccccc64)c4ccccc4-5)cc3)c3ccc(-c4cccc5ccccc45)cc3)cc2)cc1]
HT-58 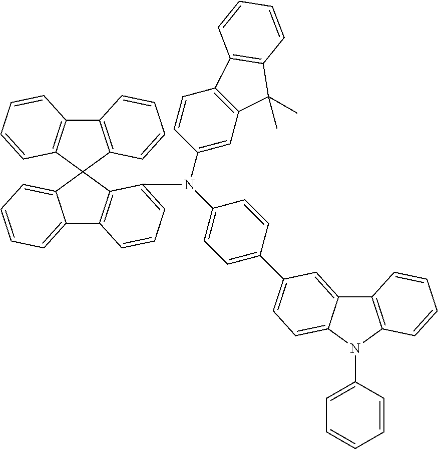 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21]
HT-59 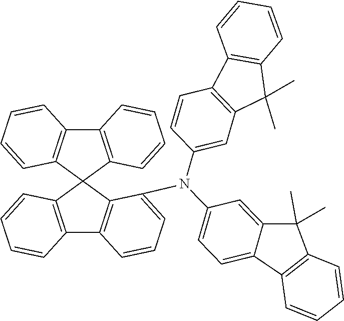 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]
HT-60 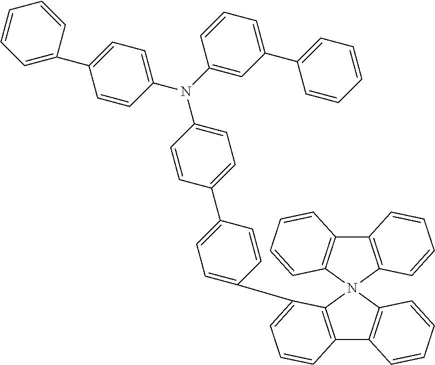
HT-61 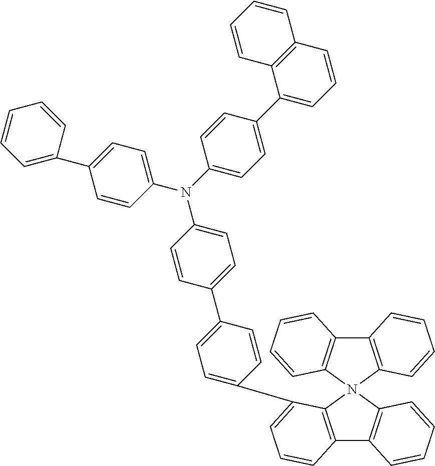
HT-62 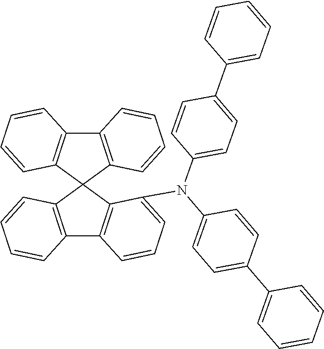 [SMILES: c1ccc(-c2ccc(N(c3ccc(-c4ccccc4)cc3)c3cccc4c3C3(c5ccccc5-c5ccccc53)c3ccccc3-4)cc2)cc1]
HT-63 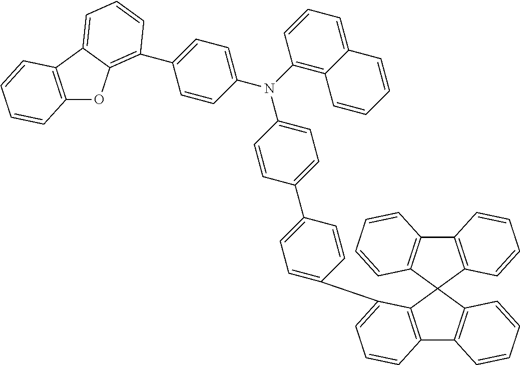 [SMILES: c1ccc2c(c1)-c1ccccc1C21c2ccccc2-c2cccc(-c3ccc(-c4ccc(N(c5ccc(-c6cccc7c6oc6ccccc67)cc5)c5cccc6ccccc56)cc4)cc3)c21]
HT-64 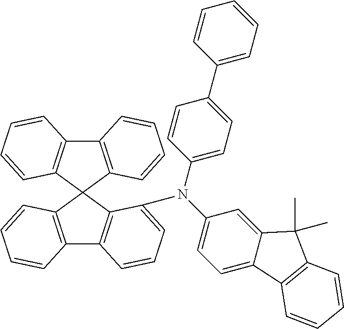 [SMILES: CC1(C)c2ccccc2-c2ccc(N(c3ccc(-c4ccccc4)cc3)c3cccc4c3C3(c5ccccc5-c5ccccc53)c3ccccc3-4)cc21]
HT-65 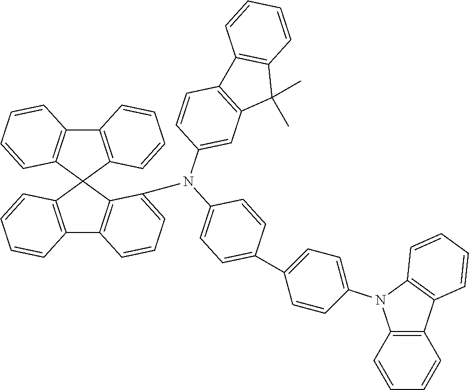 [SMILES: CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]
HT-66 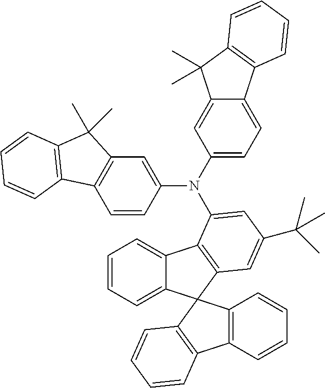 [SMILES: CC(C)(C)c1cc(N(c2ccc3c(c2)C(C)(C)c2ccccc2-3)c2ccc3c(c2)C(C)(C)c2ccccc2-3)c2c(c1)C1(c3ccccc3-c3ccccc31)c1ccccc1-2]
HT-67 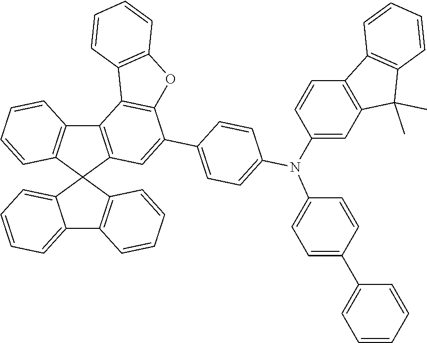 [SMILES: CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]
HT-68 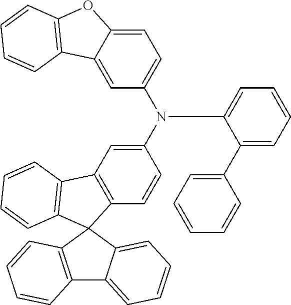 [SMILES: c1ccc(-c2ccccc2N(c2ccc3c(c2)-c2ccccc2C32c3ccccc3-c3ccccc32)c2ccc3oc4ccccc4c3c2)cc1]
HT-69 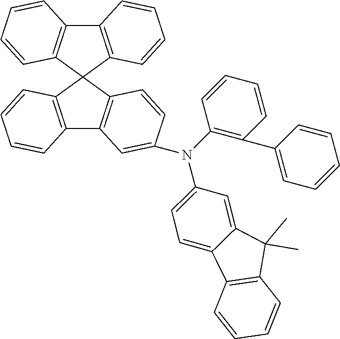 [SMILES: CC1(C)c2ccccc2-c2ccc(N(c3ccc4c(c3)-c3ccccc3C43c4ccccc4-c4ccccc43)c3ccccc3-c3ccccc3)cc21]
HT-70 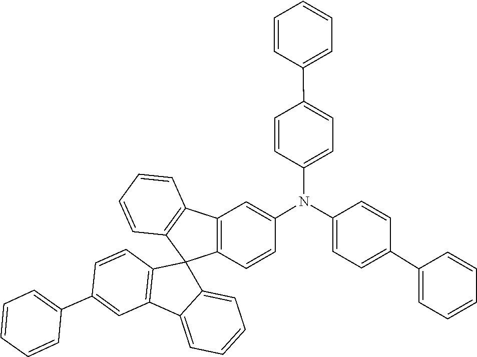 [SMILES: c1ccc(-c2ccc(N(c3ccc(-c4ccccc4)cc3)c3ccc4c(c3)-c3ccccc3C43c4ccccc4-c4cc(-c5ccccc5)ccc43)cc2)cc1]
HT-71 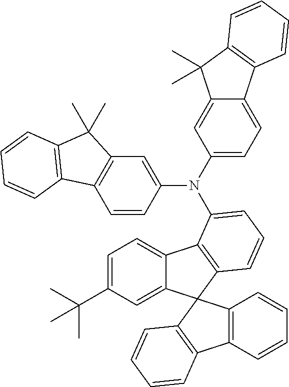 [SMILES: CC(C)(C)c1ccc2c(c1)C1(c3ccccc3-c3ccccc31)c1cccc(N(c3ccc4c(c3)C(C)(C)c3ccccc3-4)c3ccc4c(c3)C(C)(C)c3ccccc3-4)c1-2]
HT-72 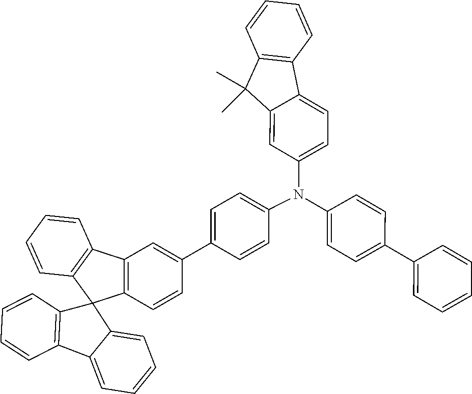 [SMILES: CC1(C)c2ccccc2-c2ccc(N(c3ccc(-c4ccccc4)cc3)c3ccc(-c4ccc5c(c4)-c4ccccc4C54c5ccccc5-c5ccccc54)cc3)cc21]
HT-73 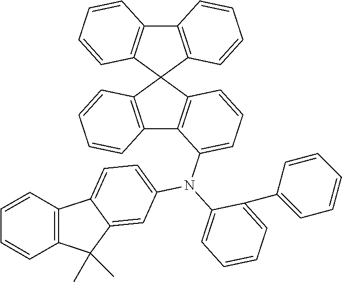 [SMILES: CC1(C)c2ccccc2-c2ccc(N(c3ccccc3-c3ccccc3)c3cccc4c3-c3ccccc3C43c4ccccc4-c4ccccc43)cc21]
HT-74 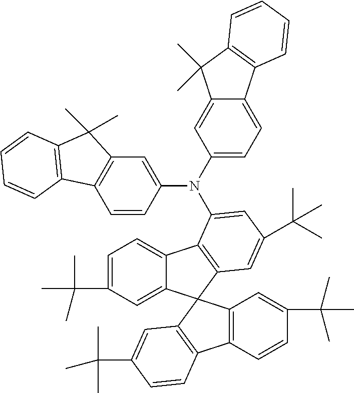 [SMILES: CC(C)(C)c1ccc2c(c1)C1(c3cc(C(C)(C)C)ccc3-2)c2cc(C(C)(C)C)ccc2-c2c(N(c3ccc4c(c3)C(C)(C)c3ccccc3-4)c3ccc4c(c3)C(C)(C)c3ccccc3-4)cc(C(C)(C)C)cc21]
HT-75 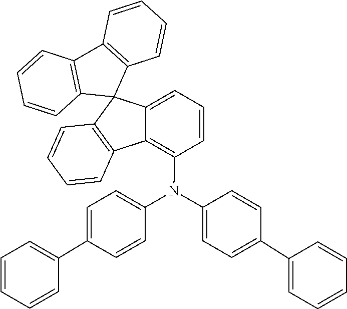 [SMILES: c1ccc(-c2ccc(N(c3ccc(-c4ccccc4)cc3)c3cccc4c3-c3ccccc3C43c4ccccc4-c4ccccc43)cc2)cc1]
HT-76 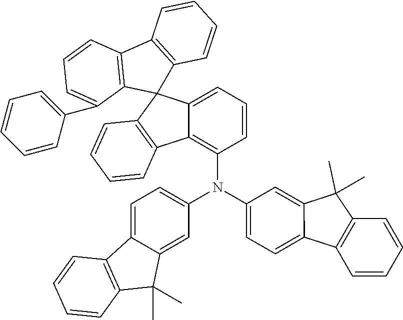 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5ccccc5)c43)cc21]
HT-77 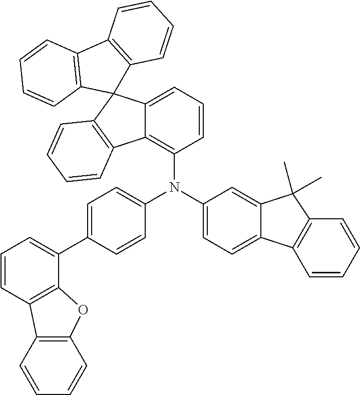 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21]
HT-78 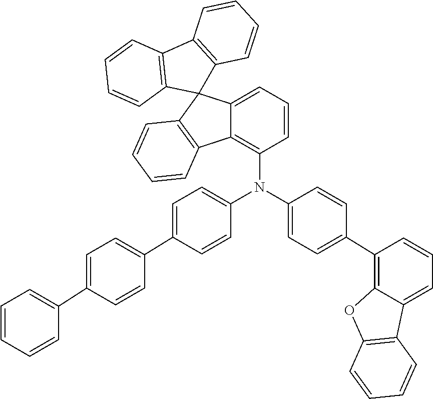 [SMILES: c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1]
HT-79 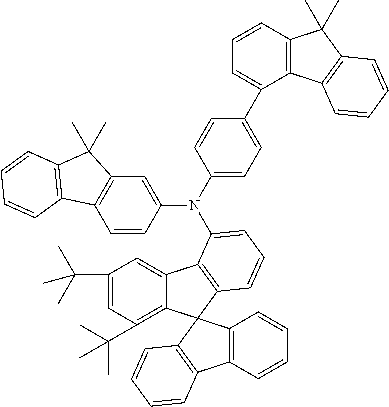 [SMILES: CC(C)(C)c1cc2c(c(C(C)(C)C)c1)C1(c3ccccc3-c3ccccc31)c1cccc(N(c3ccc(-c4cccc5c4-c4ccccc4C5(C)C)cc3)c3ccc4c(c3)C(C)(C)c3ccccc3-4)c1-2]
HT-80 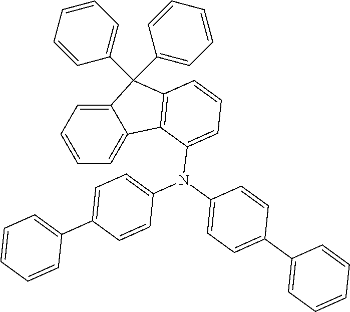 [SMILES: c1ccc(-c2ccc(N(c3ccc(-c4ccccc4)cc3)c3cccc4c3-c3ccccc3C4(c3ccccc3)c3ccccc3)cc2)cc1]
HT-81 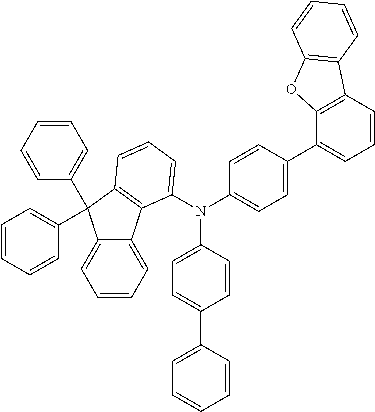 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1]
HT-82 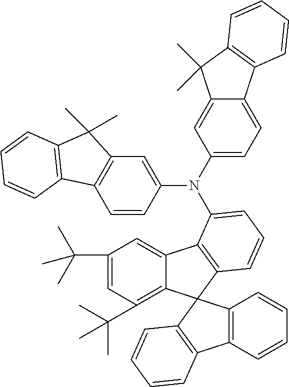 [SMILES: CC(C)(C)c1cc2c(c(C(C)(C)C)c1)C1(c3ccccc3-c3ccccc31)c1cccc(N(c3ccc4c(c3)C(C)(C)c3ccccc3-4)c3ccc4c(c3)C(C)(C)c3ccccc3-4)c1-2]
HT-83 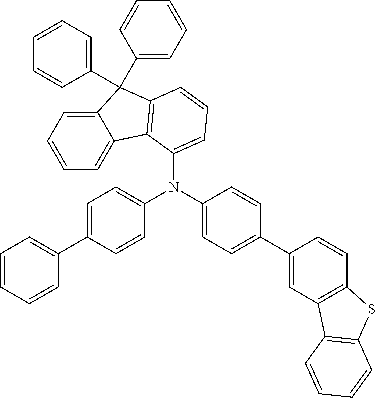 [SMILES: c1ccc(-c2ccc(N(c3ccc(-c4ccc5sc6ccccc6c5c4)cc3)c3cccc4c3-c3ccccc3C4(c3ccccc3)c3ccccc3)cc2)cc1]
HT-84 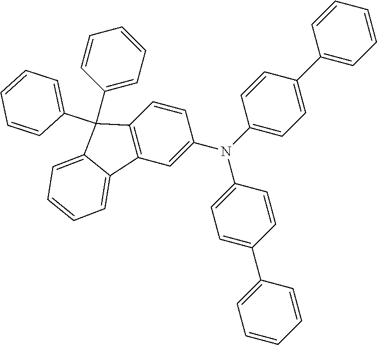 [SMILES: c1ccc(-c2ccc(N(c3ccc(-c4ccccc4)cc3)c3ccc4c(c3)-c3ccccc3C4(c3ccccc3)c3ccccc3)cc2)cc1]
HT-85 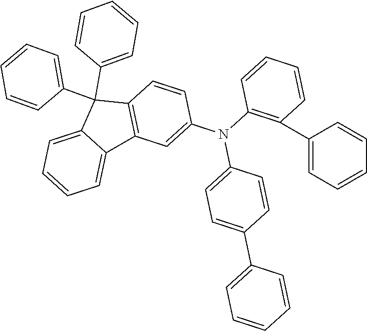 [SMILES: c1ccc(-c2ccc(N(c3ccc4c(c3)-c3ccccc3C4(c3ccccc3)c3ccccc3)c3ccccc3-c3ccccc3)cc2)cc1]
HT-86 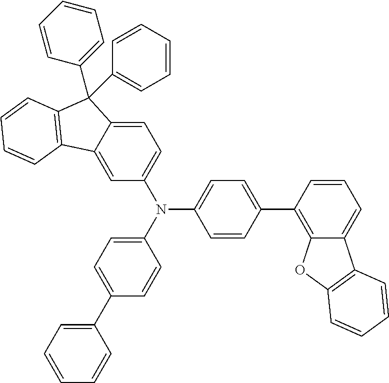 [SMILES: c1ccc(-c2ccc(N(c3ccc(-c4cccc5c4oc4ccccc45)cc3)c3ccc4c(c3)-c3ccccc3C4(c3ccccc3)c3ccccc3)cc2)cc1]
HT-87 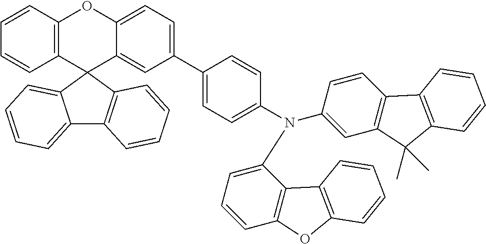 [SMILES: CC1(C)c2ccccc2-c2ccc(N(c3ccc(-c4ccc5c(c4)C4(c6ccccc6O5)c5ccccc5-c5ccccc54)cc3)c3cccc4oc5ccccc5c34)cc21]
HT-88 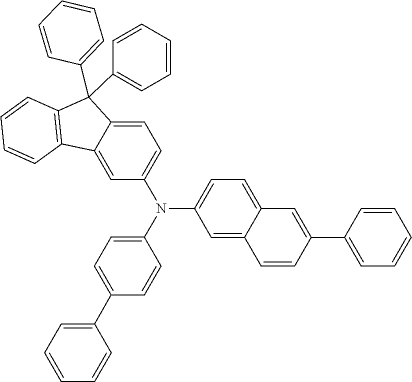 [SMILES: c1ccc(-c2ccc(N(c3ccc4c(c3)-c3ccccc3C4(c3ccccc3)c3ccccc3)c3ccc4cc(-c5ccccc5)ccc4c3)cc2)cc1]
HT-89 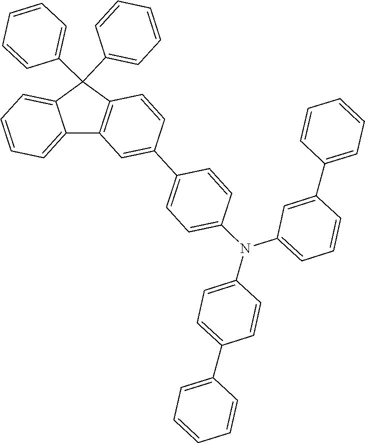 [SMILES: c1ccc(-c2ccc(N(c3ccc(-c4ccc5c(c4)-c4ccccc4C5(c4ccccc4)c4ccccc4)cc3)c3cccc(-c4ccccc4)c3)cc2)cc1]
HT-90 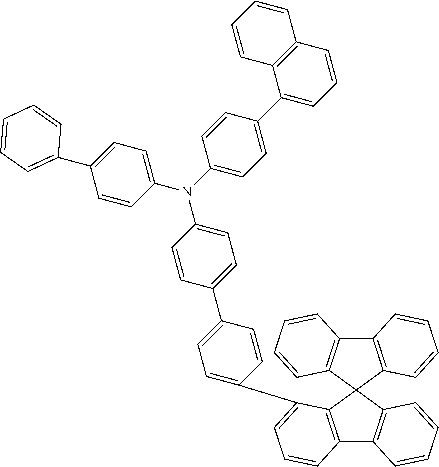 [SMILES: c1ccc(-c2ccc(N(c3ccc(-c4ccc(-c5cccc6c5C5(c7ccccc7-c7ccccc75)c5ccccc5-6)cc4)cc3)c3ccc(-c4cccc5ccccc45)cc3)cc2)cc1]
HT-91 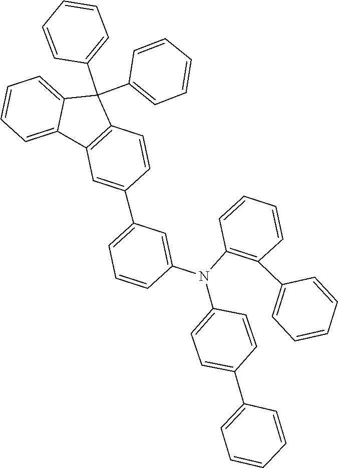 [SMILES: c1ccc(-c2ccc(N(c3cccc(-c4ccc5c(c4)-c4ccccc4C5(c4ccccc4)c4ccccc4)c3)c3ccccc3-c3ccccc3)cc2)cc1]
HT-92 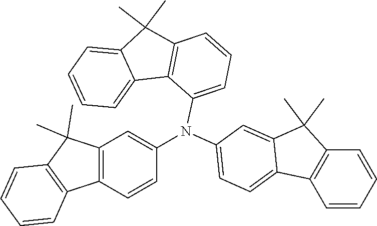 [SMILES: CC1(C)c2ccccc2-c2ccc(N(c3ccc4c(c3)C(C)(C)c3ccccc3-4)c3cccc4c3-c3ccccc3C4(C)C)cc21]
HT-93 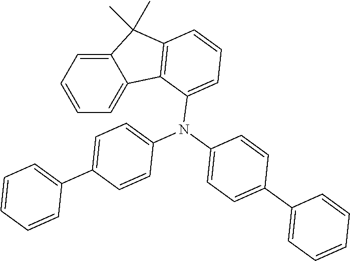 [SMILES: CC1(C)c2ccccc2-c2c(N(c3ccc(-c4ccccc4)cc3)c3ccc(-c4ccccc4)cc3)cccc21]
HT-94 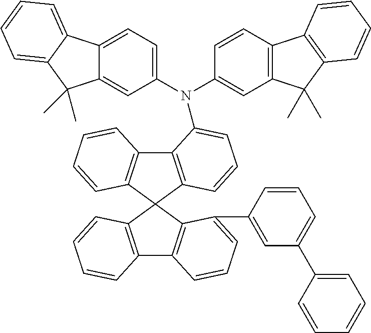 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5cccc(-c6ccccc6)c5)c43)cc21]
HT-95 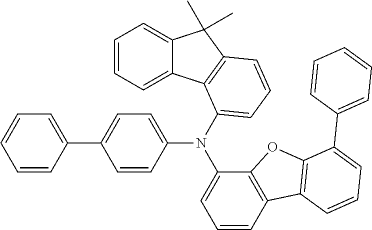 [SMILES: CC1(C)c2ccccc2-c2c(N(c3ccc(-c4ccccc4)cc3)c3cccc4c3oc3c(-c5ccccc5)cccc34)cccc21]
HT-96 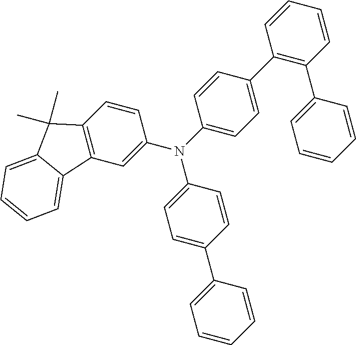 [SMILES: CC1(C)c2ccccc2-c2cc(N(c3ccc(-c4ccccc4)cc3)c3ccc(-c4ccccc4-c4ccccc4)cc3)ccc21]
HT-97 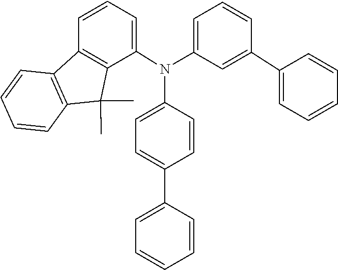 [SMILES: CC1(C)c2ccccc2-c2cccc(N(c3ccc(-c4ccccc4)cc3)c3cccc(-c4ccccc4)c3)c21]
HT-98 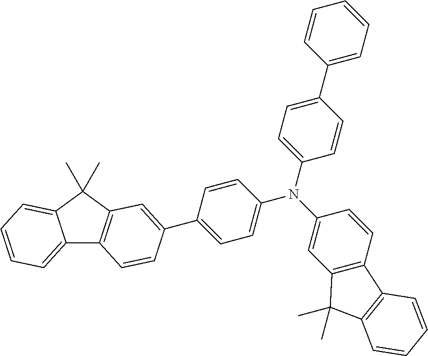 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21]
HT-99 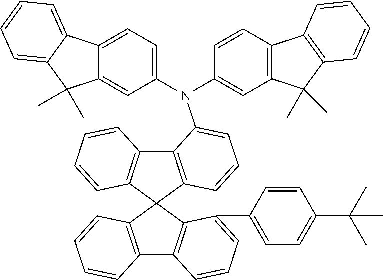 [SMILES: CC(C)(C)c1ccc(-c2cccc3c2C2(c4ccccc4-3)c3ccccc3-c3c(N(c4ccc5c(c4)C(C)(C)c4ccccc4-5)c4ccc5c(c4)C(C)(C)c4ccccc4-5)cccc32)cc1]
HT-100 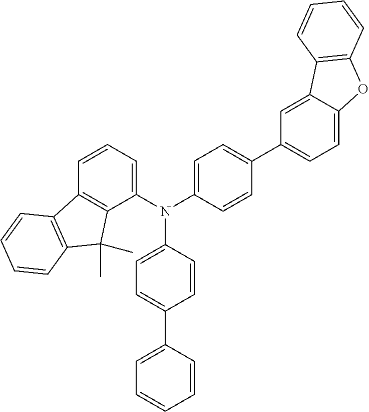 [SMILES: CC1(C)c2ccccc2-c2cccc(N(c3ccc(-c4ccccc4)cc3)c3ccc(-c4ccc5oc6ccccc6c5c4)cc3)c21]
HT-101 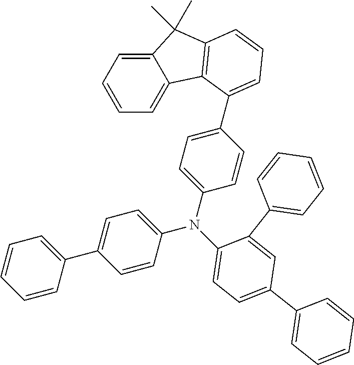 [SMILES: CC1(C)c2ccccc2-c2c(-c3ccc(N(c4ccc(-c5ccccc5)cc4)c4ccc(-c5ccccc5)cc4-c4ccccc4)cc3)cccc21]
HT-102 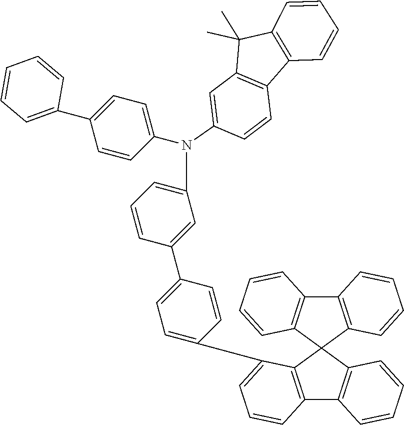 [SMILES: CC1(C)c2ccccc2-c2ccc(N(c3ccc(-c4ccccc4)cc3)c3cccc(-c4ccc(-c5cccc6c5C5(c7ccccc7-c7ccccc75)c5ccccc5-6)cc4)c3)cc21]
HT-103 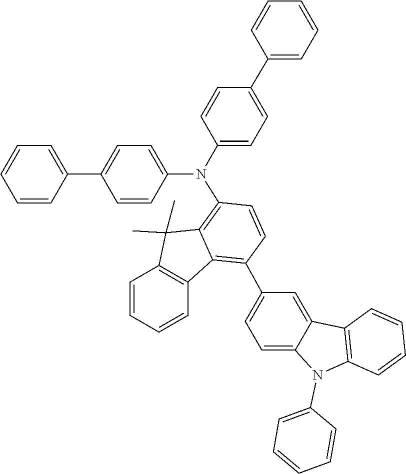 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]
HT-104 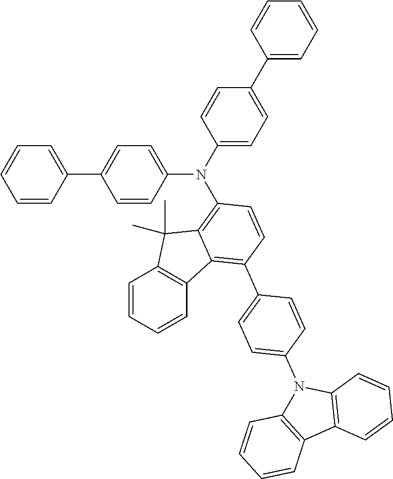 [SMILES: CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]
HT-105 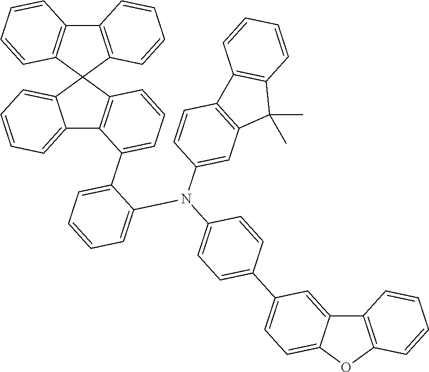 [SMILES: CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21]
HT-106 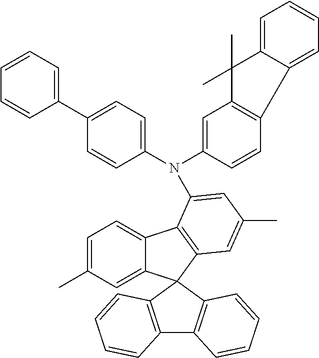 [SMILES: Cc1ccc2c(c1)C1(c3ccccc3-c3ccccc31)c1cc(C)cc(N(c3ccc(-c4ccccc4)cc3)c3ccc4c(c3)C(C)(C)c3ccccc3-4)c1-2]
HT-107 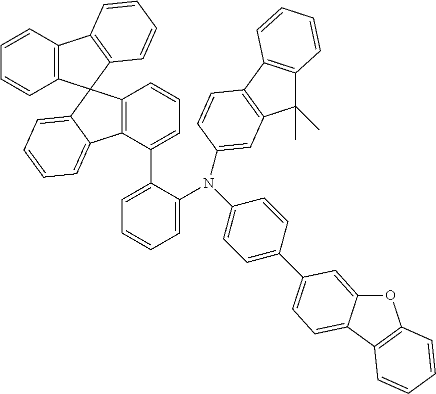 [SMILES: CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]
HT-108 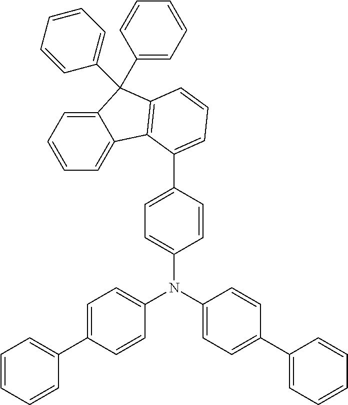 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1]
HT-109 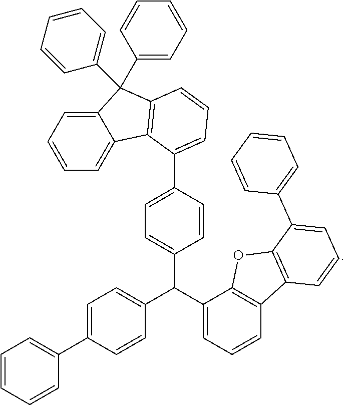 [SMILES: c1ccc(-c2ccc(C(c3ccc(-c4cccc5c4-c4ccccc4C5(c4ccccc4)c4ccccc4)cc3)c3cccc4c3oc3c(-c5ccccc5)cccc34)cc2)cc1]
HT-110

The sequence of layers in the organic electroluminescent device of the invention is preferably as follows:
anode/hole injection layer/hole transport layer/emitting layer/electron transport layer/electron injection layer/cathode.

This sequence of the layers is a preferred sequence.

At the same time, it should be pointed out again that not all the layers mentioned need be present and/or that further layers may additionally be present.

Materials used for the electron transport layer may be any materials as used according to the prior art as electron transport materials in the electron transport layer. Especially suitable are aluminum complexes, for example Alq₃, zirconium complexes, for example Zrq₄, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Suitable cathodes of the device of the invention are metals having a low work function, metal alloys or multilayer structures composed of various metals, for example alkaline earth metals, alkali metals, main group metals or lanthanoids (e.g. Ca, Ba, Mg, Al, In, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The organic electroluminescent device of the invention, in the course of production, is appropriately (according to the application) structured, contact-connected and finally sealed, since the lifetime of the devices of the invention is shortened in the presence of water and/or air.

The production of the device of the invention is not restricted here. It is possible that one or more organic layers, including the light-emitting layer, are coated by a sublimation method. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

The organic electroluminescent device of the invention is preferably characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

The organic electroluminescent device of the invention is further preferably characterized in that one or more organic layers comprising the composition of the invention are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble host materials 1 and 2 and phosphorescent emitters are needed. Processing from solution has the advantage that, for example, the light-emitting layer can be applied in a very simple and inexpensive manner. This technique is especially suitable for the mass production of organic electroluminescent devices.

In addition, hybrid methods are possible, in which, for example, one or more layers are applied from solution and one or more further layers are applied by vapor deposition.

These methods are known in general terms to those skilled in the art and can be applied to organic electroluminescent devices.

The invention therefore further provides a process for producing the organic electroluminescent device of the invention as described above or described as preferred, characterized in that the organic layer, preferably the light-emitting layer, the hole injection layer and/or hole transport layer, is applied by gas phase deposition, especially by a sublimation method and/or by an OVPD (organic vapor phase deposition) method and/or with the aid of a carrier gas sublimation, or from solution, especially by spin-coating or by a printing method.

In the case of production by means of gas phase deposition, there are in principle two ways in which the organic layer, preferably the light-emitting layer, of the invention can be applied or vapor-deposited onto any substrate or the prior layer. Firstly, the materials used can each be initially charged in a material source and ultimately evaporated from the different material sources (“co-evaporation”). Secondly, the various materials can be premixed (premix systems) and the mixture can be initially charged in a single material source from which it is ultimately evaporated (“premix evaporation”). In this way, it is possible in a simple and rapid manner to achieve the vapor deposition of the light-emitting layer with homogeneous distribution of the components without the need for precise actuation of a multitude of material sources.

The invention accordingly further provides a process for producing the device of the invention, characterized in that the light-emitting layer of the organic layer is applied by gas phase deposition, wherein the at least one compound of the formula (1) is deposited from the gas phase together with the further materials that form the light-emitting layer, successively or simultaneously from at least two material sources.

In a preferred embodiment of the present invention, the light-emitting layer is applied by means of gas phase deposition, wherein the constituents of the composition are premixed and evaporated from a single material source.

The invention accordingly further provides a process for producing the device of the invention, characterized in that the light-emitting layer of the organic layer is applied by gas phase deposition, wherein the at least one compound of the formula (1) is deposited from the gas phase together with at least one further matrix material as premix, successively or simultaneously with the light-emitting materials selected from the group of the phosphorescent emitters, fluorescent emitters and/or emitters that exhibit TADF (thermally activated delayed fluorescence).

The devices of the invention feature the following surprising advantages over the prior art:

The use of the described material combination of the host materials 1 and 2 as described above especially leads to an increase in the lifetime of the devices. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

This is true in particular with respect to similar compounds that do not have substitution or have a different substitution pattern on the diazabenzofurocarbazole or diazabenzothienocarbazole base skeleton.

The electronic devices of the invention, especially organic electroluminescent devices, are notable for one or more of the following surprising advantages over the prior art:
1. Electronic devices, especially organic electroluminescent devices, comprising compounds of formula (I) or the preferred embodiments recited above and hereinafter, especially as matrix material or as electron-conducting materials, have a very good lifetime. In this context, these compounds especially bring about low roll-off, i.e. a small drop in power efficiency of the device at high luminances.
2. Electronic devices, especially organic electroluminescent devices, comprising compounds of formula (1) or the preferred embodiments recited above and hereinafter, as electron-conducting materials and/or matrix materials, have excellent efficiency. In this context, compounds of the invention having structures of formula (1) or the preferred embodiments recited above and hereinafter bring about a low operating voltage when used in electronic devices.
3. The inventive compounds of formula (1) or the preferred embodiments recited above and hereinafter exhibit very high stability and lifetime.
4. With compounds of formula (1) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. The use of compounds of formula (1) or the preferred embodiments recited above and hereinafter in layers of electronic devices, especially organic electroluminescent devices, leads to high mobility of the electron conductor structures.
6. Compounds of formula (1) or the preferred embodiments recited above and hereinafter have excellent glass film formation.
7. Compounds of formula (1) or the preferred embodiments recited above and hereinafter form very good films from solutions.
8. The compounds of formula (1) or the preferred embodiments recited above and hereinafter have a low triplet level T₁ which may, for example, be in the range of 2.55 eV to 2.75 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Any feature disclosed in the present invention, unless stated otherwise, should therefore be considered as an example from a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

The technical teaching disclosed with the present invention may be abstracted and combined with other examples.

The invention is illustrated in detail by the examples which follow, without any intention of restricting it thereby.

### EXAMPLES

### General Methods:

In all quantum-chemical calculations, the Gaussian16 (Rev. B.01) software package is used. The neutral singlet ground state is optimized at the B3LYP/6-31G(d) level. HOMO and LUMO values are determined at the B3LYP/6-31G(d) level for the B3LYP/6-31G(d)-optimized ground state energy. Then TD-DFT singlet and triplet excitations (vertical excitations) are calculated by the same method (B3LYP/6-31G(d)) and with the optimized ground state geometry. The standard settings for SCF and gradient convergence are used.

From the energy calculation, the HOMO is obtained as the last orbital occupied by two electrons (alpha occ. eigenvalues) and LUMO as the first unoccupied orbital (alpha virt. eigenvalues) in Hartree units, where HEh and LEh represent the HOMO energy in Hartree units and the LUMO energy in Hartree units respectively. This is used to determine the HOMO and LUMO value in electron volts, calibrated by cyclic voltammetry measurements, as follows:

HOMOcorr=0.90603*HOMO−0.84836

LUMOcorr=0.99687*LUMO−0.72445

The triplet level T1 of a material is defined as the relative excitation energy (in eV) of the triplet state having the lowest energy which is found by the quantum-chemical energy calculation.

The singlet level S1 of a material is defined as the relative excitation energy (in eV) of the singlet state having the second-lowest energy which is found by the quantum-chemical energy calculation.

The energetically lowest singlet state is referred to as SO.

The method described herein is independent of the software package used and always gives the same results. Examples of frequently utilized programs for this purpose are “Gaussian09” (Gaussian Inc.) and Q-Chem 4.1 (Q-Chem, Inc.). In the present case, the energies are calculated using the software package “GaussianI6 (Rev. B.01)”.

### Synthesis Examples

The syntheses which follow, unless stated otherwise, are conducted under a protective gas atmosphere in dried solvents. The reactants can be sourced from ALDRICH (potassium fluoride (spray-dried), tri-tert-butylphosphine, palladium(II) acetate). 3-Chloro-5,6-diphenyl-1,2,4-triazine can be prepared analogously to EP 577559. 2′,7′-Di-tert-butyl-spiro-9,9′-bifluorene-2,7-bisboronic acid glycol ester can be prepared according to WO 02/077060, and 2-chloro-4,6-diphenyl-1,3,5-triazine according to U.S. Pat. No. 5,438,138. Spiro-9,9′-bifluorene-2,7-bis(boronic acid glycol ester) can be prepared analogously to WO 02/077060.

### Synthesis Example 1

### a) 2,4-Dichloro-6-dibenzofuran-2-yl-1,3,5-triazine

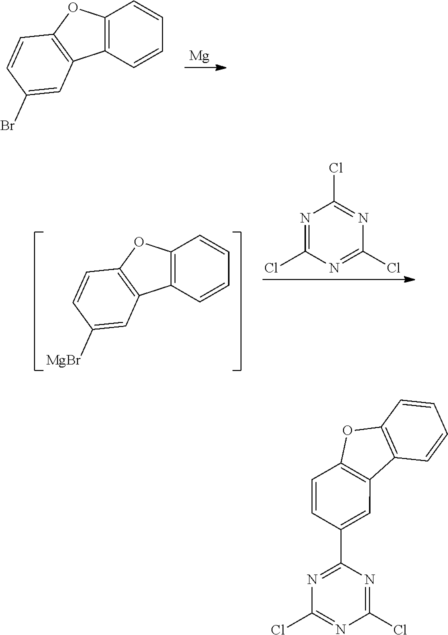

1.5 g (61 mmol, 1.12 eq) of magnesium turnings is heated in a four-neck flask for a few minutes. Then a few ml of a mixture of 14.8 g (60 mmol, 1.10 eq) of 2-bromodibenzofuran in 100 ml of dried THE is added until the Grignard reaction commences. Then the rest of the solution is added gradually in order to maintain the Grignard reaction at reflux. Once the addition is complete, the mixture is cooled down to about 0° C. with an ice bath. In a second apparatus, 10.9 g (60 mmol, 1.0 eq) of 2,4,6-trichloro-1,3,5-triazine dissolved in 60 ml of dried THE is cooled down with an ice bath. The Grignard reagent is transferred into a dropping funnel and added gradually to that solution. After stirring at room temperature overnight, the mixture is diluted with 100 ml of THF, and 50 ml of a 1 M HCl solution is added. The precipitate formed is washed with water, ethanol and heptane, and recrystallized in toluene. Yield: 12.7 g (40.4 mmol), 67% of theory, purity by ¹H NMR about 98%.

The following brominated compounds are prepared in an analogous manner:

| | Reactant 1 | Product | Yield |
|---|---|---|---|
| 1a | 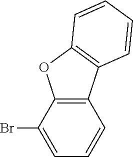 [SMILES: Brc1cccc2c1oc1ccccc12] [89827-45-2] | 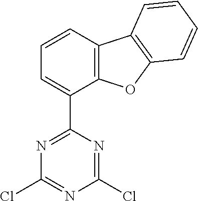 [SMILES: Clc1nc(Cl)nc(-c2cccc3c2oc2ccccc23)n1] | 67% |
| 2a | 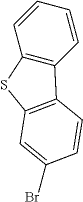 [SMILES: Brc1ccc2c(c1)sc1ccccc12] [97511-04-1] | 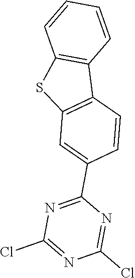 [SMILES: Clc1nc(Cl)nc(-c2ccc3c(c2)sc2ccccc23)n1] | 60% |
| 3a | 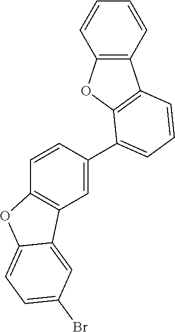 [SMILES: Brc1ccc2oc3ccc(-c4cccc5c4oc4ccccc45)cc3c2c1] [2377212-10-5] | 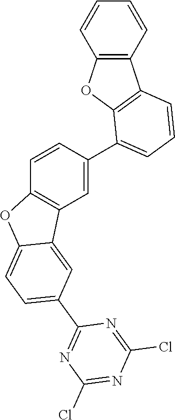 [SMILES: Clc1nc(Cl)nc(-c2ccc3oc4ccc(-c5cccc6c5oc5ccccc56)cc4c3c2)n1] | 68% |
| 4a | 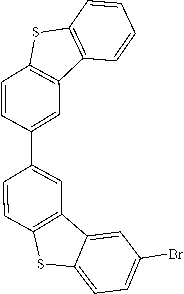 [SMILES: Brc1ccc2sc3ccc(-c4ccc5sc6ccccc6c5c4)cc3c2c1] [2377212-12-7] | 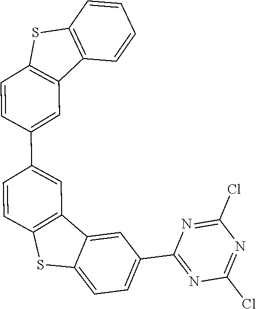 [SMILES: Clc1nc(Cl)nc(-c2ccc3sc4ccc(-c5ccc6sc7ccccc7c6c5)cc4c3c2)n1] | 70% |
| 5a | 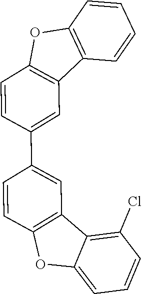 [SMILES: Clc1cccc2oc3ccc(-c4ccc5oc6ccccc6c5c4)cc3c12] | 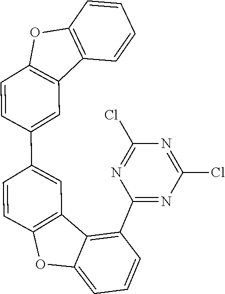 [SMILES: Clc1nc(Cl)nc(-c2cccc3oc4ccc(-c5ccc6oc7ccccc7c6c5)cc4c23)n1] | 59% |
| | [2299271-95-5] | | |
| 6a | 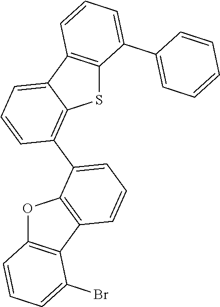 [SMILES: Brc1cccc2oc3c(-c4cccc5c4sc4c(-c6ccccc6)cccc45)cccc3c12] [1822311-26-1] | 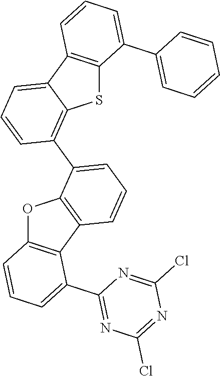 [SMILES: Clc1nc(Cl)nc(-c2cccc3oc4c(-c5cccc6c5sc5c(-c7ccccc7)cccc56)cccc4c23)n1] | 62% |
| 7a | 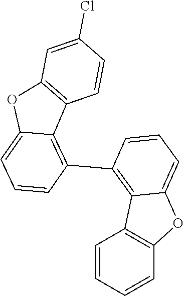 [SMILES: Clc1ccc2c(c1)oc1cccc(-c3cccc4oc5ccccc5c34)c12] | 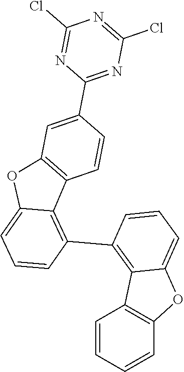 [SMILES: Clc1nc(Cl)nc(-c2ccc3c(c2)oc2cccc(-c4cccc5oc6ccccc6c45)c23)n1] | 60% |
| 8a | 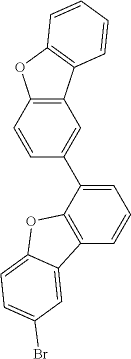 [SMILES: Brc1ccc2oc3c(-c4ccc5oc6ccccc6c5c4)cccc3c2c1] | 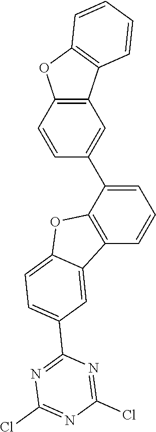 [SMILES: Clc1nc(Cl)nc(-c2ccc3oc4c(-c5ccc6oc7ccccc7c6c5)cccc4c3c2)n1] | 65% |

### b) 2-(8-Bromodibenzofuran-2-yl)-4,6-dichloro-1,3,5-triazine

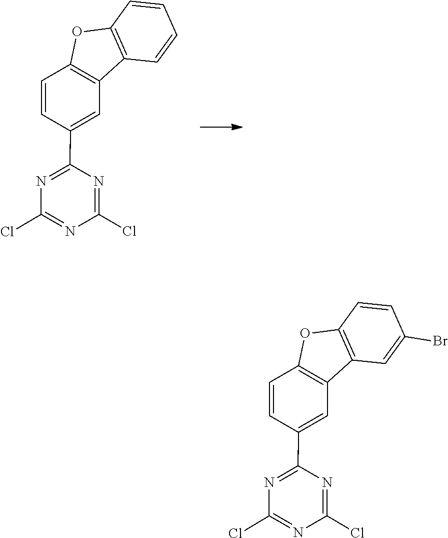 [SMILES: Clc1nc(Cl)nc(-c2ccc3oc4ccccc4c3c2)n1 ; Clc1nc(Cl)nc(-c2ccc3oc4ccc(Br)cc4c3c2)n1]

30 g (95 mmol) of 2,4-dichloro-6-dibenzofuran-2-yl-1,3,5-triazine is suspended in 1000 ml of acetic acid (100%) and 1000 ml of sulfuric acid (95-98%). To this suspension is added 17 g (95 mmol) of NBS in portions, and the mixture is stirred in the dark for 2 hours. Thereafter, water/ice is added, and the solids are separated off and washed with ethanol. The residue is recrystallized from toluene. The yield is 30 g (78 mmol), corresponding to 82% of theory.

The following brominated compounds are prepared in an analogous manner:

| | Reactant 1 | Product | Yield |
|---|---|---|---|
| 1b | 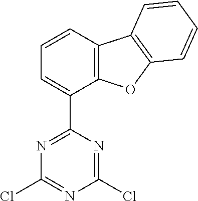 [SMILES: Clc1nc(Cl)nc(-c2cccc3c2oc2ccccc23)n1] | 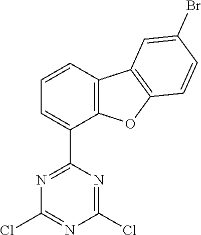 [SMILES: Clc1nc(Cl)nc(-c2cccc3c2oc2ccc(Br)cc23)n1] | 77% |
| 2b | 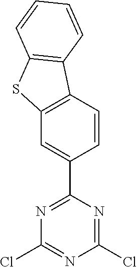 [SMILES: Clc1nc(Cl)nc(-c2ccc3c(c2)sc2ccccc23)n1] | 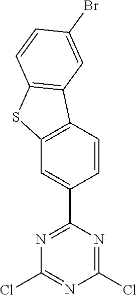 [SMILES: Clc1nc(Cl)nc(-c2ccc3c(c2)sc2ccc(Br)cc23)n1] | 62% |
| 3b | 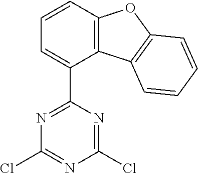 [SMILES: Clc1nc(Cl)nc(-c2cccc3oc4ccccc4c23)n1] | 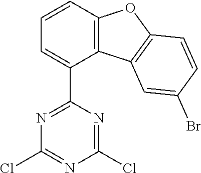 [SMILES: Clc1nc(Cl)nc(-c2cccc3oc4ccc(Br)cc4c23)n1] | 81% |
| | [2408705-92-8] | | |
| 4b | 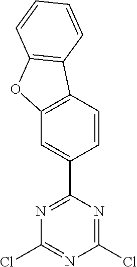 [SMILES: Clc1nc(Cl)nc(-c2ccc3c(c2)oc2ccccc23)n1] [2102042-41-9] | 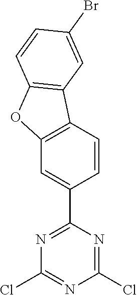 [SMILES: Clc1nc(Cl)nc(-c2ccc3c(c2)oc2ccc(Br)cc23)n1] | 61% |

### c) 2,4-Dichloro-6-(8-dibenzothiophen-4-yldibenzofuran-2-yl)-1,3,5-triazine

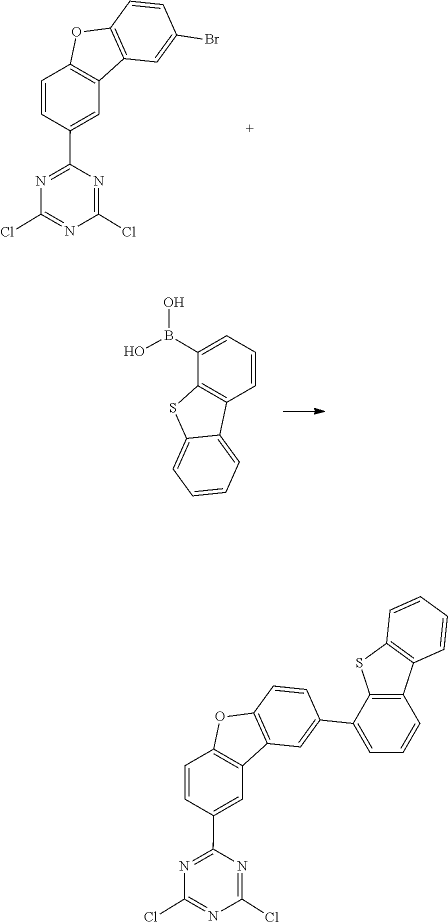 [SMILES: Clc1nc(Cl)nc(-c2ccc3oc4ccc(Br)cc4c3c2)n1 ; OB(O)c1cccc2c1sc1ccccc12 ; Clc1nc(Cl)nc(-c2ccc3oc4ccc(-c5cccc6c5sc5ccccc56)cc4c3c2)n1]

61 g (156 mmol) of 2-(8-bromodibenzofuran-2-yl)-4,6-dichloro-1,3,5-triazine, 39.2 g (172 mmol) of dibenzothiophene-4-boronic acid and 36 g (340 mmol) of sodium carbonate are suspended in 1000 ml of ethylene glycol diamine ether and 280 ml of water. To this suspension is added 1.8 g (1.5 mmol) of tetrakis(triphenylphosphine)-palladium(0), and the reaction mixture is heated under reflux for 16 h. After cooling, the organic phase is separated off, filtered through silica gel, washed three times with 200 ml of water, and then concentrated to dryness. The product is purified via column chromatography on silica gel with toluene/heptane (1:2). The yield is 50 g (101 mmol), corresponding to 65% of theory.

The following compounds can be prepared analogously:

| | Reactant 1 | Reactant 2 | Product | Yield |
|---|---|---|---|---|
| 1c | 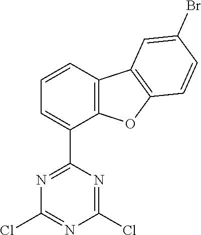 [SMILES: Clc1nc(Cl)nc(-c2cccc3c2oc2ccc(Br)cc23)n1] | 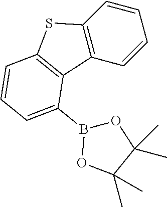 [SMILES: CC1(C)OB(c2cccc3sc4ccccc4c23)OC1(C)C] [1434286-69-7] | 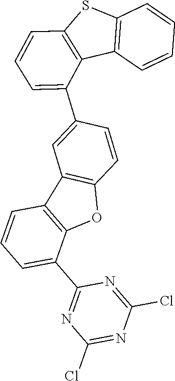 [SMILES: Clc1nc(Cl)nc(-c2cccc3c2oc2ccc(-c4cccc5sc6ccccc6c45)cc23)n1] | 57% |
| 2c | 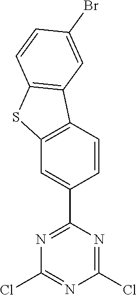 [SMILES: Clc1nc(Cl)nc(-c2ccc3c(c2)sc2ccc(Br)cc23)n1] | 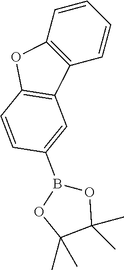 [SMILES: CC1(C)OB(c2ccc3oc4ccccc4c3c2)OC1(C)C] [947770-80-1] | 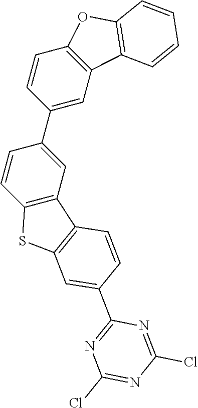 [SMILES: Clc1nc(Cl)nc(-c2ccc3c(c2)sc2ccc(-c4ccc5oc6ccccc6c5c4)cc23)n1] | 61% |
| 3c | 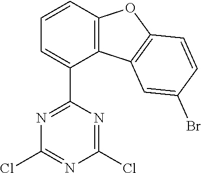 [SMILES: Clc1nc(Cl)nc(-c2cccc3oc4ccc(Br)cc4c23)n1] | 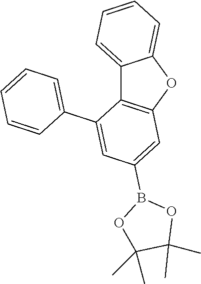 [SMILES: CC1(C)OB(c2cc(-c3ccccc3)c3c(c2)oc2ccccc23)OC1(C)C] [2361006-02-0] | 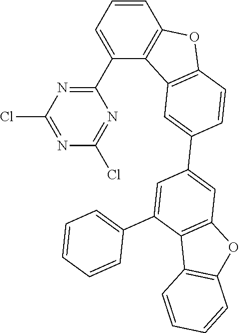 [SMILES: Clc1nc(Cl)nc(-c2cccc3oc4ccc(-c5cc(-c6ccccc6)c6c(c5)oc5ccccc56)cc4c23)n1] | 56% |
| 4c | 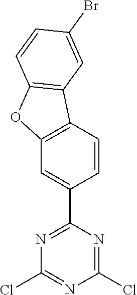 [SMILES: Clc1nc(Cl)nc(-c2ccc3c(c2)oc2ccc(Br)cc23)n1] | 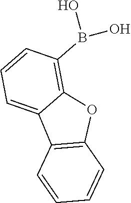 [SMILES: OB(O)c1cccc2c1oc1ccccc12] [100124-06-9] | 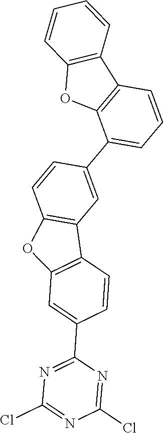 [SMILES: Clc1nc(Cl)nc(-c2ccc3c(c2)oc2ccc(-c4cccc5c4oc4ccccc45)cc23)n1] | 58% |
| 5c | 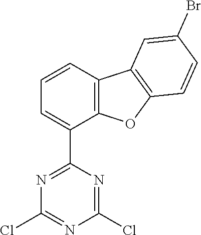 [SMILES: Clc1nc(Cl)nc(-c2cccc3c2oc2ccc(Br)cc23)n1] | 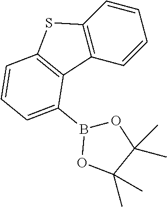 [SMILES: CC1(C)OB(c2cccc3sc4ccccc4c23)OC1(C)C] [1434286-69-7] | 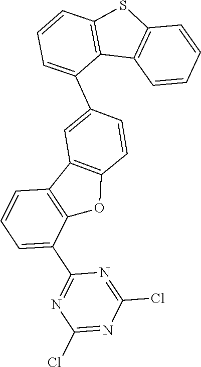 [SMILES: Clc1nc(Cl)nc(-c2cccc3c2oc2ccc(-c4cccc5sc6ccccc6c45)cc23)n1] | 66% |
| 6c | 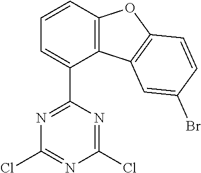 [SMILES: Clc1nc(Cl)nc(-c2cccc3oc4ccc(Br)cc4c23)n1] | 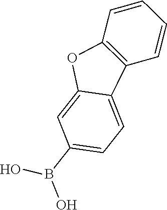 [SMILES: OB(O)c1ccc2c(c1)oc1ccccc12] [395087-89-5] | 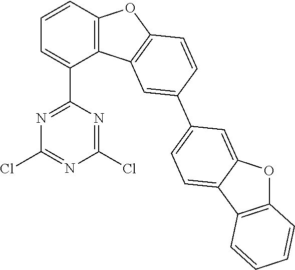 [SMILES: Clc1nc(Cl)nc(-c2cccc3oc4ccc(-c5ccc6c(c5)oc5ccccc56)cc4c23)n1] | 68% |
| 7c | 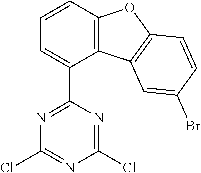 [SMILES: Clc1nc(Cl)nc(-c2cccc3oc4ccc(Br)cc4c23)n1] | 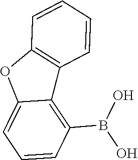 [SMILES: OB(O)c1cccc2oc3ccccc3c12] [162607-19-4] | 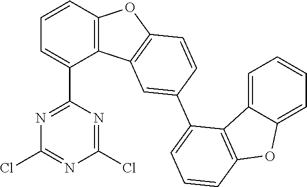 [SMILES: Clc1nc(Cl)nc(-c2cccc3oc4ccc(-c5cccc6oc7ccccc7c56)cc4c23)n1] | 60% |
| 8c | 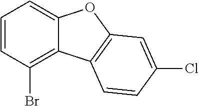 [SMILES: Clc1ccc2c(c1)oc1cccc(Br)c12] [2173555-52-5] | 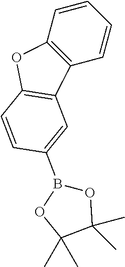 [SMILES: CC1(C)OB(c2ccc3oc4ccccc4c3c2)OC1(C)C] [947770-80-1] | 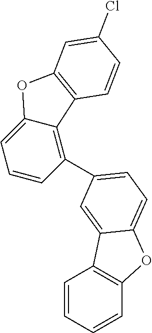 [SMILES: Clc1ccc2c(c1)oc1cccc(-c3ccc4oc5ccccc5c4c3)c12] | 58% |
| 9c | 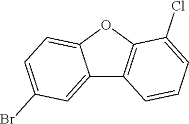 [SMILES: Clc1cccc2c1oc1ccc(Br)cc12] [2138490-84-1] | 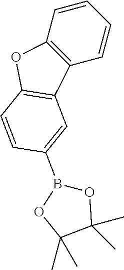 [SMILES: CC1(C)OB(c2ccc3oc4ccccc4c3c2)OC1(C)C] [947770-80-1] | 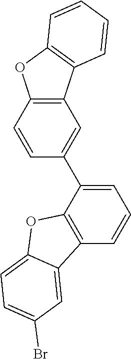 [SMILES: Brc1ccc2oc3c(-c4ccc5oc6ccccc6c5c4)cccc3c2c1] | 65% |

### d) 2-Chloro-4-(8-dibenzothiophen-4-yldibenzofuran-2-yl)-6-triphenylen-2-yl-1,3,5-triazine

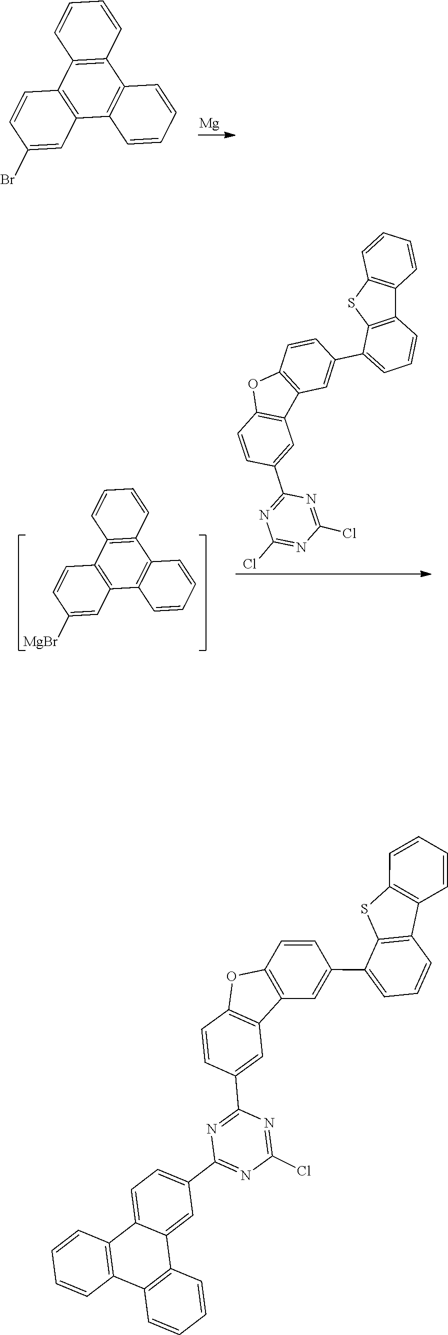

1.5 g (61 mmol, 1.12 eq) of magnesium turnings is heated in a four-neck flask for a few minutes. Then a few ml 18.6 g (60 mmol, 1.10 eq) of 2-bromotriphenylene in 100 ml of dried THE is added until the Grignard reaction commences. Then the rest of the solution is added gradually in order to keep the Grignard reaction at reflux. Once the addition is complete, the mixture is cooled to about 0° C. with an ice bath. In a second apparatus, 29.8 g (60 mmol, 1.0 eq) of 2,4-dichloro-6-(8-dibenzothiophen-4-yldibenzofuran-2-yl)-1,3,5-triazine dissolved in 60 ml of dried THE is cooled down with an ice bath. The Grignard reagent is transferred into a dropping funnel and added gradually to that solution. After stirring at room temperature overnight, the mixture is diluted with 100 ml of THF, and 50 ml of a 1 M HCl solution is added. The precipitate formed is washed with water, ethanol and heptane, and recrystallized from toluene. Yield: 29.7 g (43 mmol), 72% of theory, purity by ¹H NMR about 98%.

The following compounds can be prepared analogously:

| | Reactant 1 | Reactant e | Product | Yield |
|---|---|---|---|---|
| 1d | 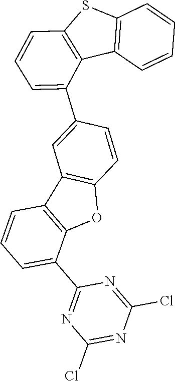 [SMILES: Clc1nc(Cl)nc(-c2cccc3c2oc2ccc(-c4cccc5sc6ccccc6c45)cc23)n1] | 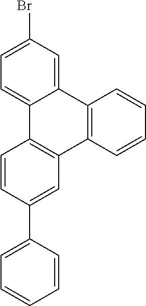 [SMILES: Brc1ccc2c3ccc(-c4ccccc4)cc3c3ccccc3c2c1] [1616514-01-2] | 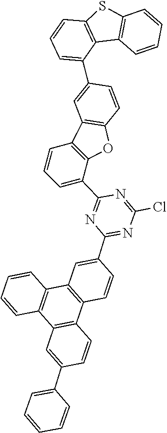 [SMILES: Clc1nc(-c2ccc3c4ccc(-c5ccccc5)cc4c4ccccc4c3c2)nc(-c2cccc3c2oc2ccc(-c4cccc5sc6ccccc6c45)cc23)n1] | 60% |
| 2d | 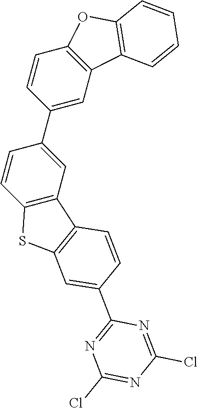 [SMILES: Clc1nc(Cl)nc(-c2ccc3c(c2)sc2ccc(-c4ccc5oc6ccccc6c5c4)cc23)n1] | 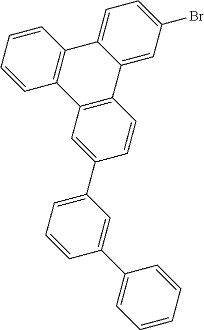 [SMILES: Brc1ccc2c3ccccc3c3cc(-c4cccc(-c5ccccc5)c4)ccc3c2c1] [1616514-20-5] | 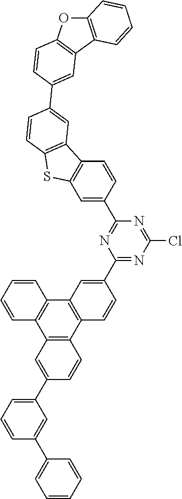 [SMILES: Clc1nc(-c2ccc3c(c2)sc2ccc(-c4ccc5oc6ccccc6c5c4)cc23)nc(-c2ccc3c4ccc(-c5cccc(-c6ccccc6)c5)cc4c4ccccc4c3c2)n1] | 62% |
| 3d | 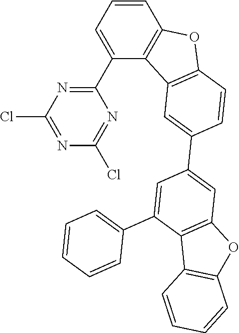 [SMILES: Clc1nc(Cl)nc(-c2cccc3oc4ccc(-c5cc(-c6ccccc6)c6c(c5)oc5ccccc56)cc4c23)n1] | 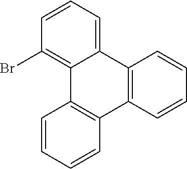 [SMILES: Brc1cccc2c3ccccc3c3ccccc3c12] [74897-21-5] | 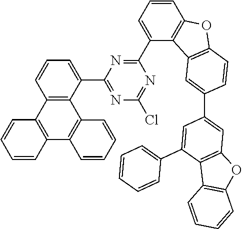 [SMILES: Clc1nc(-c2cccc3oc4ccc(-c5cc(-c6ccccc6)c6c(c5)oc5ccccc56)cc4c23)nc(-c2cccc3c4ccccc4c4ccccc4c23)n1] | 67% |
| 4d | 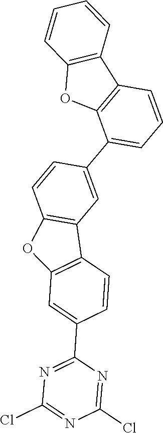 [SMILES: Clc1nc(Cl)nc(-c2ccc3c(c2)oc2ccc(-c4cccc5c4oc4ccccc45)cc23)n1] | 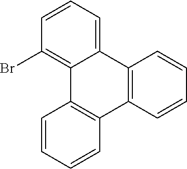 [SMILES: Brc1cccc2c3ccccc3c3ccccc3c12] [74897-21-5] | 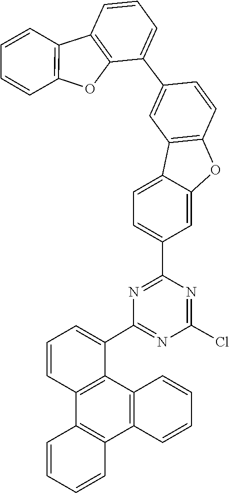 [SMILES: Clc1nc(-c2ccc3c(c2)oc2ccc(-c4cccc5c4oc4ccccc45)cc23)nc(-c2cccc3c4ccccc4c4ccccc4c23)n1] | 541% |
| 5d | 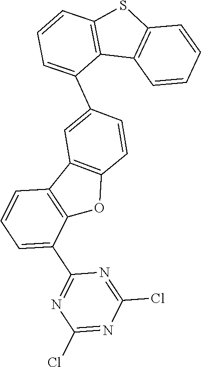 [SMILES: Clc1nc(Cl)nc(-c2cccc3c2oc2ccc(-c4cccc5sc6ccccc6c45)cc23)n1] | 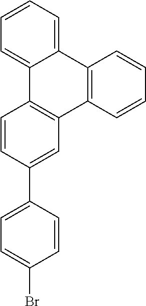 [SMILES: Brc1ccc(-c2ccc3c4ccccc4c4ccccc4c3c2)cc1] [1158227-56-5] | 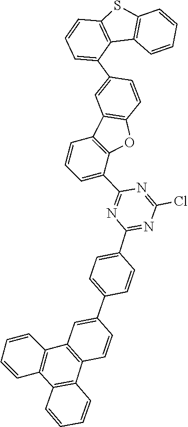 [SMILES: Clc1nc(-c2ccc(-c3ccc4c5ccccc5c5ccccc5c4c3)cc2)nc(-c2cccc3c2oc2ccc(-c4cccc5sc6ccccc6c45)cc23)n1] | 56% |
| 6d | 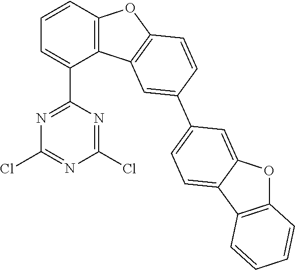 [SMILES: Clc1nc(Cl)nc(-c2cccc3oc4ccc(-c5ccc6c(c5)oc5ccccc56)cc4c23)n1] | 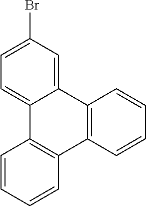 [SMILES: Brc1ccc2c3ccccc3c3ccccc3c2c1] [19111-87-6] | 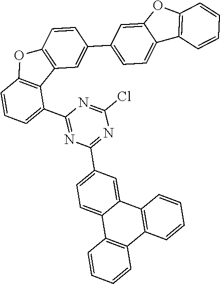 [SMILES: Clc1nc(-c2ccc3c4ccccc4c4ccccc4c3c2)nc(-c2cccc3oc4ccc(-c5ccc6c(c5)oc5ccccc56)cc4c23)n1] | 67% |
| 7d | 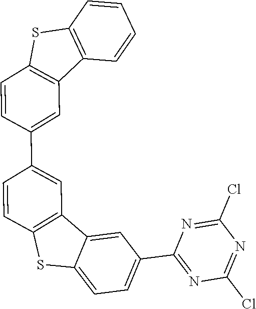 [SMILES: Clc1nc(Cl)nc(-c2ccc3sc4ccc(-c5ccc6sc7ccccc7c6c5)cc4c3c2)n1] | 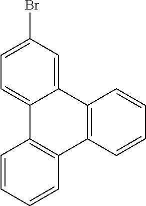 [SMILES: Brc1ccc2c3ccccc3c3ccccc3c2c1] [19111-87-6] | 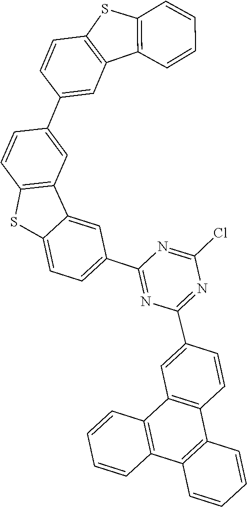 [SMILES: Clc1nc(-c2ccc3sc4ccc(-c5ccc6sc7ccccc7c6c5)cc4c3c2)nc(-c2ccc3c4ccccc4c4ccccc4c3c2)n1] | 65% |
| 8d | 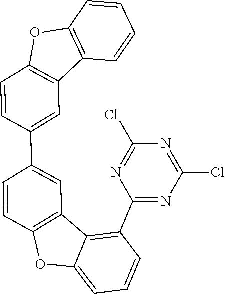 [SMILES: Clc1nc(Cl)nc(-c2cccc3oc4ccc(-c5ccc6oc7ccccc7c6c5)cc4c23)n1] | 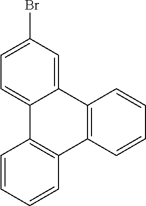 [SMILES: Brc1ccc2c3ccccc3c3ccccc3c2c1] [19111-87-6] | 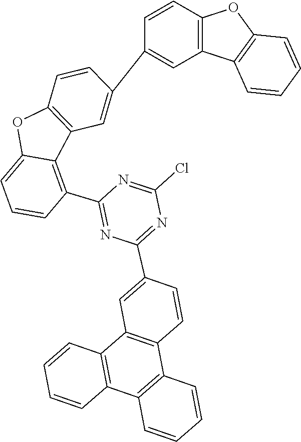 [SMILES: Clc1nc(-c2ccc3c4ccccc4c4ccccc4c3c2)nc(-c2cccc3oc4ccc(-c5ccc6oc7ccccc7c6c5)cc4c23)n1] | 67% |
| 9d | 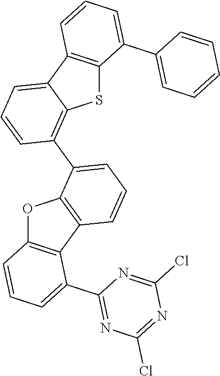 [SMILES: Clc1nc(Cl)nc(-c2cccc3oc4c(-c5cccc6c5sc5c(-c7ccccc7)cccc56)cccc4c23)n1] | 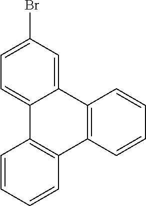 [SMILES: Brc1ccc2c3ccccc3c3ccccc3c2c1] [19111-87-6] | 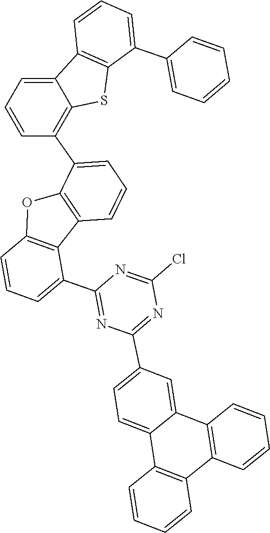 [SMILES: Clc1nc(-c2ccc3c4ccccc4c4ccccc4c3c2)nc(-c2cccc3oc4c(-c5cccc6c5sc5c(-c7ccccc7)cccc56)cccc4c23)n1] | 71% |
| 10d | 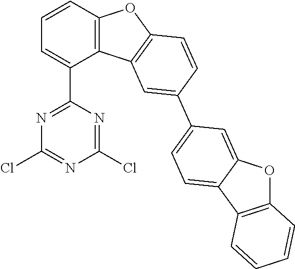 [SMILES: Clc1nc(Cl)nc(-c2cccc3oc4ccc(-c5ccc6c(c5)oc5ccccc56)cc4c23)n1] | 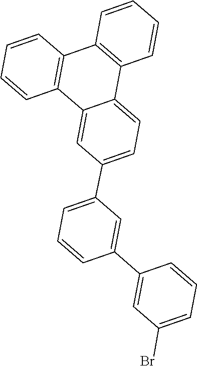 [SMILES: Brc1cccc(-c2cccc(-c3ccc4c5ccccc5c5ccccc5c4c3)c2)c1] [1365089-59-3] | 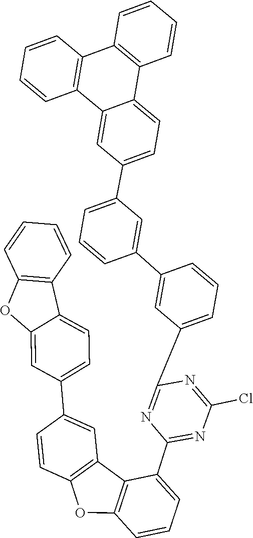 [SMILES: Clc1nc(-c2cccc(-c3cccc(-c4ccc5c6ccccc6c6ccccc6c5c4)c3)c2)nc(-c2cccc3oc4ccc(-c5ccc6c(c5)oc5ccccc56)cc4c23)n1] | 63% |
| 11d | 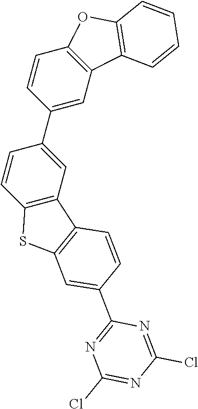 [SMILES: Clc1nc(Cl)nc(-c2ccc3c(c2)sc2ccc(-c4ccc5oc6ccccc6c5c4)cc23)n1] | 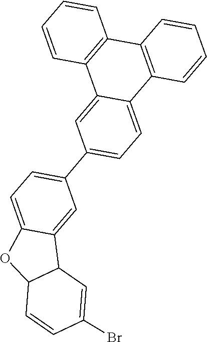 [SMILES: BrC1=CC2c3cc(-c4ccc5c6ccccc6c6ccccc6c5c4)ccc3OC2C=C1] [1964481-21-7] | 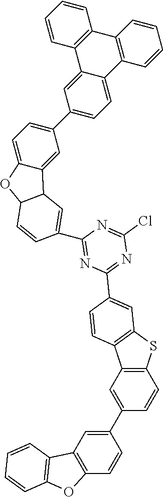 [SMILES: Clc1nc(C2=CC3c4cc(-c5ccc6c7ccccc7c7ccccc7c6c5)ccc4OC3C=C2)nc(-c2ccc3c(c2)sc2ccc(-c4ccc5oc6ccccc6c5c4)cc23)n1] | 62% |
| 12d | 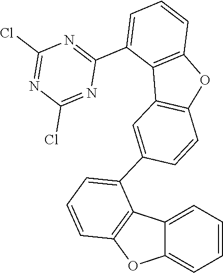 [SMILES: Clc1nc(Cl)nc(-c2cccc3oc4ccc(-c5cccc6oc7ccccc7c56)cc4c23)n1] | 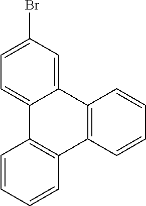 [SMILES: Brc1ccc2c3ccccc3c3ccccc3c2c1] [19111-87-6] | 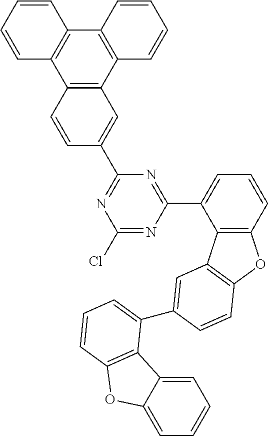 [SMILES: Clc1nc(-c2ccc3c4ccccc4c4ccccc4c3c2)nc(-c2cccc3oc4ccc(-c5cccc6oc7ccccc7c56)cc4c23)n1] | 63% |
| 13d | 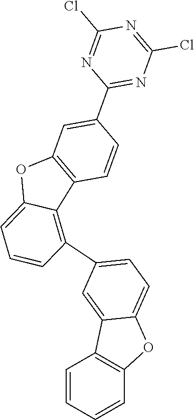 [SMILES: Clc1nc(Cl)nc(-c2ccc3c(c2)oc2cccc(-c4ccc5oc6ccccc6c5c4)c23)n1] | 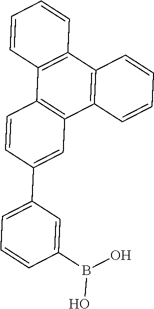 [SMILES: OB(O)c1cccc(-c2ccc3c4ccccc4c4ccccc4c3c2)c1] [1235876-72-8] | 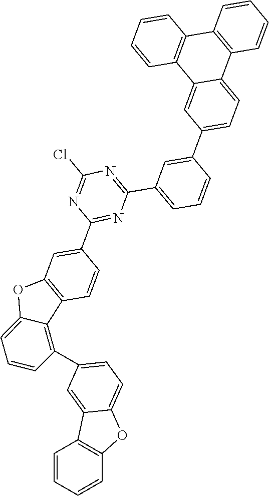 [SMILES: Clc1nc(-c2cccc(-c3ccc4c5ccccc5c5ccccc5c4c3)c2)nc(-c2ccc3c(c2)oc2cccc(-c4ccc5oc6ccccc6c5c4)c23)n1] | 65% |
| 14d | 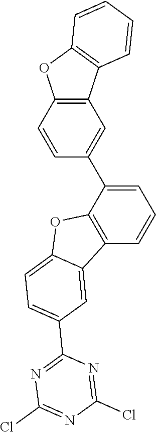 [SMILES: Clc1nc(Cl)nc(-c2ccc3oc4c(-c5ccc6oc7ccccc7c6c5)cccc4c3c2)n1] | 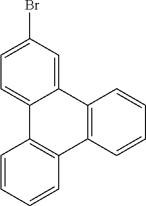 [SMILES: Brc1ccc2c3ccccc3c3ccccc3c2c1] [19111-87-6] | 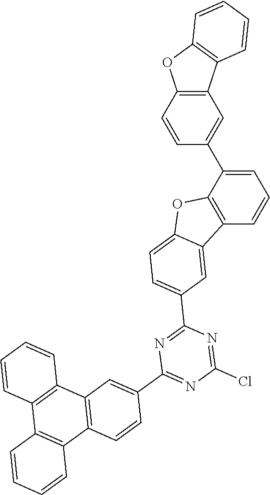 [SMILES: Clc1nc(-c2ccc3oc4c(-c5ccc6oc7ccccc7c6c5)cccc4c3c2)nc(-c2ccc3c4ccccc4c4ccccc4c3c2)n1] | 62% |

### e) 2-Dibenzofuran-1-yl-4-(6-dibenzofuran-4-yldibenzofuran-4-yl)-6-triphenylen-2-yl-1,3,5-triazine

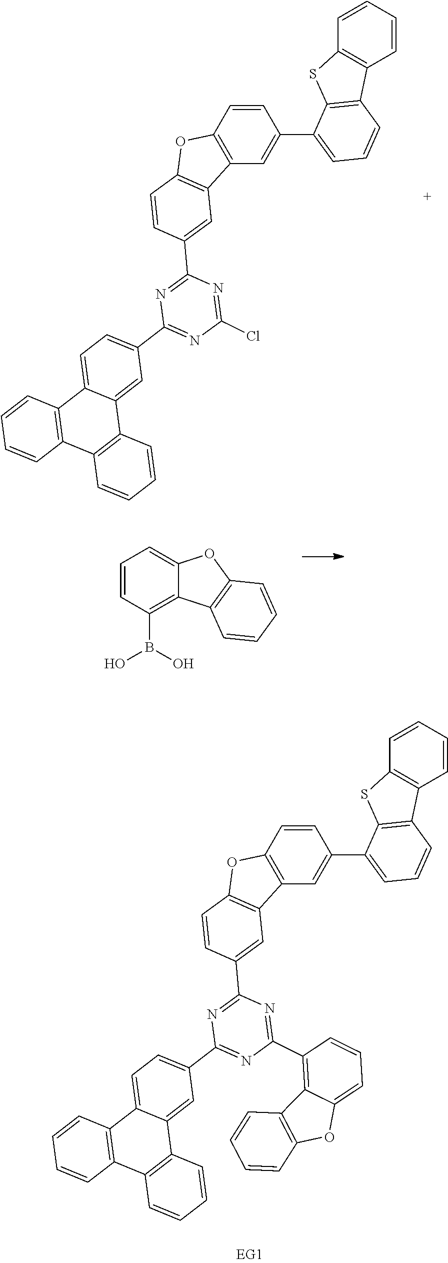 [SMILES: Clc1nc(-c2ccc3oc4ccc(-c5cccc6c5sc5ccccc56)cc4c3c2)nc(-c2ccc3c4ccccc4c4ccccc4c3c2)n1 ; OB(O)c1cccc2oc3ccccc3c12 ; c1ccc2c(c1)oc1cccc(-c3nc(-c4ccc5oc6ccc(-c7cccc8c7sc7ccccc78)cc6c5c4)nc(-c4ccc5c6ccccc6c6ccccc6c5c4)n3)c12]

55 g (80 mmol, 1.0 eq) of 2-chloro-4-(8-dibenzothiophen-4-yldibenzofuran-2-yl)-6-triphenylen-2-yl-1,3,5-triazine, 19 g (90 mmol, 1.1 eq) of dibenzofuran-1-yl-boronic acid and 17g (160 mmol, 2.0 eq) of sodium carbonate are dissolved in 400 ml of toluene, 250 ml of water and 170 ml of ethanol under an inert atmosphere. Then 0.93 g (0.80 mmol, 0.01 eq) of tetrakis(triphenylphosphine)palladium is added and the mixture is refluxed at 110° C. overnight. On conclusion of the reaction, 300 ml of water is added and the precipitated solids are filtered. The organic phase is separated off, washed with water and dried over sodium sulfate. After the solvent has evaporated, a further 5.1 g of the crude product is obtained. The combined solids are purified by hot extraction from toluene/heptane, recrystallized twice from toluene/heptane and sublimed. Yield: 48 g (59 mmol), 74% of theory, purity by ¹H NMR about 98%.

The following compounds can be prepared analogously:

| | Reactant 1 | Reactant 2 | Product | Yield |
|---|---|---|---|---|
| 1e | 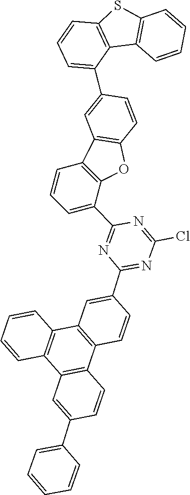 [SMILES: Clc1nc(-c2ccc3c4ccc(-c5ccccc5)cc4c4ccccc4c3c2)nc(-c2cccc3c2oc2ccc(-c4cccc5sc6ccccc6c45)cc23)n1] | 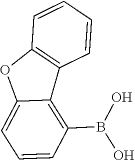 [SMILES: OB(O)c1cccc2oc3ccccc3c12] [162607-19-4] | 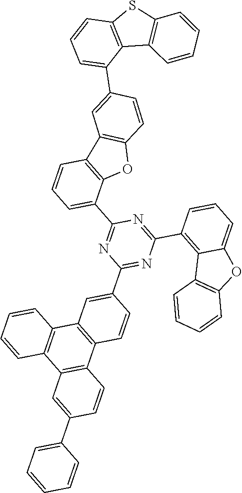 [SMILES: c1ccc(-c2ccc3c4ccc(-c5nc(-c6cccc7c6oc6ccc(-c8cccc9sc%10ccccc%10c89)cc67)nc(-c6cccc7oc8ccccc8c67)n5)cc4c4ccccc4c3c2)cc1] | 70% |
| 2e | 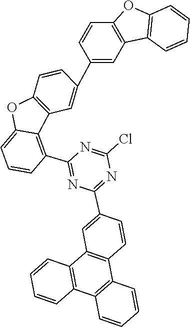 [SMILES: Clc1nc(-c2ccc3c4ccccc4c4ccccc4c3c2)nc(-c2cccc3oc4ccc(-c5ccc6oc7ccccc7c6c5)cc4c23)n1] | 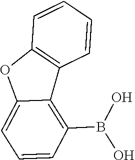 [SMILES: OB(O)c1cccc2oc3ccccc3c12] [162607-19-4] | 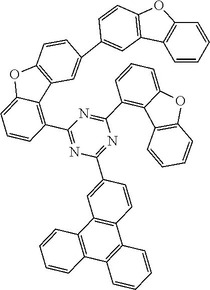 [SMILES: c1ccc2c(c1)oc1ccc(-c3ccc4oc5cccc(-c6nc(-c7ccc8c9ccccc9c9ccccc9c8c7)nc(-c7cccc8oc9ccccc9c78)n6)c5c4c3)cc12] EG10 | 77% |
| 3e | 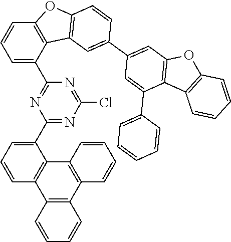 [SMILES: Clc1nc(-c2cccc3oc4ccc(-c5cc(-c6ccccc6)c6c(c5)oc5ccccc56)cc4c23)nc(-c2cccc3c4ccccc4c4ccccc4c23)n1] | 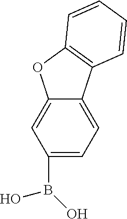 [SMILES: OB(O)c1ccc2c(c1)oc1ccccc12] [395087-89-5] | 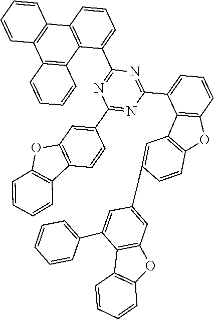 [SMILES: c1ccc(-c2cc(-c3ccc4oc5cccc(-c6nc(-c7ccc8c(c7)oc7ccccc78)nc(-c7cccc8c9ccccc9c9ccccc9c78)n6)c5c4c3)cc3oc4ccccc4c23)cc1] | 62% |
| | | | EG12 | |
| 4e | 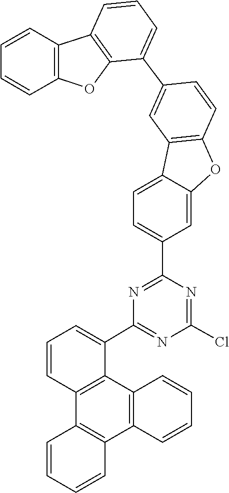 [SMILES: Clc1nc(-c2ccc3c(c2)oc2ccc(-c4cccc5c4oc4ccccc45)cc23)nc(-c2cccc3c4ccccc4c4ccccc4c23)n1] | 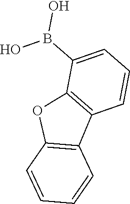 [SMILES: OB(O)c1cccc2c1oc1ccccc12] [100124-06-9] | 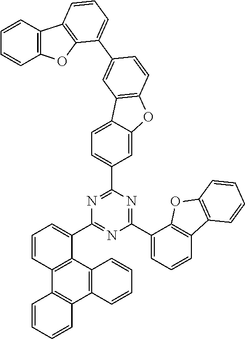 [SMILES: c1ccc2c(c1)oc1c(-c3ccc4oc5cc(-c6nc(-c7cccc8c7oc7ccccc78)nc(-c7cccc8c9ccccc9c9ccccc9c78)n6)ccc5c4c3)cccc12] EG8 | 76% |
| 5e | 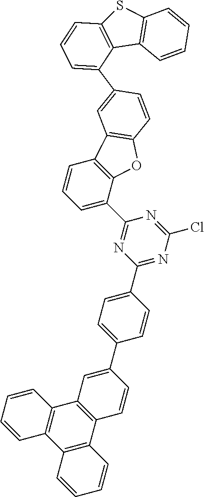 [SMILES: Clc1nc(-c2ccc(-c3ccc4c5ccccc5c5ccccc5c4c3)cc2)nc(-c2cccc3c2oc2ccc(-c4cccc5sc6ccccc6c45)cc23)n1] | 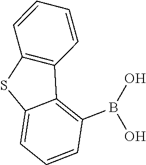 [SMILES: OB(O)c1cccc2sc3ccccc3c12] [1245943-60-5] | 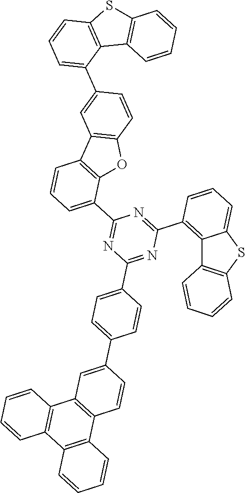 [SMILES: c1ccc2c(c1)sc1cccc(-c3ccc4oc5c(-c6nc(-c7ccc(-c8ccc9c%10ccccc%10c%10ccccc%10c9c8)cc7)nc(-c7cccc8sc9ccccc9c78)n6)cccc5c4c3)c12] | 79% |
| | | | EG4 | |
| 6e | 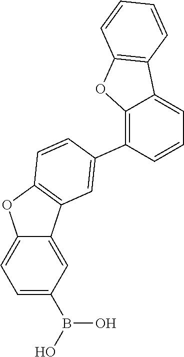 [SMILES: OB(O)c1ccc2oc3ccc(-c4cccc5c4oc4ccccc45)cc3c2c1] [2399520-37-5] | 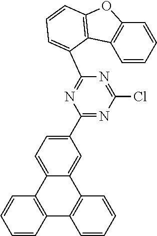 [SMILES: Clc1nc(-c2ccc3c4ccccc4c4ccccc4c3c2)nc(-c2cccc3oc4ccccc4c23)n1] [2417984-49-5] | 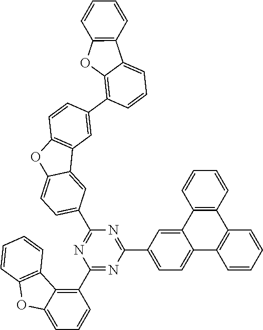 [SMILES: c1ccc2c(c1)oc1c(-c3ccc4oc5ccc(-c6nc(-c7ccc8c9ccccc9c9ccccc9c8c7)nc(-c7cccc8oc9ccccc9c78)n6)cc5c4c3)cccc12] EG11 | 68% |
| 7e | 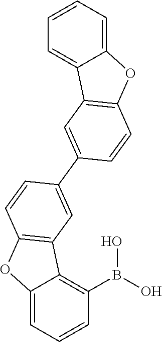 [SMILES: OB(O)c1cccc2oc3ccc(-c4ccc5oc6ccccc6c5c4)cc3c12] [1822320-55-7] | 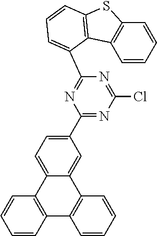 [SMILES: Clc1nc(-c2ccc3c4ccccc4c4ccccc4c3c2)nc(-c2cccc3sc4ccccc4c23)n1] [2417985-98-7] | 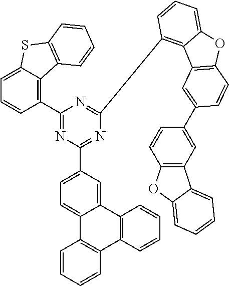 [SMILES: c1ccc2c(c1)oc1ccc(-c3ccc4oc5cccc(-c6nc(-c7ccc8c9ccccc9c9ccccc9c8c7)nc(-c7cccc8sc9ccccc9c78)n6)c5c4c3)cc12] | 67% |
| 8e | 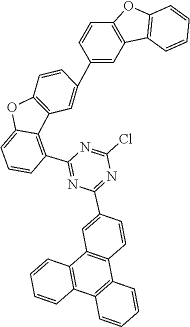 [SMILES: Clc1nc(-c2ccc3c4ccccc4c4ccccc4c3c2)nc(-c2cccc3oc4ccc(-c5ccc6oc7ccccc7c6c5)cc4c23)n1] | 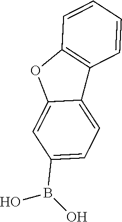 [SMILES: OB(O)c1ccc2c(c1)oc1ccccc12] [395087-89-5] | 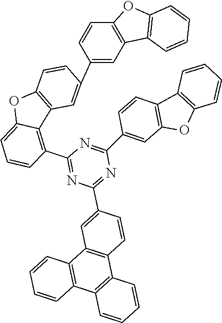 [SMILES: c1ccc2c(c1)oc1cc(-c3nc(-c4ccc5c6ccccc6c6ccccc6c5c4)nc(-c4cccc5oc6ccc(-c7ccc8oc9ccccc9c8c7)cc6c45)n3)ccc12] | 71% |
| | | | EG2 | |
| 9e | 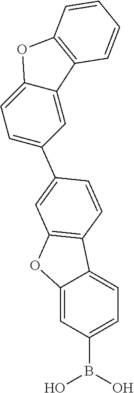 [SMILES: OB(O)c1ccc2c(c1)oc1cc(-c3ccc4oc5ccccc5c4c3)ccc12] [2235-15-4] | 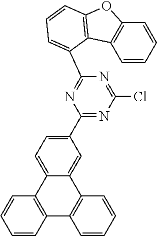 [SMILES: Clc1nc(-c2ccc3c4ccccc4c4ccccc4c3c2)nc(-c2cccc3oc4ccccc4c23)n1] [2417984-49-5] | 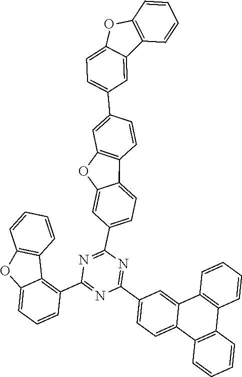 [SMILES: c1ccc2c(c1)oc1ccc(-c3ccc4c(c3)oc3cc(-c5nc(-c6ccc7c8ccccc8c8ccccc8c7c6)nc(-c6cccc7oc8ccccc8c67)n5)ccc34)cc12] | 65% |
| 10e | 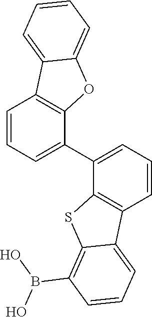 [SMILES: OB(O)c1cccc2c1sc1c(-c3cccc4c3oc3ccccc34)cccc12] [2409570-75-9] | 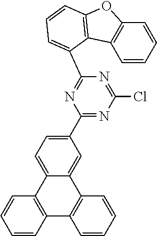 [SMILES: Clc1nc(-c2ccc3c4ccccc4c4ccccc4c3c2)nc(-c2cccc3oc4ccccc4c23)n1] [2417984-49-5] | 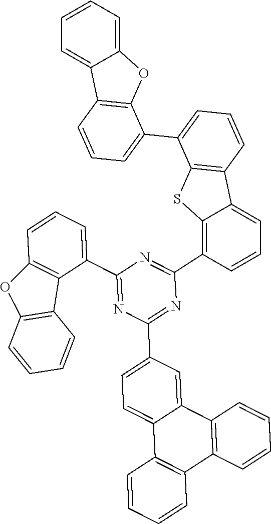 [SMILES: c1ccc2c(c1)oc1c(-c3cccc4c3sc3c(-c5nc(-c6ccc7c8ccccc8c8ccccc8c7c6)nc(-c6cccc7oc8ccccc8c67)n5)cccc34)cccc12] | 56% |
| 11e | 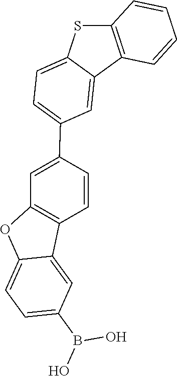 [SMILES: OB(O)c1ccc2oc3cc(-c4ccc5sc6ccccc6c5c4)ccc3c2c1] [2035080-76-1] | 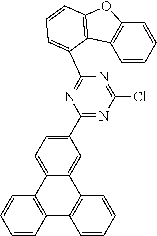 [SMILES: Clc1nc(-c2ccc3c4ccccc4c4ccccc4c3c2)nc(-c2cccc3oc4ccccc4c23)n1] [2417984-49-5] | 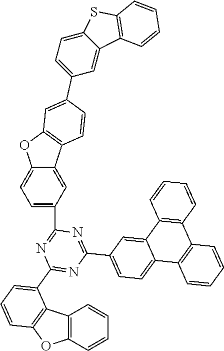 [SMILES: c1ccc2c(c1)oc1cccc(-c3nc(-c4ccc5oc6cc(-c7ccc8sc9ccccc9c8c7)ccc6c5c4)nc(-c4ccc5c6ccccc6c6ccccc6c5c4)n3)c12] | 66% |
| 12e | 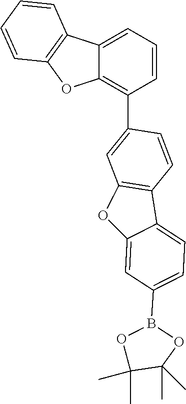 [SMILES: CC1(C)OB(c2ccc3c(c2)oc2cc(-c4cccc5c4oc4ccccc45)ccc23)OC1(C)C] [2361076-78-8] | 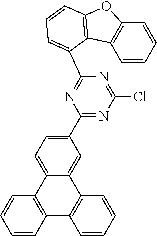 [SMILES: Clc1nc(-c2ccc3c4ccccc4c4ccccc4c3c2)nc(-c2cccc3oc4ccccc4c23)n1] [2417984-49-5] | 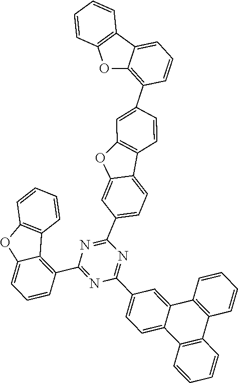 [SMILES: c1ccc2c(c1)oc1c(-c3ccc4c(c3)oc3cc(-c5nc(-c6ccc7c8ccccc8c8ccccc8c7c6)nc(-c6cccc7oc8ccccc8c67)n5)ccc34)cccc12] | 72% |
| 13e | 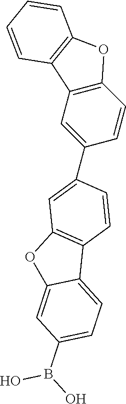 [SMILES: OB(O)c1ccc2c(c1)oc1cc(-c3ccc4oc5ccccc5c4c3)ccc12] [223538-15-4] | 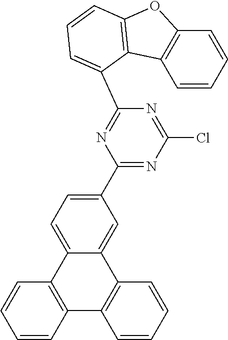 [SMILES: Clc1nc(-c2ccc3c4ccccc4c4ccccc4c3c2)nc(-c2cccc3oc4ccccc4c23)n1] [2417984-49-5] | 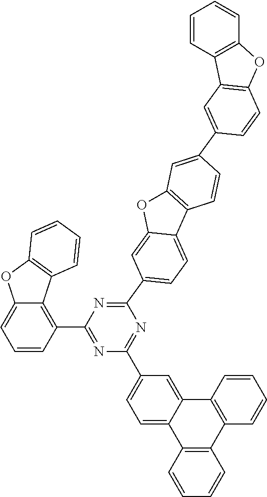 [SMILES: c1ccc2c(c1)oc1ccc(-c3ccc4c(c3)oc3cc(-c5nc(-c6ccc7c8ccccc8c8ccccc8c7c6)nc(-c6cccc7oc8ccccc8c67)n5)ccc34)cc12] | 70% |
| 14e | 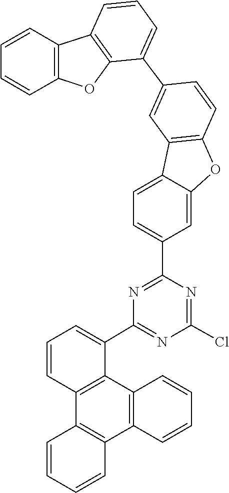 [SMILES: Clc1nc(-c2ccc3c(c2)oc2ccc(-c4cccc5c4oc4ccccc45)cc23)nc(-c2cccc3c4ccccc4c4ccccc4c23)n1] | 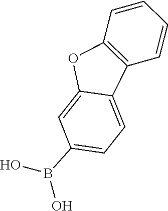 [SMILES: OB(O)c1ccc2c(c1)oc1ccccc12] [395087-89-5] | 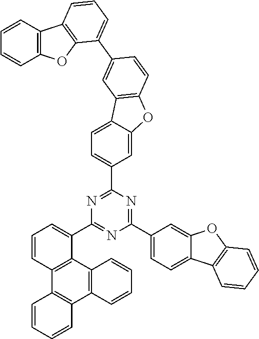 [SMILES: c1ccc2c(c1)oc1cc(-c3nc(-c4ccc5c(c4)oc4ccc(-c6cccc7c6oc6ccccc67)cc45)nc(-c4cccc5c6ccccc6c6ccccc6c45)n3)ccc12] | 63% |
| 15e | 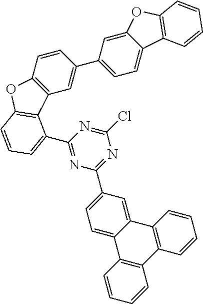 [SMILES: Clc1nc(-c2ccc3c4ccccc4c4ccccc4c3c2)nc(-c2cccc3oc4ccc(-c5ccc6c(c5)oc5ccccc56)cc4c23)n1] | 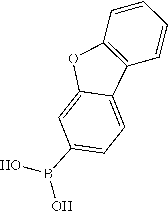 [SMILES: OB(O)c1ccc2c(c1)oc1ccccc12] [395087-89-5] | 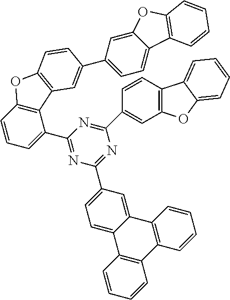 [SMILES: c1ccc2c(c1)oc1cc(-c3ccc4oc5cccc(-c6nc(-c7ccc8c(c7)oc7ccccc78)nc(-c7ccc8c9ccccc9c9ccccc9c8c7)n6)c5c4c3)ccc12] | 77% |
| | | | EG9 | |
| 16e | 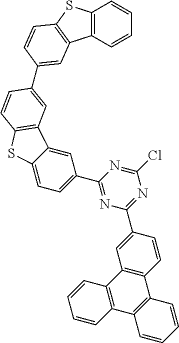 [SMILES: Clc1nc(-c2ccc3sc4ccc(-c5ccc6sc7ccccc7c6c5)cc4c3c2)nc(-c2ccc3c4ccccc4c4ccccc4c3c2)n1] | 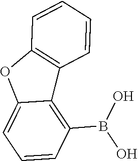 [SMILES: OB(O)c1cccc2oc3ccccc3c12] [162607-19-4] | 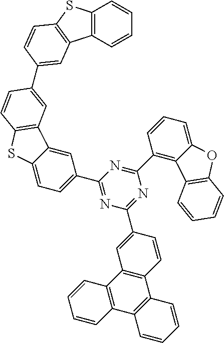 [SMILES: c1ccc2c(c1)oc1cccc(-c3nc(-c4ccc5sc6ccc(-c7ccc8sc9ccccc9c8c7)cc6c5c4)nc(-c4ccc5c6ccccc6c6ccccc6c5c4)n3)c12] | 69% |
| 17e | 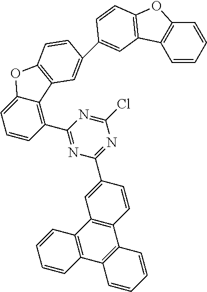 [SMILES: Clc1nc(-c2ccc3c4ccccc4c4ccccc4c3c2)nc(-c2cccc3oc4ccc(-c5ccc6oc7ccccc7c6c5)cc4c23)n1] | 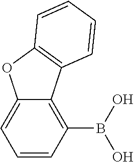 [SMILES: OB(O)c1cccc2oc3ccccc3c12] [162607-19-4] | 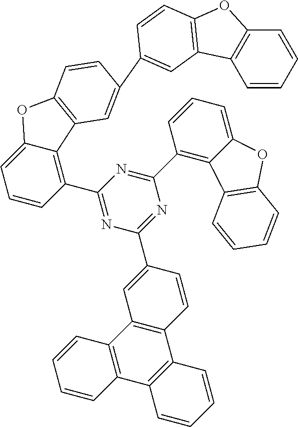 [SMILES: c1ccc2c(c1)oc1ccc(-c3ccc4oc5cccc(-c6nc(-c7ccc8c9ccccc9c9ccccc9c8c7)nc(-c7cccc8oc9ccccc9c78)n6)c5c4c3)cc12] | 74% |
| | | | EG15 | |
| 18e | 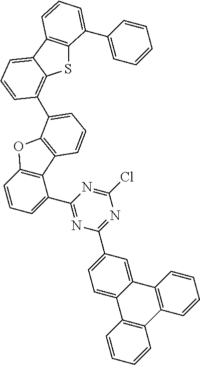 [SMILES: Clc1nc(-c2ccc3c4ccccc4c4ccccc4c3c2)nc(-c2cccc3oc4c(-c5cccc6c5sc5c(-c7ccccc7)cccc56)cccc4c23)n1] | 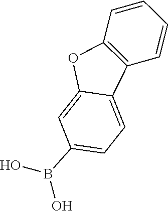 [SMILES: OB(O)c1ccc2c(c1)oc1ccccc12] [395087-89-5] | 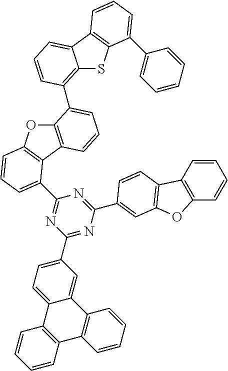 [SMILES: c1ccc(-c2cccc3c2sc2c(-c4cccc5c4oc4cccc(-c6nc(-c7ccc8c(c7)oc7ccccc78)nc(-c7ccc8c9ccccc9c9ccccc9c8c7)n6)c45)cccc23)cc1] | 70% |
| | | | EG13 | |
| 19e | 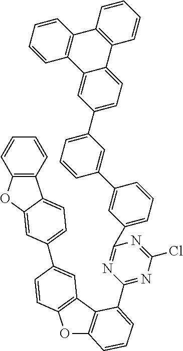 [SMILES: Clc1nc(-c2cccc(-c3cccc(-c4ccc5c6ccccc6c6ccccc6c5c4)c3)c2)nc(-c2cccc3oc4ccc(-c5ccc6c(c5)oc5ccccc56)cc4c23)n1] | 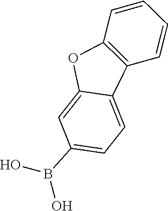 [SMILES: OB(O)c1ccc2c(c1)oc1ccccc12] [395087-89-5] | 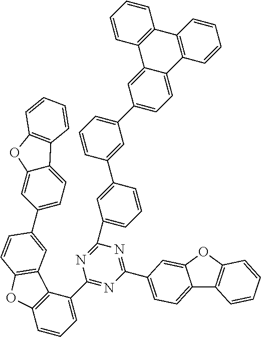 [SMILES: c1cc(-c2cccc(-c3nc(-c4ccc5c(c4)oc4ccccc45)nc(-c4cccc5oc6ccc(-c7ccc8c(c7)oc7ccccc78)cc6c45)n3)c2)cc(-c2ccc3c4ccccc4c4ccccc4c3c2)c1] | 61% |
| 20e | 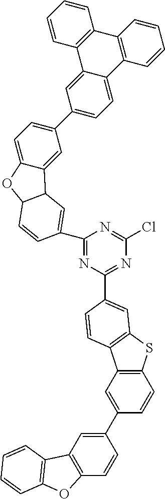 [SMILES: Clc1nc(C2=CC3c4cc(-c5ccc6c7ccccc7c7ccccc7c6c5)ccc4OC3C=C2)nc(-c2ccc3c(c2)sc2ccc(-c4ccc5oc6ccccc6c5c4)cc23)n1] | 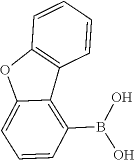 [SMILES: OB(O)c1cccc2oc3ccccc3c12] [162607-19-4] | 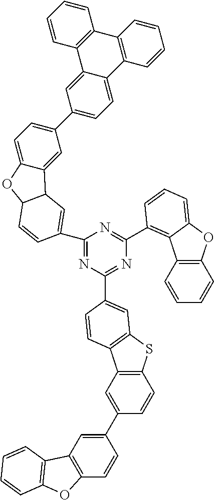 [SMILES: C1=CC2Oc3ccc(-c4ccc5c6ccccc6c6ccccc6c5c4)cc3C2C=C1c1nc(-c2ccc3c(c2)sc2ccc(-c4ccc5oc6ccccc6c5c4)cc23)nc(-c2cccc3oc4ccccc4c23)n1] | 62% |
| 21e | 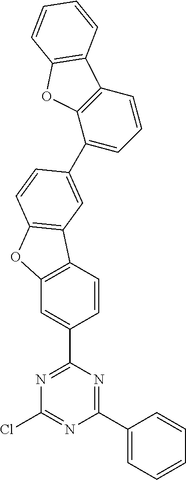 [SMILES: Clc1nc(-c2ccccc2)nc(-c2ccc3c(c2)oc2ccc(-c4cccc5c4oc4ccccc45)cc23)n1] | 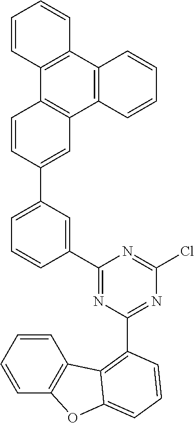 [SMILES: Clc1nc(-c2cccc(-c3ccc4c5ccccc5c5ccccc5c4c3)c2)nc(-c2cccc3oc4ccccc4c23)n1] | 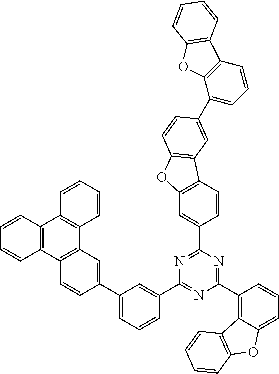 [SMILES: c1cc(-c2ccc3c4ccccc4c4ccccc4c3c2)cc(-c2nc(-c3ccc4c(c3)oc3ccc(-c5cccc6c5oc5ccccc56)cc34)nc(-c3cccc4oc5ccccc5c34)n2)c1] EG14 | 64% |
| 22e | 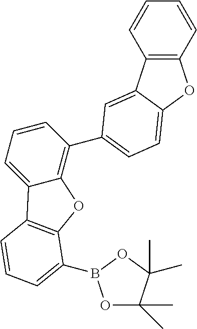 [SMILES: CC1(C)OB(c2cccc3c2oc2c(-c4ccc5oc6ccccc6c5c4)cccc23)OC1(C)C] [2550984-81-9] | 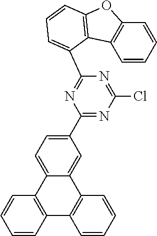 [SMILES: Clc1nc(-c2ccc3c4ccccc4c4ccccc4c3c2)nc(-c2cccc3oc4ccccc4c23)n1] [2417984-49-5] | 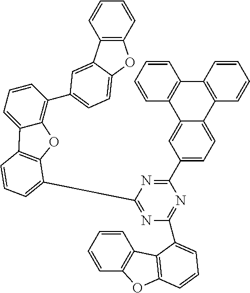 [SMILES: c1ccc2c(c1)oc1ccc(-c3cccc4c3oc3c(-c5nc(-c6ccc7c8ccccc8c8ccccc8c7c6)nc(-c6cccc7oc8ccccc8c67)n5)cccc34)cc12] EG5 | 70% |
| 23e | 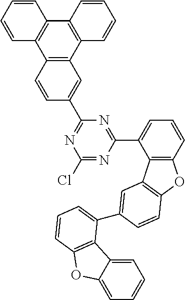 [SMILES: Clc1nc(-c2ccc3c4ccccc4c4ccccc4c3c2)nc(-c2cccc3oc4ccc(-c5cccc6oc7ccccc7c56)cc4c23)n1] | 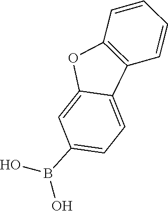 [SMILES: OB(O)c1ccc2c(c1)oc1ccccc12] [395087-89-5] | 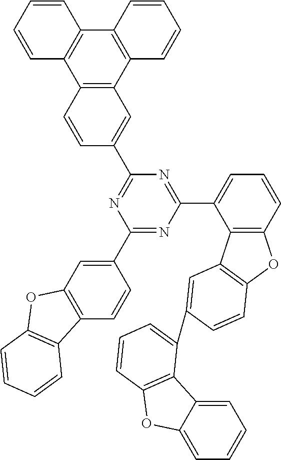 [SMILES: c1ccc2c(c1)oc1cc(-c3nc(-c4ccc5c6ccccc6c6ccccc6c5c4)nc(-c4cccc5oc6ccc(-c7cccc8oc9ccccc9c78)cc6c45)n3)ccc12] | 74% |
| | | | EG6 | |
| 24e | 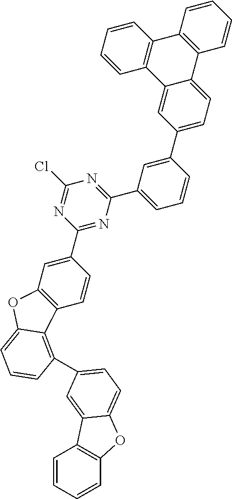 [SMILES: Clc1nc(-c2cccc(-c3ccc4c5ccccc5c5ccccc5c4c3)c2)nc(-c2ccc3c(c2)oc2cccc(-c4ccc5oc6ccccc6c5c4)c23)n1] | 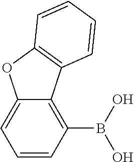 [SMILES: OB(O)c1cccc2oc3ccccc3c12] [162607-19-4] | 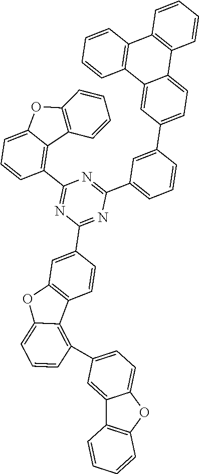 [SMILES: c1cc(-c2ccc3c4ccccc4c4ccccc4c3c2)cc(-c2nc(-c3ccc4c(c3)oc3cccc(-c5ccc6oc7ccccc7c6c5)c34)nc(-c3cccc4oc5ccccc5c34)n2)c1] EG7 | 70% |
| 25e | 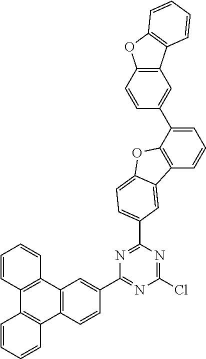 [SMILES: Clc1nc(-c2ccc3oc4c(-c5ccc6oc7ccccc7c6c5)cccc4c3c2)nc(-c2ccc3c4ccccc4c4ccccc4c3c2)n1] | 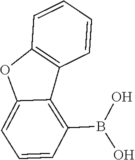 [SMILES: OB(O)c1cccc2oc3ccccc3c12] [162607-19-4] | 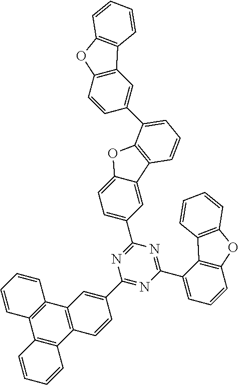 [SMILES: c1ccc2c(c1)oc1ccc(-c3cccc4c3oc3ccc(-c5nc(-c6ccc7c8ccccc8c8ccccc8c7c6)nc(-c6cccc7oc8ccccc8c67)n5)cc34)cc12] | 66% |
| | | | EG3 | |

### Synthesis Example 2

### General Deuteration:

The starting compound is dissolved in a mixture of deuterated water (99% deuterium atom) and toluene-d8 (99% deuterium atom) and heated to 16000 under pressure in the presence of dry platinum on charcoal (5%) as catalyst for 96 hours. After the reaction mixture has been cooled down, the phases are separated, and the aqueous phase is extracted twice with the tetrahydrofuran-toluene mixture. The recombined organic phases are washed with a sodium chloride solution, dried over sodium sulfate and filtered. The solvent is removed under reduced pressure in order to provide the crude deuterated compound in solid form. The compound is purified further by extraction, crystallization and sublimation.

### Example A: 1,1′,2′,3′,4′,5′,6,6′,7′,8,8′-Undecadeuterio-N-(2,3,6,7,8-pentadeuterio-9,9-dimethylfluoren-4-yl)-N-(3,4,6,7,8-pentadeuterio-9,9-dimethylfluoren-2-yl)-9,9′-spirobi[fluorene]-4-amine

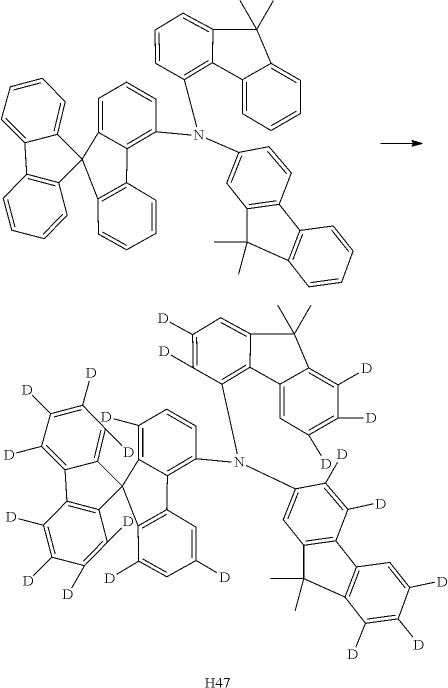

N-(9,9-Dimethylfluoren-2-yl)-N-(9,9-dimethylfluoren-4-yl)-9,9′-spirobi[fluorene]-4′-amine (22.8 g, 32 mmol), toluene-d8 (231 g, 2.31 mol), deuterated water (1300 g, 64.9 mol) and dry platinum on charcoal (5%) (30 g) are stirred at 130° C. for 24 h. The crude product is purified further by extracting twice with a mixture of heptane and toluene (4:1) and subliming twice.

Yield: 21.2 g (28 mmol, 90%) with a purity of >99.9%. Identity is demonstrated by HPLC-MS and 1H NMR.

### Example B: 1,2,3,5,6,7,8-Heptadeuterio-N-[1,2,3,5,6,7,8-heptadeuterio-9,9-bis(trideuteriomethyl)fluoren-4-yl]-9,9-bis(trideuteriomethyl)-N-[2,3,5-trideuterio-4-(2,3,4,5,6-pentadeuteriophenyl)phenyl]fluorene-4-amine

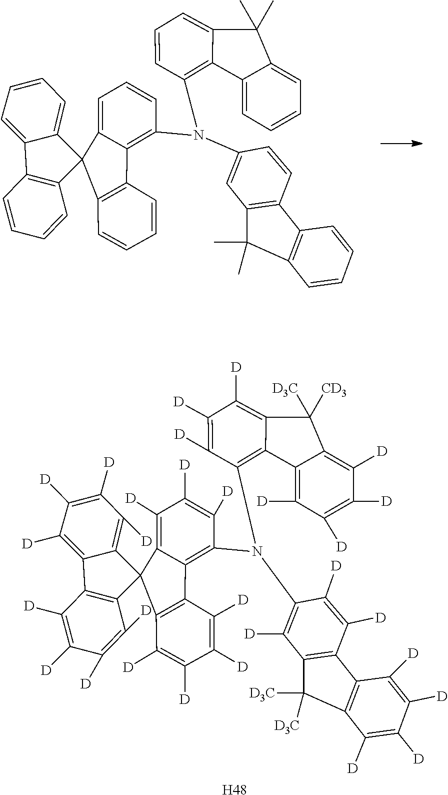 [SMILES: CC1(C)c2ccccc2-c2ccc(N(c3cccc4c3-c3ccccc3C4(C)C)c3cccc4c3-c3ccccc3C43c4ccccc4-c4ccccc43)cc21 ; [2H]c1c([2H])c([2H])c2c(c1[2H])-c1c([2H])c([2H])c([2H])c([2H])c1C21c2c([2H])c([2H])c([2H])c([2H])c2-c2c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c([2H])c4c3-c3c([2H])c([2H])c([2H])c([2H])c3C4(C([2H])([2H])[2H])C([2H])([2H])[2H])c([2H])c([2H])c([2H])c21 ; "H48"]

N-(9,9-Dimethylfluoren-2-yl)-N-(9,9-dimethylfluoren-4-yl)-9,9′-spirobi[fluorene]-4′-amine (22.8 g, 31.8 mmol), toluene-d8 (231 g, 2.31 mol), deuterated water (1300 g, 64.9 mol) and dry platinum on charcoal (5%) (30 g) are stirred at 160° C. for 96 h. The crude product is purified further by extracting twice with a mixture of heptane and toluene (4:1) and subliming twice.

Yield: 21.9 g (28.9 mmol, 95%) with a purity of >99.9%. Identity is demonstrated by HPLC-MS.

### Production of the OLEDs

In examples V1 to V11 and E1 to E18 which follow (see tables 7 and 8), the data of various OLEDs are presented.

### Pretreatment for Examples V1-V11 and E1-E18

Glass plates coated with structured ITO (indium tin oxide) of thickness 50 nm, for improved processing, are coated with 20 nm of PEDOT:PSS (poly(3,4-ethylenedioxythiophene) poly(styrenesulfonate), sourced as CLEVIOS™ P VP Al 4083 from Heraeus Precious Metals GmbH Deutschland, spun on from aqueous solution). These coated glass plates form the substrates to which the OLEDs are applied.

The OLEDs basically have the following layer structure: substrate/hole transport layer (HTL)/optional intermediate layer (IL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 8. The materials required for production of the OLEDs are shown in table 9 if not described above.

All materials are applied by thermal vapor deposition in a vacuum chamber. In this case, the emission layer always consists of at least one matrix material (host material or a mixture of host materials) and an emitting dopant (emitter) which is added to the matrix material(s) in a particular proportion by volume by co-evaporation. Details given in such a form as SdT1:H25:TEG1 (21%:72%:7%) mean here that the material SdTi is present in the layer in a proportion by volume of 21%, the material H25 in a proportion of 72% and the emitter TEG1 in a proportion of 7%. Analogously, the electron transport layer may also consist of a mixture of two materials.

The OLEDs are characterized in a standard manner. For this purpose, the electroluminescence spectra, the voltage and the external quantum efficiency (EQE, measured in percent) are determined as a function of luminance, calculated from current-voltage-luminance characteristics (IUL characteristics) assuming Lambertian radiation characteristics, and the lifetime. Electroluminescence spectra are determined at a luminance of 1000 cd/m², and these are used to calculate the CIE 1931 x and y color coordinates. The parameter U1000 in table 8 refers here to the voltage which is required for a luminance of 1000 cd/m². CE1000 denotes the current efficiency which is achieved at 1000 cd/m². Finally, EQE1000 refers to the external quantum efficiency at an operating luminance of 1000 cd/m². The lifetime LT is defined as the time after which the luminance drops from the starting luminance to a certain proportion L1 in the course of operation with constant current. A figure of L0; j0=4000 cd/m² and L1=70% in table 8 means that the lifetime reported in the LT column corresponds to the time after which the initial luminance of 4000 cd/m² drops to 2800 cd/m². Analogously, L0; j0=20 mA/cm², L1=80% means that the luminance drops to 80% of its starting value in the course of operation with 20 mA/cm² after the time LT.

The data for the various OLEDs are collated in table 8. Examples V1 to V8 are comparative examples according to the prior art; examples E1 to E18 show data of OLEDs of the invention.

There follows a detailed elucidation of some of the examples in order to illustrate the advantages of the OLEDs of the invention.

### Use of Inventive Mixtures in the Emission Layer of Phosphorescent OLEDs

The materials of the invention, when used as matrix materials in phosphorescent OLEDs, result in substantial improvements over the prior art with regard to the efficiency of the components. (Comparison of examples V1/E1, V2/E2, V3/E3, V4/V5/E4, V6/E5, V7/E6 and V8/E7). The materials of the invention, when used as electron conductors in phosphorescent OLEDs, result in substantial improvements over the prior art with regard to the efficiency of the components (comparison of examples V1 with E16, E17 and E18).

TABLE 7
Structure of the OLEDs

| | HTL | IL | EBL | EML | HBL | ETL | EIL |
|---|---|---|---|---|---|---|---|
| Ex. | thickness | thickness | thickness | thickness | thickness | thickness | thickness |
| V1 | SpA1 | HATCN | SpMA1 | SdT1:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V2 | SpA1 | HATCN | SpMA1 | SdT2:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V3 | SpA1 | HATCN | SpMA1 | SdT3:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V4 | SpA1 | HATCN | SpMA1 | SdT4:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V5 | SpA1 | HATCN | SpMA1 | SdT5:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V6 | SpA1 | HATCN | SpMA1 | SdT6:H1:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V7 | SpA1 | HATCN | SpMA1 | SdT7:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| V8 | SpA1 | HATCN | SpMA1 | SdT8:H26:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E1 | SpA1 | HATCN | SpMA1 | EG1:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E2 | SpA1 | HATCN | SpMA1 | EG2:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E3 | SpA1 | HATCN | SpMA1 | EG3:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E4 | SpA1 | HATCN | SpMA1 | EG4:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E5 | SpA1 | HATCN | SpMA1 | EG5:H25:TEG1 | ST2 | ST2:LIQ | LiQ |
| | 90 nm | 5 nm | 130 nm | (21%:72%:7%)40 nm | 10 nm | (50%:50%) | 1 nm |
| | | | | | | 30 nm | |
| E6 | SpA1 | HATCN | SpMA1 | EG6:H1:TEG1 | ST2 | ST2:LiQ | LiQ |
| | 90 nm | 5 nm | 130 nm | (21%:72%:7%)40 nm | 10 nm | (50%:50%) | 1 nm |
| | | | | | | 30 nm | |
| E7 | SpA1 | HATCN | SpMA1 | EG7:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E8 | SpA1 | HATCN | SpMA1 | EG8:H26:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E9 | SpA1 | HATCN | SpMA1 | EG9:H25:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E10 | SpA1 | HATCN | SpMA1 | EG10:H19:TEG1 | ST2 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E11 | SpA1 | HATCN | SpMA1 | EG11:H4:TEG1 | IC1 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E12 | SpA1 | HATCN | SpMA1 | EG12:H1:TEG1 | IC1 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E13 | SpA1 | HATCN | SpMA1 | EG13:H16:TEG1 | IC1 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E14 | SpA1 | HATCN | SpMA1 | EG14:H16:TEG1 | IC1 | ST2:LiQ | LiQ |
| | 70 nm | 5 nm | 90 nm | (21%:72%:7%)40 nm | 10 nm | (50%:50%) | 1 nm |
| | | | | | | 30 nm | |
| E15 | SpA1 | HATCN | SpMA1 | EG15:H25:TEG1 | IC1 | ST2:LIQ | LiQ |
| | 70 nm | 5 nm | 90 nm | (21%:72%:7%)40 nm | 10 nm | (50%:50%) | 1 nm |
| | | | | | | 30 nm | |
| V9 | HATCN | SpMA1 | SpMA2 | EG4:H25:TEG1 | — | SdT1 | LiQ |
| | 5 nm | 70 nm | 15 nm | (21%:72%:7%)40 nm | | 45 nm | 3 nm |
| V10 | HATCN | SpMA1 | SpMA2 | EG4:H25:TEG1 | — | SdT2 | LiQ |
| | 5 nm | 70 nm | 15 nm | (21%:72%:7%)40 nm | | 45 nm | 3 nm |
| V11 | HATCN | SpMA1 | SpMA2 | EG4:H25:TEG1 | — | SdT3 | LiQ |
| | 5 nm | 70 nm | 15 nm | (21%:72%:7%)40 nm | | 45 nm | 3 nm |
| E16 | HATCN | SpMA1 | SpMA2 | EG4:H25:TEG1 | — | EG2 | LiQ |
| | 5 nm | 70 nm | 15 nm | (21%:72%:7%)40 nm | | 45 nm | 3 nm |
| E17 | HATCN | SpMA1 | SpMA2 | EG4:H25:TEG1 | — | EG3 | LiQ |
| | 5 nm | 70 nm | 15 nm | (21%:72%:7%)40 nm | | 45 nm | 3 nm |
| E18 | HATCN | SpMA1 | SpMA2 | EG4:H25:TEG1 | — | EG6 | LiQ |
| | 5 nm | 70 nm | 15 nm | (21%:72%:7%)40 nm | | 45 nm | 3 nm |

TABLE 8
Data of the OLEDs

| | U1000 | SE1000 | | CIE x/y at | | | L1 | LT |
|---|---|---|---|---|---|---|---|---|
| Ex. | (V) | (cd/A) | EQE1000 | 1000 cd/m² | L₀; j₀ | | (%) | (h) |
| V1 | 4.0 | 53 | 13.7% | 0.33/0.63 | 20 | mA/cm² | 80 | 98 |
| V2 | 4.1 | 55 | 13.6% | 0.33/0.62 | 20 | mA/cm² | 80 | 99 |
| V3 | 4.2 | 52 | 13.5% | 0.33/0.64 | 20 | mA/cm² | 80 | 97 |
| V4 | 3.8 | 49 | 13.3% | 0.32/0.64 | 20 | mA/cm² | 80 | 91 |
| V5 | 4.1 | 51 | 13.1% | 0.33/0.64 | 20 | mA/cm² | 80 | 90 |
| V6 | 4.2 | 60 | 14.0% | 0.33/0.64 | 20 | mA/cm² | 80 | 91 |
| V7 | 4.1 | 59 | 14.1% | 0.33/0.64 | 20 | mA/cm² | 80 | 95 |
| V8 | 4.3 | 57 | 14.2% | 0.33/0.64 | 20 | mA/cm² | 80 | 99 |
| E1 | 3.5 | 40 | 15.9% | 0.33/0.62 | 20 | mA/cm² | 80 | 134 |
| E2 | 3.7 | 51 | 16.1% | 0.33/0.63 | 20 | mA/cm² | 80 | 149 |
| E3 | 3.4 | 55 | 15.8% | 0.32/0.62 | 20 | mA/cm² | 80 | 131 |
| E4 | 3.6 | 41 | 14.6% | 0.32/0.63 | 20 | mA/cm² | 80 | 133 |
| E5 | 3.3 | 13 | 15.7% | 0.32/0.64 | 4000 | cd/m² | 80 | 130 |
| E6 | 3.1 | 11 | 15.9% | 0.33/0.63 | 4000 | cd/m² | 80 | 134 |
| E7 | 3.1 | 59 | 15.0% | 0.33/0.63 | 20 | mA/cm² | 80 | 129 |
| E8 | 3.2 | 56 | 14.8% | 0.33/0.64 | 20 | mA/cm² | 80 | 128 |
| E9 | 3.3 | 11 | 15.9% | 0.33/0.63 | 4000 | cd/m² | 80 | 139 |
| E10 | 3.4 | 59 | 14.7% | 0.33/0.63 | 20 | mA/cm² | 80 | 134 |
| E11 | 3.2 | 56 | 14.2% | 0.33/0.63 | 20 | mA/cm² | 80 | 122 |
| E12 | 3.5 | 62 | 15.5% | 0.34/0.64 | 20 | mA/cm² | 80 | 130 |
| E13 | 3.5 | 60 | 14.1% | 0.34/0.63 | 20 | mA/cm² | 80 | 115 |
| E14 | 3.3 | 58 | 16.3% | 0.33/0.64 | 20 | mA/cm² | 80 | 131 |
| E15 | 3.3 | 64 | 16.2% | 0.34/0.63 | 20 | mA/cm² | 80 | 123 |
| V9 | 4.2 | 65 | 14.4% | 0.34/0.62 | 20 | mA/cm² | 80 | 100 |
| V10 | 4.1 | 56 | 14.1% | 0.32/0.63 | 20 | mA/cm² | 80 | 106 |
| V11 | 4.0 | 53 | 14.0% | 0.34/0.64 | 20 | mA/cm² | 80 | 107 |
| E16 | 3.5 | 76 | 16.0% | 0.34/0.65 | 20 | mA/cm² | 90 | 129 |
| E17 | 3.3 | 49 | 16.5% | 0.33/0.64 | 20 | mA/cm² | 80 | 128 |
| E18 | 3.2 | 63 | 16.7% | 0.33/0.63 | 20 | mA/cm² | 80 | 121 |

TABLE 9
Materials used that have not been described before

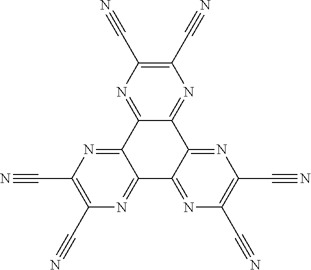 [SMILES: N#Cc1nc2c3nc(C#N)c(C#N)nc3c3nc(C#N)c(C#N)nc3c2nc1C#N]

HTCN

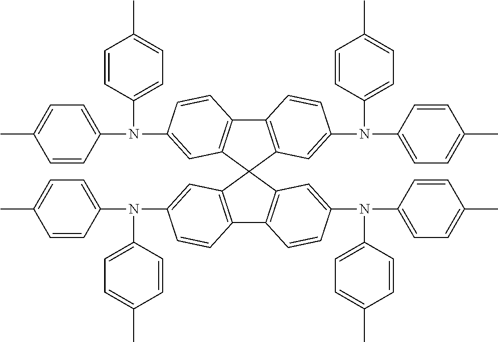

SpA1

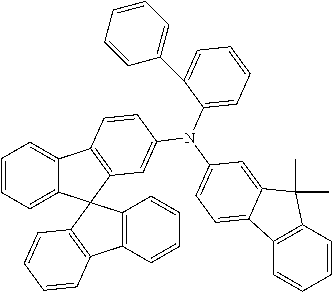 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

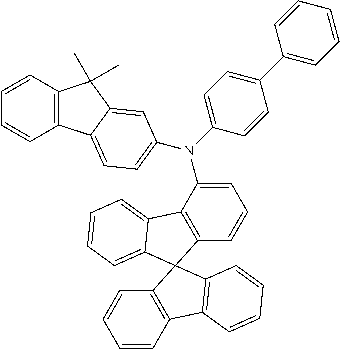 [SMILES: CC1(C)c2ccccc2-c2ccc(N(c3ccc(-c4ccccc4)cc3)c3cccc4c3-c3ccccc3C43c4ccccc4-c4ccccc43)cc21]

SpMA2

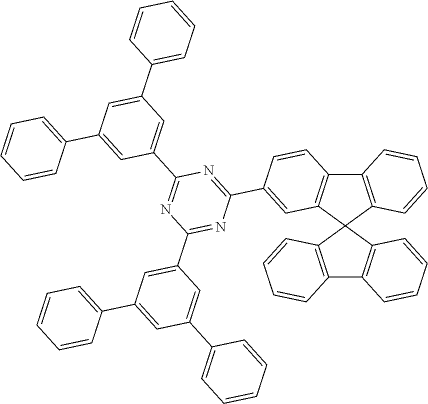 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

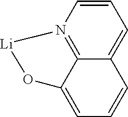

LiQ

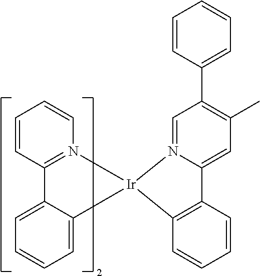

TEG1

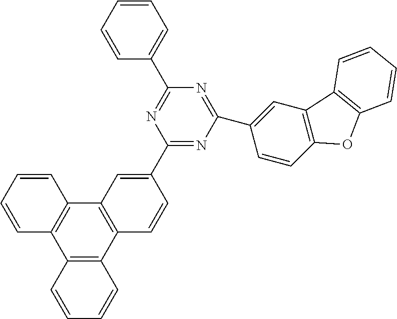 [SMILES: c1ccc(-c2nc(-c3ccc4oc5ccccc5c4c3)nc(-c3ccc4c5ccccc5c5ccccc5c4c3)n2)cc1]

SdT1 (US20150349268)

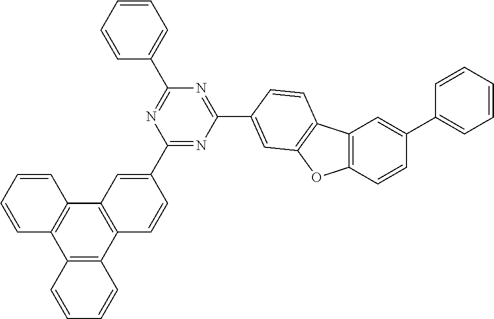 [SMILES: c1ccc(-c2ccc3oc4cc(-c5nc(-c6ccccc6)nc(-c6ccc7c8ccccc8c8ccccc8c7c6)n5)ccc4c3c2)cc1]

SdT2 (WO2019054833)

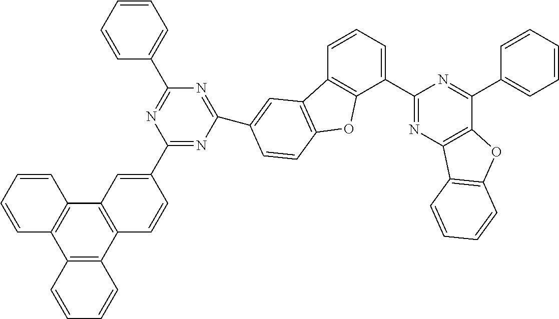 [SMILES: c1ccc(-c2nc(-c3ccc4oc5c(-c6nc(-c7ccccc7)c7oc8ccccc8c7n6)cccc5c4c3)nc(-c3ccc4c5ccccc5c5ccccc5c4c3)n2)cc1]

SdT3 (WO2019017731)

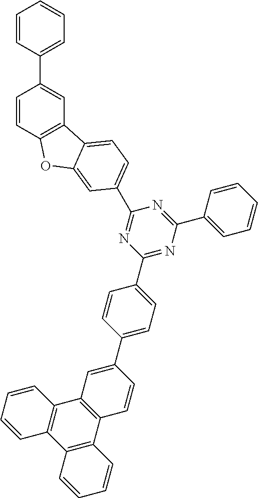 [SMILES: c1ccc(-c2ccc3oc4cc(-c5nc(-c6ccccc6)nc(-c6ccc(-c7ccc8c9ccccc9c9ccccc9c8c7)cc6)n5)ccc4c3c2)cc1]

SdT4 (WO2019054833)

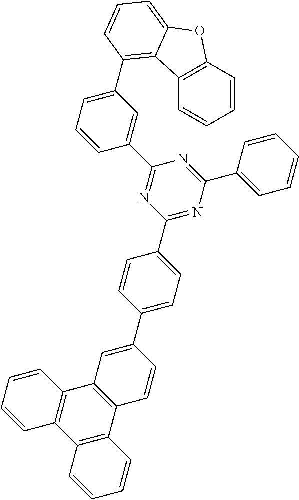 [SMILES: c1ccc(-c2nc(-c3ccc(-c4ccc5c6ccccc6c6ccccc6c5c4)cc3)nc(-c3cccc(-c4cccc5oc6ccccc6c45)c3)n2)cc1]

SdT5 (KR101959821)

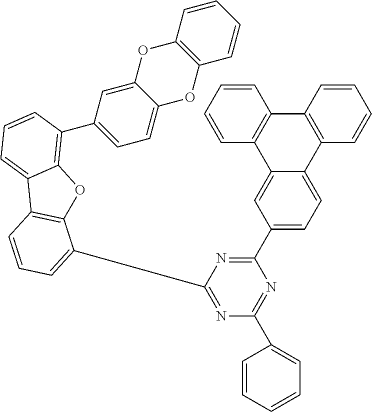 [SMILES: c1ccc(-c2nc(-c3ccc4c5ccccc5c5ccccc5c4c3)nc(-c3cccc4c3oc3c(-c5ccc6c(c5)Oc5ccccc5O6)cccc34)n2)cc1]

SdT6 (KR20200011378)

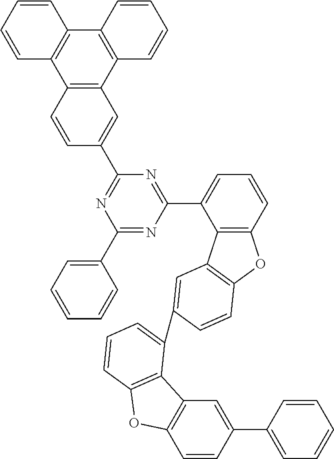 [SMILES: c1ccc(-c2ccc3oc4cccc(-c5ccc6oc7cccc(-c8nc(-c9ccccc9)nc(-c9ccc%10c%11ccccc%11c%11ccccc%11c%10c9)n8)c7c6c5)c4c3c2)cc1]

SdT7 (WO21037401)

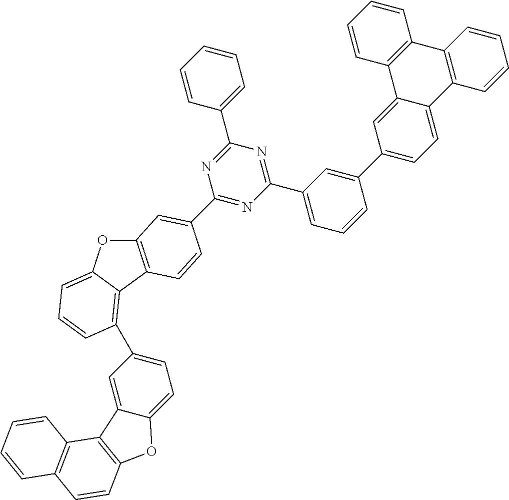 [SMILES: c1ccc(-c2nc(-c3cccc(-c4ccc5c6ccccc6c6ccccc6c5c4)c3)nc(-c3ccc4c(c3)oc3cccc(-c5ccc6oc7ccc8ccccc8c7c6c5)c34)n2)cc1]

SdT8 (WO21071247)

1. A compound of formula (1) 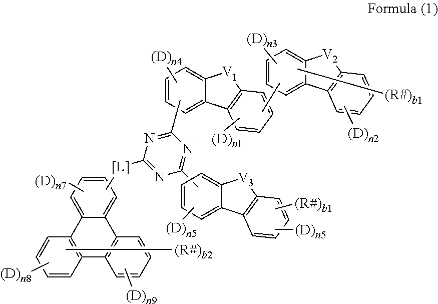
wherein the symbols and indices used are as follows:
V1, V2, V3 are each independently O or S;
[L] is a single bond or an aromatic or heteroaromatic ring system which has 5 to ring atoms and may be unsubstituted or partly or fully substituted by D;
R # is in each case independently phenyl, 1,2-biphenyl, 1,3-biphenyl or 1,4-biphenyl, which may be unsubstituted or partly or fully substituted by D;
b, b1 are each independently 0 or 1;
n1, n3, n4, n5, n7 are each independently 0, 1, 2 or 3 and
n2, n8, n9 are each independently 0, 1, 2, 3 or 4.

2. The compound as claimed in claim 1, wherein V₃ is O.

3. The compound as claimed in claim 1, wherein V2 is O.

4. A mixture comprising at least one compound as claimed in claim 1 and at least one further compound selected from the group consisting of matrix materials, phosphorescent emitters, fluorescent emitters and emitters that exhibit TADF (thermally activated delayed fluorescence).

5. A formulation comprising at least one compound as claimed in claim 1 and at least one solvent.

6. An organic electroluminescent device comprising an anode, a cathode and at least one organic layer comprising at least one compound as claimed in claim 1.

7. The organic electroluminescent device as claimed in claim 6, wherein the organic layer contains at least one light-emitting layer containing the at least one compound.

8. The organic electroluminescent device as claimed in claim 6 wherein the light-emitting layer contains a further matrix material.

9. The organic electroluminescent device as claimed in claim 8, wherein the further matrix material corresponds to a compound of the formulae (6), (7), (8), (9) or (10) 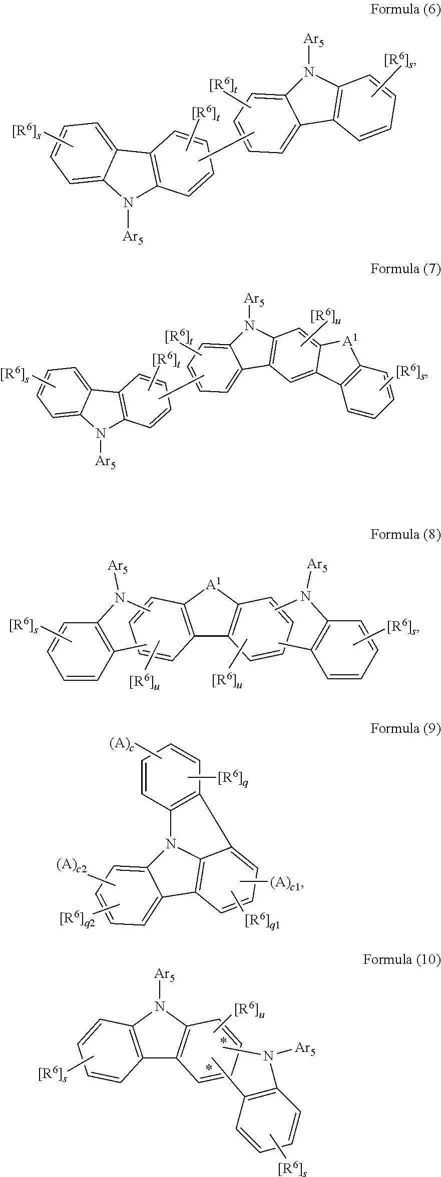
wherein the symbols and indices used are as follows:
A¹ is C(R⁷)₂, NR⁷, O or S;
A at each instance is independently a group of the formula (3) or (4), 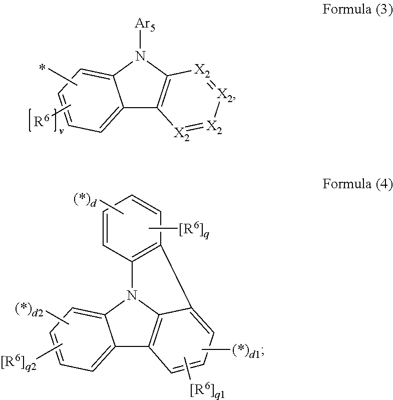
X₂ is the same or different at each instance and is CH, CR⁶ or N, wherein not more than 2 symbols X₂ can be N;
* indicates the binding site to the formula (9);
R⁶ at each instance is the same or different and is D, CN, a straight-chain alkyl group having 1 to 20 carbon atoms or an alkenyl or alkynyl group having 2 to 20 carbon atoms or a branched or cyclic alkyl group having 3 to 20 carbon atoms, wherein the alkyl, alkenyl or alkynyl group may in each case be substituted by one or more R⁷ radicals and wherein one or more nonadjacent CH₂ groups may be replaced by Si(R⁷)₂, C═O, NR⁷, O, S or CONR⁷, or an aromatic or heteroaromatic ring system which has 5 to 60 ring atoms and may be substituted in each case by one or more R⁷ radicals; and further wherein two R⁶ radicals may together form an aromatic, heteroaromatic, aliphatic or heteroaliphatic ring system;
Ar is the same or different at each instance and is an aromatic or heteroaromatic ring system which has 5 to 40 ring atoms and may be substituted by one or more R⁷ radicals;
Ar⁵ is the same or different at each instance and is an aromatic or heteroaromatic ring system which has 5 to 40 ring atoms and may be substituted by one or more R⁷ radicals;
R⁷ is the same or different at each instance and is D, F, C, Br, I, N(R⁸)₂, CN, NO₂, OR^{B}, SR^{B}, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, wherein the alkyl, alkenyl or alkynyl group may in each case be substituted by one or more R⁸ radicals, wherein one or more nonadjacent CH₂ groups may be replaced by Si(R⁸)₂, C═O, NR⁸, O, S or CONR⁸, or an aromatic or heteroaromatic ring system which has 5 to 40 ring atoms and may be substituted in each case by one or more R⁸ radicals; and further wherein at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical having 1 to 20 carbon atoms, in which one or more hydrogen atoms may also be replaced by F;
c, c1, c2 at each instance are each independently 0 or 1, wherein the sum total of the indices at each instance c+c1+c2 is 1;
d, d1, d2 at each instance are each independently 0 or 1, wherein the sum total of the indices at each instance d+d1+d2 is 1;
q, q1, q2 at each instance are each independently 0 or 1;
s is the same or different at each instance and is 0, 1, 2, 3 or 4;
t is the same or different at each instance and is 0, 1, 2 or 3;
u is the same or different at each instance and is 0, 1 or 2; and
v is 0 or 1.

10. The organic electroluminescent device as claimed in claim 8, characterized in that the further matrix material corresponds to a compound of the formula (11) 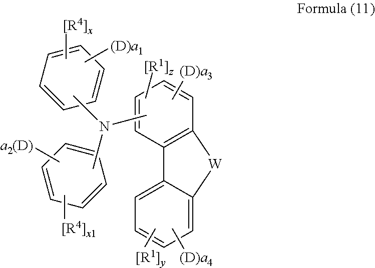
wherein the symbols and indices used are as follows:
W is O, S, C(R)₂, or N-Ar₁;
R is in each case independently a straight-chain or branched alkyl group which has 1 to 4 carbon atoms and may be partly or fully deuterated, or an unsubstituted or partly or fully deuterated aromatic ring system having 6 to 18 carbon atoms, wherein two substituents R together with the carbon atom to which they are bonded may form a mono- or polycyclic, aliphatic or aromatic or heteroaromatic, unsubstituted, partly deuterated or fully deuterated ring system which may be substituted by one or more substituents R⁵;
Ar₁ is the same or different at each instance and is an aromatic or heteroaromatic ring system which has 5 to 30 ring atoms and may be substituted by one or more R⁵ radicals; and wherein at the same time, two Ar₁ radicals bonded to the same nitrogen atom, phosphorus atom or boron atom may also be bridged to one another by a single bond or a bridge selected from C(R⁵)₂, O and S;
R¹ is the same or different at each instance and is selected from the group consisting of F, Cl, Br, I, CN, NO₂, C(═O)R′, P(═O)(Ar₁)₂, P(Ar₁)₂, B(Ar₁)₂, Si(Ar₁)₃, Si(R′)₃, a straight-chain alkyl, alkoxy or thioalkyl group having 1 to 20 carbon atoms, a branched or cyclic alkyl, alkoxy or thioalkyl group having 3 to 20 carbon atoms or an alkenyl group having 2 to 20 carbon atoms, each of which may be substituted by one or more R′ radicals, wherein one or more nonadjacent CH₂ groups may be replaced by R′C═CR′, Si(R′)₂, C═O, C═S, C═NR′, P(═O)(R′), SO, SO₂, NR′, O, S or CONR′ and wherein one or more hydrogen atoms may be replaced by D, F, C, Br, I, CN or NO₂;
R′ is the same or different at each instance and is an aliphatic, aromatic or heteroaromatic organic radical;
R⁴ is the same or different at each instance and is selected from the group consisting of F, C, Br, I, CN, NO₂, N(Ar₁)₂, NH₂, N(R⁵)₂, C(═O)Ari, C(═O)H, C(═O)R⁵, P(═O)(Ar₁)₂, a straight-chain alkyl, alkoxy or thioalkyl group having 1 to 40 carbon atoms, a branched or cyclic alkyl, alkoxy or thioalkyl group having 3 to 40 carbon atoms or an alkenyl or alkynyl group having 2 to 40 carbon atoms, each of which may be substituted by one or more R⁵ radicals, wherein one or more nonadjacent CH₂ groups may be replaced by HC═CH, R⁵C═CR⁵, CC, Si(R⁵)₂, Ge(R⁵)₂, Sn(R⁵)₂, C═O, C═S, C═Se, C═NR⁵, P(═O)(R⁵), SO, SO₂, NH, NR⁵, O, S, CONH or CONR⁵ and wherein one or more hydrogen atoms may be replaced by D, F, Cl, Br, I, CN, NO₂, an aromatic or heteroaromatic ring system which has 5 to 60 ring atoms and may be substituted in each case by one or more R⁵ radicals, an aryloxy or heteroaryloxy group which has 5 to 60 ring atoms and may be substituted by one or more R⁵ radicals, or a combination of such, wherein it is optionally possible for two or more adjacent substituents R⁴ to form a monocyclic or polycyclic, aliphatic, aromatic or heteroaromatic ring system which may be substituted by one or more R⁵ radicals;
R⁵ is the same or different at each instance and is selected from the group consisting of D, F, CN, a straight-chain alkyl group having 1 to 20 carbon atoms or a branched or cyclic alkyl group having 3 to 20 carbon atoms, wherein one or more nonadjacent CH₂ groups may be replaced by 0 or S and wherein one or more hydrogen atoms may be replaced by D, F, CN, or an aromatic or heteroaromatic ring system which has 5 to 30 ring atoms and in which one or more hydrogen atoms may be replaced by D, F, C, Br, I or CN and which may be substituted by one or more alkyl groups each having 1 to 4 carbon atoms; and wherein two or more adjacent substituents R⁵ may together form a mono- or polycyclic, aliphatic ring system;
x, x1 at each instance are independently 0, 1, 2, 3 or 4;
y, z are each independently 0, 1 or 2;
a1, a2 are each independently 0, 1, 2, 3, 4 or 5;
a3 is 0, 1, 2 or 3;
a4 is 0, 1, 2, 3 or 4.

11. The organic electroluminescent device as claimed in claim 6, wherein the light-emitting layer contains a phosphorescent emitter.

12. The organic electroluminescent device as claimed in claim 6, wherein the electroluminescent device is selected from the group consisting of organic light-emitting transistors (OLETs), organic field quench devices (OFQDs), organic light-emitting electrochemical cells (OLECs, LECs, LEECs), organic laser diodes (O-lasers) and organic light-emitting diodes (OLEDs).

13. A process for producing the electroluminescent device of claim 6 includes depositing the organic layer is-applied by gas phase deposition or from solution.

14. The process as claimed in claim 13, wherein the light-emitting layer of the organic layer is applied by gas phase deposition, wherein the at least one compound of the formula (1) is deposited from the gas phase together with the further materials that form the light-emitting layer, successively or simultaneously from at least two material sources.

15. The process as claimed in claim 13, wherein the light-emitting layer of the organic layer is applied by gas phase deposition, wherein the at least one compound of the formula (1) is deposited from the gas phase together with at least one further matrix material as premix, successively or simultaneously with the light-emitting materials selected from the group consisting of phosphorescent emitters, fluorescent emitters and emitters that exhibit TADF (thermally activated delayed fluorescence).
